SEQUENCE LISTING (210 sequences)

SEQ ID NO: 1 - DNA, 501 bp, Bos sp.
acttttcttaaactagttgcttcctctaaacttacaaagttagtggaaataaaagagaaaaatcccaaaactaaaaaaaaaaaacccaaaaaacctctttttctaattaagagcactgatgtgggagataactttagaagactttttgaaattgcagtaactgtttgggaaatgtgattataatttaccttttaggataacaaaattctcagcaagttttggagaattcgagaaattcgagcacacgtttmtaaactaaaggaaaaatctctctgcagagtaaactgataactggaaaagcattcctggaacctgatgcaaatagctaacgctgcccggctccctccctgttgatgtggcagagtttgaagctggcagtcgtcagccatttggtgggaggccgtggatccagaggaaacaatgaggatggagaaaactgaaagggaatttttaaagagggagacatttttgttccagcagttttttacacattattttgac

SEQ ID NO: 2 - DNA, 501 bp, Bos sp.
tagccactgaaccaccaggaaggtcccttgaagttccttctaaccattttcattttcaagtttttagaaactgccttttactttccgtccagggtatttgtatatttaagagaagtttgcttccaaaagtcttatgctcaaattacacactagttttaagacgatacttacttcttggcctacactgggatgtatgaatgggaaataaaagtagtatgcatgtgataactgaatggtgggcaataggaagyctacatctcttttaaaattaaaatatatgttaactttccttttaattctgagaaattataacctttcagttcagttcagtcactcagtcatgtccaactctttgcgatcccgtggactgcagcatgccaggcttccctgtccatcaccaactcctggagtttactcaaactcatttccattgagttggtgatgccagccaaccatctcaccttctgtcatccccttctcctcctgccttcagtctttccc

SEQ ID NO: 3 - DNA, 501 bp, Bos sp.
gtacaacacacccaggagtgttctcagaacacctttgatttcaccactcctgggtgttcacagagaagggatgatactgaaaggtatgcagtcaaacagtaatggttagatgtcatcttctgctgggaagtaagataattaagtaattgctttgctttcatgccaccacttacagacaccatagtctctctcgggaaaataataacctttttaagtggaccaagcccatactttatagcatatgacttttkgggggttctgcagtgtttagcatttgagattgtattttggatacatttcagaggagtcaagccaacttggcattactcagtgtctgaccagccaacctggaccccttcagaaggtgattttccaggctgaatttttcagggccttgtttcccgcctcctatttccttctccattatgttacttgcattcatggcttattagcaatgccagcagaaatgttggctgcaaaagtcaggaaaagtacaatcag

SEQ ID NO: 4 - DNA, 501 bp, Bos sp.
gaacatataatttctaaatgtctaaagcctggaattaatgtctgtgacctcggtaataccaagagatgaattgtgagaatgaagattgttctaattttctcagcaaagataatattttaaaaactacaaactgaaaatcactttttgtgacagcacgtttaatttgtgtagctattctcattgcaggctgatttgctaaaatatgagcgaacaagtctaatgtagggagttgaacagagcactatggtatygagtgtgtagtttcttgccttacgatgcctactatgattttaaagttacttaaaattgtttctaaagacttcccctgaacttgacatcaggaacttacagttatacttggttgaggcccctgttctcagtggggtattgtggggaagagaagacagaagttgataaaaggcagggttccctcagtgaggatctgggaagtgagaggggtttacacgatgaagatgttgccctccattgagtcatttatca

SEQ ID NO: 5 - DNA, 501 bp, Bos sp.
  misc_feature (389)..(501): n is a, c, g, or t
aacaccttctggttaagttgtaaatgggacatttggcctacccaccctagcactgctccattacagcccagaatccaagtgttgtaagcaagaaaaatgttagaggctgagctatgaaaatggcagctgtgatataattatgaatataacctgctggagacagaattgtgaagcctattcaccaattctagccctgctgctaagcacctgttatgctctctttgtacttgtaaaatgggtacataaaactygacttcgtgtatttcatagagttgttgctaagatacatgaaacagtgtcttctgggccatagggcattgtaaattcacgctcttagcccctgatagactccaaacaaatgttagatcaaatttgaagacagaaaaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 6 - DNA, 501 bp, Bos sp.
tttctatttgtcctatttcatatttctttttttcctcttttcttgctttgagatgattaaatattttttacttttctatttccctataatcttgttacttgtatccttttcttttagtggttatcatacagattttatcatacctctttgacatattacaattgactttttaaaagaaattttatttcttggttgtgctatgtggtgtgtgggatcttctccccaatcaggaagcatagagtcttaaccaytggaccaccaaggggaagtccctacaatctactttaacttaattactatatttcaaataatgcaaggagcttacaacattttaactccatgtatgccccatcccacttctgtggtattactgtatttcatttctacatatattttaaacttcttaagaaaatactactgctactgcttctctttcctttacaagttgtcatgctttcctcttgtatcattatttctttaatatttcttttagtgtagctc

SEQ ID NO: 7 - DNA, 501 bp, Bos sp.
gcaaaggcattttttcccatcactttaacctcctccctggcccagctttagctgatggcagattccaggccacatccatcctcctccaacgttttccccaggccgagattcttcattcttggggttcatctgagccgtttccatagtgacagtgacttgttgagttttcaacctcccatcgaggtccttgcaatttgccagctaagccaccgatcagcaggcatgtgtgccaggttagtaagacaaagtcygttttatttcatctttgccttttttgtctgccttcagttggggccagcctcacaaaagagagggccttttggctttcagaattcttcagcctctgctcattatctcaggcactagtttggactgaaccaatgtggggtgatttccttgctgtcgcccccgtaccctggccccaccctggagacaggagggagcatacctcaggcttggaaggaggtcactgggaacttcttttagtttgcatttgagtgt

SEQ ID NO: 8 - DNA, 501 bp, Bos sp.
tttctagtttaattccactgcagtcttatttgatatttgtttaaaaatctcctataatgtctactttccttttcattatattcctttgatctttgtgtattttatgttaatagagcatacaaatttaggatggttaaatctttctgttgtattaatccttctgttactatgaaatatcactctactgtgcataatacttcctgctttgaagtttattttgtttgatgttagtgtagctataacaagctttmttttaattaccatttacatggtttatatttttctatccttttattttcgatctttcctgcccttttaaggtgtgcttcttataacctgggttgagaaaatcccctggagaaaaaggaaaggctatccactccagtattctggcctggagaattctatggactctatagttcatgggatcgcaaagagtcagacatgactgagcgactttcacttcatttcacttcacttcttataaccagcatataatta

SEQ ID NO: 9 - DNA, 501 bp, Bos sp.
tgtgcttcttataacctgggttgagaaaatcccctggagaaaaaggaaaggctatccactccagtattctggcctggagaattctatggactctatagttcatgggatcgcaaagagtcagacatgactgagcgactttcacttcatttcacttcacttcttataaccagcatataattatttttattagtctgatgggttttgtttttagctgaaatatttagtccacttatatccaatgtatttattgyatgtttaattttaatattactagttgttttctatttgtcctatttcatatttctttttttcctcttttcttgctttgagatgattaaatattttttacttttctatttccctataatcttgttacttgtatccttttcttttagtggttatcatacagattttatcatacctctttgacatattacaattgactttttaaaagaaattttatttcttggttgtgctatgtggtgtgtgggatcttctccc

SEQ ID NO: 10 - DNA, 501 bp, Bos sp.
aacagaacacagtttttaatggggattttttccttttaaaattcttaaattccctttttatttaaagaatggtacatattcacttgtgttgatattcactatttgaggtttataatatcctcttaaattgttcaatcagtgattttaatctttttcaccttttattaattcattgacagatttcacactttttttcagtaaatacatttactctgatttttggaattaaaacatttttaacttctgaatcrtctaggttatattttgggcatgaataatcttgttgcctttttctcatctctaaagagtttctcatttttccttatttttctctaagattgttagtagaccactatgaaatcactggacagaatttttgttacagaggatgcctacattttgagcaattaaaattgttgtttcttggcattataaatatcatcttcattccctgatagctcagttggtaaagactacgcctgcaaagcaggagaccccagt

SEQ ID NO: 11 - DNA, 501 bp, Bos sp.
aatggaatataacctacgtttcttaaagtgcccaatcactccttggctttaggtatacttaagtattcttatatccttactgtattatttatcacctgtctatttccaatttaaatacaagcacaaatttgcagctttcaatgatttccgggtcaacatttatattcaaacagtaaattatttaaatgaagaagtcagtttactatgctttaaacttattatctattttgccttccatattatgtcatacrctccttgaggatggggagaatctgagcagatttcaatttttaaaatatccttcttacattttgcacagcaaggtactcaataaacatttgtcaaatggaatggaatagatgatctccgatgcctgtagcataaattctcacacagtgtactgcaatagtactccgggccaatgctaatgccaaaacaataaaactagtgaaaaatatagattaccttcctaatgtttactaggcaaacaatttttaagga

SEQ ID NO: 12 - DNA, 501 bp, Bos sp.
agatgagcttcagaagatcccacagggatggagacttgttttcatctgcaacgaacaagtgaaatctcctctgatattactaaagcaataggaggtttccttttatttatttgaaatgcatttttagtagcatgttattcagaatttgacactatattctgcattatggaaagtaactttaaaagatctaaataagtatgtgtatctaggagttaaatattctttaaaccacaagaatgggtgacaatctmgttgtatacctccagtctttcctttcataactgatttttcttggagaatattagtatactggtatgaaaataccagtttctcttccgaattgtgacttctttatcttttggataaatctctgtgcagccccaaggctcgtgagggtttaaattactttatttatttacttggctacatcgggtcttagctgaggcacatggcatcttttgttgcggtgcacagacgctctcacgcatctcctgtggcacg

SEQ ID NO: 13 - DNA, 81 bp, Bos sp.
gccacacccgtaccctcactgccacctccacacaggcttctctgacttgarggcaataactcaagttaggtttcttattca

SEQ ID NO: 14 - DNA, 501 bp, Bos sp.
acaaagcagaagtccaagaagctgcattcccaagattgccctggccccagcacacactctgaagaacacacttaagggctgggcagctttctaattatggggagatcttcgatttaaagttaataccagaaaggatgctgtggctggcttctgcatgaaatatgcagttaaaagcccactgccagggaaacttctgccaagccctaagtaccagccaccctgtgtttctcctttccaacgatacctgcagstcataaatgcctaggcagatgaaggccttctcttttgaggtacctcacccccagagacctagcaaagtaatctagggtggagttggcatcaggatttattttatgtgtcccaagtgatggagagaagtagccactgctgagaattgagtcctgagcaccctcctggctccaggtccaaggtgataaactgagccccctcttgtcagacacagcgcagaaggttagtttagcccccatctcacccaccact

SEQ ID NO: 15 - DNA, 501 bp, Bos sp.
tccttcctttgcttccatggggtttgatcccaagagaactcaattaaacattccagttgtaaagaacccacctgccaatgggagacataggaggcatggttcagttcctggatggggaagatgccctggaggagggcatgcaacccactccagtattcttgcctggagaatcccatggacagaggagcatggtgggctacagtccgtggagtcacagagtcggacacaacttaggtgacttagcatgaatrcactcaaactattatagtatctgtttgagagttggctttatacagaacccatattgtaacaccctctcaggcaatgagctgccttttgtactaacacatatttgtatttttctcataaatttcaattcaagtatttttccttaagatctttcataacctttctaggtcaacagagttattcttcccatgctctcccatatcttacaacataagaactttgtcataagaggtcaaatactaaataggaaat

SEQ ID NO: 16 - DNA, 501 bp, Bos sp.
aacctttcagctgtagataatagtggatatgtaggagagagaagatggacgggattgtaagagatggtattaatcttctctgtaaattataaggaaaggtgagatggtgctatatggttcatgacagaagggggaaaggtgggctcatgttacagatacaagagcattatctttattcaccacgtgccaaatacttagcacaatgcacagcacacaacagaaactcaataaatacgaactgaattcaagarttcaatttgaggaatttaggcacaccgcacggagaaggcaatggcaccccactccagtactcttacctggaaaatcccatggatgggggagcctggtaggctgcagtccatggggtcgctaggagtcagacacgactgagcgacttccctttcacttttcactttcatgcattggagaaggaaatggcaacccactccagtgttcttgcctggagaatctcagggatgggggagcctggtgggcttccgt

SEQ ID NO: 17 - DNA, 501 bp, Bos sp.
aaaaaggattgagctaggttctagttataaaagaatgaacaagatttatccctggtctcaaggaagtcgtaatctattatggtacatcttcattaaacaaagctcaatctgtatgtctctttttcattagaagcaacatactctttacgtctgctttgttggcctcctaagtatctaaacataaatgatacatgtggacagaccacacacttttgtactgctttgttttgaccaacacttttctagaacartagcccaatagctgctgttccagtaaaaatctggattctaaaactaaaatgttttcctgtttttactcatatttcctacatttatttatacaacatgtaccagaactatcagttactacattttgttacctccaattagggcttcgaaaatttattatgaaacttcagcatctcagagattgtacagacactacttactgttgaacatgttagcttcatttttactggggcaaatgttacttaaaagaaa

SEQ ID NO: 18 - DNA, 501 bp, Bos sp.
tggacattgtgatgaaatttaacctctcatctctataccacaagtttaaaaaacatatttttcctctcaagaggagtcagcagatgctatgaagatacttgtaattcttgatcacattttttgtatttattcttgtctagactacctgcactcacaagtagttttcatgcttaccaatgttttggtttccacttcctgtataaagggaattataacttagtgtatgtattgtttgtccccagaagtaagargataactgtgtatgtttgcatttaggtcatcctgtccattgaagaaggttacagactaccagctcccatgggctgtccggcatctctacaccagctgatgctccactgctggcagaaggagagaaatcacagaccaaaatttactgacattgtcagcttccttgacaaactgatccgcaatcccagtgcccttcacaccctggtagaggacatccttgtgtaagacgcataatgttgattttttcccttg

SEQ ID NO: 19 - DNA, 501 bp, Bos sp.
cttggtacttccagactcccacaaagctctgctataatctgtctcgtcagcagatcctaccttgtaccattactcagggggctgcctgctcatccatccccaaggcctgtgtgggcctctcccagctgacaactccatgtaagaagtcacttttgaaaacaagggattgaggtagtgtgacagcatggggagaactaggcattgacacccaacggtcctgggcttaggacaaagctgccaaacacttgtgygaattttctccaatactctcgatggccctaagaagttaccccgtacccatttcatgggtgtggcagatccgtgatgctcaatgtttgtccaaggtcaccctactgacccaggatttgattcagagctgcctcccagcaaaatctctatttaatgctagaatgggaccttagagaagggaggctgcagagggatttgagactcatcagataagaatcaagtggtacagggaactatattcagtaattacct

SEQ ID NO: 20 - DNA, 501 bp, Bos sp.
ctgccaaacacttgtgtgaattttctccaatactctcgatggccctaagaagttaccccgtacccatttcatgggtgtggcagatccgtgatgctcaatgtttgtccaaggtcaccctactgacccaggatttgattcagagctgcctcccagcaaaatctctatttaatgctagaatgggaccttagagaagggaggctgcagagggatttgagactcatcagataagaatcaagtggtacagggaactwtattcagtaattacctataatagaaaataatttggaaaaaaatatatatataccttgaaaccttgtgtcctgagtcctgccttgtacccttcccatatatgtatgtatgtgtatatacatactgttgttcagtcacaaagtcatgtcaactccttgtgaccccgtggactgcagcacgccaggcttccctgtccctcaccatctcctggagtttactcaagttcatgtaatacaaccttgtaaatccact

SEQ ID NO: 21 - DNA, 501 bp, Bos sp.
taagatcacccagtgagctcctccccttcaatttcccaaattaccagccagcatataaacacttgggtgacatgtaccataaagtatcatgctttggattttatataacttggtttttaaaaaatttattagtagattaatttattctggaaacaaattcaaactcctctctttcccaaattgggtatttgacccaatttgaaactagtgaaacgactttaataaatactgccaataaggctttcatgtamgacaaatattcttagatcaaaaaagcaattccactaatacaaagatctttctagatgctgtcatttacctaaaatgttctcaactagtatgtgaagtattaatttataataactattgattttatgaaatcaacctgcaaaagttcttcaggtaatggaaagatgttttggtaaacagaaattttaataaaatcacatcttgaagaaaaacctccttttgtaagtcattctgacctgctctatctgccta

SEQ ID NO: 22 - DNA, 501 bp, Bos sp.
cttcacatggaagaaaaccttttagtgtgtgtcctcaaagagataagaaacaatgctgatgcttttttaaaaaccttttaaaataacaaatacatttttcttgaatattaagggagccaatataacaaagcttgatagaaataacatatatttatatatatatatatttgaaaaaaattcaggcactggaataaaaatgcaaagtgatgggaaagagtaggcaacagataagcaccctataaaattagtcragaaagttcaaagtctgagaaaatttccagaaaatgaagacatcagacttgagaaaactatccaaaaactagtgagagaaatttttccatagttgaagggtttccagatttaaaaaactaactttatgctcagcatgatgaataaactcattataaaatgtcagataatggcagtaaaatgaaaatcctaaaaactcccagagcaaaaagcaagattacatgcacaatattagaaatgatatttgcatta

SEQ ID NO: 23 - DNA, 501 bp, Bos sp.
aaaattgttttgcaaactgttttcattcttgtgataaggctgagcaactctgtcccacaaattctagttttgcttggaaactgcaaagtagtccctgaatattttagagggaatcgatgttgatggcaaaagaaaatttgcagctatacatttgcttgttacaattccttttctataaaaccttatttttggtgatttaaataaagcattgcctgaatgtttgagattttatagctacacttggttgtgtratattatggttccttttctgtaaaatgttatttttggccatttcaaataaatgattttcgatcttattggaaagaaccaggagagagtttttactctggtcttgtccttttctctttatttgttgtaggaaaacagtaaggcaactttgattcctcaattttgtatttattttactttccctttgtgccttttttgttaaggacttttgttttcctttcctttcttttattttcctaccttggctgcctt

SEQ ID NO: 24 - DNA, 501 bp, Bos sp.
acacctgttgaccaacaaagaatggttttgagcaatgaggaactttagtaattcttatcacagtgtgaaatttaacaaggacactttagggcttcctagcacttaagtctttttcgtttttccagcaggactctaacttcaatgaggtctttattcagaatatccttcattttaactgagaacaaatgttcatttaccaacagaaacagaagatcgtgacaattgactctgtaaatctttacctgaactcmttatggagggacacatttatttgatttttcttgtattttcagtggcgagaacaccatctgagtaattctactggcccctctttgcttcctctttccttacccttagcaaaccatagtcaatttgaaaaaaggaattcagttcagttcagttcagttgctcagttgtatccgactctttgccaccccatgaatcgcagcatgccaggctttgctgtccatcaccaactcccggagttcactcaaactcaca

SEQ ID NO: 25 - DNA, 501 bp, Bos sp.
aatcctggtgtatgtaggtaaatccaacatgaaccttggcctccatgctgctctgtaagtcaaccactgttgattgctttcaaagagagggagcgatgacattgaaactttgagcatcttcttattcactccttgtacctgatgacacccatcactgacctgcaggtccaaaatccctccaacaccctcacccttggcctctctttctcagaatctagtcattcctcttatagatgtctcaaggtcatgtytgaccttcagcaagggagaatctcaggatgattaaaacctggggatgctctctgggacttggaaagttgcctgggggtcagatctcagaggtacaacaaaatactttaggcctcagggggtctgactgaaactcagcctcataggcatagaattggggatggggtagaattagttatgttccagagggagaaataagtaactgtcagaaatgtgaaagatttgtcaatagcacactgttgttgtttgttc

SEQ ID NO: 26 - DNA, 501 bp, Bos sp.
ggttatgtggatatttacaatagtgaatggggtgctattgagggtcccaaaggccagactccagagcttgtactaaatccaacaggtggtggggagccatcatgtatcctaagcagaggaaagacactgtgagagtttgtccttttaggaaaatgaatctggcaggactctggaaagaccgattaggaggtaagactactagaggtaaactttatagttatctaagaagaaaccctgaactggagtggtcrtggtaggaattaaaaagagaggacagatgcagaaaggattttagagctagattttgcatattcaagaactctgaatgtgaaggaagagagcagtttaaataatcaaagctattttgaggattcaagtctaacataccagaagaagggtagtgcctttcacagaaatggggtaaaaaagcaaaaggtgatgaaccttctcccctcagctgaaaaagagttctggaaaaaggatggttgtatctattcagtt

SEQ ID NO: 27 - DNA, 501 bp, Bos sp.
atttccttgagctccaaacctgagacatgttcactgcctacaaccatgagctctaatagcttgcctgccattacaaccaccccacattgcagctgaggcaatttgcttttcaaaagtaatagagagtaaattgcagaggaaatattttatgcaaattaaccaagcttaacattttgataaaggcctcttattttcaaatgaaccaaggaatccaatactccattgcaagtttaacacaaaagaaaattttwaaagcctgagtttttaaagtaggtgacagcaagagacattaaccacctgtaagatggaatacagaagcaaagtgcataaatctattagttcaaaacaatatttatgtgagcttatggattaaggtgtttcaataatttaaacaattctctcccttcttctgagtggactcatcgctaagccaaaacatatttgtagtgggatcaatgctacatttgcaaatgagcttgaaatgtgctgtcagagcaggaa

SEQ ID NO: 28 - DNA, 501 bp, Bos sp.
tgtgatcttgtgccttgtgaatggggaagcctagtttccagcctgtgtctgtaatactttcaccactgaaaagggtagtgagggcctcaaaggtgctatgaggtccacatagatccatggaagatccagtgatttaattacattattatggcttttaagtgattttaaagtctctttgttttcagggaaaaatagcaggctaaaaataggcgtaagtattttaactgaatttgaatgatcatctcctaatwttaggaaatcaaacttaggagtagatgattgagaaaaagaggacatttaaatccttttttttttaaatataaatttatttattttaattggaggctaattactttacaatattgtattgattttgtgtacatcaacatgaatccgccactggtgtacaggtgttccccatcctgaacccccctcccacctccctccccataccatttaaatcctttttgtgggtttttaaaaggtattactcaagttcat

SEQ ID NO: 29 - DNA, 501 bp, Bos sp.
gccttcattacttgcagtgtgtgccaggttttcaactgtaaagatacttttttttcttttaaggcttttagtgaatttgttacaatatagcttctattttatgttttggtgttttgactgcaaggcatgtgggatcttagctccccaaccagggattgaacccacaccccctggattagaaggtgaagtcttaaccactggacggccagggaagttcctaaagttactagtttccattcccatcttctgtkttttgaaagtgaatcactgggttcagctcactcaaatggatgggagatggggtggggaattaagctccacctcctgcaatcagatcagatcaatcgttcagtcctgtctgactctttgcgaccccatgaaccgcagcacgccaggcctccctgtccatcaccaactcccggagttcactcagactcatgtccatcgagtcagtgatgccatccagccatctcatcctctgtcgtccccttctcctcctgc

SEQ ID NO: 30 - DNA, 501 bp, Bos sp.
atatgctggagacttgggtttgattcctggtcgggaaaatcccctggagaaggaaatggcaacccactccagtattcttgcctggagaatcccatggacaagagcctggaagccaacagtcacagggttgcagagagtcagacatgactgagcacacacgcatgccaagtggtgtcaaagaccatgttctggatgacactgctctgcatgaagaatacttactgcccagcatggtgctgggaccaggatgyggtaagaattcagaactggggagaagaaaactaagcaggtcgggggaaaaacaagcagaggaggcggagcagaggttctgggagaagattccatcctgggatgaaaatctgatttttcaaactgcttttctcccacaaatgagatcttacatgaatctgcaacatgtaaaatgggagaattactctaactggaaggatgcggtgggagctagggagcagcaaggaagccttaacacctgcggtggcccca

SEQ ID NO: 31 - DNA, 501 bp, Bos sp.
atccaatcatctcttcctctgtctttcatcttattctttattaactaatgtaacagattgcctgcttcaaaggtgtgtatactcattaatcctttcacagttgtgccaggatgatttttctagaactcaaatattaagatatcattccttggctactaaaatcttcggttattatgaactcatcattgtctactggtaacaaactctaaacttagattgctatcaagatattaaatactatgcagcttgtstaccactccaaactcatgttccttcacttcttaaaatatgctacatgtgtatgtatttatatgcctgcaaccttattatcgcctctccccatttaccccctctcctcacaggcatcatgttgtacttatctttttatgtgtagttattatcataatatttagtgtaccatggagattaataaatgtagaatgagtggtaattaaggaaaagaagtctgatatccatgtcattcaagaggcctttccaaat

SEQ ID NO: 32 - DNA, 501 bp, Bos sp.
tagtagccatttgtcacatgtggctacttaaattaaggtaatttaattaaaaattcagttcctcaatggcactgaccacttttctggggctcaacagccacacaagctattgaattggacagtgtagctatagaatagttccattattggagaaacgtctgtgggacagcacttgacctagagtcttaggatttaaagaatctcaaaaagggcaaccaactatcctggtttccccagaatttagggattcytcagtttgaggagtttcagtgttaaaacttggataatccctgggaaatggagacagctcatcaccctaagaagtcaggtagtccaaccatatatctactgcacatatttcttcgacaacattcttttttatttttatttggtcatgcaggctctttgttgtggctcacaggctttctctatgtgccctgcagcatgtgagctcttagttacctaagcaggggtccaaccagtgtcccctggatcaacagg

SEQ ID NO: 33 - DNA, 501 bp, Bos sp.
tcttatcacagtgtgaaatttaacaaggacactttagggcttcctagcacttaagtctttttcgtttttccagcaggactctaacttcaatgaggtctttattcagaatatccttcattttaactgagaacaaatgttcatttaccaacagaaacagaagatcgtgacaattgactctgtaaatctttacctgaactcattatggagggacacatttatttgatttttcttgtattttcagtggcgagaayaccatctgagtaattctactggcccctctttgcttcctctttccttacccttagcaaaccatagtcaatttgaaaaaaggaattcagttcagttcagttcagttgctcagttgtatccgactctttgccaccccatgaatcgcagcatgccaggctttgctgtccatcaccaactcccggagttcactcaaactcacatccatcaagtcggtgatgccatccagccatctcatcctctgtcatccccttt

SEQ ID NO: 34 - DNA, 501 bp, Bos sp.
gcctcctgtgaatgcagggtgaatcagcctcagggttcacctccagctgccagcccattggccaaggtgagaaaatttctagaagatggtgcacccgccagcccagcctggatgccactggctgaccagctctttgtgggaaaagcaatgccacattcacggcctgcaccctgccagccctggacagtgtaccaactgaggtgtgaggcgttccctcactgggccttcccaggaggtcaagagcagattgrgccacaccctcctcattttctggcagaattcagcatagatcctggggtcacctccactttcctgtgaagaacccagcagatccccagcagatggttattcggtgttacttctcatcacttactatccatagccttgggcgagtgagttcaacttcttggacctcagtttcctcctctgcaaagtgggtgcaataatagtaccccctcctgagtggttataagccatcaagtgcttcaggtacacagtaag

SEQ ID NO: 35 - DNA, 501 bp, Bos sp.
attaagactttttccaatgcatgggcacaggtttaatctctggttggagaactgaaatctcatatacctcatggtcaaaacccaaagcataaaatataagaaacattgtaacaaagtcaataaagactttaaaaatagtccacaagaaaaaaaaaaaaacttaaaaaaataatgaggttatttttaaaaagaagaaggaagtaaatgaaaatcaaagacttgccctaccagatgtccagacttattctgarattgtcatcatgaagaatttggtactgtcacagagacatacaaattgacaagtggaatacagaacagagaaacagatttgcacatgtgtaaaactgtagtaaatgttgcagataccatactagaccagcaagaaaataagtgactactcaacaatgaactagaaaaaaattattatcaatatgggaaatgttcgactgggggcatattactcaacatattatgcagtaaccaaatatattggtcagtcag

SEQ ID NO: 36 - DNA, 501 bp, Bos sp.
ttggacagtgtagctatagaatagttccattattggagaaacgtctgtgggacagcacttgacctagagtcttaggatttaaagaatctcaaaaagggcaaccaactatcctggtttccccagaatttagggattcttcagtttgaggagtttcagtgttaaaacttggataatccctgggaaatggagacagctcatcaccctaagaagtcaggtagtccaaccatatatctactgcacatatttcttckacaacattcttttttatttttatttggtcatgcaggctctttgttgtggctcacaggctttctctatgtgccctgcagcatgtgagctcttagttacctaagcaggggtccaaccagtgtcccctggatcaacaggcagattcttaatccctggaccactaggaagccccttctgcaacattcttcactctggacattgctgagcctgccagctaagtgatgttaatcactgatgtggctgaagcaggca

SEQ ID NO: 37 - DNA, 501 bp, Bos sp.
taggatttaaagaatctcaaaaagggcaaccaactatcctggtttccccagaatttagggattcttcagtttgaggagtttcagtgttaaaacttggataatccctgggaaatggagacagctcatcaccctaagaagtcaggtagtccaaccatatatctactgcacatatttcttcgacaacattcttttttatttttatttggtcatgcaggctctttgttgtggctcacaggctttctctatgtgcsctgcagcatgtgagctcttagttacctaagcaggggtccaaccagtgtcccctggatcaacaggcagattcttaatccctggaccactaggaagccccttctgcaacattcttcactctggacattgctgagcctgccagctaagtgatgttaatcactgatgtggctgaagcaggcagtatgtggagaactggagtagatcaagctaaaaaaaaaaagagagagagagagaactgtttcagatttcctc

SEQ ID NO: 38 - DNA, 501 bp, Bos sp.
catcagtctagacacagactttatacccttcacaaagttaacccaaaatagatcacagacctaaaagcaaaattacaaaactcacagacagtaatgggaaaaacagacgattttggttatggtgatgtctttttagatacaacaccaaagcaagttccatgaaagaaatagttgataacctgaacattatcaaaaataaaaactgctttgtaaaagacaaactcaagacaaacaggagacaagccacagcytaagacagaatatctgcaaaagatacttctgataaaggtctgttaacaaagtatatgaagaacctttaaaattcaacaagaaaataaactgattaagaaatgggccaaagatcttatcagacaccacagcaaagaagatacacatgtggcaaacaggcatatgaaaaaatgatccccatctttcccatatgatattatggaaacgcaaattaaaacaacaatgagatagattccctgaggattcccagtc

SEQ ID NO: 39 - DNA, 501 bp, Bos sp.
tctcgtcttctctatcttagcagttctctctttctgaaatcatttgctgtggggtgtaagtatcttaaggcacgactacccttaagagtgctggtgggacaagcagggaagatatacttaacagggacaaagcctcacgctagagcagtgcttctcaagccccactgcacagtacaatcacctgagagctttacatgctgcctgtgcaaagggcccacccatgggggtcagatttagtcagctgggagagkggccagggcatcgtttgtgtctgagttcccctagcaactccaatgtgcccgtgaatatataggctagaaaacatgaaataaggggttaacaggcacacgtgaccttccatgctgcgtacagggaactgtgccgtcatggtaatgctgttcaactttacttggctccagcctgcagtttgaaagactttctcattccagcaagtctccagagtgtaatttcctataggagatcaccaggggtcctcagaga

SEQ ID NO: 40 - DNA, 501 bp, Bos sp.
tcccctagcaactccaatgtgcccgtgaatatataggctagaaaacatgaaataaggggttaacaggcacacgtgaccttccatgctgcgtacagggaactgtgccgtcatggtaatgctgttcaactttacttggctccagcctgcagtttgaaagactttctcattccagcaagtctccagagtgtaatttcctataggagatcaccaggggtcctcagagacccaaagggcctcttagaggagttggrggcacatttgccaatgtctgggggtggggagatgaatagcagtctctgaagcatttttggcatggtggcaagcagtccaaaatgcaactgcaaatcaacatgaaaattcttattatggttcatttggggagttattagcaatgctctatctggtagtcgagagtaattatttatggtgaaactaaggcttgaactcaagagtgtgaacatgatgcataaccagctttaagagggttcagtcttttaaaag

SEQ ID NO: 41 - DNA, 501 bp, Bos sp.
gcaactactgagttcacatgctgcaactactgcagccccctagagcctgcgctttgcaacaagggagccaccacaatgagagacccacacacaactaaggaagtagcccccacttgctgcaaccagagaaaagcctgtgcagcaacaaagacccaacacaataaaaaattaatatatttttttaaagcacaagtctttattcttaagcatcttaaattccagtgtggagagaagaaaataagcaaaatatrtattacacatgtgcagatggtgataaaggctaaggagacaaatgagccaaagggagagaatattcagcaaggaagagggttgcaatttcaaactgggtgatcaaggaagatttcactgaaaaagtgacacatgagtgaaaactcaggaagtaggaatgagctgtgccaatgactggcagaagtcagggcagctccccagtgaaaggaaagggaaaattcaagatcttaaagagggaataagcctgagtag

SEQ ID NO: 42 - DNA, 501 bp, Bos sp.
tatatgtgaactgtttaaaaacaacaacaaaacaggctcatagatacagagaacagatctgtggttgccagaggcaagggctacatgaaatgagtgtaggttattaaaatgtataaacttttagttataaaataggtaagtcctggtgatgtaatgtacagcatgatgattatagttaacaatactgttttacgtatttgaaagcttctgagagtagatcttaaaagttttcatcacaaaaaaatatttayagatgtgtagtatgagataagggagattaactagaatgattgtgaccatttcacaatatatacaagtaccaaataattatgttgtacatctaagactaatataatgttatatgtcaattgtatccattaaaaattcaaaaaaaataccttttcactctccttttggacagtcctgtagactttttaaaaagatctacaatgttagatgacaagagtctttctactttgataatgaatgaaagtatagtca

SEQ ID NO: 43 - DNA, 501 bp, Bos sp.
caccttggcaatgctcacataccaaagttaggacttactgggaaaagactggcaccttaagatttgagatatggatatctgtacagttgcaatgaagaagcacgcaactttaggctaccctgagttttatgagctagcataccccacctccacttgacagaagtatctagagcatctcccaggcctccgaaccgtgagagggcctcagagcagctgtctcacaggacggcacttgccatcttcaatgttgmgcctcttattctgtccaccagaccagcggtgggggtcaaacctcctgagggtcaaatagacggatcgttgataaactgctgagcccactatgggagggagtgattttctgataaaggattataggatataactagttctccaggagcttggcctagaaatgtatcctagagtgctgaatagaatgttaaactagttagaagaacttctgtcagaaataggtcactctcccgtgactctggatttaattgc

SEQ ID NO: 44 - DNA, 501 bp, Bos sp.
gaccagaagaaaacctactgagactctagaagaaaggaacagtgtagatggtggtatgtttgcagagacatgaagcagctagtatgttgaaccacaacttctgttttccatttctttgcctcttactgccctaattctcctcctcacttgcaatttaccagggattgataaatcagataattatgcttattagaattgtgcccaaaagaatgaatagctgcaaggcaaatctgtattttcaagtaagaaaygtgccagcctttcactagtctctggagcaataactgggaacaatatgcaatcctagagtttatggactttgtgacatcaactttggctgccagctcaatgatctttttcacttgcttcttggttttgttaacaatcaggatagctaagattgccagatgtggcttttgtggatttatggtgttaagatcacccagtgagctcctccccttcaatttcccaaattaccagccagcatataaacacttgggt

SEQ ID NO: 45 - DNA, 501 bp, Bos sp.
aacctattcatgccaagtggatggcatgaccttatactcagaatagcaaagaatgtaaatctaaaaataaaatatcttaaagacagaaaattaaaatcgtgacagttaaagagtaagaatgtacattagaagtcacatttctatttgtgtgtttgtttaaggctgttgctcaaaaaaaaaaattctgaaaaattaatacaattattctatgcagcttaaattatacctggtctatggctttacttagcagraatcacatcaaaatattttattttatttcttaatcatccccatcagtttagtcatgattctggggtaaaaggaaaaccagtaagtaaagcagaaagaaattaagaggagatgcaagactagcaatgtgatacaaagtgcagggcatgatctgaggtcttttacagacacttttattcacacattaaaaaattactgatggattttttactgaacatgtttatgtttgtgtaataagggtcccagaattat

SEQ ID NO: 46 - DNA, 501 bp, Bos sp.
gggagaaaatattttccattgaaaaataaaaatatatcaagacctttggcactgcacttcatttttaaacagaacacagtttttaatggggattttttccttttaaaattcttaaattccctttttatttaaagaatggtacatattcacttgtgttgatattcactatttgaggtttataatatcctcttaaattgttcaatcagtgattttaatctttttcaccttttattaattcattgacagatttsacactttttttcagtaaatacatttactctgatttttggaattaaaacatttttaacttctgaatcatctaggttatattttgggcatgaataatcttgttgcctttttctcatctctaaagagtttctcatttttccttatttttctctaagattgttagtagaccactatgaaatcactggacagaatttttgttacagaggatgcctacattttgagcaattaaaattgttgtttcttggcattata

SEQ ID NO: 47 - DNA, 501 bp, Bos sp.
tcttacaatacacaggacagctccctccacagcagacaaccatccagcccctactgttggtagtgctgaggtggagaacccctagcctgatatgaagagaaggaggccccatcgcagccttcaaggagactgcagtggggatgggagagagagagaagatcaggtggaaactgtaacgtaaggtagaatcaggttgggccatgcaagttgcatacactctgaggtggttctgctgcctggaccagttcccycgagaagtcaggatgtggcacatggaaaagtgctcatcggtgatgagtttggaggacagtgggctatagaaggttaaacacatttcttcagtgcaaggcttgcttcttgtttttaacgcacggtgtgcagtgtggtgttcaggagaagagaaggaagccttttaagcattatttggggagaagcttccagtttgcattcagtgggctgccatttcagtcagttcagttcagtcactcagtcatgcccgac

SEQ ID NO: 48 - DNA, 501 bp, Bos sp.
ggattgcatagtaaccactgatgcaaactgtccattatgttttcatgtgtttccagagttctcacataacatgttaattggaataagcatctgtcagtactggaaagtcaccaatcaagacaggagaataaaagtgaaatggtagtagctttcattgaaaaagcaccttatggtaaagtttggaagtaaggtgatgtaagacagaaggtgcagtttgggtgtgttaaattgatgagggaagatatagtggraagatacatgagggaaatctatgcttggcttctagcttcagaaaaattctggaattcacttttggtttctaaaattccagaaatctgaagtaaattaaaaaaaattatactaaattattgtgcgcctctgattatttgtaatgaatatcattttcaccaataatatgcaaataaattgcttaaaaattgctcacattatgaaattcatgctttcatttgtttttgctactctcctgctccaattaacttc

SEQ ID NO: 49 - DNA, 501 bp, Bos sp.
gagatgaagaaagataagccatgttaatctcaaaaattcataaatagcaaaattaaaaccaccagatcaataaaacagtaatcaggaagagtttactgaacatgttaagaacagtttagaactggacgcttgcaacctcaaaactaatatgaagctcagaggcaaaatgttacagaatggcttacgaagtgaaaaaatgaggaagcggtttaacctttacggtgattggttattacaatgagggtttctaragggcaggggattgctcaaaagtgattatttaataccatgttaaggaagatgacttaagtttctttgatgattatcagaggcattcacaagaagtaaactaagttaagttttgcttatgcacatgaagtaggctagatttagttttgcttatatggcttaaatggttttgtctcctcagggaattttcaagtttgatgttcattttatattttactttttacagtgatgctaaatctaaataaacctagg

SEQ ID NO: 50 - DNA, 501 bp, Bos sp.
tgaagctcctttcagcaggacttcaacctaatagcaaataatcacatcccttctggtgatgtttatgaactagagaatggttccaattggtcagtttcatgcaggtctgaatatacacattttgattgaatttgctcaaaagaagatagctgcactgttaatcatatttattagttgatcttaagaattcatgcatttaattgttctagaattagagataaaccttggaattatgactgtgagaaatggcyaagagtttcattggggatgacagaaggcaatgctgagcaagaaggaggtttgaaatcctgcattaattctagagttcatcaacctatgactatggaaatctcgatatatttgaattgcaaaaatttataaaaaactcttaaatactttttgtccttctgttttctccttcaatataagcctacaaaattataaggtacttaagtgttgggaccatgtctctgaatctttaataacaaacacactctcata

SEQ ID NO: 51 - DNA, 501 bp, Bos sp.
ccagcagtcccacagctgtcaccacgatccttttaaaatcacaatctgattccatcactccctaacctcaaacttccaaccacttcctatcacatgtagaataaaatccaaagatccttgccatagtctggagaccttacatgtagatccctgcttatcagagacctcccttccttctgtgttcaccattacagccgcagagttttcctggatgggctccaaacgtcccaggccagcattcctccagggtyggggcagagtgctttctccgacagtcctacacacaccagaaccctgctatcactctctatcccctcgctccgctttatttctctacactgcactcaggattatattacacacgtattagctatttctctgtataccacgtaaccacgagggcaggggttggatctgttttgttcacaactacatcaacaatgcttaggagaggtctggcacaagatggtctctccaaaagtatttggagagtgaatgaag

SEQ ID NO: 52 - DNA, 501 bp, Bos sp.
ccacgcgcctggagcccatgctccgcagcaagggggccaccgccgtgaggagcctgtgccctgcaaccagggagcggtcctggctcgaagcaaagaccccacacagccaaaaataaaacaaaaacacagtacaaatacaacaaagagaaaacagctgcaaacgtgatagacaatattaatccaaatacatcaataagcactttaaaggtgtatctaaatatgctgcttgaatcacagagatctttagagcygtataaaggatgaccagatccaactatttgctatctataagaaatttattttaaatgaaagattaaaactgattaaaagtaaagagatgaagaaagataagccatgttaatctcaaaaattcataaatagcaaaattaaaaccaccagatcaataaaacagtaatcaggaagagtttactgaacatgttaagaacagtttagaactggacgcttgcaacctcaaaactaatatgaagctcagaggcaaaa

SEQ ID NO: 53 - DNA, 501 bp, Bos sp.
taaaactaatcagaacagatttcctggagcatgaaaccctaactaggttctctactaaaatgtacactaaagtcagctccaaagtatgactcaactcagtggagtatttgcctaagcattccagaatagccagggtaggtgatgcacacagtttgttgacgtgagcaaggaaacatgaaagagatctgcttagagatctgggaactatgtttcatggtttgctaatatgagttattattagcaatcagcamttcttaaagatcaaaaaaaagtatacaaagaagagagaggaaagtgtgtttagcttttaaatatgtatcataaaccacaacttagatctgttttctggtgatttggctccattatttttccagatgtcattattcctattttttaacactacccatctgcagaagggctcaaatattcaaaccccaccacatagcttttcctgtctatatatctagtctgacttcatcaaacacatacacattttttttt

SEQ ID NO: 54 - DNA, 501 bp, Bos sp.
gagaaaaaattataaaacagacatacagagaataaattttagtttcaaaagccaaggctgcaaagtaatgaatatcttgataaatgagaacaatcctctcaccctggatagtaaaagattttcccagtaagaggtgaaataggaaacaagaaataataaaagagggtcagagacctataggactcagattggaaaggatatgtggggagacgacatgaggaggaacacctgttgtcaacagtcagagactrtccatgtcccattgtgacacagcccagagctgtggagaatgtaaatcagcacttgtctgtccatctcaatttgaaaatacaggtggcagagtctatgcttgccatctttattctggtcatctagaacggttctttccttcctgtagtaaatattgcttattgattactcagaagttatgtctacttgtgccctctggctttgctctacctaaaaggcactaatatttgtgctaataacacaagctctttt

SEQ ID NO: 55 - DNA, 501 bp, Bos sp.
aattatttctttcatttctgaggttataactaccaatattcatcacatttgcaattaaaacaaagacacctaaaatatttattaactagctaaaagtaacaaaaaaaaatccatttgatgttaacatgaataacattttttgttaaaaaataactatattttacaaaacaaaaaaatatttagtgagaagagtggcactattttatatgtttacaaatctctttcctatatggtttgaaagaagacagtgrattcttatatttagttgtgcatattgagagtcccttggactgcaaggagatccaaccagtccattctgaaggagatcagccctgggagttctttggaaggaatgatgctgaagctgaagctccagtactttggccacctcattcgaagagttgactcaatggaaaagactctgatgctgggagggattgggggcaggaggagaaggggacgacagaggatgagatggctggatggcatcactgactcgat

SEQ ID NO: 56 - DNA, 501 bp, Bos sp.
aaatttcttgtcatcttcaaacttttctgcagtgcttctatatgcaaattagaatcctcagttttcctgatcattttttcccatattcctatcctgaaacttatgcaatttttttcttcagagagcttttcttaatcattcaagtcctaaataattttattttttatttttatacaattttgaaaagttactttgcatttacagttattacaatatattggctgtattccccatgttgtacaaggcaaccygagtctatcatacacccaacagttagttcttcccactccacccaattatattcccccatcttctttgataaccactaattttgtccctatgtttgtgagtctgcttattttttgttatattcactagtatgacatattttttagattccttgtattaatataagtgatatcataacagtatttgcctttttctgacttctttcacttgtaaaataccctcaaagtctatccacgttgctgcacatggcaa

SEQ ID NO: 57 - DNA, 501 bp, Bos sp.
aaatcatttgctgtggggtgtaagtatcttaaggcacgactacccttaagagtgctggtgggacaagcagggaagatatacttaacagggacaaagcctcacgctagagcagtgcttctcaagccccactgcacagtacaatcacctgagagctttacatgctgcctgtgcaaagggcccacccatgggggtcagatttagtcagctgggagaggggccagggcatcgtttgtgtctgagttcccctagcractccaatgtgcccgtgaatatataggctagaaaacatgaaataaggggttaacaggcacacgtgaccttccatgctgcgtacagggaactgtgccgtcatggtaatgctgttcaactttacttggctccagcctgcagtttgaaagactttctcattccagcaagtctccagagtgtaatttcctataggagatcaccaggggtcctcagagacccaaagggcctcttagaggagttgggggcacattt

SEQ ID NO: 58 - DNA, 501 bp, Bos sp.
taacccttgataaagagaatagaaagttcataatcttgagttgatttggcccccaaattgaatataattgaactcaaccaataaaaaatgaaataaaaaagctattttagctagaagaagcaatcaaacaaaaaatattcctagccttttgaaaatttaaagtaaattaagagagaattcctaacaaatttggacttggcatatatgtgtaaaaaagatttatttttatatcagtttctatctgaaggaartagctctgtagcagcaaggtaagatctgatcttctttgcttgatagtcagcacactgaatctttattgaggtgttagaagaggcaagttcccatgccaagatttaattaaagtttaaatatttgaaagggcagtgataatgttgtctctaataaaaattttacctgtcttgggatcaatagagaaataaggttgtccctgaagaatgctgtaaacgactctggcactgtttccataggttgggtcatcag

SEQ ID NO: 59 - DNA, 501 bp, Bos sp.
atttataaaaagtgatggtttttatagcttaggctcatcatctcagagtttatgtactctttttaatcatcatgcccctgattgctgattatttactcttctgctgtttcctctcatgattacacagcctgctatggagccaggcatcatgcctctgcgagtccctgtagcatctctctccactttagtttgtcataaagcagctggtttcttctctcatccatccactaagtatgccctgcagaattgtmtaaccaagattctacatgatatggttgcattctactcacagcaacctaaaacctacatggctatagtttcaatacacgggtctacagcagaaggaagactactgttattttatagattgttctggaggatgctgctacactggtacatgatctcaaggtgttgaagatattggcctactacttgatttatacacagcttccataaattttagataatccaatgacaactttcagtgctacaaggctaatt

SEQ ID NO: 60 - DNA, 501 bp, Bos sp.
ggctggatggcatcaccgacttgatggatgtgagtttgagtgaactccgggagttggtgatggacagagaggcctggcgtgctgcaattcatggggttgcaaagagttggacacgactgagcgactgaactgaactgaaatgacgtatttgtctccatccatttgtcttcctgcctgtctatatcagtatttatatgtccaaattagcagtaggacttacccaccatcaattttattcattgatttaatgyaggtgcctaaactgttctcaaataaccagttcaactcactaaatcccaatttcaccaacccacttatttctgaccctcatggagacgtcattagttttcagtttactttgtactcctaccttggctaccagggaagaggacctttttgatgctaggaacatggcaggggggcattggccaactccacaggcccatggtttatgaagcacattttctgatggaaaattcagtctccaaagaatgtctttct

SEQ ID NO: 61 - DNA, 501 bp, Bos sp.
gtttgacagctgttcattcgatcttgaagtacttggtaagagaggcttctagtctggataagtgtgatgtttatttatttattttttaatggggtgtgagaggatagaggagaataaggtcatgtattagtatgagattttgaaaataaagttccctgggacaaggatgtcaggaaacccaggtagaagggagaattttctcttataaggactaatgtcagatatggacattcttcatctatatgttctgygtactgccaagagtgaatttgataagaataaattaaaggtatatatgtagattcggggaaaaaagcaatacttcaataagtaaacaaagggaaagttgacttggtaacaaatcaaaagtgtgacatagctttgtatttttagacaaagctgatgcgatttaaaaccagagcattacaaatgtggtaggatctaggtttaggaagtgctactgctctctgtaacatgttacttgttccatgtttgggggtc

SEQ ID NO: 62 - DNA, 501 bp, Bos sp.
taacacagtttaagttagtttttatataaggtgagaggcctgtatctaagttcaattgttatatataggcatccagttgtcctagctctatctgctgaaaactctcctttctcagttgaatccctttgtcttttgtcaaaaatcagttgactgtgtttatgtaggtctatatctgggcactcgatcatgttttattgacctcttttttagtatcttaccatcatcatgtgcttttgatttcaattgttctrtagtcttgaaattggataggaagttctcaatttgtttcttcttttatgtaatattgactatattgtgtcctttgtttttccaaatacatcttaccgtcagtctgtttatatccacaaaatattctcctgatattttgattgaaattgtcttgaattaatagatcaatttgggaggaattaacatctaacaataccaaggcttccaaccttatgtttccaatctatgcaaaatacacctccattaatttag

SEQ ID NO: 63 - DNA, 501 bp, Bos sp.
  misc_feature (434)..(501): n is a, c, g, or t
aagacaaagaaccagtagcaagacagtgggacgtgtgctctgatgttggtaggaagaagtactagggacattagcaagggggcacccagcttgatctgtctttggtggggatgatcagagaatacacccacaggaagatgaaacttcttaggccggtaatagccaggagaagattaagttcattccaggcaaagagaatagcaaagtacaaagaccaagaagcaagagacaagaatgtatataattggggragggggcaaaagaatgcacacagggtctggaagggcaaatgtgtgtatggtggtgagaggtaaggatacgggaggctacagactgaagagagatgagtttacagatgtgaatggggagaaggagagtggatgaaagattcatgtattcattcaacaaatgtactgaatgactactacgtgctnnnnnnnnnnnnnnnnnnnnnnnnnnnnnnnnnnnnnnnnnnnnnnnnnnnnnnnnnnnnnnnnnnnn

SEQ ID NO: 64 - DNA, 501 bp, Bos sp.
tatcagtggttggggcatagaattggactactatgatgttgaatgatttgccttggaaatgaactgagatccttctgtcatttttgagattgcacccaagtactgcatgttggactcttttgttgacttttttttttaatcattgtgtagttagacaaaaaattgtctctatgagaattttgctgttatcaagtaaataatacagctaaactaataatttataagaaattaataataatgtaaaccttatrtgcaattatattttcttaaagtattcagttgatttttagagattgataactaattctggagacttttaaagagtagagcagtgtttctcaaactttagtgtgcagaagattaccctggaaaaatgtaaacagattccttggcctcacttctagagatgctgctgctgctgatttctagacctgtactttgagtacctctgatatagaaaacttggaaaaatcctgaagattaatgtgcctaacctatgat

SEQ ID NO: 65 - DNA, 501 bp, Bos sp.
ttccccggatagagtctggtggtgtacatagggcttctctcagtggaaagccatatggatcacccagtctaaaccacatcctttcccctgctgcctctctttgccctcttatgctgcttcactttgctttgtggcattgcccatcatcagatacagtacaggtgtgtttattttctgctaccctgtttcacacaaagggactcccatgaagatggggatgggcatgttttgttctctgctttatccccagygtttggacaagtatctggcatatcacagacacttgttgaaagaatgacttcatagagaattcaactgaactatctcttcactgactattggctgcatgtctgcccggtgacatcattggagagacactgcagtcccttagagagaccatcaggacggctaagactgatcatatccctggtcaggattgagattcaatcaaggcacctagtatagggcttgaactcgaccactggggcaagagagaaaaag

SEQ ID NO: 66 - DNA, 501 bp, Bos sp.
tctcaatatattgagatacaattattattaaaataatatatatataaaattgctagaaatttcatgaatatgcttataccaattttaattgtgaatggcttcaataaagagaaagacagcagatctatgaataacaataaacttctaaatttcaagcgcattttcttccactgaactccaagttagccaagcttatgtagttgtgcagaaaacccatctttttgtagaaaagtatgtcattagtgtaatgrgaaatcttatatcttagttaaatcacccagatgttatattttactataatattccccaggaagttggaagaacaggtggttgattgtatgatggcagagagagaaaggaaaggatgcatctttttttttttgtaaactgtcccaagttttttggaaactggtgtttttccaagagtcacaactcttccaacaagaacatgtttctttctaagctgtttaattccactaaattctattttagcctatttca

SEQ ID NO: 67 - DNA, 501 bp, Bos sp.
  misc_feature (499)..(501): n is a, c, g, or t
ctgcagacgataaagcacttccaggcagacagcagtataaccatcagggcaccttgttcgtttctcatttttatggaatcagaatcctatgctatgtgttgtttggaaacaattgaaaccagaatattacatctatctccaatatgacagaagaataaaatattttctggtacacacagattaagaatgctgaagaaatctctagatgtagtactcaaacagggtgaaaggaaatctttatatattgcaaraataatgaacatagataattactgatgcttgccaatgatatagtctatagataatgaaataaagcaagaaaagtgaatcagtgtcatgaggataaaaagggagaaaatactgtcattatttataggtgatacaataccctacttagcgaagctaatgaatgagcaaataaattaattacataatatagcacatatatgggatacaagctcagcctacagaagtgaagtgaaatgaagtgaagtcgctnnn

SEQ ID NO: 68 - DNA, 501 bp, Bos sp.
agaggccattgcttattagaatgcagtcagcacaattacatagtaatggttggctcatcaagttggtatctgcttggaaattatgcctatgagacacagatttaattacagttggtttagtgggcaatgttgggttatggtcaatactggataaacaggtaattacgaaagcaaaaaatgaagattctgttttctgttaattatattttattagccttttttatccaccatcttttttctttgccttaatktgtctaatcacagtatccaggccttttactgtttagctgtttcattgccaaaaagctgttgacatacccagtaatgcactgaggaagaggtattttccagttttaattagtattgttattctgtcactaatgttggaaagattaatggatgcagcattatttctcagcaagtagacactggtttacttcactggcaatgcattgaattactctgcttatcagagtgaggaaactgagcaggagacattcc

SEQ ID NO: 69 - DNA, 501 bp, Bos sp.
ggcaggcaggaatgtgaatgcgggggagaggtggctgcagtttcacctcccccatggggaaagaaagtgccagacatttcttcagtaccatccatcaagtgcagacaggcccctcctccgacctctgcaagcctcttcctggagtccacgcccagcggcactggaggttaggatggtccagggagagcttggaggtctgccttggagacagcgctccttgatacacaagtggctgaagaaaggatgaattrtcctccctttgtctaatgagccttctagaatatcctgtgctccctgcccaccatttttggcctcagtcagatttgatttattgggagagggacctaacacagtcaaatcctcaccctcctcagctggggacaaatagctctgggaaggcctaacacagaagggtggttgccttggtaacacggcagcacatccaacttcctgatgggccctggtggccatgttgactcccaaacccccattccacctccc

SEQ ID NO: 70 - DNA, 501 bp, Bos sp.
tgtgatgagaatacttaggatttactctctaagcaacttttatatataacacactgcagtgttgattatatttatcatgttgtttttttatctccttagtgcttatttctcttacaactggaagtttgtactttttgactacccttctccagttccccttccttctcccttctctccagtaaccagaaatctgatctcattttctatgaatgagtcaatttatttttgaagtctaagtgacctacaacatyatgttagtaaaatctaaaacttgaaataaaaaaatatggaaatattttcataattttgaaaagagaagcatcccatccaaagccattaagggaaaaataactaaatttacatagcttttaaaaaaaaattgtatagcctgaaaagaccataataaaatccaaagaccccctctccaaaaaaatctttgacatatatgacaatgtttatgcattgataagaaaaaagactagtaactaagaaaaaggacaa

SEQ ID NO: 71 - DNA, 501 bp, Bos sp.; identical to SEQ ID NO: 61

SEQ ID NO: 72 - DNA, 501 bp, Bos sp.
agtattctggcctggaaaattccatggactgtataatccatggggtcacaaagtgtcggacacaactgagagactttcactttccctcttgttgattacagcagccatctaactgaactttccaccccaggtctgagcccttcatagtcatcttcaacacccctaagataaagagcctactaagccattctccagtttaaagtctcccaatagcttcattacaacctacagtgtaaagtccatatgtgatsttattgcatgtgaggcttctctttgtcctcagactcatgtctcatacgctctatctttaagttcgtgatccaacatgaattcctgacagtcctcttcctaattgctattccttatttagtttcttctagtcccattttcctgagataaatgtcccacccttgaaaggctaatttttacctgtcctaaaaggcagagctcaaatgttgtctcctctagatttgggtaaaatgttccgtatctgtgccctta

SEQ ID NO: 73 - DNA, 476 bp, Bos sp.
tactcagaagttatgtctacttgtgccctctggctttgctctacctaaaaggcactaatatttgtgctaataacacaagctcttttgttctacatatcccagtgacaagaagggcagtaaaccaaagaacaaagaaaataaagaaagatattgataagttctatagcagcttatatcatttaatataattgggttttctttaaagatgaactttatttcactcattacatgattatttagagtttattgtragtaagacactgaactgtcattcaggaagacatatcttaagcaggagtgcagtattcttggcatacattgaatttatttacttgtatcttattcctgtcatctcttggctgacaactgacaggatttttattcttaactttaaacacgttaccaagctccaacactcaagcttttgatgctgttactctatattaatgttgatgccattaacatctctgcaggtg

SEQ ID NO: 74 - DNA, 501 bp, Bos sp.
ctatatgaatttttagaaaaacaagacggaattttaagaaattaaaaatatgatggcagaataattatattttaaccccataaagtattagaagatacagttgggaaatctccagcaagatgaaaaacaaaaaggtgaagagaagagaaataggagacaaaaaaggtaatagattatgggtaaacataagaatgtctatgcaggaatgccaatatcagagtaattaaaggccttgaaagaaagatcacagraaatggagataaagaagtagcatgagactttcttaaaactaaaacatagaattttcaggttgaaaagacccactgaggataataaaatgaataaaaacaaaaatataatgacattaagtgatatcataatgatatttcagaacaccaagatgaaagagataatcaaaatttccagaaagaaaaaagtcccatcacaaaccaaagaccagatattaaaacactgtctttcaactgcaatactggaggtcag

SEQ ID NO: 75 - DNA, 501 bp, Bos sp.
cttggcaggctacagtccatgaggtcgcaagagtcggacatgactgagggactaaatcaccaacatggagatcaaagatatagatgaatactagagtcaaatctcaaccaaaatctcagcctctccccactctatgaccttgtgcaaatcttttattctgaaacctcagtttcttcatgtataagatagattatatattaataattatcttttatatgatttagtgaaataaaatttattagcactgagartctaggaaaacggatggattgcatgggtagctctatcaataagaaggatatgtacatataatgaattcaataatgtttcataacaacagtgagaaaagtctatgtgaggtaatcagggaagagtgtgaagaaaatatattaatctcatgtaagtacagatattgtaaacttcaaaatattgtggaaactgacatttgtatcaactttttggtctatttaagtttattcttactagattatgttttaacta

SEQ ID NO: 76 - DNA, 501 bp, Bos sp.
tgaagttacttatccaaagctatagaacacaaagtcttgcacacattaggtcttaaaatataaattacatgttagttgttatattagcaaaagaggcagacctaaaatatctggagcatttttttctcaccaaaatcactgttgtttaataggtttgttttttttgtttgtttgtttttccatttgtctggaagttcttcatttgtctggaagtgttcaaaagaaagtataaaagaaaaatgttcattatrtgaagttacttatccaaagctatagaacacaaagtcttgcacacattaggtcttaaaatataaattacatgttagttgttatattagcaaaagaggcagacctaaaatatctggagcatttttttctcaccaaaatcactgttgtttaataggtttgttttttttgtttgtttgtttttccatttgtctggaagttcttcatttgtctggaagtgttcaaaagaaagtataaaagaaaaatgttcattat

SEQ ID NO: 77 - DNA, 501 bp, Bos sp.
agagccatttgaataaatgcccagtccctaatggtgacagctttgaggggcaacgttcacttggatggatactcgtggtcttattgctttgtcatcacccatttatttgtccattcttgctacacacgtattgaacatcgattatatgttgctaaacagctgaataaaactagatcacaatcctcaagacaaagaaccagtagcaagacagtgggacgtgtgctctgatgttggtaggaagaagtactagrgacattagcaagggggcacccagcttgatctgtctttggtggggatgatcagagaatacacccacaggaagatgaaacttcttaggccggtaatagccaggagaagattaagttcattccaggcaaagagaatagcaaagtacaaagaccaagaagcaagagacaagaatgtatataattgggggagggggcaaaagaatgcacacagggtctggaagggcaaatgtgtgtatggtggtgagaggtaagg

SEQ ID NO: 78 - DNA, 501 bp, Bos sp.
tcgtggtgacatgatatatggatttactgtgaaaggttaatttactgtcagcagttttggatctctttggaccaaaatcccctcctggaccctctggcttagctggattctcgtctgcagtcacgccctgctggttctcacactccagaggacagacgttccctcacctcgtaggaggccctacatatacttaccagagatcaagttgaagagcaccaatataaaagtttaaaaaatgggaactatattcratatcttgggataagctagaatggaaacgaatactaaagagaatgtatatatgtataatataactgaataactttactatatagtagaaattaacacaacattgtaaataaactatcagttcagttcagttcagttgctcagtcgtgtccgactctgtgcgaccccatagacggcagcccaccaggctcccccgtccctggggttctccaggcaagaacactggagtgggttgccatttccttctccaat

SEQ ID NO: 79 - DNA, 501 bp, Bos sp.
tgaggaaaggatagatggggtaacccaagccaggaccacagctgaacaaacaaaagccaatggctgctcaattaagagtgagtcagtagcaccatactcctgtatcaagggtagaacatataatttctaaatgtctaaagcctggaattaatgtctgtgacctcggtaataccaagagatgaattgtgagaatgaagattgttctaattttctcagcaaagataatattttaaaaactacaaactgaaaaycactttttgtgacagcacgtttaatttgtgtagctattctcattgcaggctgatttgctaaaatatgagcgaacaagtctaatgtagggagttgaacagagcactatggtatcgagtgtgtagtttcttgccttacgatgcctactatgattttaaagttacttaaaattgtttctaaagacttcccctgaacttgacatcaggaacttacagttatacttggttgaggcccctgttctcagtggggtat

SEQ ID NO: 80 - DNA, 501 bp, Bos sp.
gggatcttctgacccagggaagatgcagggtctccagggaagacccctgacccagtgcaggggtctcctgcattgcaggaagattgtttaccttctgagtcaccaaagaagcctcaagaataggtctcaagtattttgagaaatgctagagccatttgaataaatgcccagtccctaatggtgacagctttgaggggcaacgttcacttggatggatactcgtggtcttattgctttgtcatcacccattyatttgtccattcttgctacacacgtattgaacatcgattatatgttgctaaacagctgaataaaactagatcacaatcctcaagacaaagaaccagtagcaagacagtgggacgtgtgctctgatgttggtaggaagaagtactagggacattagcaagggggcacccagcttgatctgtctttggtggggatgatcagagaatacacccacaggaagatgaaacttcttaggccggtaatagccaggag

SEQ ID NO: 81 - DNA, 501 bp, Bos sp.
aaataaaaaatatcatggtaagtcaaaagatgggagaagaaaggaaaatgctattttcctgctctttggacaaggaatccagcattttcgttttggtccttgtaagttatgctcattgaaatttgacaagctttagtataagagatggagaaggaaatagcaacccactccaatattcttgcctggaaatcccatggacagaggagcctggaggactgcaattggttgcaaaagattcaggcaggacttakcgattgagcgtgagtataaatataagagaaagagcatgggcatgatcagaagacccacatctctctctcgttcatgttttgctggtatgtgacgttgggcaagtcactagacctccttgcctcagtttcctcatctgtaaaatgagggtgatgtcttatgcccctttccaaatgggctgggtctaggttaagggcagacctggcaacatgtctttgagtaggaatgtgtccagaacacctctcatctttg

SEQ ID NO: 82 - DNA, 501 bp, Bos sp.
tgaaaaaattataagacatgtaaagtttacatcgtggtgacatgatatatggatttactgtgaaaggttaatttactgtcagcagttttggatctctttggaccaaaatcccctcctggaccctctggcttagctggattctcgtctgcagtcacgccctgctggttctcacactccagaggacagacgttccctcacctcgtaggaggccctacatatacttaccagagatcaagttgaagagcaccaayataaaagtttaaaaaatgggaactatattcgatatcttgggataagctagaatggaaacgaatactaaagagaatgtatatatgtataatataactgaataactttactatatagtagaaattaacacaacattgtaaataaactatcagttcagttcagttcagttgctcagtcgtgtccgactctgtgcgaccccatagacggcagcccaccaggctcccccgtccctggggttctccaggcaagaac

SEQ ID NO: 83 - DNA, 501 bp, Bos sp.
aaccagggtcacctagtttttttttttttttagttttaccgaggtataattgaaaaaattataagacatgtaaagtttacatcgtggtgacatgatatatggatttactgtgaaaggttaatttactgtcagcagttttggatctctttggaccaaaatcccctcctggaccctctggcttagctggattctcgtctgcagtcacgccctgctggttctcacactccagaggacagacgttccctcacctygtaggaggccctacatatacttaccagagatcaagttgaagagcaccaatataaaagtttaaaaaatgggaactatattcgatatcttgggataagctagaatggaaacgaatactaaagagaatgtatatatgtataatataactgaataactttactatatagtagaaattaacacaacattgtaaataaactatcagttcagttcagttcagttgctcagtcgtgtccgactctgtgcgaccccata

SEQ ID NO: 84 - DNA, 501 bp, Bos sp.
acgtgaccactttattctcaagaatatttgaggttggacaaacagtctccaagtacttcttcacatctaagaattgtagaaagagaattggcatgtaaaaatccttccatttgcactgagaggaccgagattctatgtcattttatatcagtcagtggaagttatttctataatctgtactaactcaattgtctgagtataaaaatattccctattgacaacatggccttctttttctcctaactttgttmcctagctgctagtagatgttaactctattgttctcagatgtgtccactcccaagatccaactcactcagcaagctttcattcattattaatggatggaatgagtattgtctcatgtctcaccattgagctaatgggcagagagggagccagcactgtttcattgaaccattactatatcaggcccttacagtaattaggattcttaactaatctagccattggagaaggaaatggcaccccactccagtg

SEQ ID NO: 85 - DNA, 501 bp, Bos sp.
tgttcaccgaaactgggtctccccatgttctttctctcaccttctggctgaattattcaggctcttttcttcactgaattttcctactgagctatccttattctattactctatatccttaattaacgtttaattaagcagttgtttcctggtcctcgccgacgccgtcctcgcttcaaattccctggatccaccggggctggacaccggcagaagagggttcccaagttcttctttttcttattcatgcrtgacccctttccagtaacatctttactgatgtcttttacacagttctttctccagcaaaacatctgtgcctcatagtttttaaaaatcaaatcagtgtacaaaaaatagcagtttttttatacacaccctcagggattgcctcctttagctggtgctgtggtatgtttccagggtacatgacatgaaggtgtcaagggcaataattcacccagctgctctttgtaaggttcatgcccttggttttgggaa

SEQ ID NO: 86 - DNA, 501 bp, Bos sp.
gcacagatggcatcagtcaaggtattggtggactctggcccagagttataattagatgaattaaatcttattaccagtagagaatgtagcttttcatttaaaggaattaaaaacatattttaaaatatactcaatagtttacttacaaaacatctaagttactcttgtcattgtaaatataacttcatttctatacctacagatgtgtattgacatgaggcagttcagttcagttcagtttattcgttcasttgtgtccggctgtctgagaccccatggattgcagcatgccaagcctccctgtccatcaccaactcccggagtttactcaaactcatgtccattgagttggtgatgccatccaaccatctcatcctctgtcatccccttctcctcatgcctttaatctttcccagcatcagggtcttttccaatgagtcagttcttcagatcaggtggccaaaatattggagttggacattattactgtaatcatcagtt

SEQ ID NO: 87 - DNA, 501 bp, Bos sp.
atcactgtggatgttagaaatcaatatagaatgatctcttatatacaaaaactaaacagtcaagatatatgcatggaatgaatgaatgaccataattgtggggatacatataatgtcataaagatgttgtggagttaaaaaagaaaatatatgtaaagtgcttaaaatagaacaatgctcgacacaataaatgttttccattgtgatatttatttctattattagaataaaactgttaagtaatcaatgamtggaatttctgattagaaggacattactgacattggggacaacgtcaatggaataagagagtaaaaagcagattgcaggaagttaggaaattagaataaaaacaatagagaaaacacaaagagacacttctgggaagtttagcagagaagagattaagaaaaggtatggtgggtcaaaggaaggctggctgattgtttattttagggaagtggcaggtaaggagagacatggaacactgcttgccccaga

SEQ ID NO: 88 - DNA, 501 bp, Bos sp.
tgtattataaacacactattgatttgattgataactacctcataaacatgaaataacaataatgaatgaaacatggcagaagaacctttattaacaaaccatataagaataatcatgacattgaaccttcacagagtgatatcaataagccaagttaaacaaaccttaaaaatgtaatttgaatggaattaccaaagttttcctgtttgtaaaaattctggaaaaataaacaataagatctaaatattagratgaagacctcagagaagtacaagtataggtgatttttcttcttttttctttggaaatcttttctaaaatgtcaacaatagtatgacacttttaatatgattagaaaaatgttggttttataagttatactgatggtattgaagtgtctagataccagaggataaaactagaggaccataatgtatggctttattatatcaaggaagtgaattatatccatcttaaattcaccatgagaaagaaatatga

SEQ ID NO: 89 - DNA, 501 bp, Bos sp.
tgtgagtctgtttctgaccagttcatttgtacatttgtatcatgtattttagattgcagatataagtatatcatatggtatttgtctttgtctgacttaatgtgctaatcgctaggttcatccattttgctacagttggcagagtttcatcctttttatggatgaataatattccattgtgcctgcacacacgtgtatacacacacaccctcccacttctttattcatgtatcggcccatggacatttagkttgcttacatgtcttggctccatcttaaccatcttcaccttgttgggcaaccatcagcaccatccatttccagaagtcttttgatcttgttaaaatgcaaactgtgtccttttgaaacagtaatttcccattctccatctcccatccctagcaaccactgatctacctttttgtctttctgaacttcgctactcgagatatttcatataagtagaatcatacagtgttatctttttgtgactggctgatt

SEQ ID NO: 90 - DNA, 501 bp, Bos sp.
tctgctgctgctgctaagtcgcttcagtcgtgtccgactctgtgcaaccccatagatggcagcccactaggctcttctgtcccctctggattacttccattttcttagagacctggaaaacaaagtcatgagctgagaatgcatgcatgcaggagagaagatagtggaggctcaaggaaaaaggagatgtataaaataatcctataggagagaagaggaaatgaaacgcaccagggactagtatatgatarggatagcattcagagccaatggaagggatgatgtggggattattcgccagactctggagtgagagaggttgaataagaacccagcctccaccagcttccacctatgttatctgggcaagttagctaacctctctctctaaccctccctttcctgctccataaattagtgtgctaagttacttcagtcatgtccgactctctatgaccccatggactgtagcccaccaggctcctctgtccatggaattct

SEQ ID NO: 91 - DNA, 501 bp, Bos sp.
ttcagtattcttggctgggaaatctcaggaacagagaagcctggtgggctacaatccatgggggtcgcgaagagtaggacatgattcagtgactgagcacatgcacatacaatattatatatttatctttataccagtaagtcataaatgtaacctgtaacatttgttgtagccctgggtaagctaagatgaattctttgaggagtccagttatctgaagttcaaggcacacgaagacattgttctaacaytgtcaggaatcacagatgtgaagcaattagttctatcttgggaagtaggaaaggctttgcagattgttgttgctgtttaggctttgcagagctgatatttacatcagttatttgaggataaatatgttttctacaggtgaagatagtgggaagggcatttctgggagaaaaagcagcataaaccaaggaatagggttatggaaagagggtgttggatcttggcagtggtcaataaccaataatgcctttc

SEQ ID NO: 92 - DNA, 501 bp, Bos sp.
catgttctttctctcaccttctggctgaattattcaggctcttttcttcactgaattttcctactgagctatccttattctattactctatatccttaattaacgtttaattaagcagttgtttcctggtcctcgccgacgccgtcctcgcttcaaattccctggatccaccggggctggacaccggcagaagagggttcccaagttcttctttttcttattcatgcgtgacccctttccagtaacatctytactgatgtcttttacacagttctttctccagcaaaacatctgtgcctcatagtttttaaaaatcaaatcagtgtacaaaaaatagcagtttttttatacacaccctcagggattgcctcctttagctggtgctgtggtatgtttccagggtacatgacatgaaggtgtcaagggcaataattcacccagctgctctttgtaaggttcatgcccttggttttgggaagacattataggcctgcccagata

SEQ ID NO: 93 - DNA, 501 bp, Bos sp.
tcaagagaagctaccaggctccccagaggaaggggatgacccagccaggtctaagccaggcacagtttgggcagcactttgaaaatcccaagagctgctaatcaatggggacccagagagcgtcagcaccctctcctccaccccccaccccctaccctcctacgtcccgcccccgcaaagccaacctctctctagcagggacgaaactgcacagtggtttgagtctgaagccaagcaacactcagcatgasagctgggggttaggtttcccatatattacaaatcaaaattaaagatagatgggatggggggtggtgcttggcgaaggggggtagggggaaaagcgatctgaggtttttctctttaacttggcaaatgtgaagttaaaatgtttcttttaagttagaacgaccatatcattccaccccctgccctgtgaacctggttctgattattagctgaagttcagcacacgtcaaaggcgtttctggttcggcctcc

SEQ ID NO: 94 - DNA, 501 bp, Bos sp.
ctgtaagaataaaaagtccccagtgtcttagggtagctggaaagagaacttgaggaaaggatagatggggtaacccaagccaggaccacagctgaacaaacaaaagccaatggctgctcaattaagagtgagtcagtagcaccatactcctgtatcaagggtagaacatataatttctaaatgtctaaagcctggaattaatgtctgtgacctcggtaataccaagagatgaattgtgagaatgaagattrttctaattttctcagcaaagataatattttaaaaactacaaactgaaaatcactttttgtgacagcacgtttaatttgtgtagctattctcattgcaggctgatttgctaaaatatgagcgaacaagtctaatgtagggagttgaacagagcactatggtatcgagtgtgtagtttcttgccttacgatgcctactatgattttaaagttacttaaaattgtttctaaagacttcccctgaacttgacat

SEQ ID NO: 95 - DNA, 501 bp, Bos sp.
cactctggcacttttatgaccctgggagtgagcacctcctcaaattttgagcactaggcatcttcccttacttccccctactcctggtactggccaagaacaccttcacctgtgactttgcctataggatatacctaggaatggaaccgatagatcacaggatccatgatgagcaatttgcagcaacatggatggacctagaaagtgtcacactgagtgaagtcagtcagacagtgaaggagaaatattgwatgacatcccctatatgtggactctaaaaagaaatgacacaaatgaacatacttacaaaacagaaagagacttaaaaaatgaacttatggttgccaggagaaaggactggggaagggatagttaggaagtttgggacggacatgtacacactgttgtatttaaaatggataaccaacaaggacctactgtatagcacatgaaactcaaatgttttgtggcagcctggaagcgaggggagtttggaggggc

SEQ ID NO: 96 - DNA, 501 bp, Bos sp.
actatagcccaccaggcttctttgtccatgggtttcccaaggaaaaatactggagtgggttgatatttccttctccaggggatcttcccagcccgggtctgggaaaaccggctcctgcttggcaggcagattctcttatcactgagccacctgggaaggtcctctgtgtagagtacaactatttatcatagcatactaggctctgtactttctagatcctgtgtgtctccgtagctcatttttctgttcartcaattcagttactcactcagttccaactctgcggcctcatggactgcagcacaccaggtttccttgtccatcaccaactcccagaacttgctcaaactcgtcaatcgagttggtaatgccatccaaccatctcatcctctgtccccttctcctcctgccttcaatctttcctagcatcagggtcttttcaaatgactcaattcttcacatcaggtagtcgagtttcagcttcagctcagcccttccaat

SEQ ID NO: 97 - DNA, 501 bp, Bos sp.
aaagtacatttatataagtcattaaatatacaattttctaaatatttgagatattcttttgttgtcttagacttatttttgtgttttcttattaatcaattcctggttactatcattaaggtacctaccaaactcatttcttatgatgactatgaagatgagctatgcctaaggctggctgcaaacagaatccctgagaagcacccccagtagagccctgcgaagtgatgccatggaccttacgtttataycaacaacctgttcatggagaaacacacctttcaaagctgggaaatacatggagatggagtgatactgttaccctcagaacagttcattctgttgccaagacagccaataagcaatgtatttttgttccctgttaatgtttccattcttgtttatccatgcatataaattatataacatatattttactgtagtcgagaaaatagtaatgcttggatttgaaagctaccagaaacattctgccccttccaa

SEQ ID NO: 98 - DNA, 501 bp, Bos sp.
caatcacctttgatctacaaaatccactgtttaatgtacttcaactaaaataaaataggcttctctcgtggctcagatggtaaagaatctgcctgcaatgcaagagacctgggctagatccctgggtggggaagatcccctcgtgtggcagcccactctagtattcttgcctggagaatcctcatggacagagaagcctggcaggctgcagtccatagagttgcaaacaaaaagtcagacatgactgagcrgctaagcataacacaactaaaagagaataagaagagatgtcataagattaatctcatgcatttcagtctgtagtcttatttagaataagaaaataccttaattaactggtaatccaaggattttcagttgtagttcatagttaaaattatctttcaaagtaaccttacatccaagacctatatatatagcaggtaaatgaagcttcctgtgattcacaccaggaacaatcctatattcttgactcccatg

SEQ ID NO: 99 - DNA, 501 bp, Bos sp.
taagaggtgaaataggaaacaagaaataataaaagagggtcagagacctataggactcagattggaaaggatatgtggggagacgacatgaggaggaacacctgttgtcaacagtcagagactatccatgtcccattgtgacacagcccagagctgtggagaatgtaaatcagcacttgtctgtccatctcaatttgaaaatacaggtggcagagtctatgcttgccatctttattctggtcatctagaayggttctttccttcctgtagtaaatattgcttattgattactcagaagttatgtctacttgtgccctctggctttgctctacctaaaaggcactaatatttgtgctaataacacaagctcttttgttctacatatcccagtgacaagaagggcagtaaaccaaagaacaaagaaaataaagaaagatattgataagttctatagcagcttatatcatttaatataattgggttttctttaaagatgaactt

SEQ ID NO: 100 - DNA, 501 bp, Bos sp.
tcatttatggaatagaatgtgtgactctttttgtgtttatgtgttaaagtaacttgtcaggcaaactaattgcaactatacctcaaaggtatttttcaaacccactccagaagaggcttatgaacacactaacttaagtatttcttaattgattcatacttttagggaaaaatagtttagataatcactactaggctgcattttaaaaaaatgtttgtgcatataccctaatgttgattatagcacttttwacaacagccgggacatggaagtaacctaaatgttcatggatggaggaatgggtaaagaagatgtggtacatttttacaatagaatatcactcagccataaaaaaagaacaaaataatgtcatttgcagcaacatggacagacctagagattgtcacactgagtgaagtaagtcagacagaaaaagacaaatatcatatgatatcacttatatgtggcgtcttaatgaacttacctccaaaacagaagtac

SEQ ID NO: 101 - DNA, 101 bp, Bos sp.
ctcttgttcctgagaactccagtgcctcctctgagcctagcttgtgagatrgaacagcagaagttgttcagactgtacagcctttgtacacaagcatggac

SEQ ID NO: 102 - DNA, 501 bp, Bos sp.
gatgggatgaatcacccagtttctgtgctttcctccagatatgcacagcagtatcagtataggttcaggagagtgagtgggtttcctaggtttttaggtatggataaacggaaggagaaaagtaggaggaaatggaaggtgctgacaagacaggggtttataagtaggagcctcacttaagagagaagaaaacaaaaaaagtttcttgaaatatttagcagcaatgaaatacagaaatgagctgcatagtktcctgatgaattatatgcattccagttatgctatgatactaaaaatttggagttaagactcctaagatggaaaagtccaatgcctggaaatgtccaaggagtcactgaaatgaagtggagatagttattggaattagacacaccaaggggccaagagcctggtataggatgtagaaagtgaaagtgttagtcacttagctatgtgaccccatggactgtaacccaccaggctcctctgtccatggaattc

SEQ ID NO: 103 - DNA, 101 bp, Bos sp.
tataatcctcgaatccaggagatccaagttgtcaaattagagaaacggctygacgatagtttgctgtacttgcgagatgcccttcctgaatacagcacttt

SEQ ID NO: 104 - DNA, 501 bp, Bos sp.
atatgtttaagatatgactaaatgactgactccctcagggacctgagatacaatatcagttaacacccaaaacatcacaaaaaaggaactggcactccactggaatagacaagccataaaaaatagcaaaaagtacttgtttccaaaatgtctcagtgattgcctcaatgcttttttgccacagagaaaaagcatggtccagtaacaatgctgcttctttacctgtcttcttacaccttattcaacaaccytaaccatggctagttcagacacctaagaacagaagacaaattggcacaattagcttatcaggtactgtgcatctatgccaagccaatggatgcttgaagaaaagtacataatacttcccctacatgaattacagcctacaaaatgagcatcattagctgaaggggtttattgaacctgtataatttagctcataactcaaagggcaaggaatattattaagaattacaatcttatgctattatttgaatt

SEQ ID NO: 105 - DNA, 501 bp, Bos sp.
atcttgattaaaaagagatcaatagcacgtgggagggaggaagggaaacatatttctccttaagactctccttccctctctcgagttattgaattttcatcttgtgtgcatttccccattgccaaactgtacaaagcacgaagcaggtgagtaaaagcaaccaactctcggagttttttccatggaggctgcctctgaaacatgccatccatagttgagtaaaaggttattaaaataattccatcagcacytctcaataataagccctagcctgcatcagcactaactagcactgcacagtgacccatacatataagacccaattgggtgggccatgacgtctgagaagagcattacagccacatctctttcatcaagagtcagatgacaacttcattcatgtctcaaagaaattccaaaaacattaaaaaatccaacacccaagagctaattccccaagaagtaaagctaggtaaactttagagagtatcataaaaatca

SEQ ID NO: 106 - DNA, 501 bp, Bos sp.
caaagacccagttcagaaagaggttatcaggtaccaacaccatagtatgttctcctgtgttcttttttaattagggtgtggaggtgaaagcctttaagacattttttaagaggacgatggaaaaagatgattctaagaagttatgctgtacattgtaaaggtaaaactctttttagttttctgcttgctctttgtcttgttctttgtatttttgagagattgcttgtttaaagattaaaattgcttttttwaagtatttttgaaattgcagacaagggtcttttatttttaaagcctgtgttgaaactggtccacacagtgacttcatcttacacagtcatgtttagatatttatatttgtttggccttttccttagagtaatttattcaatgtatttagtatttgttaagtgcaaaacactcaacctggcctgtagaaaatttaaagatatcagacaagggccctgtttccaaggagtttaagctggtataattaccatt

SEQ ID NO: 107 - DNA, 501 bp, Bos sp.
gggctgaactttttattagaattttcagtaagaagattactggttactcactgagagagctactgcagtacagcatgatgagaggagccagactgcagaggaccgaggaaggaatggatggtaaaggaggaaagggcgtattggaaatacatgctgattgaagatgcagagacaagatcagagtagacagaacaagttggtggctagatgaaagcccaatccaggaagaagtgcttaaattgggaaaatcycaagcccattagtagtctgaatctagtaagtcccttccttatgaaccttcaagttagaaacttccaaaaatacgaatgtgtgttccatcaacatcaggtgtaactgaaatttcagcttgtgctccatctcctgggcttccctcacagctcagttggtaaagaatttgcctgcagtgcaggagaccccagttcaattccttggtcgggaagatcccctggagaagagataggctacccactccagtatcct

SEQ ID NO: 108 - DNA, 501 bp, Bos sp.
aattataattggttaaatctcgaatgcatttttgggcttccctggtggctcagaggttaaagtgtctgcctgcaatgcgggagacccaggttctatccctgagttgggaagatcccctggagaaggaaacggcaacccactccagtattcgggatgcattttagttctccatcatttggcaaaaactaatactgaatcattctagaaatggtgaaataatgtacagtcaaataaacaatgacacggagaaygcaaaactgtggacaaaaaaaaaatcggatatttcattgcttcttaatatttttcagtaaaaaaaaaaaagcataaacaataagacaaatgattcaccactggtactaattgcaaaaactagaccgtgagtgagccagaagactagctgtatgatgtttggtgacactttcttcacagttttccatgttctcagtgttcctactactagagctaatgaacagttggtcctactattacacatgttccaaa

SEQ ID NO: 109 - DNA, 501 bp, Bos sp.
agaagctgtctgaagagtgaacttgggaaggagacagtggaggacagccgacctctaaggtcgcgtgatccccccaacacctcgatgtgctgaacctggacagatgtccctgtaggacagacgtacctcctgtcttcccacagtgcaggaggctgccacatccctcttcagtggcggtggtcctcagtccagtagccttccggcatcttgtgttgctctgtgccttgttggactctagagagacgatccaygacaaatgatgcacccatatgtcatccgcctcagacagtgcttggattcacacccaagcattggtgatttcactgacagttgagtagagtaacagacaaagctcccactagttgagccctgcctgtgtgctggatgctgtgctctttcatgtgcagtagctcaatcaaacatcatgatgatgaggctcagaaaagttatagcaaccatatgacccacagttgtgctttttggtatttacccagatgaatt

SEQ ID NO: 110 - DNA, 501 bp, Bos sp.
tccatggtaggtgagcagagcctctaccagtggttcaaaatctggcaaagtaagaagtgggacacagattcgtgacagggaccttactcgttcaggagacatcctatggaataaataaaatgagcagtaagtaccaaaatcttagagccacacatagttaaaggcatataaaatcttcatgctgaaagaatttaattatttctttgagttattttttgattttaatttctttgaattatttctttcatttytgaggttataactaccaatattcatcacatttgcaattaaaacaaagacacctaaaatatttattaactagctaaaagtaacaaaaaaaaatccatttgatgttaacatgaataacattttttgttaaaaaataactatattttacaaaacaaaaaaatatttagtgagaagagtggcactattttatatgtttacaaatctctttcctatatggtttgaaagaagacagtgaattcttatatttagttg

SEQ ID NO: 111 - DNA, 501 bp, Bos sp.
ctggagccaagctcttgcttcagagaagaacaagccagaaaagccccttaaaaacattggaaattaaataaaatgtaaaacctttctagagaactgctcttgaggttctgtaaactttcacagctcaaaactacctgaaagccagaatctactatgaagactctgaagacagttaacttttaagggagtattttacaagtctacggcgtctgccttgagaccttaatgtaccaatgctgtacgaacatackcattttgctgacagtggaattaaaatccagaccagttaaaagagcctcttcatctagatagtctagggagctcaagtacagaaccagaatttctgcctatttacttttgagatgtttactttaaccacttgggccattagacttcacctgtggtcagctgagattctacttgattttttgctgaacaaacttgactatccaagcaggtagtaaataacagacttggcctctcttttctggaataagcaag

SEQ ID NO: 112 - DNA, 501 bp, Bos sp.
acatcacatatactaattatcttagctgtcgttactatgacaaatgctcagtcagggacagtgccagtgacttcgtgtacaccctttcatttactcgtaacaatccggggaaggggaggggcactggcccttctcagagacactgacctgcctggcaacacacagctgccaactgcagagcagaaacaccaacccagatcatcctgaccccaaagaacacaggcgtctccgtgcaaacacgggagccacasagacaaccagtgttaccagacaacagacaagaagcccattcacccgagagtccacgcggacatctgaatcacctacaacgcctgacttaggagttccgaattctgagccaaacaccagattttgcacaccgtattgcatgagccgcagaaatgtgtgattttaaggagcatgctgggactctcgtcgaaggctgtctatagtcggattgatcagattcagatgtgaggtttgaggttggcctgcccttgg

SEQ ID NO: 113 - DNA, 501 bp, Bos sp.
gagcacacacttggttctcatgctgccctgcagtgtgtgtcttagccttgtgctgcagacgaggaaacacaagctccgataaattaagcaacatgtccaggggtatgagagctatcaaggtgggtttcagctgctatgccctatgcctttgttctttaagacgctttggggaaataaggtaaatttacatgtggctgatgtttatgtatctcattgaaggtcactctggggcatcttcctcatcaatcccyaagctccttctctgagagttaagtgtccttctcgcatactctgatctcattctgctttgtgctttttaacatttgcaaattctattttgcatgagcatggggatgggggaggttcacattcctcctagccctagaatctaccccatgagagatgatcaagaaaagtcattttctgttattcagtctgtcacctatgcctgtcatagggtaattcctgaaattcaatagttcacttgctttcctgtaaagg

SEQ ID NO: 114 - DNA, 501 bp, Bos sp.
tagtcattacttttccattattacaataactctgaaacaagttataattgtttacccaatctggttgaagtaaaagtaaccataaagaaaacaaggcaaaaacactacagatcaacacaggtaactgtaaaatatttctgttaatgggcataacctaatactgctctggtaaagccattttctcttaataggatggattttctcttttggacttttgttgccattttccttcctgctgtaggaaagacttyctggtgccaaggaactgaactcctcacaaacatactcagtgactcaaatgagataaagggagattatgacaaagattaaagatgaactcagagtactcatatagttctgaaccctcaagtggctcacagttgggagaatagtggataaaatggatcctacaaaggtatatgttcagggtatatgaactctgtacatacaagttaatgaaacttttatgatatcaactactcaatccttcatggcatcttc

SEQ ID NO: 115 - DNA, 501 bp, Bos sp.
tagtttataatccacaaaatctcctattgctcttgagtattttaatgaatgaaataactactctgtgtatcaagattaagttattatggtttataataatacctctttttctacatcacttaataattttgtttctttcattgtaactccactgaatcctctgtgaaatgtactgtgggtgttcgtaaaatttagtacatggcaaagtctgggcttgaaattggctttcctcgtatccattagcaactctmttctagcatttcttatacagctttatgattttttaaactttctttagacataaacaatggaattctcaaatacagggacagtgtcttactcctatttatttctataaagtatagaccaaggtcccatcagtagttggtacttgatacatgtttggtgggtgaatggagagatgaaattttatctgatggtaatataaattttcatctctggagtcactaaagtttacataccattttgatttccagggta

SEQ ID NO: 116 - DNA, 101 bp, Bos sp.
caggttgcatgtggacactcagccacattaacaacttggagaaaaaaaatwaaaaaatggcaatgttataatgaattctcaggtgaacttttttttcagtt

SEQ ID NO: 117 - DNA, 501 bp, Bos sp.
ctccaataatgtttattcttcaagttccagaagggccttttttttaaaaaagccatttcactttattagaaatcaacggtgccctgtgggccactaggctggtctctaaatggaacttcagggcgtctttacgtttagcaatttatcaaggactattgaagactagtgctagagatgtcgcactgagcaaacaaagcctcatagaaacaaagctacatagttatcttctgggagtttttgtgtgttctcayggtaacatctcaggtttttgaagtgtgggttgtcccacactatcctaaagtacaaaagtcatgaggacaaaaattcccttctgctgttgcagctgtagggtcacctagtgccttatttaatcagctgataaaccagcatattcctctccagccataaagcaagataagagctcagaagtcatcattcccctcccttgcttctctctctacacatgtacctgtatctattatatacttaactcagagtctg

SEQ ID NO: 118 - DNA, 501 bp, Bos sp.
ttttttctttcattcagtgatatgctggagttattgtaattcttctcattttttttatctaatctcttagaaaatatggtcaccactgtttacagtactaaaaattaaatgttatttcagtacagaacacagcagttaatccagaaagtcaaaggcaggctattgatcacaattacaacccacttcagttgtgcacttaagtcatgttacttggagtgatgcaacaatggatgatccattattgtgaactrccaattatggttgcctacatcacctaagaaacagaaagtagctgtacagtatccatttgacactaacaacagtgttacttttagcccccttttgggggctaccttagtgttttgtacttctcagagccttctgtgttcatcaccaaaaattaggacctcttgtctcctgtggatactaactttgagtactaaaattggagcagccctaggcaagttgggacttgtggaccctgtgcctcctcccaagcca

SEQ ID NO: 119 - DNA, 501 bp, Bos sp.
aagaggctgtaagccaatgtcctaaaaaaactaaatggtggatgcatttctggagtagccaagaaaaagcccatttagccataacagcttttattaacaagaaaaacagagtaagaagaaataatattaaagatacaaagcatgatatggactgtcttattcgaatgattaaaaaatattatacttacctaatccattactctctctctattactaacactaacaggcctccccactcctccttttctttsctgggacagtggcatagacattttgagttcattgcttggattactgtttgtctaccactgaatcaggtttcatagaatagttaaaataaaaagattctggctattaagaaattccctacccatgaaaagctaactgtatagcttcaagaacactgggatcttagagccttcattagactgtgagatataggtttatctatcaaatttttatttaataatatttagtatatatttaataatatttaacagt

SEQ ID NO: 120 - DNA, 501 bp, Bos sp.
aatatgcctcatgaggaagggcagccaagggagggcatttttcaggccccttcccggttcagagcatcccaaagaggtgccaatcgtcatgatggcttctgtacccatggccatctgcttactgggctttgggcagatgccagcgtatttttctcttcatgacgatgcagtcaccattaggtagcagtgctgtcttctctccattactgacctggcaagattggaccttagatctcttgctgtaaacaatrtgggaggtggttctccttgatggaattgttggcttatgaatttgaatcaattatttcaacttttgtcttcagggtcctggtaaagagggactttttaaagaccccagtgtacatggatgtttgatgccagtttatatgagaatcttaactttgaacagcaggccaatcaggagagagattttagtctacatgtagccagaacctccacgaggccatattcttcccttgctgctggactctatgtgtgtaa

SEQ ID NO: 121 - DNA, 501 bp, Bos sp.
agaatagccctcgatcaccaaaaccagagaaaagcctgtcagcaacgaagacccagcatagccaaaaataaataagggaaaaaaagcagcagctgccccaggactctagaatcagaacctgaaaccaaaccgtgcccttaacatcagtccacatacaagtgctgcctttgttttccttatctcttctcctttatatagctccattagaaattagacttatatagatgacagtaattagggaatagtgtctrgtaacaagtatataggcagctccttggtagagcagaactggagtcagtcatgcctttaggaccatctaagtgtgggaaacagccatggccaatgcaaaaccaggtaaaaagtgccagcagattggcaggaagttaatctgaggattctggattttgaacttgaagggctaaagaattcatctatgacatcctctgctcaaaacagtcatttcagaagttctcctgcccattgatttctgatgttggagac

SEQ ID NO: 122 - DNA, 501 bp, Bos sp.
agtccatggggtcgcaaagagatggacatgactgagcgactgaactgaactgaatcattcccaaagaaagcatcataaacttatctatatcaagttgtcacttaggtgtggtatcagaaacacagcagatcatgaggctattcagggagcaagagtaatgcaactggtgatgtgcttggttaagtggtgatatgcggggttaagtcatggtagcatttgcctataaagccatctggtcctggacttctgtwttttggacgctttaaagccacagtttcaattatagtacttatgatttggctgtttatattttccatttctatctttgtgtagtcttggatggttgtacctttctaagaatttgtccatttcttctaggttgtccattttattggcttatagttgctttcaggagtctcttaattctgtccatttctgtggtgtcagttgtaacttctactttatcatttctaatttcatagatttgagtcctattggctt

SEQ ID NO: 123 - DNA, 501 bp, Bos sp.
aggccaaagaggtgtccactgaaccctgcacaatgtctgccccatggcaactgtccactaggtattcgttgagtgatgaatgaacaaagaaaacaatgccagaaggaagaagaatgggaataaacaatatcaaggtccttagaagatataggaatacattaaatgacccaatggggagaatcaggaaaatcctgtattgaccctaaggagattccctagaaaggggtcacactaacttacatgttggagtyctgatgttagtgcaacaaaggttctgttaatttttaagtgaaatgaaaaagtgattcccactatgttttctctttttcatacttttgtggggtaggattgtcatttagagtgaagaaaataaaagaggttaaggttttcataggcactattaaatatatgaaaaaaaggtagaataagaatagttttttttctttttcttcctttaagttgttctgatatataagtggaggataagtaggacaagtcttc

SEQ ID NO: 124 - DNA, 501 bp, Bos sp.
tcactgctgcatggtgaattttacaaataagagtttactcaacctattctacagttgatggatatttgggtgttttgactcttatgaacactgatgccaagaccagagatgacagtaggtgaggcaagaaaggtgtttggggtgcaaaatttaaggaagcactcactgatcaagatcactctggcacttttatgaccctgggagtgagcacctcctcaaattttgagcactaggcatcttcccttacttcyccctactcctggtactggccaagaacaccttcacctgtgactttgcctataggatatacctaggaatggaaccgatagatcacaggatccatgatgagcaatttgcagcaacatggatggacctagaaagtgtcacactgagtgaagtcagtcagacagtgaaggagaaatattgaatgacatcccctatatgtggactctaaaaagaaatgacacaaatgaacatacttacaaaacagaaagagactta

SEQ ID NO: 125 - DNA, 501 bp, Bos sp.
cccaagcaggctaaatttcccgtgccagagatagttttggggttggtgtcctgttccccagtttcctcctggctcccaacctccctgctgctttctaacctactctctctgctttggtgtctgtgcttctctttgtagttccttgcctggatctctgtctttagatctctctcttcacctacactcctctgctggtctctatttctctacatttgtgcctttttctgtgggccttgttttaacactcaatragaaacacacagggaagttactactaagccctcaggtgagaagctcaccaccagacaggcagcaaaggggctgaactgacccctatttgcattaagtcactcagtgttcctcactcttctttcccagcttggctggtatactcctaggcatgtgtatgtctgtgaatgtgtgtgtatgtgtaagtgtgtgcatgaacacagatgtgctcttctgtctgaggcaagactaagactagaggtctaaataaag

SEQ ID NO: 126 - DNA, 501 bp, Bos sp.
gtgaaacagttgttaaatcttttgcccatttttaaagttgggttattttcttactgtcgtgttttaaatgtcttttctatgatctgggtacaagtccttgtccggtgctgtgcttcgcagatgttctctcacagtctgagtttttattaagctcagtttattgtttttaacttttatggattgtgcttttggtgttatatctaagaatgatttccgtaatcaagttcatgaagattttctcatgttttttycctaaaagatttgtagctttaggttttacttttatatttaaaagggcttccctggtggctcagagggtaaagtgtctgcctgcaatgcaggagactagggttcgatccctaagttgggaaaatcccctgtagcaggaaatggcaacccactccagtactcttgcctggagaatcccatagacggaggagcctggtagggaacagtccacggggtcgcacagagtcggtttatatttaagatccattttga

SEQ ID NO: 127 - DNA, 501 bp, Bos sp.
cttcccagtattttaatatcaagcctgctgaggtcacacacagtctaatccaggcttgggtgcagttctctatgtgggcagaatgcatgtgtgacaggtttcagtgggagagatctaattcttggcacttttagttatgttgttcctgggactttaatgtgtcttgagttttgatagatcatttgcaaaattcgtggtagctatttctctggcaaatataagcccaatggatgcctaaataaactttgctstccttcatattgtactgacccttcatggctccttcatgggtccctgtggcctgattcaaggcaattaaaaagaaaaatatatctgcttctcatacctcccacgtatttttgcttgaatgggacttccatgaatgatggtcaccagcttggaccatctattttctttctctttttgattgtgctgagtcttcgttgcagtacatgggctctagagtaagtgggctcagtagctgcagtatgtctgtgggct

SEQ ID NO: 128 - DNA, 501 bp, Bos sp.
agatgatttgtcaaaaaaattttcatgtgattgagagttttttattgggaattgagggtggaaggagataatcgattatacctcccgatggaggaaaaatctgtgtgtgctgttcacgcagctaaggctcctctggctgctcagggaaaatgtgacaaaatcaaaatgtctggttgtgaaattatcttccagtaagtttgttgcctttattgtgcctataaaaaataccattcatatttatgtcattaatmaacttctgggggggtagtagtatatagtatagttaagataaaaaaagacaactggtatttttttgagaattgtttggtatattactagagtatgttttataagtgtaatagtattaggtttatatatgcagttgaatcatgcatatacttgcagcccctatgaaatttgatctgtctttattataaacattatataaataaacaaagtttaaaagaaattaaataatacaataacatctagcaagtggag

SEQ ID NO: 129 - DNA, 101 bp, Bos sp.
tttatagctttcgttttgataacatttggtcagttcattttaatagttggraaagcggaagtgtatgtgagctttttgtaaaggatttaaaagattaccta

SEQ ID NO: 130 - DNA, 501 bp, Bos sp.
tttcccagcatcagggtcttttcaaatgagtcagctcttcacatcaggtctagtcaaagatgatcagaatgaatcttgggctgatccgggaatgaagctgatccaggaatgaagaaacaagggcacaggtgccctctctgaacagggccaggagcggacagggtacctggagccactgcaaccatttgctcaggccaggccaaagggcatggaaacaccagggtagatgtgcggcccaggatagtcatatrcagcaaagtcaaggtcctaatgacaccacagcttccagaccaagtgtcacctgaaggccactctacctttggccttggagagttacatgactggtacatcccctctgttgtctgagccagtccgagctgatttcactgttattttccaagattccattctgttacatattctctccctgatttgtatttacagtatgcctaaggcaaggagatgaaatgaatgtttaaaatggtcctaacacttttatct

SEQ ID NO: 131 - DNA, 501 bp, Bos sp.
aacttgttcaatcagcatgtcatatgtcaatgtaagatacacgcttacacgtagttggctttttgtaccagtttcctcactgtgaatattgagagtctagaaaagtctattctacaatatctttgatagtatttataaattctttatgactattttacacatttgtgtgtgtgattttatttaatttgcaaataaggcaatagtgctacagaattactacaaaattgaaattgctaaaataataggaagcrattgaggcagctagtttcatctcatcattttgtatccttatttgggttgccaaggcacagcttacacacttagtgtgcttccacaattctttgccattctagagcatgtacttctgacatttttattaggcttttgtgtttggtcactgtattaaatccagcaatttattcctttccacttaaattgtcttccctgtacaaggggtggagaggaattcacagtgcctgcacataaaaggccattatctta

SEQ ID NO: 132 - DNA, 501 bp, Bos sp.
aaacactacagatcaacacaggtaactgtaaaatatttctgttaatgggcataacctaatactgctctggtaaagccattttctcttaataggatggattttctcttttggacttttgttgccattttccttcctgctgtaggaaagacttcctggtgccaaggaactgaactcctcacaaacatactcagtgactcaaatgagataaagggagattatgacaaagattaaagatgaactcagagtactcwtatagttctgaaccctcaagtggctcacagttgggagaatagtggataaaatggatcctacaaaggtatatgttcagggtatatgaactctgtacatacaagttaatgaaacttttatgatatcaactactcaatccttcatggcatcttccatataatattctgccaaagaatgtctctttttatattccataactattttgccagatctcaggaagaagaaggagggagatagggaaaaaatattctg

SEQ ID NO: 133 - DNA, 501 bp, Bos sp.
gggcgatcaggaaggacactgtgagggcggccctggatggatgtgtctttgtccaggtttctgaggggtgggtaaagtgaaaacaagccccctttgggtctctgcgacctgagggcccctctgttactctcactttccctctttcagcgttttcaaatctcagatgttttgttcagacgacgtgcctctggctctcctcatcaggaagttggaaatttcaggaaggaaatctggatcactgcatatcacarggaatggtgtatttttggatttcagaaagaatggggtctctggcaaggagtcactgagatcatgccttgcgaacaggaaggatgtatcgggtcacttacattgagccactggtgaacgtttaacgttagctgggtcttggggggaaaagccccaatttgtatgtagcgtttccctattcctgtggtgtaaatactcccaccagggcacactgcagggtaccaaatagctgtccttatacacagagctggg

SEQ ID NO: 134 - DNA, 501 bp, Bos sp.
  misc_feature (442)..(501): n is a, c, g, or t
gataaatttgtcaacccatatataccttgtgcatgttgcttcaggaaattcccaaacagtaaaggaatgggacaaaggctaacatatttcggcttaggccttttggttctccaccttaaaagagccaccttagaccacttgagctcagagttctccaaatccttataaagatcccacttctgatttcattctactaagttaattctaagactatcaagggggtgttttctcttattgcaatgaggtcaaayaaactaaaaattgctctttaacagagtgtctagtgatgattcacagagttttgtaaaaagtgtgacaatctttttgaagacatgttgagtgataattaaacacaatatgtgttgttaatgtagctaagttgatttaatggtaatgtggatagcaatgattatctttgcacaagtgtaggtcacagctactnnnnnnnnnnnnnnnnnnnnnnnnnnnnnnnnnnnnnnnnnnnnnnnnnnnnnnnnnnnn

SEQ ID NO: 135 - DNA, 501 bp, Bos sp.
tgacaaaaagtgcactagagccatagccatggggctgttctttatctgctaccaagactggctgattcagaacagtcctcgctattttctgaccttggcctcttcaatgatccagttcaaggctgggcttaatgactttgttgtttatctttttaatgtatgcggaagaaaattgtctggtatcagttttcaaatgaaataaattcaccttagtcaataccttcaacttatgctgtgaaagtaactccacstatatatgatgggtcagaaaaatatttctgatcctttaccttgcccctcttttagactggcctatcaccatttcaggcttggtttatttttgcagccaactaagcagtcttctctgcttctggttcttcttcaccttgaaatacgtccatttcactgttatcaaaattataataggagttgtaataacagacgattatttctgtgctcagaagagtattctgtgccccaccatgcgtaaaacaaagaata

SEQ ID NO: 136 - DNA, 501 bp, Bos sp.
tacctgcacatgttaccagatgttttatttaacagatttggaactaaaaatcaatttggttgtcattcattgcccttttgaggatttagccataaaatgaatcaatgaaaataatagagagatatacaagagtctctatttagcattcaatataaaagggattttttgtaacataaatctataaatgtgacaggattttttatatagattaatagttaataggtaagtacacatattcataaactcctaamagttcttaagggtatatgcatgcatgctcagtcatgttggacactttgcgaccccatggactgtagccctccaggcttttctgtccatggaattttccaggcaagaatactggagtggcttgtcatttcttaaatcaggggatcttactgacccagggatcaaacccacatctcttgcatctcctgcattggcaggcagattatttaccactgagtcatctgggaagccctacttggtattaaaaatatc

SEQ ID NO: 137 - DNA, 501 bp, Bos sp.
tataggattaagaaaaaaatagctattaagtgttatctatccaatcacactttttctgcagctgaactacattcttatgactgtcagtggtgattgtgattttgcttccatctttggctgacgaaaatttggaagttacaaagctactgttgtgtacttcctgagtatggccccaggtaggcactgaaaggcttttcaattaccaacacatctaggtgtttcttaaacatacaatctatacaaaaacttcsaccaggcaatagtggcagtaaggtttatttttgaatggtcacaatcaactaagaagaatgtgcacttacacaaaatagtaaataccaaaataagtgtcagtgctgagtatcgactatgtgccatagactaaagttctctggaggttcaggagaggaaagaatagttaaacagtctctataatatactcagattttttagtttttttttttttttaatatatagtatgggagtagatgatgtgaaccaaga

SEQ ID NO: 138 - DNA, 501 bp, Bos sp.
aaacagaaataaaagcaattttgcttatagagttgtggtttccattgctactcataaattcgtatagtattagtaataatagcacctcctgaaaatttaataatacctttgcatttgaacagggctttttgagatttttcaaaggcttttaatactcattgttaagaggtcaccagactattgaaaaacattgctacaataacatgaaagaccaatgcagagaaagcctgaattctatctgtatatctagmatttattaggcatccatcatttctaagttttgtaaagaaactttaaagacgcaccctaggctcaagactcgtgccatctaagagtttagatgtgcaaacacatccagctacaaattaagagtatgaaagcactgtgcaaacaatgcctgcctaaaacatcccggcaaccaggcaacaggccaggaggtcattctaggatttttctgccttaataataatctcttcaaagtgagcagtgccttagaggttc

SEQ ID NO: 139 - DNA, 501 bp, Bos sp.
caatggacatgagtttgaataggttccaggagttggtgatggatagggaagcccagcgtgctgcagtccatggggtcgcaaagagatggacatgactgagcgactgaactgaactgaatcattcccaaagaaagcatcataaacttatctatatcaagttgtcacttaggtgtggtatcagaaacacagcagatcatgaggctattcagggagcaagagtaatgcaactggtgatgtgcttggttaagtgrtgatatgcggggttaagtcatggtagcatttgcctataaagccatctggtcctggacttctgttttttggacgctttaaagccacagtttcaattatagtacttatgatttggctgtttatattttccatttctatctttgtgtagtcttggatggttgtacctttctaagaatttgtccatttcttctaggttgtccattttattggcttatagttgctttcaggagtctcttaattctgtccatttct

SEQ ID NO: 140 - DNA, 501 bp, Bos sp.
aatgtttgcacatcctcttgatttgatccccacaatcaaccccatggtgacaccgaagcgttatctctcccattttagaaagaaggaaagaattcacatttcacagagcctttattcctagacacattctgacatttttttcccccctgagcttaaaataggtaattaaatattccatagttgtgctatggactgaactgtccccacatttataagttgaagccctgaccctcagtgtgactgtatttggrgatagggcctctaaagaggtcatgaagaggttatgtgatggggccccaatgcaaaagggttagtgtccttctaagaggagacaccagagggaatacacagggagatggcagccacctacaagacaggaagaaaggcctcactggacactgaccccatgggcaccttgatcgtggacttccagcctccagaatttgtgagaaaacaaaagtttggtttttttttggcgccatccaggctatggtacagtat

SEQ ID NO: 141 - DNA, 501 bp, Bos sp.
caaccaacactgaagggggtaaaaaacatggaatccatgtattcaatgaaatactacacagcagtgaataaacttgataacagccttaagcatcacatggattaaactaaaaaactagtggtcaacgaactgacttataagaagacttacactgacttctatataatgtttaaaatatacagattcaaataatacttggttaggactgaatagagattcataaagatggcaaagactaacatggaaagcaraagaatgatcagtataaaacaaaaagggatttactttgggaaagagcaagggagatttaacaggctgctcaggtgaggtaaagttctatttcttagcccaggtgttgggaacatagatgctcagcatattatttttccttaactgtaaggtatactttcatatactcctgtaatcttatatctcacatactggaaaaccaataaactgctgaagtaattgtaattgaagatattttaaagatcagacaac

SEQ ID NO: 142 - DNA, 501 bp, Bos sp.
caatggtgtcataacaagttgaaaagaatttttacgactgaagtgtctgaatcatgaaaaaaccttacttaacttgcatgtttgcagattcggtactgggcttcccatgacaaagaagcagctgcacacagagttcaagttgctagccaagaatactcggccaggctggagaaccttctgcctgacacccagtattttgtggaagtcagggcgtgcaatagtgcagggtgtggacctcccagtgatatgaytgagaccttcaccaagaaagcacgtgagtctcaatatgtgtgttatatacttgccaaaattcccgatggaatgatacaggtacaggtgagagaaatttactactagcaatcagcacatttgggtttcaactttagcctttaaaatcatataagttttaaaatacatcaaaaacaacaaaaccattgttagttattgtagatgaaaaaagtggcaattactttcacataattaatatccatttcagaaata

SEQ ID NO: 143 - DNA, 501 bp, Bos sp.
accacagtgttcgaattcatgatatgagtggactatttctgagggggagaaaaaaaaatcatcaatgatgatccttttgactcctactaatatttagccaaatgacatagttgccagaggctagacctagcgaaatctaatgaaaggggtcacatttcagaataattttgataactattcaagccttaatgagctcttctaaaacaagtaaagccactttcactcaaaggcataggagacaattaggaagmatttttttcttgcatttatctttttgaaaaacaattgttgattggtttagaactttaagcagtaatttactaagggatgaaaggattcctaaagtttctaaaatgtctattttgcaaccagatgtgttttaaaaggtaaagttaatctgtgcttaaatagaataatattagggcagatcagatacttgaagagccgtgtaatacactccctagtacctaagcattcaccatgtgacttggtggtcatcat

SEQ ID NO: 144 - DNA, 501 bp, Bos sp.
gattttttctttgtctttcaacatttttactattattagtctagactagaatctctttcatttatcctgtttagagttagttgagattccttcatgtgtggattaatgattttcatataattttggaaactttctgttattatttattcttctttttctgctcctgtttctctcctctgcatctggtaagatttctctgaggttttctgagtaattgttatattgctttcttttattcactttgcaaaatytttattgatatattttcatatttgctgtttctttctcctgttaacacaaatacactctttaaatttcagaatttccatttggttctctttcatatttctttctctttactgacattttctatttgataaaacatggtcattatacctttctatacacttgtacacaaaggttcttttagtttttttgaatatatttataatagtgatgtcagtattttatctgctagaacaaagcatgttttgtatgctt

SEQ ID NO: 145 - DNA, 501 bp, Bos sp.
gttcagactattggaaaatgagctctcggttggcagggatattttcatgcttttgccctcctttccatttacttgcaattagggtgatttcctattttcggccattgcgtttctgtgtgttgaggtctcacgtgaagcttgggaagaggggaggggcacacatatggcttataaccgctttatccagcaatgaccctggtcccagagaacatgactgatcttggcttcttcagttcttttctgtggccagygaagggataggttgtcctaggagtggccacttaaaggtcacacccttcgccaactgcttaagtattattctggtgatcacatcacttggcaatttctgggcagctactcaacttcctctctcaggaaaaactccaaaactgaaatggaaaagggcctgatctttttaataaaggaaaaggagctgaacgtctgttacttctgcaaaacaaaatagattctagtaaaagagagtgtttataggaaatggag

SEQ ID NO: 146 - DNA, 501 bp, Bos sp.
tacaaaagaataaatttatgtatatggcatatgggggcttccctggtggctcagtggtaaagcatctgcctgccaatgcaggagatgcagattcaatccctgggtcaggaagatcccctggagaagtaaatgcaactcaccccagtattcttgcctgggaaatcaaaggacagagaagccttacagcagacagtccacggggtcacagtgagttggacccaacttagcaactaaacaacaacaatacgtaygtggataactgaatcacttggctgtacacttgaaactaatacaacattgaaaatcaactatactttaataaaattaaaatattaaaaataaaatcaaaatattaacaaaaaaataaaaataaaaaccaagaaaacacacacacatacacatacacaagaactgattgggagaaaagtgttggaagttcagattaccagtctccagaacagagatgctctgaatttctctgggcttagttttgttttattt

SEQ ID NO: 147 - DNA, 501 bp, Bos sp.
atggattgcagcctaccaggctcctctgtccatgggatttgccaggcaagagtattgaagtggggtgccattgccttctccgatgcttcatgctaggttactgctttgggtactgggcatcttatagcaccatgcaacacctgaataatagccatcaccttaccagagatccaagtactcaaggccctgtatattttggccattgaaatgataaattattgaaaaaacagaaactaatttcttttccatgyttcagttctatgtcccaaacaaaagtacaataaattaaaattgaatgaaaattgtgataaagcctcatgtctaatgaatttgagatgagaaggaattatgctaatttgggtacttggttgtacccatcatgttaggctttcctgaatctgtgtagaaacacaggtcctatatgcaatataaacagggatattactgctactcagtcctttgccacaaagagcaaataatttgactgactatggatatgtt

SEQ ID NO: 148 - DNA, 501 bp, Bos sp.
ctttttaatcatcatgcccctgattgctgattatttactcttctgctgtttcctctcatgattacacagcctgctatggagccaggcatcatgcctctgcgagtccctgtagcatctctctccactttagtttgtcataaagcagctggtttcttctctcatccatccactaagtatgccctgcagaattgtctaaccaagattctacatgatatggttgcattctactcacagcaacctaaaacctacayggctatagtttcaatacacgggtctacagcagaaggaagactactgttattttatagattgttctggaggatgctgctacactggtacatgatctcaaggtgttgaagatattggcctactacttgatttatacacagcttccataaattttagataatccaatgacaactttcagtgctacaaggctaattaatttttttaaaggtattcggaatagcaaatttgctttatataaaggccttagcaatg

SEQ ID NO: 149 - DNA, 101 bp, Bos sp.
acagacaactgccacaagtttgttgacattcttggcttacggaccatcttycccctttttatgaagtctcccaggaagatcaagaaagtgggaaccactga

SEQ ID NO: 150 - DNA, 501 bp, Bos sp.
tttccttctccaatgcatgaaagtgaaaagtgaaagtgaagtcgctcagtcgtgtctgactcctagcgaccccatggactgcagcccaccaggctcctccatccatgggattttccaggcaagaatactggagtggggtgccattgccttctccactgaggcatgaccaaatcgtattcagcagtcagagtgatgtctaggagaatgttggaggtgtctacccatgaaaacagtgtctacccacgaaaacytgaatttggtctaaagtcttttccttgttcttagtggatctgcatctgggcaacctaaatattcattttgatttcttgctttctttcttttttttttttttttactgggaggaatggttataaagccagtgtcattttatttttaacagaaaattttctaagggagattcctagaggcacaatataacacttaacgttagatttgtgttagagaattaaatgcagatctcagatttataattgcaaaata

SEQ ID NO: 151 - DNA, 101 bp, Bos sp.
ttccgggaccagatcctggctggaagccctgaagccttcttcgtgctcaaygctgacgtctgctccgacttccccttgagcgccatgttggatgtccacag

SEQ ID NO: 152 - DNA, 501 bp, Bos sp.
cccattctgcagcatatgtgggcccctttcctgtagccacctgagggcagagcactcatgaggagatagctcagagagggtaggtgtcttgtctatgcaacacagctaacgtgtgttggaactagaatcaggacccaggtctcctgacaaaatcactgcatagttttctttacacctttgttctcagtcagccagccttcagtgtgatgatacctaagccgatgaacagaaggtactgatcattcatcgartttctctcctaccataggctctgtacacatatcacctcttttagtccaagggaagcattaatatccacattttacaaattaggaaatcagaaccctggattctggattagggctccaatagggttgttgagaaggcaatggcaacccattgcagtacgcttgcctggaaaatctcatggacagaggagcccggtaggctgcagtccatggggacacgactgattgacttcactttcacttttcactttca

SEQ ID NO: 153 - DNA, 501 bp, Bos sp.
cctggagaatcccacggacagaggagcctggtgggctacagtccatggggtcgaaaagagtcggacatgactgagagcctgagcacccactggctcttctacgggaggaaaaagttttcttctacccatttagggttcctgagtgggtctgaaagttacgctgacaaagacagaccaacaggaggaaagcatacatgttgacttaatgttttacatgaaatggaaaccttcacaaggaaacgaaggtctgyggaagtgtttggatggacgtgagggttttcacactggctctgatgaggaatggacagtcctggaggaatgcggctgatcaagcagtagcaggtaagtgtcgccgacagcgcctgttggttgggatttcttttaccttttcatcttatattgtagtacagccagttagcagtgttgtgacaatttcaggtgaacaccaaagagatcagccacacatacgcatgtatccattctcccccaaactcccctccc

SEQ ID NO: 154 - DNA, 501 bp, Bos sp.
actttattaatagacacacagacacttgtgagtattgcagatcctgcttttctagatgtaggatggagttgaggttctgcattacctgcaagttcctagatgaaccatgcttctggtccatgggccactctttaagagtataaagagatggctcccaaacttggttgcgtattagagctgcttggagagcttttaaaaatcaggatgccaggccacacccctgaccagttagatcagaatctcaaagtgaygcctaggaatcagtatttttaaattttttatagctgataagggcttcctaggtgtcgctagtgatcaggaacccacctgccaagcaggagacataagagacactgttgccatctccaggggtcaggaagatcccctggaggaggttatggccacccatttcagtattcttgcctggagaatcccatgaacagaggagtctggtgggctacagtccgtgggggtcatacagagtcggacacgactgaagcg

SEQ ID NO: 155 - DNA, 101 bp, Bos sp.
gtcattgcaatccaggcccattgccagggctggacaggaaggggctctgamgctgatcagctctaacaaagccatgattccggagatgtgattggtatatt

SEQ ID NO: 156 - DNA, 501 bp, Bos sp.
taaggtctctgtcataaccactcaactctgctgcagaaggaagcagccattaacatgtaaacaaatgagcctggttgtgttcaataaaaccggactggcagtgagccagatgtggcccacaggctattagactgccaactctgaactagaactaaactccattttaaaatctgccagatgtcttctcaaaactagctaaatggaaactgaccattctctgataaactgtttttttgacaaaaaggagtcasagcacttataatgaccattttaatcacaaggtgttaacgttaagagattaacataaacttcagatgtcatagaaacagttcaacaaaatacagaaataacttgtatcaattaaaccggagcaaacattttggagacttcaacgcactggatggaattcttcaaaaacttccccgtaagttttcttatctataaaaagaaaaatcacaaaagattttaaaatcatgtttaaaaaatcacacttcctaaaga

SEQ ID NO: 157 - DNA, 501 bp, Bos sp.
cagtctcaagactcccccagatctactgagttggatcttgcgttttaataagatcctcagatcatcaataagattcccagatccccttggctaaaaggtgactgcacaaagccctgtctcaaaaggggtgccccatgcttggggtataatgctctgtggtgactgacttgacattcttgctaattttatctttgaaattatattttgtgagtgaaatctggtgggacactgaaacttgtgagggaagcggrcttgaagtcttagttcaaacatctgtagttccacagctttccaggatggccactggctcccctgccccactcagcatccactactgccctgtgccctcggcagggaccttgaacattggtatggtgacgtcaaggtcagatacgaatgccccatgtgctcaatcatgagtgatgctttgggcacctttgaaggcctgtttctctgtcggcatccctgtatgaaagcagcacagtgttaaatggcaaataa

SEQ ID NO: 158 - DNA, 501 bp, Bos sp.
cacgctgagcatcacctccagcccatccttgatctcccccttcccctcctcgcagcacgtgagcagctcctgtgggagaccaagaccgtgagatgttccactaaggaggtcttcacagaaaccacagagtttgtgaaaatccgtgacgtttgtggtaagaacagatgtggtcaactgggcagagattctcataagggaatgcaggataaatttgaattgcctctttagccagtggatggaagggtttgtcytgctcaagtcaggatgtttcttaggcttcagaggaaaccaaatgcagtcatagttgaaggtgagtttagtgtaatgttaactactgagcctcaaaaatgacaaacaatttaatcagggtagcaaagcacagtcacctagaggttacactcgaaggtgaagtcagagtccagtgagtcctgctctgctggccacaggatccagcacgatcctgagctctatctgcctgcaatgcaggagacccaggttcaa

SEQ ID NO: 159 - DNA, 501 bp, Bos sp.
agtgactaaacaacaacaagtggataatacctatgtggataaatgagagctgatcccatctctttaaagagatctgtaccaagccatatacatatttacatgtgtacataaaaccaatttacatttataatacagtcactggagtcttatgagaatgttttaaaacgtactggacgattcacttggtctgataatttagcttcttaacattgtatttcttacatatcattataattctgagtgaacgtaaygcctattagtatgaatatgctttcaggcgggttatccaggaacggacatagtactggctaaaaagcagatgatgaaaaaaattgagaaaaaagacgacagggggtacttccctggtggtccagcggttaagactctgcacttccaacacagggggcaggagctccatccctggttggggaactaagatcccatgtgccatatggagcagccaaaccaacaaacaaaaagacaatggggacccctggacaa

SEQ ID NO: 160 - DNA, 501 bp, Bos sp.
cttccatggcagggggcacaggttccatccttggtcagggaactaagatactgaatgtcttgtggaatagccaaaaaacaaacaaagaccacctctctccttcttatagactattagtctgacagacaatccctaaaatttctgggtgtgggacctatggtgtatgccagcaaactataaatgactaacatgagttgaaataatctgatctctctcagggactcctcaaggaaagcactcatgggccaagrctgtgtattattttttgctccctcccctgcatctggcacacaatacacactcattatacatgtaggttgaagtagaaaactattccctaagaaagctgtttcttgagaattgatttggaattagaaatcttggatgatctgttgaactgttttctgtaatcttgcctcttcttacattattttacagggtatatcccacttggggaaagctgagcagggcttctggggtgcctggtcttaacctgggcag

SEQ ID NO: 161 - DNA, 403 bp, Bos sp.
ccctctctgttaatccctcacctgcctagaaacagagccacagatgtagaggactaatatatggataccagcgaggaagaggggtgggatgaaccgggagatgaggaccgacacgtacacaccactgcggataccacggggcttcccaggtggcacagtggtaaagaatcaggagatgcgggttccaaccctggatcgggaggatcccctggagtaggaaacagcaacttgctccaatattcatgcgtggraaatcccatggacagaggagcctggtgggtaacagttcatggggtcacagagagtcagacatgactgagcatgagcgtgcacacacacacacacacacacacgtacaaacagatggctaatgagcacctactatacagcacaggcgggggag

SEQ ID NO: 162 - DNA, 501 bp, Bos sp.
catctgtgttcaacattacgaaggtgagatccccggctccttcgttgactcggctggcatgtgtcgatgggcgtgttatcacacaccagacactaacacgtgaggagcggtggctcttataaaagcacgtcacgggggaccctgaccgaacgcggatctaaggcttccgtgagctgtgatgctcggatcgagttccgaaggatggctacaaagtaaccaggcatggaggggaggcacattagagggcagcrtgcttcagccctggagaaggaaggaggccgggctggaagcgcctgcaggtaaggctgggtaaatgtgagggaccagattgggccgggttcctaccatttaattctcacttacttccacaacgactctatagatggcactcccgttagacagatgaggcaacgttatgtgcacaggagagtctgctcagaagccatcatcctaccctttttccttgccatcagatcttgcctttcaccctagaaacacaaa

SEQ ID NO: 163 - DNA, 501 bp, Bos sp.
catctagaaaggggaaatcatagctgtattttaaaaaccaagtgaaataattttctaaaaacagattttaaaaaaatttaaaaattgcttaaagcctcaaaaaccaatgccaatagttaaaaatatctcatctccaagttcttgcttacctctgattgaaattctttttataaatccagtttttctttttttatctttagccgacacagaaagcaactaatcgctgttctcagagatttaacattcatacratataatgatcttactggagtttctttctttacagtttaaacagaatagattctttttattaaactgttaatcttcacaattttttaatttctgtaacaattgaacagactattctctatttatgaatcccatcactcaacatctgtgaaactgataagataaccctctctagggttttattttgtaacctttatctcctgaaacatactggtcattttgcccaactacagaatgcaggaaatgtggatt

SEQ ID NO: 164 - DNA, 501 bp, Bos sp.
aagctgtggaggagagagcgagctagggcagggtaattgaaggctttcagtgttgcttgctttcagatgtgagtgatttaagctttttactcgctgagaagaaagagaagagaaagtttttgaagacataaaaagaaaacagaaaactatgtggttagattgaggttcagagctcagataaaagaattatgcttggttggaggagagagaaactctgtgtgtgtgtgtgtgtgtgtgtgtgaaagagagaragaaagcgagcacgtgagtgagagagacagcgagagtgacaaagtgaaagaagatggatggaaaagggcaggttattttgcagctagaaactctggaagtggctcagttcagaatgcctcattttctctgaaatcacaacctccggtgtggatggtgagtggacgagatacagagactgcaaaattgttgtgaagaataagaaaaagagaaagaaaagcaatagacgaggacattcaaagactgactagg

SEQ ID NO: 165 - DNA, 501 bp, Bos sp.; identical to SEQ ID NO: 109

SEQ ID NO: 166 - DNA, 501 bp, Bos sp.
cctctaaaccaccagtgaagcccttctaatactttaaatggctctatttcatctccttgactgtgaggagcttgacagcagattctgttcttgaaataagaagctgccccaggggcttccctggtggctcagtggtaaagaatcctgccaatgcaagagacaggggttttatctctgatccaggaagatcccacatgccacggagcaactaaacccaggagccataactactgagtccatgtgccctagascccgtgctctgcaatgagaagccatgacagtgagaagcccgtgcaccgcaactggagaatagccctcgatcaccaaaaccagagaaaagcctgtcagcaacgaagacccagcatagccaaaaataaataagggaaaaaaagcagcagctgccccaggactctagaatcagaacctgaaaccaaaccgtgcccttaacatcagtccacatacaagtgctgcctttgttttccttatctcttctcctttata

SEQ ID NO: 167 - DNA, 501 bp, Bos sp.
tagcaataatgtaaagtaaaaaccaagttgtagaacaacatacatagagataccatatgtataaaacaaagagacctgtatgtgtacacatacacacacacatattttttcttcaatgtttacagaagtctggaaggatatataccaaacttaaaagcaaaatttccatataaggagaaaaatggattgcagttaaaggtaaatatagagaaaggttttattaacttgatatatactactatattattaayaaaactttttggatcaacaaccagtattgcttctcaagtaaaaagtaattttaaaattcctacctggtcaaaggtaagaaaaatctaccagcattttaaccttggattctgaagtacgaagcttttttctatcagaagtgttgagaataacaactctggtattctttctagccccacttttactcctacccccaggaaaagagtagtaactcttataattgaaatggaagacatgctgctcttttcctgc

SEQ ID NO: 168 - DNA, 501 bp, Bos sp.
ctgattgctgattatttactcttctgctgtttcctctcatgattacacagcctgctatggagccaggcatcatgcctctgcgagtccctgtagcatctctctccactttagtttgtcataaagcagctggtttcttctctcatccatccactaagtatgccctgcagaattgtctaaccaagattctacatgatatggttgcattctactcacagcaacctaaaacctacatggctatagtttcaatacaygggtctacagcagaaggaagactactgttattttatagattgttctggaggatgctgctacactggtacatgatctcaaggtgttgaagatattggcctactacttgatttatacacagcttccataaattttagataatccaatgacaactttcagtgctacaaggctaattaatttttttaaaggtattcggaatagcaaatttgctttatataaaggccttagcaatgaccacttatacccggacat

SEQ ID NO: 169 - DNA, 501 bp, Bos sp.
  misc_feature (449)..(499): n is a, c, g, or t
gacggaggagcctggtaggctgcagtccgtggggtcgctaagagtccaacacagctgagcgacttcactttcacttttcactttcatgcactggtggaggaaatagcaacccactccagtgttctcacctagagaatcccatggacggccaggcctggtgggctgccgtctatggggttgcacagagtcgggcatgactgaagcgacttagcagcagcagggaacccaagcctgaattttgcccgtcccckctgctgctattctaatggctttgtccttgggcccttgatgaggaaccacatttgagacaacacaatgttttacctgcaagtccattcattattatcctatgaaaagtcatgttttggtaagggaagtgttgcattgacaagtgtttttaagcaaaccagatgctaggggtttccccacctcttctggctctcccaggnnnnnnnnnnnnnnnnnnnnnnnnnnnnnnnnnnnnnnnnnnnnnnnnnnnca

SEQ ID NO: 170 - DNA, 501 bp, Bos sp.
ggtcacaaagagtcggacacaactgagcgactgaactaaactgagtgcagtgataaggcagagtcctcgttcctcctcaagggatgtacactgaacagtctcacacagatctctgagttcttctggaggaataaggcccccacccagatgaaggatggtgacttgggactgaacacagtatgtgaactcagaactggctggacccagaaggctgatgattgagatcctggaacctcaccctgttacctccycaccaaccagtcagaggaaggtcagacactcggcagccctcccctccaaatttgcccttccttgataaccgttggggagtttgggtcttttcagcgtgagccgccagcattctttgctctgcccaacaataaacctttctctgctccacactcagacgtgtccgtttgtttggcctcaccgtgcatcaggcacatgaatacagcccgtaaatgacaactcagtaatgctatttttcagaacagtgaactg

SEQ ID NO: 171 - DNA, 501 bp, Bos sp.
tcactagctacctggaaggcattggaggcactaagcaccattggcaaccccagttcattttgagtttctaagggagtggatgaaaaataaagctgatctcgattccatttataggacagatttgcagaaaagtaggaaaatggttgacaccccatttcattctggtgacatattggagctgcttgctcataacagtgttgtaagtgtcctacaaacaaggcgatttgcactagcaaatgatatgaatgtcrtagcccttgtgcctgactacagtgggaattccgaggaactatcttgagaattcccaaagctattttaggctaataatagaaaacccacaccaagcacagtgttttagtttggacaaaggttattctaccttaagcagtggctgaaaggcagtgattgatgtttccccatcatgaagcacagcatgttagttccttacttcgtctcaaatttattgtcacgggaggacatttgtttgttacagtatgccaa

SEQ ID NO: 172 - DNA, 501 bp, Bos sp.
cttgacccctaggtaattttttcctaaattaaaatagtcaccttatacaatttgtagtgtagattagtttaatgaattaggccagatgatatatataaagttcctggttctgggaaagatcctagtaaatacatatttttgttattgtctataatcaaaatagttaagagtagaaattaatttaaaagttctatccgtgaaaatttcaggtgcatctggaacaagagggacgccatttaaagttggaacaytgacatctgatagactctcagaataaactgggcttccctggtggctcagatggtaaaagtgtctgcctgcaatgtgggagacccgggtttgatccctgggtcaggaagatgccctggagaaggaaatggcaacccactccagtactcttgcctgggaaattccatggatggaggagcctggtgggctacaatccatggcgtcacaaagaatcagacacaactgagaggcttcattttcactttcagaata

SEQ ID NO: 173 - DNA, 501 bp, Bos sp.
  misc_feature (379)..(428): n is a, c, g, or t
tcaccatatgccataaagcattggctggcaaactttttttctataaatgggaacatagttaatgttcacacttgtgcaccatatcatctctatcacagctacttaactttgcagccaaagaagtcacaggccatgcacaaatgagtgtggctatgttacagtaattctgtattttcaacaacagacgtcaagacagattttgccctcaggtcatgctttaatgacccctctgatgtaatggaaaggttggkgtttggtgatagagtttctatcctggccggccatttgtcagctgaatggcgttggatgagtcattgaccctctctgagccaccatttctcagctgctaagtggagaaatggttacttaccttattagnnnnnnnnnnnnnnnnnnnnnnnnnnnnnnnnnnnnnnnnnnnnnnnnnncctgcaatgtgggagacctgggtttgatccctgcgttgggaagatcccttggaggaaactgtggcaacccact

SEQ ID NO: 174 - DNA, 501 bp, Bos sp.
atgtgcagaaccatctggaaacaacgacagtatgttttaatggtcaagaacttgacatccaatgagaagtaaataacccttgtagtttttaagtctcaaagcacatataaagcattcactgcattatattgattatcaatagaagctgatgatactgatttttagttcaaggtaaactgggaaacctaaaggattctggatgtacttgaggacacagagtggatgccatccaactgaactttttcatctayggttcactttgcggggaggaagttgactaaaacctatgtgtcaagtttgaggctttagggagaactgagacctcatgctctttgtttcttcctagtagaaaattagtggttaatatggaaggcaatcattgattttttaaaaaattctgaacattgttatttcatttattcaacaaggatttatagaatgtccttgttgagaacattctataagaacttatactatgttcaataaactgttcatatagac

SEQ ID NO: 175 - DNA, 501 bp, Bos sp.
atctcaaaaagcagagactgtagacaacatatgtcttcattcttccaccttctggagcacttaggtcagaagaactagtctgcttcctaatttggtaagtcacaagcttatatcatgtaatgtttagaagatctcagtaaaactaaaatgtagttacctatagattgtttttaattcttcattaagaaatcaattccagttcaactcttgttagtatgatctttgatattccatgtctagtccatagaggrgtgaagagagttccattatgaggctaaacttgaggatttagtaactgaaatgccctcaggaaagtttgggtaccttgagccctggttggattactgtgttccacctcccctggaagccagtgaggacccagcctcagactctgtcctgggggctacggcctgtttcttgcacttctgtgatttcttttcccattgtgagcagtggtgtccagcatatggccaaaagttttcagttaaacacactatatta

SEQ ID NO: 176 - DNA, 501 bp, Bos sp.
aagatttgtgtcatggatgtccaacatactcattattttccagatttccttttagccttcttcctcttctctgacatccctggttgccaaaagatcttcagtttggtaaccatccaggcacatgccctaaattctgaaaatattctgatcactaatatatattaccccattccccatttaagtctcttaatgttattgcttaaaatgcagtgttttttaaagaagcccttaatgtaagactgtatgtaatrtaaagaccccttaatgcaggggaatgcaaagcacatggactttaaacaaaatttcaactctgatactttaatttatggctgagtaaattacttaataaatgatcagtctcagtattaggctcaccatgttcaatcatggttttaggtgcttggtgcacttcaatttatgtagttctcataacaaccctctgagaaggtcctaattatgacaagagaattgagacacagagaatgtaagttactttcccag

SEQ ID NO: 177 - DNA, 501 bp, Bos sp.
tgctagaattaatgttcatgaccttgggggaatgcaaaaaaaaaaagccttaggagagtctctcaaagagtaggccttttggtagaattccaaaaggccgtctacaaaatgcttgactgggaatgctgtctagcggggtcttaggttctgtctcttacaggtatgtgttgagacggattctccgccgggctgttcgatactctcacgagaaactcaacgccagcaggggtttctttgctacgttagtagaygttgttgtccagtctctggtaggtgtatttcctgctcacctcttccgtactaaagtgcataccttaattggggtagggtgggcgatgtcacactgttaatgagaatttttagaggagtcatgatataatccttgtctttattggcctaaaatctgtgtttcccatttgccagggagatgcgtttcctgagctgaagaaggacccagatatggtgaaggacatcattaatgaagaagaagtgcagtttctc

SEQ ID NO: 178 - DNA, 501 bp, Bos sp.
cttcatgctgataagactatttccatatacaaattctgtccagagggacaatggaggaagagaagcaaggttgatggagaagaaagagggactatttggttctgcagtaggacaacctagagtagcctgaagactagaagagttaagtagaataacttaactatttggcccctttattgtttgtgactatctgtcaaggactaagaattccaagcaagtaaaagccaacttaggaaagacagcccagtgaygatttttggtggccccaaaaatctgctggcttctttatgaaatatgaacatttactgctgtctgaaaagagtctgatgctgaagctgaagctccagtactttggccacctgatgtgaagagccgactcattcaaaaagactctgatgctgggaaagactgaaggcagaagaagagggcagcagaggatgagacgattggatggcatcactgactcaatgggcaagagtttgagcaagctcaaggagatag

SEQ ID NO: 179 - DNA, 501 bp, Bos sp.
tgccccggactcgtcggcttaaaaaaaaaaaaaaacagctttattgagatataatccacataccataaataaaccactttaaagtgtacaaatcagtgatttttagtatatttctaaggttgtgcaaccttcatcactgtctaatttcaggacattttaaatcaccccaacaagacacccccccaaacccattagtagtcaatcctcactccccacttctccctagtcccttgcaaccactaatctacttwctttctcttgggatttgcctcctctggacatttcattaaatagaattaggcaacatgcaaattttctgtcttaaagtttcatttttttctgttatcaattattgttattattatcatgattattattattacttttggccacagcttgagggatctaagttccccaaccagagatctaaccctattcctcagcagtgaaagtgaggaggagtcttaaccactggactgccaggaaattctctcctgattt

SEQ ID NO: 180 - DNA, 501 bp, Bos sp.
taagccttataggagaacttgacctaaataattatggagtggtagtcagtgttctaaagtttgtgttaataaaaccttgtacatattcacagattaataatggactcagcgaatatttactgcacaggtattagcgaatgggcccctatcactggaacaaagacagactggtccttagaattcccattgttctgttagtatcacatagagtaagtgatgagtctgggataacaaaactgacagaaacagaygcagagaaacccagagaaggtgacgcttcattgtgttagcagtgggatgaggtcagaagggagggagttggggagagttgccctgaggaaatgaaatttgaacaagatcttcaggaagtttggaggtggctggggagagagggcatttcaggcaaagaggccagcacaggcccagccttggaggtgtgacccttccgtttggccttgtgagtgttgatttatggggcgagggagaggagcagaccaggag

SEQ ID NO: 181 - DNA, 501 bp, Bos sp.
actgtaatccatggggttgcaaagagctggacatgagttggtgactgaaaaacaacagcaacaatctataatatagttaccttgagactagaggttaggtgggagaagtgaagaggagggattataaaggtacatgatgaaactttttgaggtgttgcatgtacttattatcttgattatgatgatggttacatgaatatatacatatattaaaatttattaaattttatacttcagaaaaacagatcacraaaggaaacactggtctatatcatataatatcttttacattggtagattagattactgttcaaaaatattctctatattcatggaaagcgtatatttcccctatcttcaacttgaacttagctctgtcactttcttttgccattgaatattggcaggtataacacaaggaggttgaaatgttgttgtatatctggactccttctctctttactttgtatcattaggagaacggcttcttctgggtcactg

SEQ ID NO: 182 - DNA, 501 bp, Bos sp.
ggataaaattaaaccagaggacatagtaggtatttcattttatctgaggacattttgcttataccccatataatgcctgggaatcctattatttcatgcatttcaattccatctcccatgttcattaaagaaatttcatctgaagctagtatattacggtaaatccactatgttttaattttttttttaaagaatattttaaactttccccattaccactcaatctccaagggctcatctttttcccttayggttcatcagattttgtgtagaccatttcctaaatgaaacatcattttgggagtggacactttccaccgcaagatttaaagaactcttgtaggattattcattatttatgatatcatccacataaaaggaaaggagttgcctttcatctcactcgggaattcatttctctacctgtgttttcccctattctctcctggcatcagaatgtatgttataatttaaaacgcatgcactgtattttattttcat

SEQ ID NO: 183 - DNA, 501 bp, Bos sp.
gatttgcactagcaaatgatatgaatgtcgtagcccttgtgcctgactacagtgggaattccgaggaactatcttgagaattcccaaagctattttaggctaataatagaaaacccacaccaagcacagtgttttagtttggacaaaggttattctaccttaagcagtggctgaaaggcagtgattgatgtttccccatcatgaagcacagcatgttagttccttacttcgtctcaaatttattgtcacgrgaggacatttgtttgttacagtatgccaaaaagggaaaacaagttgttaaaggtcagatagttattaatgattttaactgaagaaaggatttaaaaatcaactccatggtatggactcttgaatgccttaccaatgatccattctaaaaaaaaatcttcatttataagtgacagttttctcccctagttgtcaagaggcaagatttttttctcctccttattatcctgtggtccagatgcactagttatg

SEQ ID NO: 184 - DNA, 501 bp, Bos sp.
aaaaaataccttggtgaaagaacagggtcagttctttgctcagcgtcttcactagaggataaatccacaaggtcatctgtgtcctgtacacagggaagctcttgttccttactggattcatcttcattaatctcaggacttaaagacaggcctttatctattaatacaaaacacatattattataaaaatgatagcaaaaacatttcaaaaagcacaaaggaattcacatacaaccctataaagtaaggaytatgaaactggcccaaagtgcaatttcttgatagttgagctcttctccttcatatgtaatatctttaaccgtaagccacaaaagagcataatattatgatgcctataaatcacactatattacctatcagtattcacttaaaatcttggtgaagctgaaccacataaaatttaaattctacagaaagattgttctagtctgctactatcttttcctcctctcttacttggttatctgctttaagtaatct

SEQ ID NO: 185 - DNA, 501 bp, Bos sp.
cgggcgtggcccattctgcaggcatggagagtctagaccacaggattcagcgcactgttcgtggtctgaactcaagcacgctcagggcagcccacagcaactgcaggagagtgggggagacgaaaaccacagcaggggattagctcacgcatcttgcttcatttattttattgactaaaaaagagctgagagcaaatacggttaaaattgcatttacttattcccattaagtcctgtacatatgaaacttmaagttgtgaactttcaaaagatgcaaatgtgcattccagcaatgtgaggcatgagtgacattgtagcttgccctccatctcctgtttgctgaccgtccttcagctctaccatctcccacttcctctcgctcctccagttagtaactcttcttgcctgtttactcaatgccagcccctgtatgccagctgttgtactgagctactgtactgtaagattttaaaatgttttctttattttgtgtttgttttg

SEQ ID NO: 186 - DNA, 501 bp, Bos sp.
ttgtactccaccaaaattcatgtgttgaaaccctaacttccagtgtggctatatttggaaaagggacctcttaaaaaacagtggttaaggttaaatgaggtcattagtgtggagccctgatccaagagtattagtgtccttatgagaagagacactgaagtttgctctttctctcttccatgtgaagacacagcagcaagaaggcaggtatctgcaagccaggaagatagccctcactagcaactgaatcwgataagcaccttgatcttgcacttcccaccttctagaattctgagaaagtttgtgttgtttaagccactgtggtattttgctacagcagaccaagcagatgaatacaccctcttagaggtaaagtaacttcaagaataaagcattatttgaacctcagttctgccacttgctactgggaggacctcgggcgcattgtttttttctctctgactctcagtgatctcaacacctaccatggaaatctcttgt

SEQ ID NO: 187 - DNA, 501 bp, Bos sp.
gcccctgactgattgtttaatattcacttagtaacagtatgagcttggctatatactttgctactgaacttagagatctttgtgttacatttgagcagagcatatcctacttatactgttgctactctcctgcaagagtgcataatatctgaatcaaatatattcttaggtgtggatgtgaagtcatttcagatatgtccaactgtttgcaacactatggactatagcccaccaggctccagagtccatgmttctctagtcaagaatactggagtgggatgctatttccttctccagggaattcttagaatacttgttgttaatttaaactagcaaatgcaagcccttgtatagtgctagaaacaaagataaaataaacatatgcataattatcattgcaccttaagcatatctgtgtagaaaaactctaggctaaaaagctggcaagagttagtggtttttttatgcctagagcattcatttcagttcagttgctcagtt

SEQ ID NO: 188 - DNA, 501 bp, Bos sp.
aggccttccctggctgtgctgtttacacacacccctcttcctgtgatttctttctgtttaaaatacttattgccaactgccatattttactaaaattgtctcttctcaattggagtgatgggaatgtaaaatccataggggcaagatttttttctgttttgttcactgctgtgcttccctgtaacaacaaactgttcattaactcatggtgtatgcagaggctaaggaaacaagtacacgcttacatgttrtgtgaatttggcagtgtttaccaaaattttaaatgtttacctgttgatccagtaatttcttggaatttaccctataaattagcttacacaggtgtgcaaatatgcatacatgtttctctccaaacttgtttttaattgtaaaacacctgagcagccacacaaagtatatccatttgtactaatacagaagagtccttcaaaagagtctgatcccattcttgtagaaaatagacacagagcatatacacat

SEQ ID NO: 189 - DNA, 501 bp, Bos sp.
ttagactccaaaagactaatttttaaaattttctctatgatctcaaacaatccactctattttttgattaggctgaaggattattcagaagcacacagtgaatgctttctagaagttatatcttatagaatatagtactgacaaaggaaaacatattaacaggtttatcagaataataagaaaacatccgaaagatattgaatattttaggcaaaccaaaatgtatagataataacgtaatggataacacrttttggttaaagataatctgtaatctgtgttttatttcatttatataagatgaaaagcaatgcctgcttaaataacttggtctttttttctctttctctttttttaaaaaaatttaaacaactcgtggccaaaactgtatccagtattacccagatgacaaataacatatcattttatctgtaaatattacattctgtaccttgaaaattggcttagtcattcagtcatatctgactctttgctacccca

SEQ ID NO: 190 - DNA, 501 bp, Bos sp.
attgtaagtcttcctttgagacttggagtttctttctggcttctgtgccacaattggggcactaggacatgtagcaaggccacattctcttggagtagatgtgatattctcttgtcacgggtcctgttgaaccccaggaagcatcatagacagcagcatttgcgtttggaccgcacaggtttctgtggtctctcttctgtcctctctccggccaggagcatctcttttgagtctgatggaatgtcggagarcccgatccttcctgcttccctagattttatataaaatacctttctctgtgtctggggtttcagagttcggcaagcagaatccaggatcatttgcacactaattatattctgttccaggatgggcatttctaaggccaatagaatggagctgatgactgagacgaatgaagaaaaggaaacaggggaactataggaggggagtgaagaggatttagatcaaagaccacttccatgacgcctgcccaaggga

SEQ ID NO: 191 - DNA, 501 bp, Bos sp.
attatatatactaagatatagtatagcatagtgaagtcgctcagtcgtgtccaactcttttcgaccccatggactgtagcctaccaggcttctccgtccatgggactttccaggcaagagtactggagtgggttgccatttccttctccaggggatctttctgacccagggatcgaacctgggtctcctgcattgtaggcagacgctttaccatctgagccactagggaagatgaatactaagatagatakatatatatatggaacctttaaagatctgacatataccaatgcatttgctgtctctatttacttgaataatcattaactacattctgagtgccgaagaattgatgcttttgaacttgtggtgttggagaagactcttgagagtcccttgggctggaaggagatccaaccagtccattccaaaggagattagttctgggtattctttggaaggaatgatgctaaagctgaaactccagtactttggccacct

SEQ ID NO: 192 - DNA, 501 bp, Bos sp.
gcggattctttgccatctgagccaccagggaagcacaagagtactggagtggtagctatcccttctccaggggggcttcctgacccaggaaatgaaccatggtcccctgcattgcaggcagattctttaccagctgagctaccagggaagcgccatgtaaaccatacaatgacttatttacacacacttgtgtgcatatacacaaacaactattctttcactaatatttcctgatgcgaagggaagagggmgaatactactgtgatttggttaaaatagtgcccaatgtttagaatagcattctttgaatttatctatttgtaaaaaaaaaaaaattaagctcagtttctatgaactgtacatacagtatgttgttgaaggacatcttccgtctggacctctagatatttctacatgctcttgaaataacctaaaatatagaatacaggaaaaaataccaggatgcacaagtgtttatacaatattcaatatttcttaagc

SEQ ID NO: 193 - DNA, 501 bp, Bos sp.
gtacaggcaggcactcaggcacagtgtgcactcttctccttgccactgttaaacttctacctaattaatgtctgtggggaaatgttttctcttgcactcaagcaaactaacataaaaggagatggtttccaaggcgactgcctctgctgaggttacaggagaaaattaggggagagctttctgaagtcatgtccccaagagggtgggggtaaagatagaggagggctaagatgttagttagtgagctgtckccaaggcagccaatttctgtacctggggagttccttgaagcactgactcttgctttggaattgggaaaactgctctggtacccggtatcaaggtcagtgtagaatcttctgcagatgctatcctgactcttggaaaacatcaaaagtagtcatacacaactaaagattcatttttaaagtctaaagtataaaaaatggcagtttcataacacagggaccaattctgactcctgactcttgtgaaactttg

SEQ ID NO: 194 - DNA, 501 bp, Bos sp.
gtctttgattttggtgaagtttcaaaattttaaatctctgctctgttctgcgtgtgcttagacccttaattaccagtgcattgccaggttaaattctcctcctcaagaccaagtttagatgggcaagccattctgctgatcatatccccgtcctttgttctaaacccactccttgttcttccagctccagaattccaaacctgcttaacccacatccttcacttggaaacaaaagaattttatcctagccrtaagtatgaagaactgtgaagagctggggtcagttatggggaatgccatgaatatttaggacactaaataatatctttatcacagaatcattgacctgaatttaatatgtaagtttgtgatcttggaattttcttcacccatacaatgacgggatttacctaaattacccctgagatctctttcagtatgaaaattcagtggtaaatttcctgtgtcagacaggacagtttgcagtttgcagatttctgc

SEQ ID NO: 195 - DNA, 501 bp, Bos sp.
ggttctctccctgctctaaccatgaagatgctaacaaagggcaagagtcagtactcgacctcaggtcccttccgggaagcctgagccacagctggtgtggtcatctctagctgaataaagccgccactcccaggcagcctggctcatggctgctccacttgaaacatgatccctcgtgcagggggcttgagaatcacaagggagattttcttcttactttgacttcataaacaaccaatacttctatttcrcagcttctagaaaactgctcagagaccccaatccccagggtcagtgtcacccccaggatccaggtcacaaggtcacaggtccttccagttaatgggctctttgccggacttctgcagcagctcgttggttttagagaaatgcctgcatccgaagaagcccctgtacaccgacagcggcgaggggtgcgccgtctgcagcacgtggtgccgcttctgaaaaagggggagacgggtggtgagaccggggggg

SEQ ID NO: 196 - DNA, 501 bp, Bos sp.
agttgctccaatctccctcacgttctcctcatcaccactccccacctttgctcaaccctgaaaattgtattgatttgatgatcatggggcccatgtttattttattttatttatttattttttttgagatgtagggtgtggctcctgtcagcatctatgtggtcctggacaagtagttaaccaaactctcagtgtctagtggggatggcagctctagtttcataaggcggctgcagagaaatgagtcagtrcatgtgaaagacttacctatatttccacattgttggctatcactaacgttctccacttcaccctcatcaccacagaattttgttccttctttcccgggcattcctaacatctttcttacaacctttctttgaaactcaagccccctgatctcatttccttaaactttgacaaagatgatagctcttgagttcatacctgattttattcttcagaggtttgctatgtccttgaggctggatctgagttgtc

SEQ ID NO: 197 - DNA, 101 bp, Bos sp.
acctggatggtgtgtcgagagcgttgtccctgccttggggcagtcctgcartccctctggtgtgggggtggggaggatgaactagtgcctttggctcctgt

SEQ ID NO: 198 - DNA, 501 bp, Bos sp.
gaatccgcctaccaatgcaggagacacaagggccctgggttctatcccttggttgggattccccctggaggaggaaatggcaagccactccagtattcttgcctggaaaattccatggacagaggagcgtggtgggctacagcccacggggtcacagagtcagacacagctgagcgcacacacaaaggataccagtgtatgtataactgaatcatgtcgctggacgccttgaccaacgctacgtgatcaakcaagcatatgccaaaatacaagtctaacaaaaagaaaataaatccctattgtattttttttcaggcttctggcaccttggcctgtgtcacagccctggagcctgagcaggcttggtctcatggccagagggtggtaaagctaagggggctagggaggctgtgtcatcagagaacctcccttggccctgtagtgcctgctcccctaagatcacagtctccagatccagtcagaccctcctatagctaaggc

SEQ ID NO: 199 - DNA, 501 bp, Bos sp.
gatctggagggagcagaaggaagggactacgtgagttgcctctggtccctcaggtggaaacgctcaccagacatccaatgggcagctgggtgtatgggggtagaatctggtcagagtattggagggagatatttaatttggatgtcaattggcaaataaatggactgaagcctggaaactggataagatcacccagagggtgcaaacagatctgagaaggacagagcccaggcctccagcagaatcagtcragaggatgaggggtaatgggtgaggaccttgggaaggagcagcgggtgaagtgggagcctggggcctgggggaggaggtgtcctgggagagggggaaggatcccaggaggcgggcgtggcccattctgcaggcatggagagtctagaccacaggattcagcgcactgttcgtggtctgaactcaagcacgctcagggcagcccacagcaactgcaggagagtgggggagacgaaaaccacagcaggggat

SEQ ID NO: 200 - DNA, 501 bp, Bos sp.
ttaaataaatgatgatgttaggctaaaattgttttaactttcaaagaaaagggtttgtaattaaaataatgatagactctccttggaaaacaggttgtttctttttaaggggagtaagaaattgaattttgccctccaattaaattttcaaaataataatgaaaaatgtggtaaaaaatgataagtatgatcatggaaaaatgcctgatttcattctggatgcagaatttgttcaaactggaaaacaagaraggaaacaagaaataaacatctttgtaagtcagggacaaactgaaaaatggcattcagcagagtgatagtttgtattacaaattaaatacccaactaaaatgcctgcagaagaaaggttaatttagcttcttgcattcaattctgataagtaaagcttgagtcattttataaattgaagtacatctgacatataaaactgttttagggcagaatttctctctttaaaatttttcagtattatcagtaaac

SEQ ID NO: 201 - DNA, 501 bp, Bos sp.
ggcagattcttaaccattggaccaccagagaagtccctcctcctcctttttgcttcttaattcctgaatgttgggcctggccgctattccaagttcacattgtcaaaaaaatctcagcatgtcagagcaagaggggtcagtgggtttccagcctgctcagtccgtcctgcacttcaagagttccaagaatcaaggtccaaataatcaaaattgccctttagcagtggagttactcttcagatgaaaccccratacataaatcagataaaagtagagttagtgccctctctctggagaggaggtgccaccctaggaccccagggccttccaggtcagtttgtgcagtttgattcctttcctccccctccatggaggaggaaacaggcagagtcttaaagggggcagagcttggattcctgctctgactctctctgccctgatgtgtggctgggttggatcttgggtccaaccaaagtgatcatcaggttcacctgatgaagc

SEQ ID NO: 202 - DNA, 501 bp, Bos sp.
aagaaaaatcatcaagcaaagtacaacatccctacttatccacaaagacagttcattttaatagatcctgtgacccactcaggtttcctgagaaacaacaaagttatttctctgagctactattcattttccatttgatatttatttcccaaacccatgtctgaactgcaattcccactgaaatgcaaacataactatttgatgttcagaacaaatacattgttctcagaataaaacatcaatttacacayacttttaaaaaatcattttaatgagtcagatacccagagctaagcatcagcgcatcactgtttccagtccagcttttgcagttttccctgccctctccacagtattattatgactgtgctctgtgtcttatgctcttttcaaacagggaaccaagtttggagtgatcctctggatcaggatttgttttactgagtctgaagttgaaatgagcaaattagccatgagaaaatacaatcttattttcttaac

SEQ ID NO: 203 - DNA, 501 bp, Bos sp.
gcccgcaggagctagaggacgcccatgcgcagcaacaaagaccgcagccaacaaaatgtatgtgcataggcgcctcctaggaaccggtagcaccatgtactcgccttagatggctgagagtgatgtgaacattgtaacacttgaccttggagaagacctgaggttcacctgtggacatgggtgggggaaggttgcagcctgcgggtcactgtgaggtaccaggagggagtttcctaaaatgtgatgggagratgtcgccccagattggaagggtggctcctgacagtcgagggagggtgtgtgtgtgtcgggtggcgtgcgtttcccgcatggcctggctcagccaggtatacttctccacacacaccccttgttactcctgggcaaaatcctgtcttattagcgaatgcagaatccacaccacgctcagatcattctttagcatttgtcccaaacagatgtctgtttaaaacaggccttggagcatggctgactctttct

SEQ ID NO: 204 - DNA, 501 bp, Bos sp.
  misc_feature (422)..(501): n is a, c, g, or t
tcatcagtgagaggtccaacatttcaggtcctcagtctggcttgactggcgtcagtgtgggccttagcgctgcataggagatcctcagttggcctctggttgtcccagagacttgcttagcaaatagtagacacataaacacatgccttcatgaccaaagcggtgaatgatgatgttctaaaatcatcatactttttttttaatgctaatctcccagtgaattttggaagtggcttttaaaaggtaaagastggagtagatttttgaaagcaatgtgtctgtgtgtatatgaaggaagcgagggctgaagtggagaaagagaaaggtttgaggacttgaggcttctcttggcagtcaccatcctgtcagccacctccgcctccaggcaggtcaccctgcaggccaaaagcctagccagtctnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 205 - DNA, 501 bp, Bos sp.
atgaacaaaaacaccttcaaaagaacaaagagtcacaattttatagatcaaagaatatatttagggcttggtttttagggatattttccaaaactgggtcacaatatgccccagcctgagagacatgaggtgacagagtccatgctttatcccaggtggatgagaaagaggggagttagaaaacttcctatgtcatttctgaagttatcctgggaaggcaggtgtaaaaagaaattgacatggcccaattyagattcacccagactggatgaagcagaaggagttccttttattagttattgttgcagaactcttaatcagcttaagattttgtggatcttttaaaatcattatcacagaaacacacaaacaggtcctgtggtttaactatctgtgtgttgaggagtgggactgggtgagcgaggctgggagggtggtccgtaggactgagctcttgggactgatatttacttttgacccttggcccttcaaactgccctg

SEQ ID NO: 206 - DNA, 501 bp, Bos sp.
tgagatatgaacatttggagttatgtgtttttgaaagaaaatgaaatgtagatgggtattttgtaccttcaaaataagaaacaaatagcactttctaatatgtttgaaaattgtttcagattcaaatttgttttgcagagtcaaacaagtgttgcttgtcatctttattgtattaattgtttggaaaatgagctaggctccgcagtgtgctctgactcacagcatttgcatcctgtttttttcttatacasttgctcttggtggagggaagactgtcagaagtggtcagcccgaccttactgactgtgggctgcacgtggctgttggccagctaggtgaccgtgttgattttatatgaaatacagtgaaagaaacgaaatgggagccgctgcaactgctgcttccctgtgagctcttttcctttgttgctcagaatccagatggagacagcatctttgaaacttgagaaaatatgacattttcctctgcctctaccttctc

SEQ ID NO: 207 - DNA, 501 bp, Bos sp.
tcaaaaactcaaacctctgaatccacaattacaagttggtaagagcatattgattttgtctatgaagttagccactttaaagcttccaaagttgccctctgacaaaggcaacatctggtctgccgaggactgtcccccagttgacctccaataacctaatattgctggatgctcagtcactcagggctttgctgagtttgttccacataaattctatgcagctcctaagagaaggctgagtttccagattsgtcaagagaaaaggaatctgccttcagaatgacaaatattcttttacctggagaaactgtcatcccagttatgcagtgacatccttagagctttattttcaaagcgaactttgagaacgcttggaagccaactctgtctgggagcgtgctctgccatctcctgatcctgcagttacattctgaattttccacatcttttgcattaaagaaacattattccctctaactctgtctgttggcctctttccag

SEQ ID NO: 208 - DNA, 501 bp, Bos sp.
tgattggtattttgtgaatctatagacatctgaggaaaactggcatctttattgcgttgattctgctaattcatataactcagtatgttcatttatttacatcttcttgggttatttcatcagcattttgtatttttcagaattcagcttctgtacatgtcttattatatatataccttgttagatgtatacctaagtcctttggaggaactgttgtgtcttctaaaatttgatttccaattgttctttgwatttagctttaaactctttgattataatgtgaccagaattggattttttgggtttgttctgtttggggtccactcagcttctttttatttgtatgtttatgtcagataattggccacatttggaacattttcagtcattatttctttgaatactctttcagcaccatcatttttttcttgtccttttgagactttgattatataaagtttagacactttgtgctgtcctacaggtccctaaggcttcatt

SEQ ID NO: 209 - DNA, 501 bp, Bos sp.
agttgatcaactagaaatctaatgcgtatgtaaaaatgatgtccgtcatgatacaattgcacaaatgaaataagtacctcattttggtccccagtagtaaaagatttctttgtaaatataaatgagataaaataccagtgaaaaattcttctctaatttaccataagcaaacttccatagaacaatctgggatttttgagttgcatgaggcaaacaactgaggtagtagggcttgccctgaagttcttggrtatactggacatttaatcccactacatgggatgtgagtttatttcagtcatgtttttataaatgagaaatgttatattatagttatgaaaagcccatgaattccttgtcacacactcctgagtcagactgttggtcaatatttagtgaatatacactctggtaccaggcatgatatcaatattaagacaaatgtggagtattcatttattcgttttttctaaatctagtaagaaagttaagtttttgttg

SEQ ID NO: 210 - DNA, 501 bp, Bos sp.
cagatgaagcagctttagtgaaaggagccaagaaacttggctttgttttcactacaaggatgccgaactcggtcaccatagaagctgtgagtaatacttgatatactttctagcggtttacatgcacctgctcatccagagacacacgtgtacaatttttctaatcatgctgattttagattggagaggtgtttcaatcttaatctgttgctataggaagcacttttgttaatactgtccaggttgcaaayaatattcttaccagaggagggcactgtcatctcatcagacttgcaggaaagctttattttgaaaaagatatgtatttggagggtgaaacttgttcatgattgggagagtgagtgactttgtgagacaaatacttttttcctaaggtaagaatatgttgtcttattttcctgtagatgggagaagaactcacatttgaaattcttaatgtactggaattttctaggtaagctatctttttgatctgtatgt

(19) United States
(12) Patent Application Publication
(10) Pub. No.: US 2009/0191549 A1
(43) Pub. Date: Jul. 30, 2009

(54) SINGLE NUCLEOTIDE POLYMORPHISMS (SNP) AND THEIR ASSOCIATION WITH TICK RESISTANCE IN BOVINE ANIMALS

(75) Inventor: William Barendse, Moggill (AU)

Correspondence Address: SUGHRUE MION, PLLC, 2100 PENNSYLVANIA AVENUE, N.W., SUITE 800, WASHINGTON, DC, 20037, US

(73) Assignees: COMMONWEALTH SCIENTIFIC AND INDUSTRIAL RESEARCH ORGANISATION, Campbell, Act (AU); DEPT OF PRIMARY INDUSTRIES FOR AND ON BEHALF OF THE STATE OF NEW SOUTH WALES, Orange, New South Wales (AU); THE STATE OF QUEENSLAND THROUGH ITS DEPT OF PRIMIARY INDUSTRIES AND FISHERIES, Brisbane, Queensland (AU); THE UNIVERSITY OF NEW ENGLAND, New South Wales (AU)

(21) Appl. No.: 12/092,397

(22) PCT Filed: Nov. 2, 2006
(86) PCT No.: PCT/AU06/01641

§ 371 Date: Oct. 8, 2008

(30) Foreign Application Priority Data
Nov. 2, 2005 (AU) 2005906058
Nov. 2, 2005 (AU) 2005906059

(51) Int. Cl. C12Q 1/68

(52) U.S. Cl. 435/6

(57) ABSTRACT

A method for assessing tick resistance in a bovine animal, comprising the steps of:
(1) providing a nucleic acid from the bovine animal;
(2) assaying for the occurrence of a single nucleotide polymorphism (SNP) identified in any one of SEQ ID Nos: 1 to 210,
wherein the identification of said nucleotide occurrence is associated with increased tick resistance in the animal.

### TECHNICAL FIELD

This invention relates to methods and nucleic acid probes for assessing tick resistance in cattle. The methods of the invention are useful in selection of cattle for tick resistance.

### BACKGROUND ART

DNA markers for tick resistance (the host resistance of cattle Bos taurus to the cattle tick Boophilus microplus) have mainly addressed the Major Histocompatibility Complex and while some suggestive associations between tick resistance and DNA markers have been reported none of these has been turned into a genetic test, mainly due to inconsistency and small size of effect. Moreover, the heritability of tick resistance is low to moderate depending upon the breed and the experiment, which makes finding DNA markers for selection of the trait of some importance if control of tick infestation by genetic means is to be achieved in a reasonable period of time.

### SUMMARY OF THE INVENTION

The present inventor has identified a number of single nucleotide polymorphisms (SNP) which act as genetic markers for tick resistance. Thus the invention allows a rapid and precise test for genetic variation for the trait, which would allow many cattle to be tested over a short period of time, in a cost effective manner, to establish this characteristic.

In a first aspect of the present invention there is provided a method for assessing tick resistance in a bovine animal, comprising the steps of:

(1) providing a nucleic acid from the bovine animal;

(2) assaying for the occurrence of a single nucleotide polymorphism (SNP) identified in any one of SEQ ID Nos: 1 to 210,

wherein the identification of said nucleotide occurrence is associated with increased tick resistance in the animal.

It will be appreciated that the association of an allele or genotype with increased value will often apply across breeds and families within breeds. However, a particular allele or genotype may not always be associated with increased values across breeds; in one breed the allele or genotype might be associated with an increased value but in another breed it might be associated with decreased value or not be associated with difference in value. The person skilled in the art will be able to establish the direction of the association.

Advantageously the assay is a quantitative assay which is capable of determining the number of copies of each form of the SNP in the nucleic acid sample. In an embodiment the assay is a polymerase chain reaction (PCR) which employs unique primers designed to amplify the DNA molecules set forth in SEQ ID Nos: 1 to 210 or a portion of these which contains the SNP, and complements thereof. However, other DNA based methods such as primer extension and oligonucleotide ligation assays could be used. Suitable methods for amplification of DNA of known sequence are well understood by the person skilled in the art, and application of such techniques is widely described, for example, in WO03/031592 (the contents of which are incorporated herein by reference).

According to a further aspect of the present invention there is provided a method for selecting a bovine animal with increased tick resistance from within a population of bovine animals, comprising the steps of:

(1) providing a nucleic acid sample from the bovine animal;

(2) assaying for the occurrence of a single nucleotide polymorphism (SNP) identified in any one of SEQ ID Nos: 1 to 210, wherein the identification of said nucleotide occurrence is associated with increased tick resistance in the animal; and

(3) selecting a bovine animal exhibiting increased tick resistance.

The single nucleotide polymorphisms of the invention are set forth in the Tables which follow, and a sequence listing providing a description of the polymorphism and giving 3′ & 5′ flanking sequence has been filed electronically. The SEQ ID Nos follow the order of the polymorphisms recited in Table 1, with SEQ ID NO: 1 equating to the first entry in Table 1 (identified by the locus designator 349624), SEQ ID NO: 2 corresponding to the second entry (locus designator 352775), and so on. Therefore, in an embodiment the present invention involves detecting the sequence set forth in SEQ ID Nos: 1 to 210, or a fragment thereof of at least 10 contiguous nucleotides which contains the polymorphism.

According to a further aspect of the present invention there is provided a solid substrate or surface comprising a plurality of nucleic acids in separate physical locations, including at least one nucleic acid as set forth in SEQ ID NO: 1 to 210, or fragments of at least 10 contiguous nucleotides which contain the polymorphism, immobilised thereon. For example, the nucleic acids of the present invention, or part of their sequence, may be used as a part or the whole of a microarray.

Additionally, primers or probes may be designed as described therein to hybridize to any one of SEQ ID NO: 1 to 210 or a complementary sequence thereto.

According to a further aspect of the present invention there is provided a kit for assessing tick resistance in a bovine animal through detection of the occurrence of a single nucleotide polymorphism (SNP), wherein the identification of said nucleotide occurrence as set forth in any one of SEQ ID NOs: 1 to 210 is associated with increased tick resistance, comprising an oligonucleotide probe, primer or primer pair, or combinations thereof, for determining the nucleotide occurrence of the SNP.

Advantageously the kit further comprises one or more detectable labels.

According to a further aspect of the present invention there is provided an oligonucleotide probe, primer or primer pair for detecting the occurrence of a single nucleotide polymorphism as set forth in any one of SEQ ID Nos: 1 to 210.

According to a still further aspect of the invention there is provided a method for assessing tick resistance in a bovine animal, comprising the steps of:

(1) providing a nucleic acid from the bovine animal;

(2) assaying for the occurrence of a polymorphism in a gene, including in the coding sequences, the introns, promoters and other regulatory sequences of said gene, or a polymorphism in linkage disequilibrium with a polymorphism in said gene, wherein said gene is selected from the group consisting of COL1A2, SSFA2, ITGA4, TSTA, TSTA3, RPS13, RDH10, ELTD1, RNASE1, SEMA3C, FLJ12572, NELL1, RIPK2, GPR39, LAMP2 and ARL6, wherein the identification of said nucleotide occurrence is associated with increased tick resistance.

According to a still further aspect of the invention there is provided a method for selecting a bovine animal with increased tick resistance from within a population of bovine animals, comprising the steps of, comprising the steps of:

(1) providing a nucleic acid sample from the bovine animal;

(2) assaying for the occurrence of a polymorphism in a gene, including in the coding sequences, the introns, promotors and other regulatory sequences of said gene, or a polymorphism in linkage disequilibrium with a polymorphism in said gene, wherein said gene is selected from the group consisting of COL1A2, SSFA2, ITGA4, TSTA, TSTA3, RPS13, RDH10, ELTD1, RNASE1, SEMA3C, FLJ12572, NELL1, RIPK2, GPR39, LAMP2 and ARL6, and wherein the identification of said nucleotide occurrence is associated with increased tick resistance; and

(3) selecting a bovine animal exhibiting increased tick resistance.

According to a still further aspect of the invention there is provided a kit for assessing tick resistance in a bovine animal through detection of the occurrence of a single nucleotide polymorphism (SNP) in a gene, including in the coding sequences, the introns, promotors and other regulatory sequences of said gene, or a polymorphism in linkage disequilibrium with a polymorphism in said gene, wherein said gene is selected from the group consisting of COL1A2, SSFA2, ITGA4, TSTA, TSTA3, RPS13, RDH10, ELTD1, RNASE1, SEMA3C, FLJ12572, NELL1, RIPK2, GPR39, LAMP2 and ARL6, and wherein the identification of said nucleotide occurrence is associated with increased tick resistance, comprising an oligonucleotide probe, primer or primer pair, or combinations thereof, for determining said nucleotide occurrence.

Animal selected by the methods of the invention may be selected for purposes of breeding from said animal, or a progenitor cell from an animal which exhibits this characteristic may be used in a method for cloning bovine animals. The invention is therefore also concerned, in further aspects, with animals when selected by the methods of the invention, their progeny and the use of both selected animals and their progeny for breeding.

The methods of the invention are applicable to bovine animals including but not limited to cattle, water buffalo and bison.

This specification contains nucleotide and amino acid sequence information prepared using PatentIn Version 3.3. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (e.g., SEQ ID NO: 1 refers to the sequence in the sequence listing designated as <210>1 and the sequence information immediately follows the identifier <400>1). In the sequences the symbols Y, R, M, K, S and W have been used to indicate the polymorphism. Thus the symbol “M” represents an A/T polymorphism, and so on. The sequence flanking the polymorphism is derived from publicly available sequence information. In most cases 250 residues to either side of the polymorphism have been set forth, hence the polymorphism resides at position 251. The present invention is not restricted to detection of the entire nucleotide sequence or in any way restricted to use of the entire nucleotide sequence. This information is presented to assist in the design of oligonucleotide premiers and probes, but the person skilled in the art will recognise that such sequence may contain errors and will adjust their design accordingly.

The designation of nucleotide residues referred to herein are those recommended by the IUPAC-IUB Biochemical Nomenclature Commission, wherein A represents Adenine, C represents Cytosine, G represents Guanine, T represents Thymine, Y represents a pyrimidine residue, R presents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

Throughout this specification, unless specifically states otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein any range of numerals includes all within the range and the term “at least one” means one, two, three, four, etc. up to the possible maximum. Therefore a reference to one or more SNPs includes one SNP or any number of SNPs from two to the total number of SNPs set forth herein. The SNPs set forth herein can be used alone or in any combination, therefore the invention envisages detection of any of the possible combinations of SNPs set forth herein.

As used herein the term “cow’ is used to refer to an individual animal without an intention to limit by gender and should not be taken to do so unless it is necessary from the context to infer that a female animal is referred to. The term should also be taken to encompass a young animal of either gender.

The present invention is not be limited in scope by the specific embodiments described herein, which are intended for the purpose of exemplification only. Functionally-equivalent products, compositions and methods are clearly within the scope of the invention, as described herein.

The present invention may be performed following the description herein without undue experimentation using, unless otherwise indicated, conventional techniques of molecular biology. Such procedures are described in various publications referred to throughout the specification, and the content of each such publication is incorporated herein by reference.

Throughout this specification and the claims, the words “comprise”, “comprises” and “comprising” are used in a non-exclusive sense, except where the context requires otherwise.

It will be clearly understood that, although a number of prior art publications are referred to herein, this reference does not constitute an admission that any of these documents forms part of the common general knowledge in the art, in Australia or in any other country.

Those skilled in the art will appreciate that the invention described herein is susceptible to variations and modifications other than those specifically described. It is to be understood that the invention includes all such variations and compounds referred to or indicated in this specification, individually or collectively, and any and all combinations or any two or more of said steps or features.

It will be appreciated that animals may be selected for greater or lesser tick resistance using the methods of the invention.

### MODES FOR PERFORMING THE INVENTION

Single nucleotide polymorphisms are allelic variants that occur in a population where a single nucleotide difference is present at a locus. The methods of the invention can involve detection of one single nucleotide polymorphism or more than one single nucleotide polymorphism, and can involve detection of single nucleotide polymorphisms which form or are a part of a haplotype which, as used herein, refers to groupings of two or more single nucleotide polymorphisms that are physically present in the same chromosome and tend to be inherited together except when recombination occurs. Methods for identifying haplotype alleles in nucleic acid samples are known to the person skilled in the art. This is from methods for haplotyping are described in WO 2005/040400, the contents of which are incorporated herein by reference.

A preferred sample for performing the methods of the invention is a readily accessible sample that comprises genomic DNA. For example, genetic testing of cattle is often performed using a hair follicle, for example, isolated from the tail of an animal to be tested.

Other examples of readily accessible samples include, for example, bodily fluids or an extract thereof or a fraction thereof. For example, a readily accessible bodily fluid includes, for example, whole blood, saliva, semen or urine.

In another embodiment, a biological sample comprises a cell or cell extract or mixture thereof derived from a tissue such as, for example, skin.

Preferably, a biological sample has been isolated or derived previously from a subject by, for example, surgery, or using a syringe or swab.

Cell preparations or nucleic acid preparation derived from such tissues or cells are not to be excluded. The sample can be prepared on a solid matrix for histological analyses, or alternatively, in a suitable solution such as, for example, an extraction buffer or suspension buffer, and the present invention clearly extends to the testing of biological solutions thus prepared.

Analysis of the sample may be carried out by a number of methods. The present invention has identified a number of SNPs associated with tick resistance in bovine animals, and subsequently detecting the presence or absence of the favourable allelic form of each SNP, or a plurality of these SNPs can be done using methods known in the art. Such methods may employ one or more oligonucleotide probes or primers including, for example, an amplification primer pair that selectively hybridize to a target polynucleotide which comprises a part or all of the sequence set forth in any one of SEQ ID Nos:1 to 210. Oligonucleotide probes useful in an embodiment of the invention comprise an oligonucleotide which is complementary to and spans a portion of the polynucleotide including the SNP in question. Therefore, the presence of a specific nucleotide at the position (i.e. one of the allelic forms of the SNP) is detected by the ability or otherwise for the probe to hybridize. Such a method can further include contacting the target polynucleotide and hybridized oligonucleotide with an endonuclease and detecting the presence or absence of a cleavage product of the probe.

An oligonucleotide ligation assay also can be used to identify a nucleotide occurrence at a polymorphic position, wherein a pair of probes that selectively hybridize upstream and adjacent to and downstream and adjacent to the site of the SNP are prepared, and wherein one of the probes includes a terminal nucleotide complementary to a nucleotide occurrence of the SNP. Where the terminal nucleotide of the probe is complementary to the nucleotide occurrence, selective hybridization includes the terminal nucleotide such that, in the presence of a ligase, the upstream and downstream oligonucleotides are ligated. As such, the presence or absence of a ligation product is indicative of the nucleotide occurrence at the SNP site.

An oligonucleotide also can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP site can be useful, wherein the amplification product is examined to determine the nucleotide occurrence at the SNP site. Particularly useful methods include those that are readily adaptable to a high throughput format, to a multiplex format, or to both. The primer extension or amplification product can be detected directly or indirectly and/or can be sequenced using various methods known in the art. Amplification products which span a SNP loci can be sequenced using traditional sequence methodologies (e.g., the “dideoxy-mediated chain termination method,” also known as the “Sanger Method” (Sanger, F., et al. J. Molec. Biol. 94:441 (1975); Prober et al. Science 238:336-340 (1987)) and the “chemical degradation method,” “also known as the “Maxam-Gilbert method” (Maxam, A. M., et al. Proc. Natl, Acad. Sci (U.S.A.) 74:560 (1977)), the contents of which are herein incorporated by reference to determine the nucleotide occurrence at the SNP loci.

As will be apparent to the person skilled in the art, the specific probe or primer used in an assay of the present invention will depend upon the assay format used. Methods of designing probes and/or primers for example, PCR or hybridization are known in the art and described, for example, in Dieffenbach and Dveksler (Eds) (In: PCR Primer: A Laboratory Manual, Cold Spring Harbour Laboratories, NY, 1995) and more recently in P.-Y. Kwok (Ed) (In: Methods in Molecular Biology Vol 212 Human Press, Totowa N.J., 2003). The various categories of polymorphism have been systematized and the various methods used to detect them have been thoroughly overviewed (Barendse and Fries 1999). In short, there are only two kinds of polymorphism, those due to changes of DNA bases and those due to insertion and deletion of bases. Furthermore, the detection of these polymorphisms uses essentially the same technology and it is a rare technique that can be used for only one or the other of these kinds of polymorphism. Polymorphisms can also be divided into those that are in or near a sequence that is transcribed into RNA (type I polymorphisms) and those that are in DNA that is never translated into RNA (type II polymorphisms). However, all of these polymorphisms can be detected using the same kinds of methods. The methods for detecting DNA polymorphisms revolve around 3 major aspects, not all of which are used in every detection method, and some methods use techniques that are not one of these 3 major kinds. There is a large and ever increasing range of methods for detecting polymorphisms, and a particular DNA sequence will lend itself to one or other method of detection. Most of these methods are highly dependent upon the polymerase chain reaction (PCR), although it is possible to detect sequence differences easily without using the PCR. The three technologies are 1) separation of DNA by size or by composition, 2) oligonucleotide hybridisation to recognise specific DNA sequences, and 3) DNA visualisation. The DNA separation may be performed on solid matrices but may be performed in liquid matrices. The recognition of DNA sequence is usually performed by oligonucleotides of predefined sequence but may be performed enzymatically since some enzymes recognise specific DNA sequence motifs. DNA visualisation can be performed directly on the DNA which binds to some elements such as silver when it is visible in ordinary light, it may fluoresce under ultra violet light when it is bound to some molecules such as ethidium bromide, or it may be visualised through autoradiography when radiactive nucleotides are incorporated into the sequence. More usually, the DNA is visualised when it is bound to a DNA oligonucleotide which has a previously attached reporter molecule which may then be detected after laser excitation. Many methods depend on reporting the result of a specific reaction on the DNA, and may not even detect the DNA itself but remnants of the successful detection. These descriptions are merely to indicate the wide range and the many possible permutations of DNA detection, and do not exclude methods that have not been specifically referred to.

Some of the methods that might be used to detect the polymorphisms are described below, but they are not the only possible methods. While the specific hybridization of the probe or primer or other method for detecting variability to any nucleic acid can be predicted using well known rules, the probe or primer may not be unique if it is designed to bind to repetitive DNA sequence or to sequence common to members of a gene family, and so precautionary screening of probes and primers should be performed using, for example, BLAST against the cow and other genomes. In many cases this will not be sufficient and the adequacy of probes or primers may need to be confirmed empirically using methods known in the art. This same proviso will apply to all methods of detecting DNA that uses short probes and primers as would be appreciated by anyone skilled in the art.

The following is a list of some of the more useful current high throughput methods of detecting polymorphisms in cattle, but these should not be taken as an exhaustive list or be used to exclude new methods yet to be developed where they are essentially being used to identify the underlying DNA sequence. In that regard, if the DNA sequence has been transcribed to RNA and the RNA is tested for variation, or if the RNA has been translated to protein and the protein is tested for variation, these RNA and protein detection methods will also be methods of detecting the underlying DNA sequence. As indicated above, sometimes the detection method reports the result of a successful reaction without directly detecting the DNA molecule, and obviously this would apply for RNA and protein as well.

Some of the useful DNA based methods of detecting polymorphisms are the Taqman assay (Livak 2003), which uses competitive hybridisation of probes specific for the alternative DNA sequences and where a successful reaction is detected through the liberation of a reporter dye, the SNPlex assay (Applied Biosystems Incorporated, Foster City, Calif.) which uses the oligonucleotide ligation assay and where a successful reaction is reported via Zipchute probes that are separated on a capillary DNA sequencer, high throughput molecular inversion probes associated with generic microarray technologies (Hardenbol et al. 2003; Hardenbol et al. 2005), the MASSextend (Storm et al. 2002) or generic primer extension technologies (Chen et al. 1997) which use mass spectrometry or laser fluorescence of the probe modified by an enzyme reaction respectively.

The present invention also relates to kits which can be used in the above described methods. Such kits typically contain an oligonucleotide probe, primer, or primer pair, or combinations thereof, depending upon the method to be employed. Such oligonucleotides are useful, for example, to identify a SNP as set forth herein. In addition, the kit may contain a control comprising oligonucleotides corresponding to the nucleotide sequence of the non-desired allelic form. In addition, the kit may contain reagents for performing a method of the invention such as buffers, detectable labels, one or more polymerases, which can be useful for a method that includes a primer extension or amplification procedure, and are nucleases for digesting hybridization products or a ligase which can be useful for performing an oligonucleotide ligation assay. The primers or probes can be included in the kit in labelled form.

The kit may also include instructions for use.

Management of tick prevention and treatment strategies such as chemical treatment and/or vaccination of animals can be tailored to suit the individual animal based on an assessment using the methods of the invention.

As well as management of the individual animal, the present invention allows for selection of animals for breeding programs. Thus a herd may be developed with increased tick resistance. The progeny of the mating of selected parents are likely to contain the trait, thus creating a line of animals with specific characteristics. The progeny can then be used to breed and so on in order to continue the line, which may be monitored for purity using the original SNP markers.

Furthermore, the methods of the invention allows for in vitro methods of producing animals. In general terms the method involves identification of one or more SNPs as set forth in SEQ ID-Nos: 1 to 210 in a bovine animal, isolating a progenitor cell from the animal and generating an animal from the progenitor cell. Methods of cloning bovine animals are well known to the person skilled in the art. For methods involved in cloning of cattle known methods may be used directly. As set forth, for example, in Willadsen “Cloning of sheep and cow embryos,” Genome 31:956 (1989), the contents of which are incorporated herein by reference.

In an embodiment, following development of an embryo, one or more cells is/are isolated therefrom and screened as described above. An embryo having or likely to have possess increased tick resistance is then selected and implanted into a suitable recipient.

In an embodiment the selected animals are used to produce offspring using in vitro fertilization. In this process, ova are harvested from a cow comprising one or more SNP of the invention by, for example, transvaginal ovum pick-up (OPU) or by laparoscopic aspiration. The recovered ovum is then matured prior to fertilization. Zygotes are then cultured for a time and under conditions suitable for embryo development. For example, zygotes are cultured in a ligated oviduct of a temporary recipient (sheep or rabbit). Alternatively, zygotes are co-cultured in vitro with somatic cells (e.g., oviduct epithelial cells, granulosa cells, etc) in a defined medium. Alternatively, zygotes are cultured in vitro in a simple medium such as synthetic oviductal fluid without any somatic cell support.

The method is amenable to screening embryos produced using any assisted breeding technology and/or for screening embryos produced using an ovum and/or sperm from an animal that has not been screened using the methods of the invention.

### EXAMPLE 1

Farmers were approached throughout Queensland, Australia to be part of the study. Ticks were counted, mainly in the period January-May of 2004 and 2005. Ticks were counted in the size range 4.5-8 mm. Ticks were counted by two individuals, and 10 ml of blood collected into EDTA vacutainers. Ticks counts and herd records were collected into a database that included the following information—National Herd Id, Property No. National Cow Id, Birth Date, Sire, Dam, Breed, Calving Date, Observation Date (for TICK), Parity, Last Herd Recording Date. So far, 824 cows have full datasets, representing 316 sires and 582 dams of 63 breeds or taurine breed crosses. In the breed crosses, each grandparental breed is specified as follows.

| | | | | | |
|---|---|---|---|---|---|
| AAII | AAUU | BBBB | BBBF | BBFF | BBJF |
| BBUF | BBXX | EEXX | FFFF | FFFG | FFFI |
| FFFX | FFGG | FFIF | FFJF | FFSF | FFUI |
| FFUU | FFXF | FFXX | GGFF | GGGF | GGGG |
| IIAX | IIFF | IIII | IIIX | IIUU | IIUX |
| IIXI | ISII | JJBF | JJFF | JJFG | JJGG |
| JJIF | JJJF | JJJJ | JJJX | JJXJ | JJXX |
| TTJJ | UUBB | UUBF | UUFF | UUFX | UUII |
| UUIU | UUIX | UUJJ | UUUI | UUUU | UUUX |
| UUXI | UUXX | WWFF | WWJJ | XXBX | XXJF |
| XXJJ | XXXF | XXXX | | | |

The breed code required is a four character string, capable of defining the parental breeds, i.e.

XX XX

| | |
|---|---|
| Sire Breed | Dam Breed |
| X X | X X |

| | | | |
|---|---|---|---|
| Paternal | Paternal | Maternal | Maternal |
| Grand Sire | Grand Dam | Grand Sire | Grand Dam |
| Breed | Breed | Breed | Breed |

The ADHIS single alpha character breed codes are:

| | |
|---|---|
| A | AYRSHIRE |
| B | BROWN SWISS |
| C | AUSTRALIAN COMMERCIAL DAIRY COW |
| D | DAIRY SHORTHORN |
| E | BEEF BREEDS |
| F | HOLSTEIN |
| G | GUERNSEY |
| H | SAHIWAL |
| I | ILLAWARRA |
| J | JERSEY |
| M | MEUSE-RHINE-ISSEL |
| R | RED POLL |
| S | SIMMENTAL |
| U | AUSTRALIAN RED BREED |
| W | AUSTRALIAN FRIESIAN SAHIWAL |
| Z | AUSTRALIAN MILKING ZEBU |
| X | UNKNOWN |

Example of the four alpha character breed code required by ADHIS.

FFFF ‘PURE’ Holstein, resulting from the mating of a Holstein Bull to a Holstein cow.

FFJJ Holstein-Jersey cross, resulting from the mating of a Holstein bull to a Jersey cow.

JJFF Jersey-Holstein cross, resulting from the mating of a Jersey bull to a Holstein cow.

FFFJ Three Quarter Holstein, resulting from the mating of a Holstein bull to a Holstein-Jersey cross cow.

FFXX Holstein cross, resulting from the mating of a Holstein bull to a cow of unknown breed.

FFFX Three Quarter Holstein, resulting from the mating of a Holstein bull to a Holstein cross cow whose sire was Holstein and whose dam was of unknown breed.

There were 20 herd-test days, 73 herd-parities and 431 different days in milk (with a range 0-1463). Tick counts were transformed using a log transform of tick counts +1. The traits were analysed using univariate analysis first and then bivariate analysis to derive variance-covariance matrices for genetic parameter estimation. General Linear Mixed Models of ASReml v1.99 using an animal model with the relationship matrix was applied in the analyses. The tick counts were modelled using the equation:

log a˜mu+animal+dam+ide(animal)+breed+herdtestdate+testage+error.

in which animal, dam, ide(animal) and breed were random effects, ide(animal) is a permanent environmental effect, herdtestdate was a fixed effect, and testage is a covariate.

Not all of the animals were chosen for genotyping. Of the available animals, 189 were chosen for genotyping representing similar numbers from each of 5 properties, for 138 sires, 174 dams, and 40 of the breed combinations. The animals were not chosen on the basis of their trait values, but mainly to minimise relatedness to increase the map resolution.

DNA was extracted from 200 microlitres of blood and purified through a QIAGEN column following the manufacturers instructions. The DNA was genotyped using the ParAllele 10K standard SNP panel. The method of SNP genotyping is documented in Hardenbol, P., Baner, J., Jain, M., Nilsson, M., Namsaraev, E. A., Karlin-Neumann, G. A., Fakhrai-Rad, H., Ronaghi, M., Willis, T. D., Landegren, U. and Davis, R. W. “Muliplex genotyping with sequence-tagged molecular inversion probes” Nature Biotechnology 5 May 2003. The panel of approximately 10,000 bovine SNP was derived from the publicly available DNA sequence and is called the MegAllele Genotyping Bovine 10K SNP Panel. The SNPs allow researchers to perform linkage mapping studies on bovine breeds with no bias towards either beef or dairy. The MegAllele Genotyping Bovine 10K SNP Panel is designed to work with the Affymetrix GeneChip Scanner 3000. ParAllele Part #8-0015”. The residual tick count for each individual was compared to all of its genotypes. Mean values for each genotype were computed and the means compared using at test and the statistical significance evaluated using 100,000 permutations.

Table 1 contains data showing associations for tick resistance, and Table 2 contains correlates associations for tick resistance with the locations of the SNP on the bovine genome.

To determine which genotype is favourable, for tick resistance the genotype or group of genotypes with lower values should be chosen. The genotypes can be clearly identified since if the DNA sequence bases are ranked in alphabetical order for the SNP, i.e., C/G, A/C, G/T, A/T, etc, then genotype 0 is always first in alphabetical order while genotype 2 is always last in alphabetical order at the locus.

We see several SNP that are close together that show associations with high levels of statistical significance. This can most easily be seen by looking at the chromosome location (scaffold-v2). Scaffold-v2, the version 2 scaffolds of the bovine genome, show the bovine chromosome number and scaffold within chromosome, for example scaffold-v2 1.112 means bovine chromosome 1 scaffold 112. Some scaffold can only be assigned to a human chromosome, see HumChr in Table 2. When combined with scaffold-v2 information, these locations show quite clearly clusters of markers in a region for each trait. These clusters could be used to select animals.

The method of determining whether a measured allele or genotype has an increased value compared to others at that locus or more broadly within the gene or genetic region would be familiar to the person skilled in the art but will be described briefly. In essence we partition the variance associated with the trait into that due to the Mendelian component associated with the locus under discussion as well as a polygenic component due to shared family. Before this is done, the trait values must be adjusted for fixed environmental and genetic effects, for covariates, and for random genetic effects such as the sire or dam. This is usually performed using a General Linear Mixed Model. Then the genotypes can be compared using at test or a one-way analysis of variance, and the statistical significance can be assessed using permutation tests, particularly where the trait distribution is non-normal. The average effect of allele substitution at the locus is derived from the allele frequency, the difference between homozygotes and the degree of dominance, where alpha=a[1+k(p−(1−p)] where a is half the difference between homozygotes, p is the allele frequency and k is d/a where d is the difference between the heterozygote and half the distance between the homozygotes. Boerwinkle et al. 1986 Ann. Hum. Genet. 50, 181-194 and Lynch and Walsh, 1997 (Sinauer Associates).

The association of an allele or genotype with increased value will often apply across breeds and families within breeds. However, a particular allele or genotype may not always be associated with increased value across breeds, in one breed the allele or genotype might be associated with increased value but in another breed it might be associated with decreased value or not be associated with differences in value. The results presented here are the associations aggregated across the breeds, which represents the alleles or genotypes that will be associated with increased value for most breeds. Since the values have been adjusted for the breeds, the associations can be pooled across breeds, and differences in allele frequency in the breeds will not cause the generation of spurious associations due to Simpsons Paradox.

When presented in this way, firstly, the aggregated statistics give more realistic values because they are based on larger numbers, and secondly, there are many cattle breeds and so it is important to obtain the general association between the allele or the genotype and the trait, which will have applicability in most or all breeds. Clearly, the person skilled in the art will know that some breeds may have different associations between the allele or genotype and the trait due to one of several real biological causes. The first and probably most common is that the measured allele or genotype is not causative, so it is in linkage disequilibrium with the causative allele or genotype. There will be cases where the allele or genotype being measured is in opposite genetic phase to the causative allele or genotype, and this might be reflected in some breed differences. The second is that there may be more than one causative mutation in the gene, with different frequencies in different breeds, hence the measured allele or genotype may show different predictive efficiencies in different breeds and show opposite genetic phase relationships due to complex associations between the measured allele or genotype and the different causative mutations. The third is that a causative mutation in a gene may be affected by genes elsewhere in the genome. These epistatic or background effects have been known for decades, and some of these may have an impact upon the association between the measured allele or genotype and the trait value.

TABLE 1
Associations between SNP and tick resistance.

| | scaffold- | loca- | Base- | | | | | | | | | | | | SEQ |
|---|---|---|---|---|---|---|---|---|---|---|---|---|---|---|---|
| Locus | v2 | tion | change | N | Mean0 | SE | N | Mean1 | SE | N | Mean2 | SE | tmax | PermP | ID |
| 349624 | 4.14 | 285504 | A/C | 125 | −0.0034 | 0.01 | 24 | 0.0173 | 0.01 | 12 | −0.0357 | 0.01 | 3.91 | 0 | 1 |
| 352775 | Un.5392 | 43292 | C/T | 74 | −0.0047 | 0.01 | 41 | 0.0304 | 0.01 | 47 | −0.0247 | 0.01 | 3.69 | 0 | 2 |
| 349705 | Un.51 | 655288 | G/T | 17 | −0.0685 | 0.03 | 72 | 0.007 | 0.01 | 85 | 0.0037 | 0.01 | 3.89 | 0.00002 | 3 |
| 347700 | Un.895 | 65002 | C/T | 59 | 0.019 | 0.01 | 76 | 0.0011 | 0.01 | 35 | −0.0394 | 0.02 | 3.57 | 0.00008 | 4 |
| 351771 | NULL | NULL | C/T | 12 | −0.0773 | 0.03 | 63 | 0.0083 | 0.01 | 99 | 0.0006 | 0.01 | 3.66 | 0.00011 | 5 |
| 345751 | Un.734 | 193986 | C/T | 16 | 0.072 | 0.02 | 63 | −0.0063 | 0.01 | 95 | −0.0116 | 0.01 | 4.01 | 0.00012 | 6 |
| 351349 | Un.10044 | 18624 | C/T | 62 | −0.0137 | 0.01 | 80 | −0.0105 | 0.01 | 32 | 0.0419 | 0.01 | 3.61 | 0.00016 | 7 |
| 353220 | Un.734 | 193387 | A/C | 67 | −0.0098 | 0.01 | 61 | −0.005 | 0.01 | 15 | 0.0768 | 0.02 | 4.26 | 0.00016 | 8 |
| 354299 | Un.734 | 193708 | C/T | 106 | −0.0101 | 0.01 | 52 | −0.0082 | 0.01 | 14 | 0.0793 | 0.03 | 4.04 | 0.00016 | 9 |
| 353165 | 3.108 | 21885 | A/G | 45 | 0.0256 | 0.01 | 80 | −0.0028 | 0.01 | 49 | −0.026 | 0.01 | 3.78 | 0.00017 | 10 |
| 346367 | 4.42 | 1548870 | A/G | 57 | 0.0129 | 0.01 | 82 | 0.0024 | 0.01 | 35 | −0.0367 | 0.01 | 3.58 | 0.00026 | 11 |
| 349425 | 21.6 | 958026 | A/C | 34 | 0.0304 | 0.01 | 82 | −0.0026 | 0.01 | 58 | −0.0202 | 0.01 | 3.64 | 0.00029 | 12 |
| 347051 | 10.41 | 71349 | A/G | 4 | 0.0204 | 0.02 | 42 | 0.036 | 0.01 | 128 | −0.0152 | 0.01 | 3.83 | 0.00034 | 13 |
| 344395 | Un.856 | 42123 | C/G | 37 | 0.04 | 0.01 | 86 | −0.0084 | 0.01 | 50 | −0.0216 | 0.01 | 3.52 | 0.00037 | 14 |
| 351066 | Un.8255 | 16212 | A/G | 104 | −0.0189 | 0.01 | 58 | 0.0231 | 0.01 | 12 | 0.0231 | 0.02 | 3.41 | 0.00037 | 15 |
| 351896 | 23.78 | 164911 | A/G | 4 | 0.0974 | 0.04 | 61 | −0.008 | 0.01 | 109 | −0.0023 | 0.01 | 3.75 | 0.0004 | 16 |
| 345776 | 14.112 | 653790 | A/G | 0 | NaN | NaN | 4 | −0.137 | 0.02 | 170 | 0.0012 | 0.01 | 3.65 | 0.00045 | 17 |
| 350060 | 1.47 | 18797 | A/G | 34 | 0.0379 | 0.02 | 71 | −0.0104 | 0.01 | 69 | −0.0129 | 0.01 | 3.3 | 0.00045 | 18 |
| 346463 | 2.101 | 164846 | C/T | 46 | −0.0015 | 0.01 | 87 | −0.0173 | 0.01 | 40 | 0.0304 | 0.01 | 3.42 | 0.00047 | 19 |
| 352671 | 2.101 | 164612 | A/T | 36 | 0.0352 | 0.01 | 88 | −0.017 | 0.01 | 46 | −0.0015 | 0.01 | 3.6 | 0.00048 | 20 |
| 351705 | Un.801 | 84560 | A/C | 132 | −0.0006 | 0.01 | 39 | −0.0163 | 0.01 | 3 | 0.1209 | 0.08 | 4.2 | 0.00057 | 21 |
| 351575 | 19.3 | 538807 | A/G | 27 | 0.0078 | 0.01 | 91 | −0.0201 | 0.01 | 56 | 0.0228 | 0.01 | 3.36 | 0.00074 | 22 |
| 351738 | Un.695 | 96764 | A/G | 0 | NaN | NaN | 17 | −0.0632 | 0.02 | 157 | 0.0046 | 0.01 | 3.54 | 0.00078 | 23 |
| 351752 | Un.192 | 116354 | A/C | 47 | −0.0065 | 0.01 | 93 | 0.0131 | 0.01 | 34 | −0.0372 | 0.01 | 3.3 | 0.00078 | 24 |
| 346327 | Un.3744 | 69312 | C/T | 148 | 0.0072 | 0.01 | 25 | −0.049 | 0.02 | 1 | −0.1914 | NaN | 3.51 | 0.00081 | 25 |
| 346753 | 11.127 | 170758 | A/G | 12 | −0.0012 | 0.01 | 75 | −0.0236 | 0.01 | 87 | 0.0166 | 0.01 | 3.3 | 0.00081 | 26 |
| 352832 | 7.107 | 376818 | A/T | 57 | 0.0055 | 0.01 | 85 | −0.0182 | 0.01 | 30 | 0.0319 | 0.01 | 3.24 | 0.00082 | 27 |
| 352428 | 13.96 | 260322 | A/T | 146 | 0.0062 | 0.01 | 27 | −0.0463 | 0.02 | 1 | −0.0065 | NaN | 3.31 | 0.00083 | 28 |
| 348202 | 1.142 | 936211 | G/T | 11 | −0.0773 | 0.04 | 55 | 0.0099 | 0.01 | 108 | −0.0004 | 0.01 | 3.3 | 0.00084 | 29 |
| 344940 | Un.7186 | 17526 | C/T | 124 | −0.013 | 0.01 | 47 | 0.0276 | 0.01 | 3 | −0.0127 | 0.01 | 3.11 | 0.0009 | 30 |
| 350422 | Un.5767 | 41054 | C/G | 143 | −0.0023 | 0.01 | 28 | −0.0124 | 0.01 | 3 | 0.1078 | 0.05 | 3.66 | 0.0009 | 31 |
| 348071 | 17.33 | 262897 | C/T | 38 | 0.0263 | 0.01 | 89 | −0.0018 | 0.01 | 47 | −0.0253 | 0.01 | 3.25 | 0.00102 | 32 |
| 348632 | Un.192 | 116302 | C/T | 47 | −0.0065 | 0.01 | 91 | 0.0112 | 0.01 | 34 | −0.0372 | 0.01 | 3.22 | 0.00102 | 33 |
| 344009 | 7.104 | 281726 | A/G | 3 | −0.0298 | 0.02 | 17 | −0.0618 | 0.02 | 153 | 0.0055 | 0.01 | 3.48 | 0.00108 | 34 |
| 350139 | Un.6146 | 9805 | A/G | 63 | 0.0119 | 0.01 | 86 | −0.0008 | 0.01 | 25 | −0.041 | 0.01 | 3.21 | 0.00112 | 35 |
| 353745 | 17.33 | 263011 | G/T | 47 | −0.0253 | 0.01 | 89 | −0.0018 | 0.01 | 38 | 0.0263 | 0.01 | 3.25 | 0.00112 | 36 |
| 343247 | 17.33 | 263083 | C/G | 47 | −0.0253 | 0.01 | 89 | −0.0018 | 0.01 | 38 | 0.0263 | 0.01 | 3.25 | 0.00118 | 37 |
| 346144 | Un.3077 | 69958 | C/T | 52 | 0.0016 | 0.01 | 83 | −0.0182 | 0.01 | 39 | 0.0277 | 0.01 | 3.03 | 0.00121 | 38 |
| 343545 | 10.2 | 438426 | G/T | 19 | −0.0128 | 0.02 | 83 | 0.0183 | 0.01 | 72 | −0.0226 | 0.01 | 3.33 | 0.00122 | 39 |
| 344231 | 10.2 | 438703 | A/G | 72 | −0.0226 | 0.01 | 83 | 0.0183 | 0.01 | 19 | −0.0128 | 0.02 | 3.33 | 0.00125 | 40 |
| 350644 | 17.34 | 1342784 | A/G | 6 | −0.038 | 0.04 | 59 | 0.0244 | 0.01 | 102 | −0.0143 | 0.01 | 3.11 | 0.0013 | 41 |
| 353781 | 4.41 | 64324 | C/T | 14 | −0.054 | 0.02 | 59 | −0.0089 | 0.01 | 100 | 0.01 | 0.01 | 3.29 | 0.00143 | 42 |
| 345817 | Un.465 | 81872 | A/C | 45 | 0.0032 | 0.01 | 83 | −0.0193 | 0.01 | 45 | 0.0257 | 0.01 | 3.12 | 0.0016 | 43 |
| 349461 | Un.801 | 84127 | C/T | 5 | 0.0775 | 0.05 | 43 | −0.0155 | 0.01 | 126 | −0.0005 | 0.01 | 3.49 | 0.0016 | 44 |
| 353486 | 2.162 | 917389 | A/G | 102 | 0.0136 | 0.01 | 61 | −0.0234 | 0.01 | 9 | −0.0417 | 0.02 | 3 | 0.0016 | 45 |
| 354151 | 3.108 | 21818 | C/G | 54 | 0.0182 | 0.01 | 76 | −0.0017 | 0.01 | 44 | −0.0273 | 0.01 | 3.06 | 0.00162 | 46 |
| 343049 | 22.17 | 34174 | C/T | 26 | 0.0426 | 0.02 | 74 | −0.0073 | 0.01 | 73 | −0.0126 | 0.01 | 3.21 | 0.00163 | 47 |
| 349905 | 29.37 | 615376 | A/G | 51 | −0.0044 | 0.01 | 56 | 0.0229 | 0.01 | 67 | −0.0209 | 0.01 | 3.35 | 0.00164 | 48 |
| 346722 | Un.417 | 22607 | A/G | 60 | −0.0075 | 0.01 | 88 | 0.0113 | 0.01 | 26 | −0.0341 | 0.01 | 2.95 | 0.00165 | 49 |
| 348572 | Un.516 | 289241 | C/T | 71 | 0.0184 | 0.01 | 83 | −0.0218 | 0.01 | 20 | 0.0078 | 0.02 | 3.32 | 0.00165 | 50 |
| 342524 | Un.10647 | 15369 | C/T | 163 | 0.0002 | 0.01 | 3 | −0.115 | 0.02 | 8 | −0.0053 | 0.02 | 3.66 | 0.0017 | 51 |
| 349447 | Un.417 | 22941 | C/T | 26 | −0.0341 | 0.01 | 87 | 0.0114 | 0.01 | 61 | −0.0074 | 0.01 | 2.94 | 0.00171 | 52 |
| 353318 | 28.47 | 47391 | A/C | 55 | 0.0176 | 0.01 | 78 | −0.0243 | 0.01 | 21 | 0.0211 | 0.01 | 2.99 | 0.00173 | 53 |
| 346469 | Un.3712 | 62136 | A/G | 52 | 0.0271 | 0.01 | 73 | −0.0174 | 0.01 | 49 | −0.0099 | 0.01 | 3.15 | 0.00174 | 54 |
| 352706 | 8.112 | 6860 | A/G | 120 | −0.0131 | 0.01 | 49 | 0.0255 | 0.01 | 4 | 0.014 | 0.01 | 2.97 | 0.00182 | 55 |
| 347184 | Un.365 | 40903 | C/T | 4 | −0.0992 | 0.04 | 19 | −0.0056 | 0.01 | 151 | 0.001 | 0.01 | 3.36 | 0.00201 | 56 |
| 343022 | 10.2 | 438462 | A/G | 26 | −0.0018 | 0.01 | 76 | 0.0174 | 0.01 | 72 | −0.0226 | 0.01 | 3.17 | 0.00205 | 57 |
| 347810 | 20.7 | 11786 | A/G | 119 | −0.0036 | 0.01 | 16 | 0.0567 | 0.02 | 1 | −0.0663 | NaN | 2.94 | 0.00208 | 58 |
| 349191 | 4.151 | 1307783 | A/C | 5 | −0.1153 | 0.06 | 1 | 0.0347 | NaN | 168 | 0.0012 | 0.01 | 3.4 | 0.00209 | 59 |
| 352220 | 3.11 | 11369 | C/T | 35 | −0.0339 | 0.01 | 81 | −0.0016 | 0.01 | 58 | 0.0167 | 0.01 | 3.12 | 0.00209 | 60 |
| 350717 | 5.96 | 100368 | C/T | 102 | 0.0042 | 0.01 | 65 | −0.0034 | 0.01 | 6 | −0.0942 | 0.06 | 3.32 | 0.00212 | 61 |
| 351918 | Un.5320 | 21735 | A/G | 89 | 0.003 | 0.01 | 71 | −0.019 | 0.01 | 14 | 0.0523 | 0.02 | 3.29 | 0.00213 | 62 |
| 348558 | NULL | NULL | A/G | 4 | −0.1075 | 0.03 | 61 | 0.0051 | 0.01 | 108 | −0.0021 | 0.01 | 3.15 | 0.00221 | 63 |
| 354434 | 8.112 | 9163 | A/G | 4 | 0.014 | 0.01 | 49 | 0.0255 | 0.01 | 120 | −0.0136 | 0.01 | 3.01 | 0.00221 | 64 |
| 345723 | 13.44 | 470480 | C/T | 71 | 0.0014 | 0.01 | 73 | 0.0078 | 0.01 | 29 | −0.0356 | 0.01 | 3.08 | 0.00232 | 65 |
| 353961 | Un.3536 | 56217 | A/G | 90 | 0.0023 | 0.01 | 70 | −0.0184 | 0.01 | 14 | 0.0523 | 0.02 | 3.25 | 0.00232 | 66 |
| 353960 | Un.3536 | 57283 | A/G | 88 | 0.0024 | 0.01 | 70 | −0.0184 | 0.01 | 14 | 0.0523 | 0.02 | 3.25 | 0.00233 | 67 |
| 350052 | Un.254 | 50541 | G/T | 20 | −0.0046 | 0.01 | 75 | −0.009 | 0.01 | 38 | 0.0273 | 0.01 | 2.77 | 0.00236 | 68 |
| 347164 | 3.22 | 20347 | A/G | 4 | 0.122 | 0.06 | 38 | 0.0094 | 0.01 | 130 | −0.009 | 0.01 | 3.84 | 0.00237 | 69 |
| 353150 | 3.109 | 13111 | C/T | 113 | 0.0107 | 0.01 | 53 | −0.0218 | 0.01 | 7 | −0.072 | 0.03 | 3.22 | 0.00239 | 70 |
| 350716 | 5.96 | 100368 | C/T | 103 | 0.0043 | 0.01 | 65 | −0.0034 | 0.01 | 6 | −0.0942 | 0.06 | 3.34 | 0.00242 | 71 |
| 348284 | 3.82 | 657898 | C/G | 141 | −0.006 | 0.01 | 27 | −0.0034 | 0.01 | 6 | 0.0977 | 0.04 | 3.38 | 0.00243 | 72 |
| 352676 | Un.3712 | 61721 | A/G | 44 | −0.0171 | 0.01 | 73 | −0.0146 | 0.01 | 56 | 0.0255 | 0.01 | 2.88 | 0.00245 | 73 |
| 349084 | 8.27 | 65020 | A/G | 97 | 0.0027 | 0.01 | 66 | 0.0018 | 0.01 | 11 | −0.066 | 0.02 | 3.16 | 0.00248 | 74 |
| 351882 | Un.2371 | 36832 | A/G | 6 | 0.0013 | 0.03 | 70 | −0.0228 | 0.01 | 98 | 0.0126 | 0.01 | 2.98 | 0.00251 | 75 |
| 353906 | Un.1526 | 32213 | A/G | 29 | 0.0037 | 0.01 | 92 | 0.0124 | 0.01 | 52 | −0.0304 | 0.01 | 3.14 | 0.00252 | 76 |
| 346707 | 16.129 | 410662 | A/G | 4 | −0.1075 | 0.03 | 61 | 0.0051 | 0.01 | 109 | −0.0021 | 0.01 | 3.15 | 0.00256 | 77 |
| 353270 | Un.3982 | 3762 | A/G | 152 | −0.0094 | 0.01 | 19 | 0.0456 | 0.02 | 3 | 0.0733 | 0.06 | 3.02 | 0.00258 | 78 |
| 352344 | Un.895 | 64889 | C/T | 12 | −0.0538 | 0.02 | 82 | −0.0105 | 0.01 | 70 | 0.0161 | 0.01 | 3.13 | 0.00259 | 79 |
| 347611 | 16.129 | 410809 | C/T | 4 | −0.1075 | 0.03 | 61 | 0.0051 | 0.01 | 109 | −0.0021 | 0.01 | 3.15 | 0.00263 | 80 |
| 345696 | Un.498 | 297878 | G/T | 58 | −0.0126 | 0.01 | 67 | −0.0106 | 0.01 | 29 | 0.0402 | 0.01 | 3.14 | 0.00267 | 81 |
| 352298 | Un.3982 | 3793 | C/T | 152 | −0.0094 | 0.01 | 19 | 0.0456 | 0.02 | 3 | 0.0733 | 0.06 | 3.02 | 0.00267 | 82 |
| 345859 | Un.3982 | 3843 | C/T | 3 | 0.0733 | 0.06 | 19 | 0.0456 | 0.02 | 152 | −0.0094 | 0.01 | 3.02 | 0.00271 | 83 |
| 348454 | Un.1889 | 129804 | A/C | 5 | −0.1048 | 0.07 | 47 | 0.0006 | 0.01 | 121 | 0.0011 | 0.01 | 3.31 | 0.00283 | 84 |
| 350374 | 8.119 | 103491 | A/G | 123 | −0.0052 | 0.01 | 42 | −0.011 | 0.01 | 9 | 0.0832 | 0.04 | 3.33 | 0.00293 | 85 |
| 345742 | Un.42582 | 606 | C/G | 9 | 0.0691 | 0.03 | 75 | −0.0065 | 0.01 | 90 | −0.0053 | 0.01 | 3.06 | 0.00297 | 86 |
| 352752 | 6.85 | 115198 | A/C | 5 | 0.0647 | 0.04 | 21 | −0.0052 | 0.01 | 148 | −0.0038 | 0.01 | 3.16 | 0.00298 | 87 |
| 353446 | Un.3281 | 68445 | A/G | 49 | −0.0036 | 0.01 | 15 | −0.0436 | 0.02 | 85 | 0.0088 | 0.01 | 2.89 | 0.00299 | 88 |
| 344286 | 12.81 | 869389 | G/T | 5 | −0.1213 | 0.07 | 47 | 0.0106 | 0.01 | 122 | −0.002 | 0.01 | 3.47 | 0.00306 | 89 |
| 346919 | Un.8 | 1240769 | A/G | 110 | 0.003 | 0.01 | 41 | 0.0001 | 0.01 | 4 | −0.1204 | 0.08 | 3.31 | 0.00307 | 90 |
| 347396 | Un.4539 | 42246 | C/T | 5 | −0.0079 | 0.02 | 53 | −0.0266 | 0.01 | 116 | 0.0095 | 0.01 | 2.83 | 0.00308 | 91 |
| 349561 | 8.119 | 103514 | C/T | 120 | −0.0048 | 0.01 | 45 | −0.0114 | 0.01 | 9 | 0.0832 | 0.04 | 3.41 | 0.00311 | 92 |
| 344385 | Un.1584 | 157206 | C/G | 76 | 0.0092 | 0.01 | 83 | −0.0032 | 0.01 | 15 | −0.0524 | 0.02 | 3 | 0.00313 | 93 |
| 352343 | Un.895 | 64839 | A/G | 26 | −0.0379 | 0.02 | 89 | −0.0055 | 0.01 | 59 | 0.019 | 0.01 | 3.12 | 0.00326 | 94 |
| 348965 | Un.2614 | 107060 | A/T | 63 | 0.011 | 0.01 | 80 | −0.0015 | 0.01 | 30 | −0.0291 | 0.01 | 2.91 | 0.00327 | 95 |
| 349048 | Un.497 | 113544 | A/G | 143 | −0.0032 | 0.01 | 26 | −0.0151 | 0.01 | 4 | 0.1286 | 0.03 | 3.73 | 0.00329 | 96 |
| 347878 | Un.78 | 27091 | C/T | 80 | −0.0132 | 0.01 | 84 | 0.0023 | 0.01 | 9 | 0.0575 | 0.02 | 2.96 | 0.0033 | 97 |
| 347662 | 9.8 | 378527 | A/G | 27 | −0.0073 | 0.01 | 74 | −0.0195 | 0.01 | 71 | 0.0166 | 0.01 | 2.8 | 0.00333 | 98 |
| 350012 | Un.3712 | 62009 | C/T | 52 | 0.0271 | 0.01 | 76 | −0.0112 | 0.01 | 45 | −0.0159 | 0.01 | 2.86 | 0.00339 | 99 |
| 347705 | 10.136 | 524355 | A/T | 60 | −0.0235 | 0.01 | 73 | 0.014 | 0.01 | 41 | 0.001 | 0.01 | 2.97 | 0.00347 | 100 |
| 345331 | 25.54 | 9023 | A/G | 42 | 0.0193 | 0.01 | 81 | −0.0196 | 0.01 | 50 | 0.0046 | 0.01 | 2.86 | 0.00363 | 101 |
| 348114 | Un.316 | 184472 | G/T | 58 | 0.0158 | 0.01 | 79 | −0.0016 | 0.01 | 36 | −0.0316 | 0.01 | 2.89 | 0.00368 | 102 |
| 348008 | 11.84 | 71010 | C/T | 5 | −0.092 | 0.05 | 62 | 0.0017 | 0.01 | 106 | −0.0001 | 0.01 | 3.33 | 0.0037 | 103 |
| 349469 | 2.57 | 84580 | C/T | 6 | −0.0892 | 0.06 | 64 | −0.0186 | 0.01 | 103 | 0.0132 | 0.01 | 2.93 | 0.00382 | 104 |
| 351890 | Un.316 | 249349 | C/T | 110 | 0.0111 | 0.01 | 51 | −0.0251 | 0.01 | 13 | −0.0221 | 0.02 | 2.79 | 0.00386 | 105 |
| 353496 | 6.114 | 159195 | A/T | 79 | −0.0189 | 0.01 | 71 | 0.0188 | 0.01 | 23 | −0.0083 | 0.01 | 2.98 | 0.00387 | 106 |
| 348966 | 1.244 | 747120 | C/T | 11 | −0.0063 | 0.03 | 83 | 0.0139 | 0.01 | 80 | −0.0179 | 0.01 | 2.77 | 0.00397 | 107 |
| 350113 | Un.9542 | 25156 | C/T | 23 | 0.0019 | 0.01 | 62 | 0.0202 | 0.01 | 89 | −0.0185 | 0.01 | 3.02 | 0.00404 | 108 |
| 346777 | 11.128 | 169026 | C/T | 93 | 0.0107 | 0.01 | 65 | −0.0222 | 0.01 | 15 | 0.0159 | 0.02 | 2.66 | 0.0041 | 109 |
| 354015 | 8.112 | 6627 | C/T | 125 | −0.0124 | 0.01 | 45 | 0.0255 | 0.01 | 4 | 0.014 | 0.01 | 2.84 | 0.00415 | 110 |
| 346294 | 14.62 | 162410 | G/T | 23 | 0.0005 | 0.02 | 72 | −0.019 | 0.01 | 79 | 0.0127 | 0.01 | 2.66 | 0.00416 | 111 |
| 344344 | Un.783 | 15272 | C/G | 50 | −0.0254 | 0.01 | 86 | 0.0008 | 0.01 | 38 | 0.0224 | 0.01 | 2.92 | 0.00418 | 112 |
| 345199 | Un.268 | 436225 | C/T | 49 | 0.0248 | 0.01 | 92 | −0.0182 | 0.01 | 32 | 0.003 | 0.01 | 2.97 | 0.00418 | 113 |
| 344104 | 6.128 | 110109 | C/T | 6 | −0.1033 | 0.05 | 55 | 0.0226 | 0.01 | 113 | −0.0086 | 0.01 | 3.36 | 0.00425 | 114 |
| 348749 | 2.45 | 1167052 | A/C | 116 | 0.0007 | 0.01 | 51 | 0.0028 | 0.01 | 6 | −0.095 | 0.05 | 3.06 | 0.00437 | 115 |
| 353947 | 1.1 | 46224 | A/T | 70 | −0.0127 | 0.01 | 74 | −0.004 | 0.01 | 30 | 0.0279 | 0.01 | 2.8 | 0.00441 | 116 |
| 349492 | Un.1441 | 108562 | C/T | 73 | −0.0223 | 0.01 | 68 | 0.0153 | 0.01 | 33 | 0.0074 | 0.01 | 2.87 | 0.00451 | 117 |
| 349894 | 9.17 | 25220 | A/G | 6 | 0.0004 | 0.03 | 62 | −0.0244 | 0.01 | 106 | 0.011 | 0.01 | 2.9 | 0.00452 | 118 |
| 345270 | 10.18 | 213730 | C/G | 33 | 0.0229 | 0.02 | 59 | −0.0229 | 0.01 | 81 | 0.0035 | 0.01 | 2.6 | 0.00455 | 119 |
| 347032 | Un.265 | 532743 | A/G | 31 | −0.0296 | 0.02 | 90 | −0.0055 | 0.01 | 53 | 0.0202 | 0.01 | 2.97 | 0.00466 | 120 |
| 350408 | 21.54 | 135072 | A/G | 41 | 0.0041 | 0.01 | 39 | 0.0255 | 0.01 | 86 | −0.0178 | 0.01 | 2.92 | 0.00466 | 121 |
| 344006 | Un.497 | 237927 | A/T | 26 | −0.0408 | 0.02 | 71 | 0.0019 | 0.01 | 77 | 0.0075 | 0.01 | 2.81 | 0.00478 | 122 |
| 349539 | Un.1796 | 1214 | C/T | 57 | 0.0165 | 0.01 | 77 | −0.0209 | 0.01 | 40 | 0.008 | 0.01 | 2.77 | 0.00488 | 123 |
| 344585 | Un.2614 | 106884 | C/T | 63 | 0.011 | 0.01 | 82 | −0.0019 | 0.01 | 29 | −0.0305 | 0.01 | 2.97 | 0.00491 | 124 |
| 348608 | 20.13 | 197921 | A/G | 3 | −0.0243 | 0.05 | 16 | −0.057 | 0.02 | 154 | 0.0039 | 0.01 | 3.05 | 0.00494 | 125 |
| 352602 | 21.108 | 7809 | C/T | 16 | −0.0464 | 0.02 | 75 | −0.0074 | 0.01 | 81 | 0.0124 | 0.01 | 2.87 | 0.00507 | 126 |
| 349734 | 5.125 | 690761 | C/G | 81 | 0.0137 | 0.01 | 74 | −0.0067 | 0.01 | 18 | −0.0537 | 0.03 | 3.12 | 0.00514 | 127 |
| 351979 | Un.2565 | 24132 | A/C | 74 | −0.0178 | 0.01 | 77 | 0.0021 | 0.01 | 23 | 0.0352 | 0.01 | 2.87 | 0.0054 | 128 |
| 348875 | 13.13 | 175493 | A/G | 61 | −0.0007 | 0.01 | 86 | −0.0153 | 0.01 | 27 | 0.0374 | 0.02 | 2.91 | 0.00544 | 129 |
| 344551 | 22.4 | 199537 | A/G | 0 | NaN | NaN | 5 | −0.1017 | 0.04 | 169 | 0.001 | 0.01 | 2.99 | 0.00545 | 130 |
| 349124 | 6.61 | 144726 | A/G | 0 | NaN | NaN | 7 | 0.0812 | 0.04 | 167 | −0.0055 | 0.01 | 2.96 | 0.00551 | 131 |
| 350131 | 6.128 | 110208 | A/T | 99 | −0.0002 | 0.01 | 58 | 0.0066 | 0.01 | 17 | −0.0417 | 0.02 | 2.82 | 0.00557 | 132 |
| 350504 | Un.1608 | 217751 | A/G | 71 | −0.0168 | 0.01 | 63 | −0.0022 | 0.01 | 40 | 0.0245 | 0.01 | 2.78 | 0.00565 | 133 |
| 351458 | NULL | NULL | C/T | 75 | −0.0067 | 0.01 | 78 | −0.0093 | 0.01 | 20 | 0.0444 | 0.02 | 2.69 | 0.0057 | 134 |
| 351719 | 14.42 | 170510 | C/G | 96 | 0.0108 | 0.01 | 62 | −0.0264 | 0.01 | 15 | 0.0096 | 0.02 | 3.03 | 0.00593 | 135 |
| 351968 | Un.3164 | 9905 | A/C | 13 | −0.0561 | 0.02 | 62 | −0.0026 | 0.01 | 99 | 0.0055 | 0.01 | 2.88 | 0.00594 | 136 |
| 349807 | Un.118 | 7387 | C/G | 111 | −0.0088 | 0.01 | 52 | −0.0021 | 0.01 | 11 | 0.0668 | 0.03 | 2.98 | 0.00612 | 137 |
| 352770 | Un.15028 | 4499 | A/C | 13 | 0.0438 | 0.02 | 70 | −0.0062 | 0.01 | 90 | −0.0044 | 0.01 | 2.91 | 0.00615 | 138 |
| 345571 | Un.497 | 237863 | A/G | 26 | −0.0408 | 0.02 | 57 | 0.0041 | 0.01 | 76 | 0.0074 | 0.01 | 2.79 | 0.00621 | 139 |
| 347013 | 6.185 | 105423 | A/G | 23 | −0.0372 | 0.01 | 92 | 0.003 | 0.01 | 58 | 0.0054 | 0.01 | 2.65 | 0.00634 | 140 |
| 352481 | Un.7250 | 18952 | A/G | 94 | −0.0007 | 0.01 | 64 | 0.0098 | 0.01 | 15 | −0.0526 | 0.03 | 2.77 | 0.00637 | 141 |
| 346806 | Un.5923 | 708 | C/T | 4 | −0.0318 | 0.03 | 40 | −0.0298 | 0.01 | 129 | 0.0082 | 0.01 | 2.75 | 0.00641 | 142 |
| 352035 | Un.1825 | 122859 | A/C | 50 | 0.0011 | 0.01 | 78 | 0.0116 | 0.01 | 46 | −0.0285 | 0.01 | 2.75 | 0.00647 | 143 |
| 354080 | Un.4087 | 21229 | C/T | 61 | 0.0136 | 0.01 | 65 | −0.0231 | 0.01 | 46 | 0.007 | 0.01 | 2.55 | 0.00662 | 144 |
| 345391 | 27.51 | 265911 | C/T | 67 | 0.0165 | 0.01 | 79 | −0.0086 | 0.01 | 28 | −0.0278 | 0.01 | 2.93 | 0.00665 | 145 |
| 350770 | 27.6 | 1468442 | C/T | 40 | 0.0235 | 0.01 | 82 | −0.0117 | 0.01 | 52 | −0.0063 | 0.01 | 2.57 | 0.00677 | 146 |
| 350931 | Un.830 | 189264 | C/T | 91 | 0.0146 | 0.01 | 70 | −0.0191 | 0:01 | 12 | −0.023 | 0.01 | 2.72 | 0.00684 | 147 |
| 348309 | 4.151 | 1307725 | C/T | 0 | NaN | NaN | 6 | −0.0903 | 0.06 | 168 | 0.0012 | 0.01 | 2.9 | 0.00688 | 148 |
| 346200 | 13.134 | 388592 | C/T | 68 | −0.0195 | 0.01 | 82 | 0.0114 | 0.01 | 24 | 0.0019 | 0.02 | 2.47 | 0.00694 | 149 |
| 350054 | 10.48 | 151751 | C/T | 3 | 0.1448 | 0.07 | 57 | −0.0189 | 0.01 | 111 | 0.0065 | 0.01 | 3.5 | 0.00704 | 150 |
| 342661 | 2.154 | 245428 | C/T | 50 | 0.0243 | 0.01 | 82 | −0.0131 | 0.01 | 41 | −0.0133 | 0.01 | 2.78 | 0.00706 | 151 |
| 347669 | 11.163 | 228823 | A/G | 5 | 0.0459 | 0.05 | 54 | 0.0198 | 0.01 | 115 | −0.0143 | 0.01 | 2.77 | 0.0071 | 152 |
| 345058 | Un.12226 | 5567 | C/T | 47 | 0.0033 | 0.01 | 74 | −0.018 | 0.01 | 53 | 0.0157 | 0.01 | 2.64 | 0.00719 | 153 |
| 350721 | 25.32 | 172400 | C/T | 104 | −0.0017 | 0.01 | 60 | −0.0135 | 0.01 | 10 | 0.0644 | 0.02 | 2.83 | 0.00723 | 154 |
| 344481 | 11.8 | 165588 | A/C | 59 | −0.0242 | 0.01 | 83 | 0.0138 | 0.01 | 30 | −0.0035 | 0.01 | 2.76 | 0.00731 | 155 |
| 352723 | Un.14191 | 4098 | C/G | 10 | 0.035 | 0.03 | 65 | −0.0225 | 0.01 | 99 | 0.0078 | 0.01 | 2.51 | 0.00731 | 156 |
| 346573 | Un.498 | 298373 | A/G | 27 | 0.0374 | 0.02 | 81 | −0.0128 | 0.01 | 65 | −0.0053 | 0.01 | 2.86 | 0.00734 | 157 |
| 346994 | 20.77 | 1008737 | C/T | 74 | −0.0204 | 0.01 | 74 | 0.0099 | 0.01 | 22 | 0.0153 | 0.02 | 2.55 | 0.00742 | 158 |
| 352253 | 23.91 | 79876 | C/T | 143 | 0.0027 | 0.01 | 26 | −0.0043 | 0.01 | 3 | −0.1488 | 0.1 | 3.36 | 0.00745 | 159 |
| 345744 | 8.6 | 243128 | A/G | 114 | 0.0082 | 0.01 | 50 | −0.0273 | 0.01 | 10 | 0.0083 | 0.02 | 2.71 | 0.00746 | 160 |
| 345999 | 3.225 | 182942 | A/G | 2 | 0.1096 | 0.11 | 34 | −0.0343 | 0.02 | 128 | 0.0037 | 0.01 | 2.67 | 0.00754 | 161 |
| 342582 | 18.11 | 1892602 | A/G | 79 | −0.003 | 0.01 | 55 | −0.0196 | 0.01 | 30 | 0.0346 | 0.01 | 2.76 | 0.00772 | 162 |
| 353285 | Un.13617 | 8021 | A/G | 14 | 0.0495 | 0.02 | 66 | −0.0002 | 0.01 | 93 | −0.0116 | 0.01 | 2.77 | 0.00785 | 163 |
| 344225 | Un.1079 | 22572 | C/G | 10 | 0.0663 | 0.02 | 42 | −0.0033 | 0.01 | 121 | −0.0071 | 0.01 | 2.95 | 0.00791 | 164 |
| 346776 | 11.128 | 169026 | C/T | 92 | 0.0092 | 0.01 | 65 | −0.0222 | 0.01 | 15 | 0.0159 | 0.02 | 2.49 | 0.00793 | 165 |
| 343060 | 21.54 | 134766 | C/G | 16 | 0.005 | 0.01 | 63 | 0.0175 | 0.01 | 94 | −0.0161 | 0.01 | 2.64 | 0.0081 | 166 |
| 354110 | 16.88 | 251873 | C/T | 3 | −0.1208 | 0.06 | 18 | −0.0101 | 0.02 | 152 | 0.0013 | 0.01 | 2.74 | 0.00814 | 167 |
| 347357 | 4.151 | 1307706 | C/T | 167 | 0.0015 | 0.01 | 6 | −0.0903 | 0.06 | 0 | NaN | NaN | 2.91 | 0.00819 | 168 |
| 343987 | 3.29 | 344695 | G/T | 8 | 0.0718 | 0.04 | 65 | −0.0148 | 0.01 | 101 | 0.0004 | 0.01 | 2.83 | 0.00829 | 169 |
| 343337 | 27.6 | 1472116 | C/T | 40 | 0.0235 | 0.01 | 82 | −0.0117 | 0.01 | 52 | −0.0063 | 0.01 | 2.57 | 0.00831 | 170 |
| 346674 | Un.1142 | 48580 | A/G | 82 | 0.0014 | 0.01 | 74 | −0.014 | 0.01 | 18 | 0.0317 | 0.02 | 2.6 | 0.0084 | 171 |
| 347830 | 21.56 | 413745 | C/T | 134 | −0.0056 | 0.01 | 36 | −0.0004 | 0.01 | 3 | 0.138 | 0.03 | 3.22 | 0.00848 | 172 |
| 350100 | NULL | NULL | G/T | 138 | 0.0056 | 0.01 | 33 | −0.0336 | 0.01 | 3 | −0.0019 | 0.04 | 2.64 | 0.00848 | 173 |
| 346790 | Un.6329 | 19492 | C/T | 135 | −0.0096 | 0.01 | 35 | 0.0271 | 0.01 | 4 | −0.0019 | 0.03 | 2.52 | 0.00856 | 174 |
| 348375 | Un.1468 | 37764 | A/G | 4 | −0.0006 | 0.02 | 19 | −0.0424 | 0.02 | 151 | 0.0031 | 0.01 | 2.42 | 0.00856 | 175 |
| 350976 | 9.86 | 594360 | A/G | 31 | 0.0284 | 0.01 | 90 | −0.0072 | 0.01 | 52 | −0.0103 | 0.01 | 2.46 | 0.00863 | 176 |
| 347335 | 18.5 | 12668 | C/T | 58 | −0.0258 | 0.01 | 91 | 0.0092 | 0.01 | 23 | 0.0127 | 0.01 | 2.65 | 0.00866 | 177 |
| 345053 | Un.7584 | 27151 | C/T | 117 | 0.0084 | 0.01 | 52 | −0.0258 | 0.01 | 4 | −0.0064 | 0 | 2.65 | 0.00869 | 178 |
| 347260 | Un.29 | 891126 | A/T | 1 | 0.0308 | NaN | 29 | −0.0348 | 0.02 | 144 | 0.0044 | 0.01 | 2.52 | 0.00883 | 179 |
| 348171 | Un.518 | 163723 | C/T | 3 | 0.0026 | 0.02 | 34 | −0.0332 | 0.01 | 137 | 0.0057 | 0.01 | 2.64 | 0.00912 | 180 |
| 352009 | Un.1573 | 179968 | A/G | 51 | 0.0141 | 0.01 | 64 | 0.0001 | 0:01 | 58 | −0.0187 | 0.01 | 2.47 | 0.00918 | 181 |
| 348433 | Un.431 | 22021 | C/T | 83 | −0.0026 | 0.01 | 66 | 0.0109 | 0.01 | 25 | −0.0341 | 0.02 | 2.47 | 0.00921 | 182 |
| 350229 | Un.1142 | 48801 | A/G | 82 | 0.0014 | 0.01 | 74 | −0.014 | 0:01 | 18 | 0.0317 | 0.02 | 2.6 | 0.00939 | 183 |
| 349668 | 20.17 | 112619 | C/T | 52 | 0.0106 | 0.01 | 84 | −0.019 | 0.01 | 38 | 0.0185 | 0.01 | 2.44 | 0.00953 | 184 |
| 352468 | Un.762 | 163069 | A/C | 37 | 0.0211 | 0.01 | 86 | 0.0003 | 0.01 | 50 | −0.0248 | 0.01 | 2.67 | 0.00962 | 185 |
| 346765 | 26.3 | 100354 | A/T | 42 | 0.0181 | 0.01 | 84 | 0.0027 | 0.01 | 48 | −0.0278 | 0.01 | 2.72 | 0.00967 | 186 |
| 345883 | Un.248 | 26719 | A/C | 65 | 0.0112 | 0.01 | 84 | −0.0008 | 0.01 | 21 | −0.0357 | 0.02 | 2.55 | 0.00984 | 187 |
| 347412 | Un.1468 | 39091 | A/G | 4 | −0.0006 | 0.02 | 19 | −0.0424 | 0.02 | 151 | 0.0031 | 0.01 | 2.42 | 0.00989 | 188 |
| 353889 | 27.31 | 401645 | A/G | 0 | NaN | NaN | 8 | −0.0725 | 0.05 | 166 | 0.0014 | 0.01 | 2.68 | 0.01003 | 189 |
| 343366 | Un.3371 | 62206 | A/G | 4 | 0.069 | 0.05 | 27 | −0.0058 | 0.01 | 142 | −0.0037 | 0.01 | 2.86 | 0.0104 | 190 |
| 354069 | Un.10 | 133300 | G/T | 148 | 0.0036 | 0.01 | 3 | −0.1224 | 0.05 | 0 | NaN | NaN | 2.73 | 0.0104 | 191 |
| 348745 | Un.1350 | 19656 | A/C | 42 | −0.0029 | 0.01 | 90 | 0.0087 | 0.01 | 42 | −0.024 | 0.01 | 2.46 | 0.01042 | 192 |
| 345345 | 21.26 | 655280 | G/T | 134 | −0.0008 | 0.01 | 33 | 0.0071 | 0.01 | 6 | −0.0852 | 0.04 | 2.64 | 0.01064 | 193 |
| 351367 | 20.47 | 356625 | A/G | 8 | 0.0168 | 0.03 | 61 | 0.0169 | 0.01 | 104 | −0.0148 | 0.01 | 2.58 | 0.01107 | 194 |
| 350048 | 17.96 | 376293 | A/G | 2 | 0.0158 | 0.01 | 27 | 0.0312 | 0.02 | 145 | −0.0084 | 0.01 | 2.46 | 0.01127 | 195 |
| 350311 | 20.85 | 52419 | A/G | 19 | −0.0441 | 0.03 | 79 | 0.009 | 0.01 | 76 | −0.0029 | 0.01 | 2.52 | 0.0116 | 196 |
| 342784 | 19.55 | 711099 | A/G | 7 | −0.0025 | 0.03 | 58 | −0.0222 | 0.01 | 108 | 0.0067 | 0.01 | 2.37 | 0.01189 | 197 |
| 346622 | 22.79 | 852700 | G/T | 14 | −0.0268 | 0.01 | 59 | 0.0195 | 0.01 | 97 | −0.0097 | 0.01 | 2.39 | 0.01202 | 198 |
| 344417 | Un.762 | 163430 | A/G | 37 | 0.0211 | 0.01 | 86 | 0.0003 | 0.01 | 51 | −0.0226 | 0.01 | 2.53 | 0.01228 | 199 |
| 351991 | Un.11570 | 5291 | A/G | 17 | 0.0432 | 0.02 | 84 | −0.0082 | 0.01 | 73 | −0.0054 | 0.01 | 2.61 | 0.01244 | 200 |
| 352036 | 19.42 | 2700408 | A/G | 10 | −0.059 | 0.03 | 45 | 0.0106 | 0.01 | 116 | −0.0021 | 0.01 | 2.5 | 0.01252 | 201 |
| 354022 | 5.9 | 234958 | C/T | 59 | −0.0108 | 0.01 | 84 | 0.0122 | 0.01 | 31 | −0.0238 | 0.01 | 2.57 | 0.01254 | 202 |
| 346155 | 7.18 | 638724 | A/G | 49 | 0.0217 | 0.01 | 85 | −0.0128 | 0.01 | 40 | −0.008 | 0.01 | 2.49 | 0.01284 | 203 |
| 349426 | NULL | NULL | C/G | 26 | −0.0391 | 0.02 | 68 | 0.007 | 0.01 | 80 | 0.0024 | 0.01 | 2.41 | 0.01292 | 204 |
| 347424 | 18.79 | 33699 | C/T | 46 | −0.023 | 0.01 | 94 | 0.0004 | 0.01 | 34 | 0.0199 | 0.01 | 2.59 | 0.01352 | 205 |
| 346793 | Un.7963 | 19974 | C/G | 49 | −0.0245 | 0.01 | 83 | 0.0051 | 0.01 | 42 | 0.0103 | 0.01 | 2.38 | 0.0138 | 206 |
| 347268 | 11.173 | 33171 | C/G | 55 | 0.0118 | 0.01 | 89 | −0.0191 | 0.01 | 30 | 0.0236 | 0.01 | 2.6 | 0.01383 | 207 |
| 353660 | Un.6489 | 18236 | A/T | 35 | −0.0309 | 0.02 | 68 | 0.0046 | 0.01 | 71 | 0.0059 | 0.01 | 2.49 | 0.01489 | 208 |
| 348539 | 6.115 | 1073339 | A/G | 5 | −0.0689 | 0.04 | 36 | 0.0137 | 0.01 | 133 | −0.0037 | 0.01 | 2.59 | 0.01506 | 209 |
| 347616 | 12.46 | 349109 | C/T | 120 | −0.0081 | 0.01 | 50 | 0.0061 | 0.01 | 4 | 0.0807 | 0.06 | 2.56 | 0.01909 | 210 |

Locus is the ParAllele SNP designator, scaffold-v2 is the version 2 draft bovine genome sequence scaffold, location is the base pair location within the scaffold, Base-change is the alternative bases for the SNP, N is the number of animals for genotype 0, mean0 is the mean phenotype for genotype 0, SE is the standard error of the mean, mean1 is the mean phenotype for genotype 1, mean2 is the mean phenotype for genotype 2, tmax is the t test for the genotypes with the greatest difference, and PermP is the P value determined from 100,000 permutations of the data. For each locus, genotypes 0 and 2 are the opposite homozygotes, genotype 1 is always the heterozygote. Genotype 0 is the allele with the first alphabetic designation, so for locus 349624 with an A/C polymorphism genotype 0 is AA while genotype 2 is CC.

TABLE 2
The genes and genic regions associated with certain of the SNP listed in Table 1 associated with tick resistance.

| | scaffold- | | | | | | | |
|---|---|---|---|---|---|---|---|---|
| Locus | v2 | bp | ibiss4 | scaffold-v1 | tmax | PermP | HumChr | Gene |
| 349624 | 4.14 | 285504 | Null | SCAFFOLD51473_4481 | 3.91 | 0 | Hsa7 | COL1A2 collagen 1 alpha 2 |
| 352775 | Un.5392 | 43292 | Null | SCAFFOLD255152_4868 | 3.69 | 0 | Hsa1 | not near gene |
| 349705 | Un.51 | 655288 | Null | SCAFFOLD85870_4758 | 3.89 | 0.00003 | Hsa2 | SSFA2 to ITGA4 (CD49D) |
| | | | | | | | | Integrin alpha 4 |
| 351771 | Un.11015 | NULL | Null | SCAFFOLD313482_2366 | 3.66 | 0.00006 | Hsa4 | no gene id |
| 347700 | Un.895 | 65002 | Null | SCAFFOLD305461_2727 | 3.57 | 0.00008 | Hsa10 | no gene id |
| 345751 | Un.734 | 193986 | Null | SCAFFOLD246162_4918 | 4.01 | 0.00011 | Hsa8 | near TSTA −> TSTA3 |
| | | | | | | | | tissue specific trans- |
| | | | | | | | | plantation antigen P35B |
| 351349 | Un.10044 | 18624 | Null | SCAFFOLD100910_4645 | 3.61 | 0.00013 | Hsa10 | RPS13 |
| 353220 | Un.734 | 193387 | Null | SCAFFOLD246162_5517 | 4.26 | 0.00015 | Hsa8 | near TSTA and RDH10 |
| 354299 | Un.734 | 193708 | Null | SCAFFOLD246162_5196 | 4.04 | 0.00016 | Hsa8 | near TSTA and RDH10 |
| 353165 | 3.108 | 21885 | Null | SCAFFOLD19865_500 | 3.78 | 0.00024 | Hsa1 | ELTD1, EGF, Latrophilin |
| | | | | | | | | and seven transmembrane |
| | | | | | | | | containing 1 |
| 347051 | 10.41 | 71349 | IBISS4snp990 | Null | 3.83 | 0.00026 | Hsa4? | RNASE1, pancreatic ribo- |
| | | | | | | | | nuclease, pos cand |
| | | | | | | | | region CBX Bta 10 RNASE |
| | | | | | | | | cluster known on Hsa14 |
| 346367 | 4.42 | 1548870 | Null | SCAFFOLD155969_6061 | 3.58 | 0.00032 | Hsa7 | SEMA3C (semaphorin 3C, |
| | | | | | | | | non-multidrug |
| | | | | | | | | resistance?) |
| | | | | | | | | and CD36 antigen |
| 349425 | 21.6 | 958026 | Null | SCAFFOLD271702_39983 | 3.64 | 0.00036 | Hsa15 | FLJ12572, near Desmuslin |
| | | | | | | | | and IGF1R |
| 344395 | Un.856 | 42123 | Null | SCAFFOLD75120_12641 | 3.52 | 0.00038 | Hsa11 | near NELL1 NEL-like 1 |
| 351066 | Un.8255 | 16212 | Null | SCAFFOLD295013_6808 | 3.41 | 0.00038 | Hsa7 | no genes - Bta4 & 25 |
| 351896 | 23.78 | 164911 | Null | SCAFFOLD75180_16508 | 3.75 | 0.0004 | Hsa6 | one scaffold up from PRL |
| | | | | | | | | Prolactin |
| 345776 | 14.112 | 653790 | Null | SCAFFOLD255022_2946 | 3.65 | 0.00044 | Hsa8 | near RIPK2 (affect T cells) |
| | | | | | | | | Receptor-interacting serine/ |
| | | | | | | | | threonine kinase 2, Hsa8 |
| 346463 | 2.101 | 164846 | Null | SCAFFOLD185228_7995 | 3.42 | 0.00047 | Hsa2 | near GPR39 G-protein- |
| | | | | | | | | coupled receptor 39 |
| | | | | | | | | (related to GHSR) |
| 351705 | Un.801 | 84560 | Null | SCAFFOLD286772_8833 | 4.2 | 0.0005 | HsaX | LAMP2 BtaX, Lysosomal- |
| | | | | | | | | associated membrane |
| | | | | | | | | protein 2 |
| 350060 | 1.47 | 18797 | Null | SCAFFOLD20440_9960 | 3.3 | 0.00056 | Hsa3 | ARL6, ADP-ribosylation |
| | | | | | | | | factor-like 6, intra- |
| | | | | | | | | cellular traffic regulator |

Locus is the ParAllele SNP identifier, scaffold-v2 is the location which gives the bovine chromosomal location (4.14 means bovine chromosome 4, scaffold 14), bp is the location with the scaffold, ibiss4 is the ibiss4 reference, scaffold-v1 is the original designation on the version 1 scaffolds, tmax is the t test for the maximum difference between genotypes, PermP is the P value derived from 100,000 permuation tests, HumChr is the location on a human chromosome of the homologous gene and Gene is the closest gene to the SNP in the scaffold.

TABLE 3
DNA Sequences for SNP

| | V1 Scaffold | | | |
|---|---|---|---|---|
| | or IBISS4 | | Alternative | |
| Locus | No. | 5′ Flanking Sequence | Bases | 3′ Flanking Sequence |
| 349624 | SCAFFOLD | ACTTTTCTTAAACTAGTTGCTTCCTCTAAACTTACA | A/C | TAAACTAAAGGAAAAATCTCTCTGCAGAGTAAACTG |
| | 51473_4481 | AAGTTAGTGGAAATAAAAGAGAAAAATCCCAAAACT | | ATAACTGGAAAAGCATTCCTGGAACCTGATGCAAAT |
| | | AAAAAAAAAAAACCCAAAAAACCTCTTTTTCTAATT | | AGCTAACGCTGCCCGGCTCCCTCCCTGTTGATGTGG |
| | | AAGAGCACTGATGTGGGAGATAACTTTAGAAGACTT | | CAGAGTTTGAAGCTGGCAGTCGTCAGCCATTTGGTG |
| | | TTTGAAATTGCAGTAACTGTTTGGGAAATGTGATTA | | GGAGGCCGTGGATCCAGAGGAAACAATGAGGATGGA |
| | | TAATTTACCTTTTAGGATAACAAAATTCTCAGCAAG | | GAAAACTGAAAGGGAATTTTTAAAGAGGGAGACATT |
| | | TTTTGGAGAATTCGAGAAATTCGAGCACACGTTT | | TTTGTTCCAGCAGTTTTTTACACATTATTTTGAC |
| 352775 | SCAFFOLD | TAGCCACTGAACCACCAGGAAGGTCCCTTGAAGTTC | T/C | CTACATCTCTTTTAAAATTAAAATATATGTTAACTT |
| | 255152_4868 | CTTCTAACCATTTTCATTTTCAAGTTTTTAGAAACT | | TCCTTTTAATTCTGAGAAATTATAACCTTTCAGTTC |
| | | GCCTTTTACTTTCCGTCCAGGGTATTTGTATATTTA | | AGTTCAGTCACTCAGTCATGTCCAACTCTTTGCGAT |
| | | AGAGAAGTTTGCTTCCAAAAGTCTTATGCTCAAATT | | CCCGTGGACTGCAGCATGCCAGGCTTCCCTGTCCAT |
| | | ACACACTAGTTTTAAGACGATACTTACTTCTTGGCC | | CACCAACTCCTGGAGTTTACTCAAACTCATTTCCAT |
| | | TACACTGGGATGTATGAATGGGAAATAAAAGTAGTA | | TGAGTTGGTGATGCCAGCCAACCATCTCACCTTCTG |
| | | TGCATGTGATAACTGAATGGTGGGCAATAGGAAG | | TCATCCCCTTCTCCTCCTGCCTTCAGTCTTTCCC |
| 349705 | SCAFFOLD | GTACAACACACCCAGGAGTGTTCTCAGAACACCTTT | G/T | GGGGGTTCTGCAGTGTTTAGCATTTGAGATTGTATT |
| | 85870_4758 | GATTTCACCACTCCTGGGTGTTCACAGAGAAGGGAT | | TTGGATACATTTCAGAGGAGTCAAGCCAACTTGGCA |
| | | GATACTGAAAGGTATGCAGTCAAACAGTAATGGTTA | | TTACTCAGTGTCTGACCAGCCAACCTGGACCCCTTC |
| | | GATGTCATCTTCTGCTGGGAAGTAAGATAATTAAGT | | AGAAGGTGATTTTCCAGGCTGAATTTTTCAGGGCCT |
| | | AATTGCTTTGCTTTCATGCCACCACTTACAGACACC | | TGTTTCCCGCCTCCTATTTCCTTCTCCATTATGTTA |
| | | ATAGTCTCTCTCGGGAAAATAATAACCTTTTTAAGT | | CTTGCATTCATGGCTTATTAGCAATGCCAGCAGAAA |
| | | GGACCAAGCCCATACTTTATAGCATATGACTTTT | | TGTTGGCTGCAAAAGTCAGGAAAAGTACAATCAG |
| 347700 | SCAFFOLD | GAACATATAATTTCTAAATGTCTAAAGCCTGGAATT | C/T | GAGTGTGTAGTTTCTTGCCTTACGATGCCTACTATG |
| | 305461_2727 | AATGTCTGTGACCTCGGTAATACCAAGAGATGATTG | | ATTTTAAAGTTACTTAAAATTGTTTCTAAAGACTTC |
| | | TGAGAATGAAGATTGTTCTAATTTTCTCAGCAAAGA | | CCCTGAACTTGACATCAGGAACTTACAGTTATACTT |
| | | TAATATTTTAAAAACTACAAACTGAAAATCACTTTT | | GGTTGAGGCCCCTGTTCTCAGTGGGGTATTGTGGGG |
| | | TGTGACAGCACGTTTAATTTGTGTAGCTATTCTCAT | | AAGAGAAGACAGAAGTTGATAAAAGGCAGGGTTCCC |
| | | TGCAGGCTGATTTGCTAAAATATGAGCGAACAAGTC | | TCAGTGAGGATCTGGGAAGTGAGAGGGGTTTACACG |
| | | TAATGTAGGGAGTTGAACAGAGCACTATGGTAT | | ATGAAGATGTTGCCCTCCATTGAGTCATTTATCA |
| 351771 | SCAFFOLD | AACACCTTCTGGTTAAGTTGTAAATGGGACATTTGG | T/C | GACTTCGTGTATTTCATAGAGTTGTTGCTAAGATAC |
| | 313482_2366 | CCTACCCACCCTAGCACTGCTCCATTACAGCCCAGA | | ATGAAACAGTGTCTTCTGGGCCATAGGGCATTGTAA |
| | | ATCCAAGTGTTGTAAGCAAGAAAAATGTTAGAGGCT | | ATTCACGCTCTTAGCCCCTGATAGACTCCAAACAAA |
| | | GAGCTATGAAAATGGCAGCTGTGATATAATTATGAA | | TGTTAGATCAAATTTGAAGACAGAAAAACNNNNNNN |
| | | TATAACCTGCTGGAGACAGAATTGTGAAGCCTATTC | | NNNNNNNNNNNNNNNNNNNNNNNNNNNNNNNNNNNN |
| | | ACCAATTCTAGCCCTGCTGCTAAGCACCTGTTATGC | | NNNNNNNNNNNNNNNNNNNNNNNNNNNNNNNNNNNN |
| | | TCTCTTTGTACTTGTAAAATGGGTACATAAAACT | | NNNNNNNNNNNNNNNNNNNNNNNNNNNNNNNNNN |
| 345751 | SCAFFOLD | TTTCTATTTGTCCTATTTCATATTTCTTTTTTTCCT | T/C | TGGACCACCAAGGGGAAGTCCCTACAATCTACTTTA |
| | 246162_4918 | CTTTTCTTGCTTTGAGATGATTAAATATTTTTTACT | | ACTTAATTACTATATTTCAAATAATGCAAGGAGCTT |
| | | TTTCTATTTCCCTATAATCTTGTTACTTGTATCCTT | | ACAACATTTTAACTCCATGTATGCCCCATCCCACTT |
| | | TTCTTTTAGTGGTTATCATACAGATTTTATCATACC | | CTGTGGTATTACTGTATTTCATTTCTACATATATTT |
| | | TCTGCTATGTGGTGTGTGGGATCTTCTCCCCAATCA | | TAAACTTCTTAAGAAAATACTACTGCTACTGCTTCT |
| | | GGAAGCATAGAGTGCTATGTGGTGTGTGGGATCTTC | | CTTTCCTTTACAAGTTGTCATGCTTTCCTCTTGTAT |
| | | TCCCCAATCAGGAAGCATAGAGTCTTAACCA | | CATTATTTCTTTAATATTTCTTTTAGTGTAGCTC |
| 351349 | SCAFFOLD | GCAAAGGCATTTTTTCCCATCACTTTAACCTCCTCC | C/T | GTTTTATTTCATCTTTGCCTTTTTTGTCTGCCTTCA |
| | 100910_4645 | CTGGCCCAGCTTTAGCTGATGGCAGATTCCAGGCCA | | GTTGGGGCCAGCCTCACAAAAGAGAGGGCCTTTTGG |
| | | CATCCATCCTCCTCCAACGTTTTCCCCAGGCCGAGA | | CTTTCAGAATTCTTCAGCCTCTGCTCATTATCTCAG |
| | | TTCTTCATTCTTGGGGTTCATCTGAGCCGTTTCCAT | | GCACTAGTTTGGACTGAACCAATGTGGGGTGATTTC |
| | | AGTGACAGTGACTTGTTGAGTTTTCAACCTCCCATC | | CTTGCTGTCGCCCCCGTACCCTGGCCCCACCCTGGA |
| | | GAGGTCCTTGCAATTTGCCAGCTAAGCCACCGATCA | | GACAGGAGGGAGCATACCTCAGGCTTGGAAGGAGGT |
| | | GCAGGCATGTGTGCCAGGTTAGTAAGACAAAGTC | | CACTGGGAACTTCTTTTAGTTTGCATTTGAGTGT |
| 353220 | SCAFFOLD | TTTCTAGTTTAATTCCACTGCAGTCTTATTTGATAT | A/C | TTTTAATTACCATTTACATGGTTTATATTTTTCTAT |
| | 246162_5517 | TTGTTTAAAAATCTCCTATAATGTCTACTTTCCTTT | | CCTTTTATTTTCGATCTTTCCTGCCCTTTTAAGGTG |
| | | TCATTATATTCCTTTGATCTTTGTGTATTTTATGTT | | TGCTTCTTATAACCTGGGTTGAGAAAATCCCCTGGA |
| | | AATAGAGCATACAAATTTAGGATGGTTAAATCTTTC | | GAAAAAGGAAAGGCTATCCACTCCAGTATTCTGGCC |
| | | TGTTGTATTAATCCTTCTGTTACTATGAAATATCAC | | TGGAGAATTCTATGGACTCTATAGTTCATGGGATCG |
| | | TCTACTGTGCATAATACTTCCTGCTTTGAAGTTTAT | | CAAAGAGTCAGACATGACTGAGCGACTTTCACTTCA |
| | | TTTGTTTGATGTTAGTGTAGCTATAACAAGCTTT | | TTTCACTTCACTTCTTATAACCAGCATATAATTA |
| 354299 | SCAFFOLD | TGTGCTTCTTATAACCTGGGTTGAGAAAATCCCCTG | C/T | ATGTTTAATTTTAATATTACTAGTTGTTTTCTATTT |
| | 246162_5196 | GAGAAAAAGGAAAGGCTATCCACTCCAGTATTCTGG | | GTCCTATTTCATATTTCTTTTTTTCCTCTTTTCTTG |
| | | CCTGGAGAATTCTATGGACTCTATAGTTCATGGGAT | | CTTTGAGATGATTAAATATTTTTTACTTTTCTATTT |
| | | CGCAAAGAGTCAGACATGACTGAGCGACTTTCACTT | | CCCTATAATCTTGTTACTTGTATCCTTTTCTTTTAG |
| | | CATTTCACTTCACTTCTTATAACCAGCATATAATTA | | TGGTTATCATACAGATTTTATCATACCTCTTTGACA |
| | | TTTTTATTAGTCTGATGGGTTTTGTTTTTAGCTGAA | | TATTACAATTGACTTTTTAAAAGAAATTTTATTTCT |
| | | ATATTTAGTCCACTTATATCCAATGTATTTATTG | | TGGTTGTGCTATGTGGTGTGTGGGATCTTCTCCC |
| 353165 | SCAFFOLD | AACAGAACACAGTTTTTAATGGGGATTTTTTCCTTT | A/G | TCTAGGTTATATTTTGGGCATGAATAATCTTGTTGC |
| | 19865_500 | TAAAATTCTTAAATTCCCTTTTTATTTAAAGAATGG | | CTTTTTCTCATCTCTAAAGAGTTTCTCATTTTTCCT |
| | | TACATATTCACTTGTGTTGATATTCACTATTTGAGG | | TATTTTTCTCTAAGATTGTTAGTAGACCACTATGAA |
| | | TTTATAATATCCTCTTAAATTGTTCAATCAGTGATT | | ATCACTGGACAGAATTTTTGTTACAGAGGATGCCTA |
| | | TTAATCTTTTTCACCTTTTATTAATTCATTGACAGA | | CATTTTGAGCAATTAAAATTGTTGTTTCTTGGCATT |
| | | TTTCACACTTTTTTTCAGTAAATACATTTACTCTGA | | ATAAATATCATCTTCATTCCCTGATAGCTCAGTTGG |
| | | TTTTTGGAATTAAAACATTTTTAACTTCTGAATC | | TAAAGACTACGCCTGCAAAGCAGGAGACCCCAGT |
| 346367 | SCAFFOLD | AATGGAATATAACCTACGTTTCTTAAAGTGCCCAAT | A/G | CTCCTTGAGGATGGGGAGAATCTGAGCAGATTTCAA |
| | 155969_6061 | CACTCCTTGGCTTTAGGTATACTTAAGTATTCTTAT | | TTTTTAAAATATCCTTCTTACATTTTGCACAGCAAG |
| | | ATCCTTACTGTATTATTTATCACCTGTCTATTTCCA | | GTACTCAATAAACATTTGTCAAATGGAATGGAATAG |
| | | ATTTAAATACAAGCACAAATTTGCAGCTTTCAATGA | | ATGATCTCCGATGCCTGTAGCATAAATTCTCACACA |
| | | TTTCCGGGTCAACATTTATATTCAAACAGTAAATTA | | GTGTACTGCAATAGTACTCCGGGCCAATGCTAATGC |
| | | TTTAAATGAAGAAGTCAGTTTACTATGCTTTAAACT | | CAAAACAATAAAACTAGTGAAAAATATAGATTACCT |
| | | TATTATCTATTTTGCCTTCCATATTATGTCATAC | | TCCTAATGTTTACTAGGCAAACAATTTTTAAGGA |
| 349425 | SCAFFOLD | AGATGAGCTTCAGAAGATCCCACAGGGATGGAGACT | C/A | GTTGTATACCTCCAGTCTTTCCTTTCATAACTGATT |
| | 271702_39983 | TGTTTTCATCTGCAACGAACAAGTGAAATCTCCTCT | | TTTCTTGGAGAATATTAGTATACTGGTATGAAAATA |
| | | GATATTACTAAAGCAATAGGAGGTTTCCTTTTATTT | | CCAGTTTCTCTTCCGAATTGTGACTTCTTTATCTTT |
| | | ATTTGAAATGCATTTTTAGTAGCATGTTATTCAGAA | | TGGATAAATCTCTGTGCAGCCCCAAGGCTCGTGAGG |
| | | TTTGACACTATATTCTGCATTATGGAAAGTAACTTT | | GTTTAAATTACTTTATTTATTTACTTGGCTACATCG |
| | | AAAAGATCTAAATAAGTATGTGTATCTAGGAGTTAA | | GGTCTTAGCTGAGGCACATGGCATCTTTTGTTGCGG |
| | | ATATTCTTTAAACCACAAGAATGGGTGACAATCT | | TGCACAGACGCTCTCACGCATCTCCTGTGGCACG |
| 347051 | IBISS4snp990 | GCCACACCCGTACCCTCACTGCCACCTCCACACAGG | A/G | GGCAATAACTCAAGTTAGGTTTCTTATTCA |
| | | CTTCTCTGACTTGA | | |
| 344395 | SCAFFOLD | ACAAAGCAGAAGTCCAAGAAGCTGCATTCCCAAGAT | G/C | TCATAAATGCCTAGGCAGATGAAGGCCTTCTCTTTT |
| | 75120_12641 | TGCCCTGGCCCCAGCACACACTCTGAAGAACACACT | | GAGGTACCTCACCCCCAGAGACCTAGCAAAGTAATC |
| | | TAAGGGCTGGGCAGCTTTCTAATTATGGGGAGATCT | | TAGGGTGGAGTTGGCATCAGGATTTATTTTATGTGT |
| | | TCGATTTAAAGTTAATACCAGAAAGGATGCTGTGGC | | CCCAAGTGATGGAGAGAAGTAGCCACTGCTGAGAAT |
| | | TGGCTTCTGCATGAAATATGCAGTTAAAAGCCCACT | | TGAGTCCTGAGCACCCTCCTGGCTCCAGGTCCAAGG |
| | | GCCAGGGAAACTTCTGCCAAGCCCTAAGTACCAGCC | | TGATAAACTGAGCCCCCTCTTGTCAGACACAGCGCA |
| | | ACCCTGTGTTTCTCCTTTCCAACGATACCTGCAG | | GAAGGTTAGTTTAGCCCCCATCTCACCCACCACT |
| 351066 | SCAFFOLD | TCCTTCCTTTGCTTCCATGGGGTTTGATCCCAAGAG | G/A | CACTCAAACTATTATAGTATCTGTTTGAGAGTTGGC |
| | 295013_6808 | AACTCAATTAAACATTCCAGTTGTAAAGAACCCACC | | TTTATACAGAACCCATATTGTAACACCCTCTCAGGC |
| | | TGCCAATGGGAGACATAGGAGGCATGGTTCAGTTCC | | AATGAGCTGCCTTTTGTACTAACACATATTTGTATT |
| | | TGGATGGGGAAGATGCCCTGGAGGAGGGCATGCAAC | | TTTCTCATAAATTTCAATTCAAGTATTTTTCCTTAA |
| | | CCACTCCAGTATTCTTGCCTGGAGAATCCCATGGAC | | GATCTTTCATAACCTTTCTAGGTCAACAGAGTTATT |
| | | AGAGGAGCATGGTGGGCTACAGTCCGTGGAGTCACA | | CTTCCCATGCTCTCCCATATCTTACAACATAAGAAC |
| | | GAGTCGGACACAACTTAGGTGACTTAGCATGAAT | | TTTGTCATAAGAGGTCAAATACTAAATAGGAAAT |
| 351896 | SCAFFOLD | AACCTTTCAGCTGTAGATAATAGTGGATATGTAGGA | A/G | TTCAATTTGAGGAATTTAGGCACACCGCACGGAGAA |
| | 75180_16508 | GAGAGAAGATGGACGGGATTGTAAGAGATGGTATTA | | GGCAATGGCACCCCACTCCAGTACTCTTACCTGGAA |
| | | ATCTTCTCTGTAAATTATAAGGAAAGGTGAGATGGT | | AATCCCATGGATGGGGGAGCCTGGTAGGCTGCAGTC |
| | | GCTATATGGTTCATGACAGAAGGGGGAAAGGTGGGC | | CATGGGGTCGCTAGGAGTCAGACACGACTGAGCGAC |
| | | TCATGTTACAGATACAAGAGCATTATCTTTATTCAC | | TTCCCTTTCACTTTTCACTTTCATGCATTGGAGAAG |
| | | CACGTGCCAAATACTTAGCACAATGCACAGCACACA | | GAAATGGCAACCCACTCCAGTGTTCTTGCCTGGAGA |
| | | ACAGAAACTCAATAAATACGAACTGAATTCAAGA | | ATCTCAGGGATGGGGGAGCCTGGTGGGCTTCCGT |
| 345776 | SCAFFOLD | AAAAAGGATTGAGCTAGGTTCTAGTTATAAAAGAAT | G/A | TAGCCCAATAGCTGCTGTTCCAGTAAAAATCTGGAT |
| | 255022_2946 | GAACAAGATTTATCCCTGGTCTCAAGGAAGTCGTAA | | TCTAAAACTAAAATGTTTTCCTGTTTTTACTCATAT |
| | | TCTATTATGGTACATCTTCATTAAACAAAGCTCAAT | | TTCCTACATTTATTTATACAACATGTACCAGAACTA |
| | | CTGTATGTCTCTTTTTCATTAGAAGCAACATACTCT | | TCAGTTACTACATTTTGTTACCTCCAATTAGGGCTT |
| | | TTACGTCTGCTTTGTTGGCCTCCTAAGTATCTAAAC | | CGAAAATTTATTATGAAACTTCAGCATCTCAGAGAT |
| | | ATAAATGATACATGTGGACAGACCACACACTTTTGT | | TGTACAGACACTACTTACTGTTGAACATGTTAGCTT |
| | | ACTGCTTTGTTTTGACCAACACTTTTCTAGAACA | | CATTTTTACTGGGGCAAATGTTACTTAAAAGAAA |
| 350060 | SCAFFOLD | TGGACATTGTGATGAAATTTAACCTCTCATCTCTAT | A/G | GATAACTGTGTATGTTTGCATTTAGGTCATCCTGTC |
| | 20440_9960 | ACCACAAGTTTAAAAAACATATTTTTCCTCTCAAGA | | CATTGAAGAAGGTTACAGACTACCAGCTCCCATGGG |
| | | GGAGTCAGCAGATGCTATGAAGATACTTGTAATTCT | | CTGTCCGGCATCTCTACACCAGCTGATGCTCCACTG |
| | | TGATCACATTTTTTGTATTTATTCTTGTCTAGACTA | | CTGGCAGAAGGAGAGAAATCACAGACCAAAATTTAC |
| | | CCTGCACTCACAAGTAGTTTTCATGCTTACCAATGT | | TGACATTGTCAGCTTCCTTGACAAACTGATCCGCAA |
| | | TTTGGTTTCCACTTCCTGTATAAAGGGAATTATAAC | | TCCCAGTGCCCTTCACACCCTGGTAGAGGACATCCT |
| | | TTAGTGTATGTATTGTTTGTCCCCAGAAGTAAGA | | TGTGTAAGACGCATAATGTTGATTTTTTCCCTTG |
| 346463 | SCAFFOLD | CTTGGTACTTCCAGACTCCCACAAAGCTCTGCTATA | T/C | GAATTTTCTCCAATACTCTCGATGGCCCTAAGAAGT |
| | 185228_7995 | ATCTGTCTCGTCAGCAGATCCTACCTTGTACCATTA | | TACCCCGTACCCATTTCATGGGTGTGGCAGATCCGT |
| | | CTCAGGGGGCTGCCTGCTCATCCATCCCCAAGGCCT | | GATGCTCAATGTTTGTCCAAGGTCACCCTACTGACC |
| | | GTGTGGGCCTCTCCCAGCTGACAACTCCATGTAAGA | | CAGGATTTGATTCAGAGCTGCCTCCCAGCAAAATCT |
| | | AGTCACTTTTGAAAACAAGGGATTGAGGTAGTGTGA | | CTATTTAATGCTAGAATGGGACCTTAGAGAAGGGAG |
| | | CAGCATGGGGAGAACTAGGCATTGACACCCAACGGT | | GCTGCAGAGGGATTTGAGACTCATCAGATAAGAATC |
| | | CCTGGGCTTAGGACAAAGCTGCCAAACACTTGTG | | AAGTGGTACAGGGAACTATATTCAGTAATTACCT |
| 352671 | SCAFFOLD | CTGCCAAACACTTGTGTGAATTTTCTCCAATACTCT | A/T | TATTCAGTAATTACCTATAATAGAAAATAATTTGGA |
| | 185228_8229 | CGATGGCCCTAAGAAGTTACCCCGTACCCATTTCAT | | AAAAAATATATATATACCTTGAAACCTTGTGTCCTG |
| | | GGGTGTGGCAGATCCGTGATGCTCAATGTTTGTCCA | | AGTCCTGCCTTGTACCCTTCCCATATATGTATGTAT |
| | | AGGTCACCCTACTGACCCAGGATTTGATTCAGAGCT | | GTGTATATACATACTGTTGTTCAGTCACAAAGTCAT |
| | | GCCTCCCAGCAAAATCTCTATTTAATGCTAGAATGG | | GTCAACTCCTTGTGACCCCGTGGACTGCAGCACGCC |
| | | GACCTTAGAGAAGGGAGGCTGCAGAGGGATTTGAGA | | AGGCTTCCCTGTCCCTCACCATCTCCTGGAGTTTAC |
| | | CTCATCAGATAAGAATCAAGTGGTACAGGGAACT | | TCAAGTTCATGTAATACAACCTTGTAAATCCACT |
| 351705 | SCAFFOLD | TAAGATCACCCAGTGAGCTCCTCCCCTTCAATTTCC | A/C | GACAAATATTCTTAGATCAAAAAAGCAATTCCACTA |
| | 286772_8833 | CAAATTACCAGCCAGCATATAAACACTTGGGTGACA | | ATACAAAGATCTTTCTAGATGCTGTCATTTACCTAA |
| | | TGTACCATAAAGTATCATGCTTTGGATTTTATATAA | | AATGTTCTCAACTAGTATGTGAAGTATTAATTTATA |
| | | CTTGGTTTTTAAAAAATTTATTAGTAGATTAATTTA | | ATAACTATTGATTTTATGAAATCAACCTGCAAAAGT |
| | | TTCTGGAAACAAATTCAAACTCCTCTCTTTCCCAAA | | TCTTCAGGTAATGGAAAGATGTTTTGGTAAACAGAA |
| | | TTGGGTATTTGACCCAATTTGAAACTAGTGAAACGA | | ATTTTAATAAAATCACATCTTGAAGAAAAACCTCCT |
| | | CTTTAATAAATACTGCCAATAAGGCTTTCATGTA | | TTTGTAAGTCATTCTGACCTGCTCTATCTGCCTA |
| 351575 | SCAFFOLD | CTTCACATGGAAGAAAACCTTTTAGTGTGTGTCCTC | A/G | AGAAAGTTCAAAGTCTGAGAAAATTTCCAGAAAATG |
| | 225004_47983 | AAAGAGATAAGAAACAATGCTGATGCTTTTTTAAAA | | AAGACATCAGACTTGAGAAAACTATCCAAAAACTAG |
| | | ACCTTTTAAAATAACAAATACATTTTTCTTGAATAT | | TGAGAGAAATTTTTCCATAGTTGAAGGGTTTCCAGA |
| | | TAAGGGAGCCAATATAACAAAGCTTGATAGAAATAA | | TTTAAAAAACTAACTTTATGCTCAGCATGATGAATA |
| | | CATATATTTATATATATATATATTTGAAAAAAATTC | | AACTCATTATAAAATGTCAGATAATGGCAGTAAAAT |
| | | AGGCACTGGAATAAAAATGCAAAGTGATGGGAAAGA | | GAAAATCCTAAAAACTCCCAGAGCAAAAAGCAAGAT |
| | | GTAGGCAACAGATAAGCACCCTATAAAATTAGTC | | TACATGCACAATATTAGAAATGATATTTGCATTA |
| 351738 | SCAFFOLD | AAAATTGTTTTGCAAACTGTTTTCATTCTTGTGATA | A/G | ATATTATGGTTCCTTTTCTGTAAAATGTTATTTTTG |
| | 300917_15746 | AGGCTGAGCAACTCTGTCCCACAAATTCTAGTTTTG | | GCCATTTCAAATAAATGATTTTCGATCTTATTGGAA |
| | | CTTGGAAACTGCAAAGTAGTCCCTGAATATTTTAGA | | AGAACCAGGAGAGAGTTTTTACTCTGGTCTTGTCCT |
| | | GGGAATCGATGTTGATGGCAAAAGAAAATTTGCAGC | | TTTCTCTTTATTTGTTGTAGGAAAACAGTAAGGCAA |
| | | TATACATTTGCTTGTTACAATTCCTTTTCTATAAAA | | CTTTGATTCCTCAATTTTGTATTTATTTTACTTTCC |
| | | CCTTATTTTTGGTGATTTTAATAAAGCATTGCCTGA | | CTTTGTGCCTTTTTTGTTAAGGACTTTTGTTTTCCT |
| | | ATGTTTGAGATTTTATAGCTACACTTGGTTGTGT | | TTCCTTTCTTTTATTTTCCTACCTTGGCTGCCTT |
| 351752 | SCAFFOLD | ACACCTGTTGACCAACAAAGAATGGTTTTGAGCAAT | A/C | TTATGGAGGGACACATTTATTTGATTTTTCTTGTAT |
| | 305584_12610 | GAGGAACTTTAGTAATTCTTATCACAGTGTGAAATT | | TTTCAGTGGCGAGAACACCATCTGAGTAATTCTACT |
| | | TAACAAGGACACTTTAGGGCTTCCTAGCACTTAAGT | | GGCCCCTCTTTGCTTCCTCTTTCCTTACCCTTAGCA |
| | | CTTTTTCGTTTTTCCAGCAGGACTCTAACTTCAATG | | AACCATAGTCAATTTGAAAAAAGGAATTCAGTTCAG |
| | | AGGTCTTTATTCAGAATATCCTTCATTTTAACTGAG | | TTCAGTTCAGTTGCTCAGTTGTATCCGACTCTTTGC |
| | | AACAAATGTTCATTTACCAACAGAAACAGAAGATCG | | CACCCCATGAATCGCAGCATGCCAGGCTTTGCTGTC |
| | | TGACAATTGACTCTGTAAATCTTTACCTGAACTC | | CATCACCAACTCCCGGAGTTCACTCAAACTCACA |
| 346327 | SCAFFOLD | AATCCTGGTGTATGTAGGTAAATCCAACATGAACCT | T/C | TGACCTTCAGCAAGGGAGAATCTCAGGATGATTAAA |
| | 143346_5166 | TGGCCTCCATGCTGCTCTGTAAGTCAACCACTGTTG | | ACCTGGGGATGCTCTCTGGGACTTGGAAAGTTGCCT |
| | | ATTGCTTTCAAAGAGAGGGAGCGATGACATTGAAAC | | GGGGGTCAGATCTCAGAGGTACAACAAAATACTTTA |
| | | TTTGAGCATCTTCTTATTCACTCCTTGTACCTGATG | | GGCCTCAGGGGGTCTGACTGAAACTCAGCCTCATAG |
| | | ACACCCATCACTGACCTGCAGGTCCAAAATCCCTCC | | GCATAGAATTGGGGATGGGGTAGAATTAGTTATGTT |
| | | AACACCCTCACCCTTGGCCTCTCTTTCTCAGAATCT | | CCAGAGGGAGAAATAAGTAACTGTCAGAAATGTGAA |
| | | AGTCATTCCTCTTATAGATGTCTCAAGGTCATGT | | AGATTTGTCAATAGCACACTGTTGTTGTTTGTTC |
| 346753 | SCAFFOLD | GGTTATGTGGATATTTACAATAGTGAATGGGGTGCT | G/A | TGGTAGGAATTAAAAAGAGAGGACAGATGCAGAAAG |
| | 290418_1006 | ATTGAGGGTCCCAAAGGCCAGACTCCAGAGCTTGTA | | GATTTTAGAGCTAGATTTTGCATATTCAAGAACTCT |
| | | CTAAATCCAACAGGTGGTGGGGAGCCATCATGTATC | | GAATGTGAAGGAAGAGAGCAGTTTAAATAATCAAAG |
| | | CTAAGCAGAGGAAAGACACTGTGAGAGTTTGTCCTT | | CTATTTTGAGGATTCAAGTCTAACATACCAGAAGAA |
| | | TTAGGAAAATGAATCTGGCAGGACTCTGGAAAGACC | | GGGTAGTGCCTTTCACAGAAATGGGGTAAAAAAGCA |
| | | GATTAGGAGGTAAGACTACTAGAGGTAAACTTTATA | | AAAGGTGATGAACCTTCTCCCCTCAGCTGAAAAAGA |
| | | GTTATCTAAGAAGAAACCCTGAACTGGAGTGGTC | | GTTCTGGAAAAAGGATGGTTGTATCTATTCAGTT |
| 352832 | SCAFFOLD | ATTTCCTTGAGCTCCAAACCTGAGACATGTTCACTG | T/A | AAAGCCTGAGTTTTTAAAGTAGGTGACAGCAAGAGA |
| | 290169_15087 | CCTACAACCATGAGCTCTAATAGCTTGCCTGCCATT | | CATTAACCACCTGTAAGATGGAATACAGAAGCAAAG |
| | | ACAACCACCCCACATTGCAGCTGAGGCAATTTGCTT | | TGCATAAATCTATTAGTTCAAAACAATATTTATGTG |
| | | TTCAAAAGTAATAGAGAGTAAATTGCAGAGGAAATA | | AGCTTATGGATTAAGGTGTTTCAATAATTTAAACAA |
| | | TTTTATGCAAATTAACCAAGCTTAACATTTTGATAA | | TTCTCTCCCTTCTTCTGAGTGGACTCATCGCTAAGC |
| | | AGGCCTCTTATTTTCAAATGAACCAAGGAATCCAAT | | CAAAACATATTTGTAGTGGGATCAATGCTACATTTG |
| | | ACTCCATTGCAAGTTTAACACAAAAGAAAATTTT | | CAAATGAGCTTGAAATGTGCTGTCAGAGCAGGAA |
| 352428 | SCAFFOLD | TGTGATCTTGTGCCTTGTGAATGGGGAAGCCTAGTT | A/T | TTAGGAAATCAAACTTAGGAGTAGATGATTGAGAAA |
| | 60595_8082 | TCCAGCCTGTGTCTGTAATACTTTCACCACTGAAAA | | AAGAGGACATTTAAATCCTTTTTTTTTTAAATATAA |
| | | GGGTAGTGAGGGCCTCAAAGGTGCTATGAGGTCCAC | | ATTTATTTATTTTAATTGGAGGCTAATTACTTTACA |
| | | ATAGATCCATGGAAGATCCAGTGATTTAATTACATT | | ATATTGTATTGATTTTGTGTACATCAACATGAATCC |
| | | ATTATGGCTTTTAAGTGATTTTAAAGTCTCTTTGTT | | GCCACTGGTGTACAGGTGTTCCCCATCCTGAACCCC |
| | | TTCAGGGAAAAATAGCAGGCTAAAAATAGGCGTAAG | | CCTCCCACCTCCCTCCCCATACCATTTAAATCCTTT |
| | | TATTTTAACTGAATTTGAATGATCATCTCCTAAT | | TTGTGGGTTTTTAAAAGGTATTACTCAAGTTCAT |
| 348202 | SCAFFOLD | GCCTTCATTACTTGCAGTGTGTGCCAGGTTTTCAAC | G/T | TTTTGAAAGTGAATCACTGGGTTCAGCTCACTCAAA |
| | 153044_4148 | TGTAAAGATACTTTTTTTTCTTTTAAGGCTTTTAGT | | TGGATGGGAGATGGGGTGGGGAATTAAGCTCCACCT |
| | | GAATTTGTTACAATATAGCTTCTATTTTATGTTTTG | | CCTGCAATCAGATCAGATCAATCGTTCAGTCCTGTC |
| | | GTGTTTTGACTGCAAGGCATGTGGGATCTTAGCTCC | | TGACTCTTTGCGACCCCATGAACCGCAGCACGCCAG |
| | | CCAACCAGGGATTGAACCCACACCCCCTGGATTAGA | | GCCTCCCTGTCCATCACCAACTCCCGGAGTTCACTC |
| | | AGGTGAAGTCTTAACCACTGGACGGCCAGGGAAGTT | | AGACTCATGTCCATCGAGTCAGTGATGCCATCCAGC |
| | | CCTAAAGTTACTAGTTTCCATTCCCATCTTCTGT | | CATCTCATCCTCTGTCGTCCCCTTCTCCTCCTGC |
| 344940 | SCAFFOLD | ATATGCTGGAGACTTGGGTTTGATTCCTGGTCGGGA | T/C | GGTAAGAATTCAGAACTGGGGAGAAGAAAACTAAGC |
| | 260286_10872 | AAATCCCCTGGAGAAGGAAATGGCAACCCACTCCAG | | AGGTCGGGGGAAAAACAAGCAGAGGAGGCGGAGCAG |
| | | TATTCTTGCCTGGAGAATCCCATGGACAAGAGCCTG | | AGGTTCTGGGAGAAGATTCCATCCTGGGATGAAAAT |
| | | GAAGCCAACAGTCACAGGGTTGCAGAGAGTCAGACA | | CTGATTTTTCAAACTGCTTTTCTCCCACAAATGAGA |
| | | TGACTGAGCACACACGCATGCCAAGTGGTGTCAAAG | | TCTTACATGAATCTGCAACATGTAAAATGGGAGAAT |
| | | ACCATGTTCTGGATGACACTGCTCTGCATGAAGAAT | | TACTCTAACTGGAAGGATGCGGTGGGAGCTAGGGAG |
| | | ACTTACTGCCCAGCATGGTGCTGGGACCAGGATG | | CAGCAAGGAAGCCTTAACACCTGCGGTGGCCCCA |
| 350422 | SCAFFOLD | ATCCAATCATCTCTTCCTCTGTCTTTCATCTTATTC | C/G | TACCACTCCAAACTCATGTTCCTTCACTTCTTAAAA |
| | 45483_9360 | TTTATTAACTAATGTAACAGATTGCCTGCTTCAAAG | | TATGCTACATGTGTATGTATTTATATGCCTGCAACC |
| | | GTGTGTATACTCATTAATCCTTTCACAGTTGTGCCA | | TTATTATCGCCTCTCCCCATTTACCCCCTCTCCTCA |
| | | GGATGATTTTTCTAGAACTCAAATATTAAGATATCA | | CAGGCATCATGTTGTACTTATCTTTTTATGTGTAGT |
| | | TTCCTTGGCTACTAAAATCTTCGGTTATTATGAACT | | TATTATCATAATATTTAGTGTACCATGGAGATTAAT |
| | | CATCATTGTCTACTGGTAACAAACTCTAAACTTAGA | | AAATGTAGAATGAGTGGTAATTAAGGAAAAGAAGTC |
| | | TTGCTATCAAGATATTAAATACTATGCAGCTTGT | | TGATATCCATGTCATTCAAGAGGCCTTTCCAAAT |
| 348071 | SCAFFOLD | TAGTAGCCATTTGTCACATGTGGCTACTTAAATTAA | T/C | TCAGTTTGAGGAGTTTCAGTGTTAAAACTTGGATAA |
| | 110776_5578 | GGTAATTTAATTAAAAATTCAGTTCCTCAATGGCAC | | TCCCTGGGAAATGGAGACAGCTCATCACCCTAAGAA |
| | | TGACCACTTTTCTGGGGCTCAACAGCCACACAAGCT | | GTCAGGTAGTCCAACCATATATCTACTGCACATATT |
| | | ATTGAATTGGACAGTGTAGCTATAGAATAGTTCCAT | | TCTTCGACAACATTCTTTTTTATTTTTATTTGGTCA |
| | | TATTGGAGAAACGTCTGTGGGACAGCACTTGACCTA | | TGCAGGCTCTTTGTTGTGGCTCACAGGCTTTCTCTA |
| | | GAGTCTTAGGATTTAAAGAATCTCAAAAAGGGCAAC | | TGTGCCCTGCAGCATGTGAGCTCTTAGTTACCTAAG |
| | | CAACTATCCTGGTTTCCCCAGAATTTAGGGATTC | | CAGGGGTCCAACCAGTGTCCCCTGGATCAACAGG |
| 348632 | SCAFFOLD | TCTTATCACAGTGTGAAATTTAACAAGGACACTTTA | C/T | ACCATCTGAGTAATTCTACTGGCCCCTCTTTGCTTC |
| | 305584_12662 | GGGCTTCCTAGCACTTAAGTCTTTTTCGTTTTTCCA | | CTCTTTCCTTACCCTTAGCAAACCATAGTCAATTTG |
| | | GCAGGACTCTAACTTCAATGAGGTCTTTATTCAGAA | | AAAAAAGGAATTCAGTTCAGTTCAGTTCAGTTGCTC |
| | | TATCCTTCATTTTAACTGAGAACAAATGTTCATTTA | | AGTTGTATCCGACTCTTTGCCACCCCATGAATCGCA |
| | | CCAACAGAAACAGAAGATCGTGACAATTGACTCTGT | | GCATGCCAGGCTTTGCTGTCCATCACCAACTCCCGG |
| | | AAATCTTTACCTGAACTCATTATGGAGGGACACATT | | AGTTCACTCAAACTCACATCCATCAAGTCGGTGATG |
| | | TATTTGATTTTTCTTGTATTTTCAGTGGCGAGAA | | CCATCCAGCCATCTCATCCTCTGTCATCCCCTTT |
| 344009 | SCAFFOLD | GCCTCCTGTGAATGCAGGGTGAATCAGCCTCAGGGT | A/G | GCCACACCCTCCTCATTTTCTGGCAGAATTCAGCAT |
| | 182035_3645 | TCACCTCCAGCTGCCAGCCCATTGGCCAAGGTGAGA | | AGATCCTGGGGTCACCTCCACTTTCCTGTGAAGAAC |
| | | AAATTTCTAGAAGATGGTGCACCCGCCAGCCCAGCC | | CCAGCAGATCCCCAGCAGATGGTTATTCGGTGTTAC |
| | | TGGATGCCACTGGCTGACCAGCTCTTTGTGGGAAAA | | TTCTCATCACTTACTATCCATAGGCTTGGGCGAGTG |
| | | GCAATGCCACATTCACGGCCTGCACCCTGCCAGCCC | | AGTTCAACTTCTTGGACCTCAGTTTCCTCCTCTGCA |
| | | TGGACAGTGTACCAACTGAGGTGTGAGGCGTTCCCT | | AAGTGGGTGCAATAATAGTACCCCCTCCTGAGTGGT |
| | | CACTGGGCCTTCCCAGGAGGTCAAGAGCAGATTG | | TATAAGCCATCAAGTGCTTCAGGTACACAGTAAG |
| 350139 | SCAFFOLD | ATTAAGACTTTTTCCAATGCATGGGCACAGGTTTAA | G/A | ATTGTCATCATGAAGAATTTGGTACTGTCACAGAGA |
| | 235137_19618 | TCTCTGGTTGGAGAACTGAAATCTCATATACCTCAT | | CATACAAATTGACAAGTGGAATACAGAACAGAGAAA |
| | | GGTCAAAACCCAAAGCATAAAATATAAGAAACATTG | | CAGATTTGCACATGTGTAAAACTGTAGTAAATGTTG |
| | | TAACAAAGTCAATAAAGACTTTAAAAATAGTCCACA | | CAGATACCATACTAGACCAGCAAGAAAATAAGTGAC |
| | | AGAAAAAAAAAAAAACTTAAAAAAATAATGAGGTTA | | TACTCAACAATGAACTAGAAAAAAATTATTATCAAT |
| | | TTTTTAAAAAGAAGAAGGAAGTAAATGAAAATCAAA | | ATGGGAAATGTTCGACTGGGGGCATATTACTCAACA |
| | | GACTTGCCCTACCAGATGTCCAGACTTATTCTGA | | TATTATGCAGTAACCAAATATATTGGTCAGTCAG |
| 353745 | SCAFFOLD | TTGGACAGTGTAGCTATAGAATAGTTCCATTATTGG | G/T | ACAACATTCTTTTTTATTTTTATTTGGTCATGCAGG |
| | 110776_5692 | AGAAACGTCTGTGGGACAGCACTTGACCTAGAGTCT | | CTCTTTGTTGTGGCTCACAGGCTTTCTCTATGTGCC |
| | | TAGGATTTAAAGAATCTCAAAAAGGGCAACCAACTA | | CTGCAGCATGTGAGCTCTTAGTTACCTAAGCAGGGG |
| | | TCCTGGTTTCCCCAGAATTTAGGGATTCTTCAGTTT | | TCCAACCAGTGTCCCCTGGATCAACAGGCAGATTCT |
| | | GAGGAGTTTCAGTGTTAAAACTTGGATAATCCCTGG | | TAATCCCTGGACCACTAGGAAGCCCCTTCTGCAACA |
| | | GAAATGGAGACAGCTCATCACCCTAAGAAGTCAGGT | | TTCTTCACTCTGGACATTGCTGAGCCTGCCAGCTAA |
| | | AGTCCAACCATATATCTACTGCACATATTTCTTC | | GTGATGTTAATCACTGATGTGGCTGAAGCAGGCA |
| 343247 | SCAFFOLD | TAGGATTTAAAGAATCTCAAAAAGGGCAACCAACTA | C/G | CTGCAGCATGTGAGCTCTTAGTTACCTAAGCAGGGG |
| | 110776_5764 | TCCTGGTTTCCCCAGAATTTAGGGATTCTTCAGTTT | | TCCAACCAGTGTCCCCTGGATCAACAGGCAGATTCT |
| | | GAGGAGTTTCAGTGTTAAAACTTGGATAATCCCTGG | | TAATCCCTGGACCACTAGGAAGCCCCTTCTGCAACA |
| | | GAAATGGAGACAGCTCATCACCCTAAGAAGTCAGGT | | TTCTTCACTCTGGACATTGCTGAGCCTGCCAGCTAA |
| | | AGTCCAACCATATATCTACTGCACATATTTCTTCGA | | GTGATGTTAATCACTGATGTGGCTGAAGCAGGCAGT |
| | | CAACATTCTTTTTTATTTTTATTTGGTCATGCAGGC | | ATGTGGAGAACTGGAGTAGATCAAGCTAAAAAAAAA |
| | | TCTTTGTTGTGGCTCACAGGCTTTCTCTATGTGC | | AAGAGAGAGAGAGAGAACTGTTTCAGATTTCCTC |
| 346144 | SCAFFOLD | CATCAGTCTAGACACAGACTTTATACCCTTCACAAA | T/C | TAAGACAGAATATCTGCAAAAGATACTTCTGATAAA |
| | 87323_347 | GTTAACCCAAAATAGATCACAGACCTAAAAGCAAAA | | GGTCTGTTAACAAAGTATATGAAGAACCTTTAAAAT |
| | | TTACAAAACTCACAGACAGTAATGGGAAAAACAGAC | | TCAACAAGAAAATAAACTGATTAAGAAATGGGCCAA |
| | | GATTTTGGTTATGGTGATGTCTTTTTAGATACAACA | | AGATCTTATCAGACACCACAGCAAAGAAGATACACA |
| | | CCAAAGCAAGTTCCATGAAAGAAATAGTTGATAACC | | TGTGGCAAACAGGCATATGAAAAAATGATCCCCATC |
| | | TGAACATTATCAAAAATAAAAACTGCTTTGTAAAAG | | TTTCCCATATGATATTATGGAAACGCAAATTAAAAC |
| | | ACAAACTCAAGACAAACAGGAGACAAGCCACAGC | | AACAATGAGATAGATTCCCTGAGGATTCCCAGTC |
| 343545 | SCAFFOLD | TCTCGTCTTCTCTATCTTAGCAGTTCTCTCTTTCTG | G/T | GGCCAGGGCATCGTTTGTGTCTGAGTTCCCCTAGCA |
| | 290159_9107 | AAATCATTTGCTGTGGGGTGTAAGTATCTTAAGGCA | | ACTCCAATGTGCCCGTGAATATATAGGCTAGAAAAC |
| | | CGACTACCCTTAAGAGTGCTGGTGGGACAAGCAGGG | | ATGAAATAAGGGGTTAACAGGCACACGTGACCTTCC |
| | | AAGATATACTTAACAGGGACAAAGCCTCACGCTAGA | | ATGCTGCGTACAGGGAACTGTGCCGTCATGGTAATG |
| | | GCAGTGCTTCTCAAGCCCCACTGCACAGTACAATCA | | CTGTTCAACTTTACTTGGCTCCAGCCTGCAGTTTGA |
| | | CCTGAGAGCTTTACATGCTGCCTGTGCAAAGGGCCC | | AAGACTTTCTCATTCCAGCAAGTCTCCAGAGTGTAA |
| | | ACCCATGGGGGTCAGATTTAGTCAGCTGGGAGAG | | TTTCCTATAGGAGATCACCAGGGGTCCTCAGAGA |
| 344231 | SCAFFOLD | TCCCCTAGCAACTCCAATGTGCCCGTGAATATATAG | G/A | GGCACATTTGCCAATGTCTGGGGGTGGGGAGATGAA |
| | 290159_9384 | GCTAGAAAACATGAAATAAGGGGTTAACAGGCACAC | | TAGCAGTCTCTGAAGCATTTTTGGCATGGTGGCAAG |
| | | GTGACCTTCCATGCTGCGTACAGGGAACTGTGCCGT | | CAGTCCAAAATGCAACTGCAAATCAACATGAAAATT |
| | | CATGGTAATGCTGTTCAACTTTACTTGGCTCCAGCC | | CTTATTATGGTTCATTTGGGGAGTTATTAGCAATGC |
| | | TGCAGTTTGAAAGACTTTCTCATTCCAGCAAGTCTC | | TCTATCTGGTAGTCGAGAGTAATTATTTATGGTGAA |
| | | CAGAGTGTAATTTCCTATAGGAGATCACCAGGGGTC | | ACTAAGGCTTGAACTCAAGAGTGTGAACATGATGCA |
| | | CTCAGAGACCCAAAGGGCCTCTTAGAGGAGTTGG | | TAACCAGCTTTAAGAGGGTTCAGTCTTTTAAAAG |
| 350644 | SCAFFOLD | GCAACTACTGAGTTCACATGCTGCAACTACTGCAGC | G/A | TATTACACATGTGCAGATGGTGATAAAGGCTAAGGA |
| | 110535_1540 | CCCCTAGAGCCTGCGCTTTGCAACAAGGGAGCCACC | | GACAAATGAGCCAAAGGGAGAGAATATTCAGCAAGG |
| | | ACAATGAGAGACCCACACACAACTAAGGAAGTAGCC | | AAGAGGGTTGCAATTTCAAACTGGGTGATCAAGGAA |
| | | CCCACTTGCTGCAACCAGAGAAAAGCCTGTGCAGCA | | GATTTCACTGAAAAAGTGACACATGAGTGAAAACTC |
| | | ACAAAGACCCAACACAATAAAAAATTAATATATTTT | | AGGAAGTAGGAATGAGCTGTGCCAATGACTGGCAGA |
| | | TTTAAAGCACAAGTCTTTATTCTTAAGCATCTTAAA | | AGTCAGGGCAGCTCCCCAGTGAAAGGAAAGGGAAAA |
| | | TTCCAGTGTGGAGAGAAGAAAATAAGCAAAATAT | | TTCAAGATCTTAAAGAGGGAATAAGCCTGAGTAG |
| 353781 | SCAFFOLD | TATATGTGAACTGTTTAAAAACAACAACAAAACAGG | C/T | AGATGTGTAGTATGAGATAAGGGAGATTAACTAGAA |
| | 15547_11058 | CTCATAGATACAGAGAACAGATCTGTGGTTGCCAGA | | TGATTGTGACCATTTCACAATATATACAAGTACCAA |
| | | GGCAAGGGCTACATGAAATGAGTGTAGGTTATTAAA | | ATAATTATGTTGTACATCTAAGACTAATATAATGTT |
| | | ATGTATAAACTTTTAGTTATAAAATAGGTAAGTCCT | | ATATGTCAATTGTATCCATTAAAAATTCAAAAAAAA |
| | | GGTGATGTAATGTACAGCATGATGATTATAGTTAAC | | TACCTTTTCACTCTCCTTTTGGACAGTCCTGTAGAC |
| | | AATACTGTTTTACGTATTTGAAAGCTTCTGAGAGTA | | TTTTTAAAAAGATCTACAATGTTAGATGACAAGAGT |
| | | GATCTTAAAAGTTTTCATCACAAAAAAATATTTA | | CTTTCTACTTTGATAATGAATGAAAGTATAGTCA |
| 345817 | SCAFFOLD | CACCTTGGCAATGCTCACATACCAAAGTTAGGACTT | C/A | GCCTCTTATTCTGTCCACCAGACCAGCGGTGGGGGT |
| | 265188_23332 | ACTGGGAAAAGACTGGCACCTTAAGATTTGAGATAT | | CAAACCTCCTGAGGGTCAAATAGACGGATCGTTGAT |
| | | GGATATCTGTACAGTTGCAATGAAGAAGCACGCAAC | | AAACTGCTGAGCCCACTATGGGAGGGAGTGATTTTC |
| | | TTTAGGCTACCCTGAGTTTTATGAGCTAGCATACCC | | TGATAAAGGATTATAGGATATAACTAGTTCTCCAGG |
| | | CACCTCCACTTGACAGAAGTATCTAGAGCATCTCCC | | AGCTTGGCCTAGAAATGTATCCTAGAGTGCTGAATA |
| | | AGGCCTCCGAACCGTGAGAGGGCCTCAGAGCAGCTG | | GAATGTTAAACTAGTTAGAAGAACTTCTGTCAGAAA |
| | | TCTCACAGGACGGCACTTGCCATCTTCAATGTTG | | TAGGTCACTCTCCCGTGACTCTGGATTTAATTGC |
| 349461 | SCAFFOLD | GACCAGAAGAAAACCTACTGAGACTCTAGAAGAAAG | T/C | GTGCCAGCCTTTCACTAGTCTCTGGAGCAATAACTG |
| | 286772_8400 | GAACAGTGTAGATGGTGGTATGTTTGCAGAGACATG | | GGAACAATATGCAATCCTAGAGTTTATGGACTTTGT |
| | | AAGCAGCTAGTATGTTGAACCACAACTTCTGTTTTC | | GACATCAACTTTGGCTGCCAGCTCAATGATCTTTTT |
| | | CATTTCTTTGCCTCTTACTGCCCTAATTCTCCTCCT | | CACTTGCTTCTTGGTTTTGTTAACAATCAGGATAGC |
| | | CACTTGCAATTTACCAGGGATTGATAAATCAGATAA | | TAAGATTGCCAGATGTGGCTTTTGTGGATTTATGGT |
| | | TTATGCTTATTAGAATTGTGCCCAAAAGAATGAATA | | GTTAAGATCACCCAGTGAGCTCCTCCCCTTCAATTT |
| | | GCTGCAAGGCAAATCTGTATTTTCAAGTAAGAAA | | CCCAAATTACCAGCCAGCATATAAACACTTGGGT |
| 353486 | SCAFFOLD | AACCTATTCATGCCAAGTGGATGGCATGACCTTATA | G/A | AATCACATCAAAATATTTTATTTTATTTCTTAATCA |
| | 147091_1735 | CTCAGAATAGCAAAGAATGTAAATCTAAAAATAAAA | | TCCCCATCAGTTTAGTCATGATTCTGGGGTAAAAGG |
| | | TATCTTAAAGACAGAAAATTAAAATCGTGACAGTTA | | AAAACCAGTAAGTAAAGCAGAAAGAAATTAAGAGGA |
| | | AAGAGTAAGAATGTACATTAGAAGTCACATTTCTAT | | GATGCAAGACTAGCAATGTGATACAAAGTGCAGGGC |
| | | TTGTGTGTTTGTTTAAGGCTGTTGCTCAAAAAAAAA | | ATGATCTGAGGTCTTTTACAGACACTTTTATTCACA |
| | | AATTCTGAAAAATTAATACAATTATTCTATGCAGCT | | CATTAAAAAATTACTGATGGATTTTTTACTGAACAT |
| | | TAAATTATACCTGGTCTATGGCTTTACTTAGCAG | | GTTTATGTTTGTGTAATAAGGGTCCCAGAATTAT |
| 354151 | SCAFFOLD | GGGAGAAAATATTTTCCATTGAAAAATAAAAATATA | C/G | ACACTTTTTTTCAGTAAATACATTTACTCTGATTTT |
| | 19865_433 | TCAAGACCTTTGGCACTGCACTTCATTTTTAAACAG | | TGGAATTAAAACATTTTTAACTTCTGAATCATCTAG |
| | | AACACAGTTTTTAATGGGGATTTTTTCCTTTTAAAA | | GTTATATTTTGGGCATGAATAATCTTGTTGCCTTTT |
| | | TTCTTAAATTCCCTTTTTATTTAAGAATGGTACATA | | TCTCATCTCTAAAGAGTTTCTCATTTTTCCTTATTT |
| | | TTCACTTGTGTTGATATTCACTATTTGAGGTTTATA | | TTCTCTAAGATTGTTAGTAGACCACTATGAAATCAC |
| | | ATATCCTCTTAAATTGTTCAATCAGTGATTTTAATC | | TGGACAGAATTTTTGTTACAGAGGATGCCTACATTT |
| | | TTTTTCACCTTTTATTTATTCATTGACAGATTT | | TGAGCAATTAAAATTGTTGTTTCTTGGCATTATA |
| 343049 | SCAFFOLD | TCTTACAATACACAGGACAGCTCCCTCCACAGCAGA | T/C | CGAGAAGTCAGGATGTGGCACATGGAAAAGTGCTCA |
| | 316779_17543 | CAACCATCCAGCCCCTACTGTTGGTAGTGCTGAGGT | | TCGGTGATGAGTTTGGAGGACAGTGGGCTATAGAAG |
| | | GGAGAACCCCTAGCCTGATATGAAGAGAAGGAGGCC | | GTTAAACACATTTCTTCAGTGCAAGGCTTGCTTCTT |
| | | CCATCGCAGCCTTCAAGGAGACTGCAGTGGGGATGG | | GTTTTTAACGCACGGTGTGCAGTGTGGTGTTCAGGA |
| | | GAGAGAGAGAGAAGATCAGGTGGAAACTGTAACGTA | | GAAGAGAAGGAAGCCTTTTAAGCATTATTTGGGGAG |
| | | AGGTAGAATCAGGTTGGGCCATGCAAGTTGCATACA | | AAGCTTCCAGTTTGCATTCAGTGGGCTGCCATTTCA |
| | | CTCTGAGGTGGTTCTGCTGCCTGGACCAGTTCCC | | GTCAGTTCAGTTCAGTCACTCAGTCATGCCCGAC |
| 349905 | SCAFFOLD | GGATTGCATAGTAACCACTGATGCAAACTGTCCATT | G/A | AAGATACATGAGGGAAATCTATGCTTGGCTTCTAGC |
| | 145192_9213 | ATGTTTTCATGTGTTTCCAGAGTTCTCACATAACAT | | TTCAGAAAAATTCTGGAATTCACTTTTGGTTTCTAA |
| | | GTTAATTGGAATAAGCATCTGTCAGTACTGGAAAGT | | AATTCCAGAAATCTGAAGTAAATTAAAAAAAATTAT |
| | | CACCAATCAAGACAGGAGAATAAAAGTGAAATGGTA | | ACTAAATTATTGTGCGCCTCTGATTATTTGTAATGA |
| | | GTAGCTTTCATTGAAAAAGCACCTTATGGTAAAGTT | | ATATCATTTTCACCAATAATATGCAPATAAATTGCT |
| | | TGGAAGTAAGGTGATGTAAGACAGAAGGTGCAGTTT | | TAAAAATTGCTCACATTATGAAATTCATGCTTTCAT |
| | | GGGTGTGTTAAATTGATGAGGGAAGATATAGTGG | | TTGTTTTTGCTACTCTCCTGCTCCAATTAACTTC |
| 346722 | SCAFFOLD | GAGATGAAGAAAGATAAGCCATGTTAATCTCAAAAA | A/G | AGGGCAGGGGATTGCTCAAAAGTGATTATTTAATAC |
| | 281042_10378 | TTCATAAATAGCAAAATTAAAACCACCAGATCAATA | | CATGTTAAGGAAGATGACTTAAGTTTCTTTGATGAT |
| | | AAACAGTAATCAGGAAGAGTTTACTGAACATGTTAA | | TATCAGAGGCATTCACAAGAAGTAAACTAAGTTAAG |
| | | GAACAGTTTAGAACTGGACGCTTGCAACCTCAAAAC | | TTTTGCTTATGCACATGAAGTAGGCTAGATTTAGTT |
| | | TAATATGAAGCTCAGAGGCAAAATGTTACAGAATGG | | TTGCTTATATGGCTTAAATGGTTTTGTCTCCTCAGG |
| | | CTTACGAAGTGAAAAAATGAGGAAGCGGTTTAACCT | | GAATTTTCAAGTTTGATGTTCATTTTATATTTTACT |
| | | TTACGGTGATTGGTTATTACAATGAGGGTTTCTA | | TTTTACAGTGATGCTAAATCTAAATAAACCTAGG |
| 348572 | SCAFFOLD | TGAAGCTCCTTTCAGCAGGACTTCAACCTAATAGCA | C/T | AAGAGTTTCATTGGGGATGACAGAAGGCAATGCTGA |
| | 285028_33789 | AATAATCACATCCCTTCTGGTGATGTTTATGAACTA | | GCAAGAAGGAGGTTTGAAATCCTGCATTAATTCTAG |
| | | GAGAATGGTTCCAATTGGTCAGTTTCATGCAGGTCT | | AGTTCATCAACCTATGACTATGGAAATCTCGATATA |
| | | GAATATACACATTTTGATTGAATTTGCTCAAAAGAA | | TTTGAATTGCAAAAATTTATAAAAAACTCTTAAATA |
| | | GATAGCTGCACTGTTAATCATATTTATTAGTTGATC | | CTTTTTGTCCTTCTGTTTTCTCCTTCAATATAAGCC |
| | | TTAAGAATTCATGCATTTAATTGTTCTAGAATTAGA | | TACAAAATTATAAGGTACTTAAGTGTTGGGACCATG |
| | | GATAAACCTTGGAATTATGACTGTGAGAAATGGC | | TCTCTGAATCTTTAATAACAAACACACTCTCATA |
| 342524 | SCAFFOLD | CCAGCAGTCCCACAGCTGTCACCACGATCCTTTTAA | C/T | GGGGCAGAGTGCTTTCTCCGACAGTCCTACACACAC |
| | 164825_5474 | AATCACAATCTGATTCCATCACTCCCTAACCTCAAA | | CAGAACCCTGCTATCACTCTCTATCCCCTCGCTCCG |
| | | CTTCCAACCACTTCCTATCACATGTAGAATAAAATC | | CTTTATTTCTCTACACTGCACTCAGGATTATATTAC |
| | | CAAAGATCCTTGCCATAGTCTGGAGACCTTACATGT | | ACACGTATTAGCTATTTCTCTGTATACCACGTAACC |
| | | AGATCCCTGCTTATCAGAGACCTCCCTTCCTTCTGT | | ACGAGGGCAGGGGTTGGATCTGTTTTGTTCACAACT |
| | | GTTCACCATTACAGCCGCAGAGTTTTCCTGGATGGG | | ACATCAACAATGCTTAGGAGAGGTCTGGCACAAGAT |
| | | CTCCAAACGTCCCAGGCCAGCATTCCTCCAGGGT | | GGTCTCTCCAAAAGTATTTGGAGAGTGAATGAAG |
| 349447 | SCAFFOLD | CCACGCGCCTGGAGCCCATGCTCCGCAGCAAGGGGG | T/C | GTATAAAGGATGACCAGATCCAACTATTTGCTATCT |
| | 281042_10044 | CCACCGCCGTGAGGAGCCTGTGCCCTGCAACCAGGG | | ATAAGAAATTTATTTTAAATGAAAGATTAAAACTGA |
| | | AGCGGTCCTGGCTCGAAGCAAAGACCCCACACAGCC | | TTAAAAGTAAAGAGATGAAGAAAGATAAGCCATGTT |
| | | AAAAATAAAACAAAAACACAGTACAAATACAACAAA | | AATCTCAAAAATTCATAAATAGCAAAATTAAAACCA |
| | | GAGAAAACAGCTGCAAACGTGATAGACAATATTAAT | | CCAGATCAATAAAACAGTAATCAGGAAGAGTTTACT |
| | | CCAAATACATCAATAAGCACTTTAAAGGTGTATCTA | | GAACATGTTAAGAACAGTTTAGAACTGGACGCTTGC |
| | | AATATGCTGCTTGAATCACAGAGATCTTTAGAGC | | AACCTCAAAACTAATATGAAGCTCAGAGGCAAAA |
| 353318 | SCAFFOLD | TAAAACTAATCAGAACAGATTTCCTGGAGCATGAAA | A/C | TTCTTAAAGATCAAAAAAAAGTATACAAAGAAGAGA |
| | 316764_15803 | CCCTAACTAGGTTCTCTACTAAAATGTACACTAAAG | | GAGGAAAGTGTGTTTAGCTTTTAAATATGTATCATA |
| | | TCAGCTCCAAAGTATGACTCAACTCAGTGGAGTATT | | AACCACAACTTAGATCTGTTTTCTGGTGATTTGGCT |
| | | TGCCTAAGCATTCCAGAATAGCCAGGGTAGGTGATG | | CCATTATTTTTCCAGATGTCATTATTCCTATTTTTT |
| | | CACACAGTTTGTTGACGTGAGCAAGGAAACATGAAA | | AACACTACCCATCTGCAGAAGGGCTCAAATATTCAA |
| | | GAGATCTGCTTAGAGATCTGGGAACTATGTTTCATG | | ACCCCACCACATAGCTTTTCCTGTCTATATATCTAG |
| | | GTTTGCTAATATGAGTTATTATTAGCAATCAGCA | | TCTGACTTCATCAAACACATACACATTTTTTTTT |
| 346469 | SCAFFOLD | GAGAAAAAATTATAAAACAGACATACAGAGAATAAA | A/G | TCCATGTCCCATTGTGACACAGCCCAGAGCTGTGGA |
| | 185915_641 | TTTTAGTTTCAAAAGCCAAGGCTGCAAAGTAATGAA | | GAATGTAAATCAGCACTTGTCTGTCCATCTCAATTT |
| | | TATCTTGATAAATGAGAACAATCCTCTCACCCTGGA | | GAAAATACAGGTGGCAGAGTCTATGCTTGCCATCTT |
| | | TAGTAAAAGATTTTCCCAGTAAGAGGTGAAATAGGA | | TATTCTGGTCATCTAGAACGGTTCTTTCCTTCCTGT |
| | | AACAAGAAATAATAAAAGAGGGTCAGAGACCTATAG | | AGTAAATATTGCTTATTGATTACTCAGAAGTTATGT |
| | | GACTCAGATTGGAAAGGATATGTGGGGAGACGACAT | | CTACTTGTGCCCTCTGGCTTTGCTCTACCTAAAAGG |
| | | GAGGAGGAACACCTGTTGTCAACAGTCAGAGACT | | CACTAATATTTGTGCTAATAACACAAGCTCTTTT |
| 352706 | SCAFFOLD | AATTATTTCTTTCATTTCTGAGGTTATAACTACCAA | A/G | ATTCTTATATTTAGTTGTGCATATTGAGAGTCCCTT |
| | 20780_6570 | TATTCATCACATTTGCAATTAAAACAAAGACACCTA | | GGACTGCAAGGAGATCCAACCAGTCCATTCTGAAGG |
| | | AAATATTTATTAACTAGCTAAAAGTAACAAAAAAAA | | AGATCAGCCCTGGGAGTTCTTTGGAAGGAATGATGC |
| | | ATCCATTTGATGTTAACATGAATAACATTTTTGTTA | | TGAAGCTGAAGCTCCAGTACTTTGGCCACCTCATTC |
| | | AAAAATAACTATATTTTACAAAACAAAAAAATATTT | | GAAGAGTTGACTCAATGGAAAAGACTCTGATGCTGG |
| | | AGTGAGAAGAGTGGCACTATTTTATATGTTTACAAA | | GAGGGATTGGGGGCAGGAGGAGAAGGGGACGACAGA |
| | | TCTCTTTCCTATATGGTTTGAAAGAAGACAGTG | | GGATGAGATGGCTGGATGGCATCACTGACTCGAT |
| 347184 | SCAFFOLD | AAATTTCTTGTCATCTTCAAACTTTTCTGCAGTGCT | T/C | GAGTCTATCATACACCCAACAGTTAGTTCTTCCCAC |
| | 135343_20976 | TCTATATGCAAATTAGAATCCTCAGTTTTCCTGATC | | TCCACCCAATTATATTCCCCCATCTTCTTTGATAAC |
| | | ATTTTTTCCCATATTCCTATCCTGAAACTTATGCAA | | CACTAATTTTGTCCCTATGTTTGTGAGTCTGCTTAT |
| | | TTTTTTCTTCAGAGAGCTTTTCTTAATCATTCAAGT | | TTTTTGTTATATTCACTAGTATGACATATTTTTTAG |
| | | CCTAAATAATTTTATTTTTTATTTTTATACAATTTT | | ATTCCTTGTATTAATATAAGTGATATCATAACAGTA |
| | | GAAAAGTTACTTTGCATTTACAGTTATTACAATATA | | TTTGCCTTTTTCTGACTTCTTTCACTTGTAAAATAC |
| | | TTGGCTGTATTCCCCATGTTGTACAAGGCAACC | | CCTCAAAGTCTATCCACGTTGCTGCACATGGCAA |
| 343022 | SCAFFOLD | AAATCATTTGCTGTGGGGTGTAAGTATCTTAAGGCA | A/G | ACTCCAATGTGCCCGTGAATATATAGGCTAGAAAAC |
| | 290159_9143 | CGACTACCCTTAAGAGTGCTGGTGGGACAAGCAGGG | | ATGAAATAAGGGGTTAACAGGCACACGTGACCTTCC |
| | | AAGATATACTTAACAGGGACAAAGCCTCACGCTAGA | | ATGCTGCGTACAGGGAACTGTGCCGTCATGGTAATG |
| | | GCAGTGCTTCTCAAGCCCCACTGCACAGTACAATCA | | CTGTTCAACTTTACTTGGCTCCAGCCTGCAGTTTGA |
| | | CCTGAGAGCTTTACATGCTGCCTGTGCAAAGGGCCC | | AAGACTTTCTCATTCCAGCAAGTCTCCAGAGTGTAA |
| | | ACCCATGGGGGTCAGATTTAGTCAGCTGGGAGAGGG | | TTTCCTATAGGAGATCACCAGGGGTCCTCAGAGACC |
| | | GCCAGGGCATCGTTTGTGTCTGAGTTCCCCTAGC | | CAAAGGGCCTCTTAGAGGAGTTGGGGGCACATTT |
| 347810 | SCAFFOLD | TAACCCTTGATAAAGAGAATAGAAAGTTCATAATCT | G/A | TAGCTCTGTAGCAGCAAGGTAAGATCTGATCTTCTT |
| | 48045_789 | TGAGTTGATTTGGCCCCCAAATTGAATATAATTGAA | | TGCTTGATAGTCAGCACACTGAATCTTTATTGAGGT |
| | | CTCAACCAATAAAAAATGAAATAAAAAAGCTATTTT | | GTTAGAAGAGGCAAGTTCCCATGCCAAGATTTAATT |
| | | AGCTAGAAGAAGCAATCAAACAAAAAATATTCCTAG | | AAAGTTTAAATATTTGAAAGGGCAGTGATAATGTTG |
| | | CCTTTGAAAATTTAAAGTAAATTAAGAGAGAATTCC | | TCTCTAATAAAAATTTTACCTGTCTTGGGATCAATA |
| | | TAACAAATTTGGACTTGGCATATATGTGTAAAAAAG | | GAGAAATAAGGTTGTCCCTGAAGAATGCTGTAAACG |
| | | ATTTATTTTTATATCAGTTTCTATCTGAAGGAA | | ACTCTGGCACTGTTTCCATAGGTTGGGTCATCAG |
| 349191 | SCAFFOLD | ATTTATAAAAAGTGATGGTTTTTATAGCTTAGGCTC | C/A | TAACCAAGATTCTACATGATATGGTTGCATTCTACT |
| | 196149_5528 | ATCATCTCAGAGTTTATGTACTCTTTTTAATCATCA | | CACAGCAACCTAAAACCTACATGGCTATAGTTTCAA |
| | | TGCCCCTGATTGCTGATTATTTACTCTTCTGCTGTT | | TACACGGGTCTACAGCAGAAGGAAGACTACTGTTAT |
| | | TCCTCTCATGATTACACAGCCTGCTATGGAGCCAGG | | TTTATAGATTGTTCTGGAGGATGCTGCTACACTGGT |
| | | CATCATGCCTCTGCGAGTCCCTGTAGCATCTCTCTC | | ACATGATCTCAAGGTGTTGAAGATATTGGCCTACTA |
| | | CACTTTAGTTTGTCATAAAGCAGCTGGTTTCTTCTC | | CTTGATTTATACACAGCTTCCATAAATTTTAGATAA |
| | | TCATCCATCCACTAAGTATGCCCTGCAGAATTGT | | TCCAATGACAACTTTCAGTGCTACAAGGCTAATT |
| 352220 | SCAFFOLD | GGCTGGATGGCATCACCGACTTGATGGATGTGAGTT | C/T | AGGTGCCTAAACTGTTCTCAAATAACCAGTTCAACT |
| | 236388_4534 | TGAGTGAACTCCGGGAGTTGGTGATGGACAGAGAGG | | CACTAAATCCCAATTTCACCAACCCACTTATTTCTG |
| | | CCTGGCGTGCTGCAATTCATGGGGTTGCAAAGAGTT | | ACCCTCATGGAGACGTCATTAGTTTTCAGTTTACTT |
| | | GGACACGACTGAGCGACTGAACTGAACTGAAATGAC | | TGTACTCCTACCTTGGCTACCAGGGAAGAGGACCTT |
| | | GTATTTGTCTCCATCCATTTGTCTTCCTGCCTGTCT | | TTTGATGCTAGGAACATGGCAGGGGGGCATTGGCCA |
| | | ATATCAGTATTTATATGTCCAAATTAGCAGTAGGAC | | ACTCCACAGGCCCATGGTTTATGAAGCACATTTTCT |
| | | TTACCCACCATCAATTTTATTCATTGATTTAATG | | GATGGAAAATTCAGTCTCCAAAGAATGTCTTTCT |
| 350717 | SCAFFOLD | GTTTGACAGCTGTTCATTCGATCTTGAAGTACTTGG | C/T | GTACTGCCAAGAGTGAATTTGATAAGAATAAATTAA |
| | 145586_11232 | TAAGAGAGGCTTCTAGTCTGGATAAGTGTGATGTTT | | AGGTATATATGTAGATTCGGGGAAAAAAGCAATACT |
| | | ATTTATTTATTTTTTAATGGGGTGTGAGAGGATAGA | | TCAATAAGTAAACAAAGGGAAAGTTGACTTGGTAAC |
| | | GGAGAATAAGGTCATGTATTAGTATGAGATTTTGAA | | AAATCAAAAGTGTGACATAGCTTTGTATTTTTAGAC |
| | | AATAAAGTTCCCTGGGACAAGGATGTCAGGAAACCC | | AAAGCTGATGCGATTTAAAACCAGAGCATTACAAAT |
| | | AGGTAGAAGGGAGAATTTTCTCTTATAAGGACTAAT | | GTGGTAGGATCTAGGTTTAGGAAGTGCTACTGCTCT |
| | | GTCAGATATGGACATTCTTCATCTATATGTTCTG | | CTGTAACATGTTACTTGTTCCATGTTTGGGGGTC |
| 351918 | SCAFFOLD | TAACACAGTTTAAGTTAGTTTTTATATAAGGTGAGA | G/A | TAGTCTTGAAATTGGATAGGAAGTTCTCAATTTGTT |
| | 85248_5248 | GGCCTGTATCTAAGTTCAATTGTTATATATAGGCAT | | TCTTCTTTTATGTAATATTGACTATATTGTGTCCTT |
| | | CCAGTTGTCCTAGCTCTATCTGCTGAAAACTCTCCT | | TGTTTTTCCAAATACATCTTACCGTCAGTCTGTTTA |
| | | TTCTCAGTTGAATCCCTTTGTCTTTTGTCAAAAATC | | TATCCACAAAATATTCTCCTGATATTTTGATTGAAA |
| | | AGTTGACTGTGTTTATGTAGGTCTATATCTGGGCAC | | TTGTCTTGAATTAATAGATCAATTTGGGAGGAATTA |
| | | TCGATCATGTTTTATTGACCTCTTTTTTAGTATCTT | | ACATCTAACAATACCAAGGCTTCCAACCTTATGTTT |
| | | ACCATCATCATGTGCTTTTGATTTCAATTTGTTCT | | CCAATCTATGCAAAATACACCTCCATTAATTTAG |
| 348558 | SCAFFOLD | AAGACAAAGAACCAGTAGCAAGACAGTGGGACGTGT | G/A | AGGGGGCAAAAGAATGCACACAGGGTCTGGAAGGGC |
| | 275605_3060 | GCTCTGATGTTGGTAGGAAGAAGTACTAGGGACATT | | AAATGTGTGTATGGTGGTGAGAGGTAAGGATACGGG |
| | | AGCAAGGGGGCACCCAGCTTGATCTGTCTTTGGTGG | | AGGCTACAGACTGAAGAGAGATGAGTTTACAGATGT |
| | | GGATGATCAGAGAATACACCCACAGGAAGATGAAAC | | GAATGGGGAGAAGGAGAGTGGATGAAAGATTCATGT |
| | | TTCTTAGGCCGGTAATAGCCAGGAGAAGATTAAGTT | | ATTCATTCAACAAATGTACTGAATGACTACTACGTG |
| | | CATTCCAGGCAAAGAGAATAGCAAAGTACAAAGACC | | CTNNNNNNNNNNNNNNNNNNNNNNNNNNNNNNNNNN |
| | | AAGAAGCAAGAGACAAGAATGTATATAATTGGGG | | NNNNNNNNNNNNNNNNNNNNNNNNNNNNNNNNNN |
| 354434 | SCAFFOLD | TATCAGTGGTTGGGGCATAGAATTGGACTACTATGA | G/A | TGCAATTATATTTTCTTAAAGTATTCAGTTGATTTT |
| | 20780_8873 | TGTTGAATGATTTGCCTTGGAAATGAACTGAGATCC | | TAGAGATTGATAACTAATTCTGGAGACTTTTAAAGA |
| | | TTCTGTCATTTTTGAGATTGCACCCAAGTACTGCAT | | GTAGAGCAGTGTTTCTCAAACTTTAGTGTGCAGAAG |
| | | GTTGGACTCTTTTGTTGACTTTTTTTTTTAATCATT | | ATTACCCTGGAAAAATGTAAACAGATTCCTTGGCCT |
| | | GTGTAGTTAGACAAAAAATTGTCTCTATGAGAATTT | | CACTTCTAGAGATGCTGCTGCTGCTGATTTCTAGAC |
| | | TGCTGTTATCAAGTAAATAATACAGCTAAACTAATA | | CTGTACTTTGAGTACCTCTGATATAGAAAACTTGGA |
| | | ATTTATAAGAAATTAATAATAATGTAAACCTTAT | | AAAATCCTGAAGATTAATGTGCCTAACCTATGAT |
| 345723 | SCAFFOLD | TTCCCCGGATAGAGTCTGGTGGTGTACATAGGGCTT | T/C | GTTTGGACAAGTATCTGGCATATCACAGACACTTGT |
| | 237108_7683 | CTCTCAGTGGAAAGCCATATGGATCACCCAGTCTAA | | TGAAAGAATGACTTCATAGAGAATTCAACTGAACTA |
| | | ACCACATCCTTTCCCCTGCTGCCTCTCTTTGCCCTC | | TCTCTTCACTGACTATTGGCTGCATGTCTGCCCGGT |
| | | TTATGCTGCTTCACTTTGCTTTGTGGCATTGCCCAT | | GACATCATTGGAGAGACACTGCAGTCCCTTAGAGAG |
| | | CATCAGATACAGTACAGGTGTGTTTATTTTCTGCTA | | ACCATCAGGACGGCTAAGACTGATCATATCCCTGGT |
| | | CCCTGTTTCACACAAAGGGACTCCCATGAAGATGGG | | CAGGATTGAGATTCAATCAAGGCACCTAGTATAGGG |
| | | GATGGGCATGTTTTGTTCTCTGCTTTATCCCCAG | | CTTGAACTCGACCACTGGGGCAAGAGAGAAAAAG |
| 353961 | SCAFFOLD | TCTCAATATATTGAGATACAATTATTATTAAAATAA | G/A | GAAATCTTATATCTTAGTTAAATCACCCAGATGTTA |
| | 100908_8920 | TATATATATAAAATTGCTAGAAATTTCATGAATATG | | TATTTTACTATAATATTCCCCAGGAAGTTGGAAGAA |
| | | CTTATACCAATTTTAATTGTGAATGGCTTCAATAAA | | CAGGTGGTTGATTGTATGATGGCAGAGAGAGAAAGG |
| | | GAGAAAGACAGCAGATCTATGAATAACAATAAACTT | | AAAGGATGCATCTTTTTTTTTTTGTAAACTGTCCCA |
| | | CTAAATTTCAAGCGCATTTTCTTCCACTGAACTCCA | | AGTTTTTTGGAAACTGGTGTTTTTCCAAGAGTCACA |
| | | AGTTAGCCAAGCTTATGTAGTTGTGCAGAAAACCCA | | ACTCTTCCAACAAGAACATGTTTCTTTCTAAGCTGT |
| | | TCTTTTTGTAGAAAAGTATGTCATTAGTGTAATG | | TTAATTCCACTAAATTCTATTTTAGCCTATTTCA |
| 353960 | SCAFFOLD | CTGCAGACGATAAAGCACTTCCAGGCAGACAGCAGT | G/A | AATAATGAACATAGATAATTACTGATGCTTGCCAAT |
| | 100908_7806 | ATAACCATCAGGGCACCTTGTTCGTTTCTCATTTTT | | GATATAGTCTATAGATAATGAAATAAAGCAAGAAAA |
| | | ATGGAATCAGAATCCTATGCTATGTGTTGTTTGGAA | | GTGAATCAGTGTCATGAGGATAAAAAGGGAGAAAAT |
| | | ACAATTGAAACCAGAATATTACATCTATCTCCAATA | | ACTGTCATTATTTATAGGTGATACAATACCCTACTT |
| | | TGACAGAAGAATAAAATATTTTCTGGTACACACAGA | | AGCGAAGCTAATGAATGAGCAAATAAATTAATTACA |
| | | TTAAGAATGCTGAAGAAATCTCTAGATGTAGTACTC | | TAATATAGCACATATATGGGATACAAGCTCAGCCTA |
| | | AAACAGGGTGAAAGGAAATCTTTATATATTGCAA | | CAGAAGTGAAGTGAAATGAAGTGAAGTCGCTNNN |
| 350052 | SCAFFOLD | AGAGGCCATTGCTTATTAGAATGCAGTCAGCACAAT | G/T | TGTCTAATCACAGTATCCAGGCCTTTTACTGTTTAG |
| | 200312_7695 | TACATAGTAATGGTTGGCTCATCAAGTTGGTATCTG | | CTGTTTCATTGCCAAAAAGCTGTTGACATACCCAGT |
| | | CTTGGAAATTATGCCTATGAGACACAGATTTAATTA | | AATGCACTGAGGAAGAGGTATTTTCCAGTTTTAATT |
| | | CAGTTGGTTTAGTGGGCAATGTTGGGTTATGGTCAA | | AGTATTGTTATTCTGTCACTAATGTTGGAAAGATTA |
| | | TACTGGATAAACAGGTAATTACGAAAGCAAAAAATG | | ATGGATGCAGCATTATTTCTCAGCAAGTAGACACTG |
| | | AAGATTCTGTTTTCTGTTAATTATATTTTATTAGCC | | GTTTACTTCACTGGCAATGCATTGAATTACTCTGCT |
| | | TTTTTTATCCACCATCTTTTTTCTTTGCCTTAAT | | TATCAGAGTGAGGAAACTGAGCAGGAGACATTCC |
| 347164 | SCAFFOLD | GGCAGGCAGGAATGTGAATGCGGGGGAGAGGTGGCT | G/A | TCCTCCCTTTGTCTAATGAGCCTTCTAGAATATCCT |
| | 130114_24383 | GCAGTTTCACCTCCCCCATGGGGAAAGAAAGTGCCA | | GTGCTCCCTGCCCACCATTTTTGGCCTCAGTCAGAT |
| | | GACATTTCTTCAGTACCATCCATCAAGTGCAGACAG | | TTGATTTATTGGGAGAGGGACCTAACACAGTCAAAT |
| | | GCCCCTCCTCCGACCTCTGCAAGCCTCTTCCTGGAG | | CCTCACCCTCCTCAGCTGGGGACAAATAGCTCTGGG |
| | | TCCACGCCCAGCGGCACTGGAGGTTAGGATGGTCCA | | AAGGCCTAACACAGAAGGGTGGTTGCCTTGGTAACA |
| | | GGGAGAGCTTGGAGGTCTGCCTTGGAGACAGCGCTC | | CGGCAGCACATCCAACTTCCTGATGGGCCCTGGTGG |
| | | CTTGATACACAAGTGGCTGAAGAAAGGATGAATT | | CCATGTTGACTCCCAAACCCCCATTCCACCTCCC |
| 353150 | SCAFFOLD | TGTGATGAGAATACTTAGGATTTACTCTCTAAGCAA | C/T | ATGTTAGTAAAATCTAAAACTTGAAATAAAAAAATA |
| | 190014_28020 | CTTTTATATATAACACACTGCAGTGTTGATTATATT | | TGGAAATATTTTCATAATTTTGAAAAGAGAAGCATC |
| | | TATCATGTTGTTTTTTTATCTCCTTAGTGCTTATTT | | CCATCCAAAGCCATTAAGGGAAAAATAACTAAATTT |
| | | CTCTTACAACTGGAAGTTTGTACTTTTTGACTACCC | | ACATAGCTTTTAAAAAAAAATTGTATAGCCTGAAAA |
| | | TTCTCCAGTTCCCCTTCCTTCTCCCTTCTCTCCAGT | | GACCATAATAAAATCCAAAGACCCCCTCTCCAAAAA |
| | | AACCAGAAATCTGATCTCATTTTCTATGAATGAGTC | | AATCTTTGACATATATGACAATGTTTATGCATTGAT |
| | | AATTTATTTTTGAAGTCTAAGTGACCTACAACAT | | AAGAAAAAAGACTAGTAACTAAGAAAAAGGACAA |
| 350716 | SCAFFOLD | GTTTGACAGCTGTTCATTCGATCTTGAAGTACTTGG | C/T | GTACTGCCAAGAGTGAATTTGATAAGAATAAATTAA |
| | 145586_11232 | TAAGAGAGGCTTCTAGTCTGGATAAGTGTGATGTTT | | AGGTATATATGTAGATTCGGGGAAAAAAGCAATACT |
| | | ATTTATTTATTTTTTAATGGGGTGTGAGAGGATAGA | | TCAATAAGTAAACAAAGGGAAAGTTGACTTGGTAAC |
| | | GGAGAATAAGGTCATGTATTAGTATGAGATTTTGAA | | AAATCAAAAGTGTGACATAGCTTTGTATTTTTAGAC |
| | | AATAAAGTTCCCTGGGACAAGGATGTCAGGAAACCC | | AAAGCTGATGCGATTTAAAACCAGAGCATTACAAAT |
| | | AGGTAGAAGGGAGAATTTTCTCTTATAAGGACTAAT | | GTGGTAGGATCTAGGTTTAGGAAGTGCTACTGCTCT |
| | | GTCAGATATGGACATTCTTCATCTATATGTTCTG | | CTGTAACATGTTACTTGTTCCATGTTTGGGGGTC |
| 348284 | SCAFFOLD | AGTATTCTGGCCTGGAAAATTCCATGGACTGTATAA | G/C | TTATTGCATGTGAGGCTTCTCTTTGTCCTCAGACTC |
| | 185349_3781 | TCCATGGGGTCACAAAGTGTCGGACACAACTGAGAG | | ATGTCTCATACGCTCTATCTTTAAGTTCGTGATCCA |
| | | ACTTTCACTTTCCCTCTTGTTGATTACAGCAGCCAT | | ACATGAATTCCTGACAGTCCTCTTCCTAATTGCTAT |
| | | CTAACTGAACTTTCCACCCCAGGTCTGAGCCCTTCA | | TCCTTATTTAGTTTCTTCTAGTCCCATTTTCCTGAG |
| | | TAGTCATCTTCAACACCCCTAAGATAAAGAGCCTAC | | ATAAATGTCCCACCCTTGAAAGGCTAATTTTTACCT |
| | | TAAGCCATTCTCCAGTTTAAAGTCTCCCAATAGCTT | | GTCCTAAAAGGCAGAGCTCAAATGTTGTCTCCTCTA |
| | | CATTACAACCTACAGTGTAAAGTCCATATGTGAT | | GATTTGGGTAAAATGTTCCGTATCTGTGCCCTTA |
| 352676 | SCAFFOLD | TACTCAGAAGTTATGTCTACTTGTGCCCTCTGGCTT | G/A | AGTAAGACACTGAACTGTCATTCAGGAAGACATATC |
| | 185915_226 | TGCTCTACCTAAAAGGCACTAATATTTGTGCTAATA | | TTAAGCAGGAGTGCAGTATTCTTGGCATACATTGAA |
| | | ACACAAGCTCTTTTGTTCTACATATCCCAGTGACAA | | TTTATTTACTTGTATCTTATTCCTGTCATCTCTTGG |
| | | GAAGGGCAGTAAACCAAAGAACAAAGAAAATAAAGA | | CTGACAACTGACAGGATTTTTATTCTTAACTTTAAA |
| | | AAGATATTGATAAGTTCTATAGCAGCTTATATCATT | | CACGTTACCAAGCTCCAACACTCAAGCTTTTGATGC |
| | | TAATATAATTGGGTTTTCTTTAAAGATGAACTTTAT | | TGTTACTCTATATTAATGTTGATGCCATTAACATCT |
| | | TTCACTCATTACATGATTATTTAGAGTTTATTGT | | CTGCAGGTG |
| 349084 | SCAFFOLD | CTATATGAATTTTTAGAAAAACAAGACGGAATTTTA | A/G | AAATGGAGATAAAGAAGTAGCATGAGACTTTCTTAA |
| | 16083_20878 | AGAAATTAAAAATATGATGGCAGAATAATTATATTT | | AACTAAAACATAGAATTTTCAGGTTGAAAAGACCCA |
| | | TAACCCCATAAAGTATTAGAAGATACAGTTGGGAAA | | CTGAGGATAATAAAATGAATAAAAACAAAAATATAA |
| | | TCTCCAGCAAGATGAAAAACAAAAAGGTGAAGAGAA | | TGACATTAAGTGATATCATAATGATATCAGAACACC |
| | | GAGAAATAGGAGACAAAAAAGGTAATAGATTATGGG | | AAGATGAAAGAGATAATCAAAATTTCCAGAAAGAAA |
| | | TAAACATAAGAATGTCTATGCAGGAATGCCAATATC | | AAAGTCCCATCACAAACCAAAGACCAGATATTAAAA |
| | | AGAGTAATTAAAGGCCTTGAAAGAAAGATCACAG | | CACTGTCTTTCAACTGCAATACTGGAGGTCAG |
| 351882 | SCAFFOLD | CTTGGCAGGCTACAGTCCATGAGGTCGCAAGAGTCG | A/G | TCTAGGAAAACGGATGGATTGCATGGGTAGCTCTAT |
| | 66277_3361 | GACATGACTGAGGGACTAAATCACCAACATGGAGAT | | CAATAAGAAGGATATGTACATATAATGAATTCAATA |
| | | CAAAGATATAGATGAATACTAGAGTCAAATCTCAAC | | ATGTTTCATAACAACAGTGAGAAAAGTCTATGTGAG |
| | | CAAAATCTCAGCCTCTCCCCACTCTATGACCTTGTG | | GTAATCAGGGAAGAGTGTGAAGAAAATATATTAATC |
| | | CAAATCTTTTATTCTGAAACCTCAGTTTCTTCATGT | | TCATGTAAGTACAGATATTGTAAACTTCAAAATATT |
| | | ATAAGATAGATTATATATTAATAATTATCTTTTATA | | GTGGAAACTGACATTTGTATCAACTTTTTGGTCTAT |
| | | TGATTTAGTGAAATAAAATTTATTAGCACTGAGA | | TTAAGTTTATTCTTACTAGATTATGTTTTAACTA |
| 353906 | SCAFFOLD | TGAAGTTACTTATCCAAAGCTATAGAACACAAAGTC | G/A | AAAATCAACCCCAAAAAGTCTAGGGGCAGGTCAAGA |
| | 41654_3423 | TTGCACACATTAGGTCTTAAAATATAAATTACATGT | | AATGTATTAAAAAAATATGGAAGAGATCTCAGCATG |
| | | TAGTTGTTATATTAGCAAAAGAGGCAGACCTAAAAT | | TTATCCTGAGGGAAGGACAAAATATTCAGTTCAAGT |
| | | ATCTGGAGCATTTTTTTCTCACCAAAATCACTGTTG | | ACCACTACTGCACAGATCTGGTGGGACCCAGCTGTT |
| | | TTTAATAGGTTTGTTTTTTTTGTTTGTTTGTTTTTC | | ACTAGAACCATGAATGCTTCATTTTGCACAGAGAGG |
| | | CATTTGTCTGGAAGTTCTTCATTTGTCTGGAAGTGT | | TATTTACAGGGAAGTAATTTCCTAAGAGCAAGAGCT |
| | | TCAAAAGAAAGTATAAAAGAAAAATGTTCATTAT | | CTGGTCAACTCGGATTGGGTTGAAGGCATGCTGT |
| 346707 | SCAFFOLD | AGAGCCATTTGAATAAATGCCCAGTCCCTAATGGTG | G/A | GACATTAGCAAGGGGGCACCCAGCTTGATCTGTCTT |
| | 275605_2857 | ACAGCTTTGAGGGGCAACGTTCACTTGGATGGATAC | | TGGTGGGGATGATCAGAGAATACACCCACAGGAAGA |
| | | TCGTGGTCTTATGCTTTGTCATCACCCATTTATTTG | | TGAAACTTCTTAGGCCGGTAATAGCCAGGAGAAGAT |
| | | TCCATTCTTGCTACACACGTATTGAACATCGATTAT | | TAAGTTCATTCCAGGCAAAGAGAATAGCAAAGTACA |
| | | ATGTTGCTAAACAGCTGAATAAAACTAGATCACAAT | | AAGACCAAGAAGCAAGAGACAAGAATGTATATAATT |
| | | CCTCAAGACAAAGAAACCAGTAGCAAGACAGTGGGA | | GGGGGAGGGGGCAAAAGAATGCACACAGGGTCTGGA |
| | | CGTGTGCTCTGATGTTGGTAGGAAGAAGTACTAG | | AGGGCAAATGTGTGTATGGTGGTGAGAGGTAAGG |
| 353270 | SCAFFOLD | TCGTGGTGACATGATATATGGATTTACTGTGAAAGG | G/A | ATATCTTGGGATAAGCTAGAATGGAAACGAATACTA |
| | 280729_3072 | TTAATTTACTGTCAGCAGTTTTGGATCTCTTTGGAC | | AAGAGAATGTATATATGTATAATATAACTGAATAAC |
| | | CAAAATCCCCTCCTGGACCCTCTGGCTTAGCTGGAT | | TTTACTATATAGTAGAAATTAACACAACATTGTAAA |
| | | TCTCGTCTGCAGTCACGCCCTGCTGGTTCTCACACT | | TAAACTATCAGTTCAGTCAGTTCAGTTGCTCAGTCG |
| | | CCAGAGGACAGACGTTCCCTCACCTCGTAGGAGGCC | | TGTCCGACTCTGTGCGACCCCATAGACGGCAGCCCA |
| | | CTACATATACTTACCAGAGATCAAGTGAAGAGCACC | | CCAGGCTCCCCCGTCCCTGGGGTTCTCCAGGCAAGA |
| | | AATATAAAAGTTTAAAAAATGGGAACTATATTC | | ACACTGGAGTGGGTTGCCATTTCCTTCTCCAAT |
| 352344 | SCAFFOLD | TGAGGAAAGGATAGATGGGGTAACCCAAGCCAGGAC | T/C | CACTTTTTGTGACAGCACGTTTAATTTGTGTAGCTA |
| | 305461_2840 | CACAGCTGAACAAACAAAAGCCAATGGCTGCTCAAT | | TTCTCATTGCAGGCTGATTTGCTAAAATATGAGCGA |
| | | TAAGAGTGAGTCAGTAGCACCATACTCCTGTATCAA | | ACAAGTCTAATGTAGGGAGTTGAACAGAGCACTATG |
| | | GGGTAGAACATATAATTTCTAAATGTCTAAAGCCTG | | GTATCGAGTGTGTAGTTTCTTGCCTTACGATGCCTA |
| | | GAATTAATGTCTGTGACCTCGGTAATACCAAGAGAT | | CTATGATTTTAAAGTTACTTAAAATTGTTTCTAAAG |
| | | GAATTGTGAGAATGAAGATTGTTCTAATTTTCTCAG | | ACTTCCCCTGAACTTGACATCAGGAACTTACAGTTA |
| | | CAAAGATAATATTTTAAAAACTACAAACTGAAAA | | TACTTGGTTGAGGCCCCTGTTCTCAGTGGGGTAT |
| 347611 | SCAFFOLD | GGGATCTTCTGACCCAGGGAAGATGCAGGGTCTCCA | T/C | ATTTGTCCATTCTTGCTACACACGTATTGAACATCG |
| | 275605_2728 | GGGAAGACCCCTGACCCAGTGCAGGGGTCTCCTGCA | | ATTATATGTTGCTAAACAGCTGAATAAAACTAGATC |
| | | TTGCAGGAAGATTGTTTACCTTCTGAGTCACCAAAG | | ACAATCCTCAAGACAAAGAACCAGTAGCAAGACAGT |
| | | AAGCCTCAAGAATAGGTCTCAAGTATTTTGAGAAAT | | GGGACGTGTGCTCTGATGTTGGTAGGAAGAAGTACT |
| | | GCTAGAGCCATTTGAATAAATGCCCAGTCCCTAATG | | AGGGACATTAGCAAGGGGGCACCCAGCTTGATCTGT |
| | | GTGACAGCTTTGAGGGGCAACGTTCACTTGGATGGA | | CTTTGGTGGGGATGATCAGAGAATACACCCACAGGA |
| | | TACTCGTGGTCTTATTGCTTTGTCATCACCCATT | | AGATGAAACTTCTTAGGCCGGTAATAGCCAGGAG |
| 345696 | SCAFFOLD | AAATAAAAAATATCATGGTAAGTCAAAAGATGGGAG | T/G | CGATTGAGCGTGAGTATAAATATAAGAGAAAGAGCA |
| | 226597_1590 | AAGAAAGGAAAATGCTATTTTCCTGCTCTTTGGACA | | TGGGCATGATCAGAAGACCCACATCTCTCTCTCGTT |
| | | AGGAATCCAGCATTTTCGTTTTGGTCCTTGTAAGTT | | CATGTTTTGCTGGTATGTGACGTTGGGCAAGTCACT |
| | | ATGCTCATTGAAATTTGACAAGCTTTAGTATAAGAG | | AGACCTCCTTGCCTCAGTTTCCTCATCTGTAAAATG |
| | | ATGGAGAAGGAAATAGCAACCCACTCCAATATTCTT | | AGGGTGATGTCTTATGCCCCTTTCCAAATGGGCTGG |
| | | GCCTGGAAATCCCATGGACAGAGGAGCCTGGAGGAC | | GTCTAGGTTAAGGGCAGACCTGGCAACATGTCTTTG |
| | | TGCAATTGGTTGCAAAAGATTCAGGCAGGACTTA | | AGTAGGAATGTGTCCAGAACACCTCTCATCTTTG |
| 352298 | SCAFFOLD | TGAAAAAATTATAAGACATGTAAAGTTTACATCGTG | T/C | ATAAAAGTTTAAAAAATGGGAACTATATTCGATATC |
| | 280729_3103 | GTGACATGATATATGGATTTACTGTGAAAGGTTAAT | | TTGGGATAAGCTAGAATGGAAACGAATACTAAAGAG |
| | | TTACTGTCAGCAGTTTTGGATCTCTTTGGACCAAAA | | AATGTATATATGTATAATATAACTGAATAACTTTAC |
| | | TCCCCTCCTGGACCCTCTGGCTTAGCTGGATTCTCG | | TATATAGTAGAAATTAACACAACATTGTAAATAAAC |
| | | TCTGCAGTCACGCCCTGCTGGTTCTCACACTCCAGA | | TATCAGTTCAGTTCAGTTCAGTTGCTCAGTCGTGTC |
| | | GGACAGACGTTCCCTCACCTCGTAGGAGGCCCTACA | | CGACTCTGTGCGACCCCATAGACGGCAGCCCACCAG |
| | | TATACTTACCAGAGATCAAGTTGAAGAGCACCAA | | GCTCCCCCGTCCCTGGGGTTCTCCAGGCAAGAAC |
| 345859 | SCAFFOLD | AACCAGGGTCACCTAGTTTTTTTTTTTTTTTAGTTT | C/T | GTAGGAGGCCCTACATATACTTACCAGAGATCAAGT |
| | 280729_3153 | TACCGAGGTATAATTGAAAAAATTATAAGACATGTA | | TGAAGAGCACCAATATAAAAGTTTAAAAAATGGGAA |
| | | AAGTTTACATCGTGGTGACATGATATATGGATTTAC | | CTATATTCGATATCTTGGGATAAGCTAGAATGGAAA |
| | | TGTGAAAGGTTAATTTACTGTCAGCAGTTTTGGATC | | CGAATACTAAAGAGAATGTATATATGTATAATATAA |
| | | TCTTTGGACCAAAATCCCCTCCTGGACCCTCTGGCT | | CTGAATAACTTTACTATATAGTAGAAATTAACACAA |
| | | TAGCTGGATTCTCGTCTGCAGTCACGCCCTGCTGGT | | CATTGTAAATAAACTATCAGTTCAGTTCAGTTCAGT |
| | | TCTCACACTCCAGAGGACAGACGTTCCCTCACCT | | TGCTCAGTCGTGTCCGACTCTGTGCGACCCCATA |
| 348454 | SCAFFOLD | ACGTGACCACTTTATTCTCAAGAATATTTGAGGTTG | C/A | CCTAGCTGCTAGTAGATGTTAACTCTATTGTTCTCA |
| | 245025_31911 | GACAAACAGTCTCCAAGTACTTCTTCACATCTAAGA | | GATGTGTCCACTCCCAAGATCCAACTCACTCAGCAA |
| | | ATTGTAGAAAGAGAATTGGCATGTAAAAATCCTTCC | | GCTTTCATTCATTATTAATGGATGGAATGAGTATTG |
| | | ATTTGCACTGAGAGGACCGAGATTCTATGTCATTTT | | TCTCATGTCTCACCATTGAGCTAATGGGCAGAGAGG |
| | | ATATCAGTCAGTGGAAGTTATTTCTATAATCTGTAC | | GAGCCAGCACTGTTTCATTGAACCATTACTATATCA |
| | | TAACTCAATTGTCTGAGTATAAAAATATTCCCTATT | | GGCCCTTACAGTAATTAGGATTCTTAACTAATCTAG |
| | | GACAACATGGCCTTCTTTTTCTCCTAACTTTGTT | | CCATTGGAGAAGGAAATGGCACCCCACTCCAGTG |
| 350374 | SCAFFOLD | TGTTCACCGAAACTGGGTCTCCCCATGTTCTTTCTC | G/A | TGACCCCTTTCCAGTAACATCTTTACTGATGTCTTT |
| | 321295_853 | TCACCTTCTGGCTGAATTATTCAGGCTCTTTTCTTC | | TACACAGTTCTTTCTCCAGCAAAACATCTGTGCCTC |
| | | ACTGAATTTTCCTACTGAGCTATCCTTATTCTATTA | | ATAGTTTTAAAAATCAAATCAGTGTACAAAAAATAG |
| | | CTCTATATCCTTAATTAACGTTTAATTAAGCAGTTG | | CAGTTTTTTTATACACACCCTCAGGGATTGCCTCCT |
| | | TTTCCTGGTCCTCGCCGACGCCGTCCTCGCTTCAAA | | TTAGCTGGTGCTGTGGTATGTTTCCAGGGTACATGA |
| | | TTCCCTGGATCCACCGGGGCTGGACACCGGCAGAAG | | CATGAAGGTGTCAAGGGCAATAATTCACCCAGCTGC |
| | | AGGGTTCCCAAGTTCTTCTTTTTCTTATTCATGC | | TCTTTGTAAGGTTCATGCCCTTGGTTTTGGGAA |
| 345742 | SCAFFOLD | GCACAGATGGCATCAGTCAAGGTATTGGTGGACTCT | C/G | TTGTGTCCGGCTGTCTGAGACCCCATGGATTGCAGC |
| | 243592_693 | GGCCCAGAGTTATAATTAGATGAATTAAATCTTATT | | ATGCCAAGCCTCCCTGTCCATCACCAACTCCCGGAG |
| | | ACCAGTAGAGAATGTAGCTTTTCATTTAAAGGAATT | | TTTACTCAAACTCATGTCCATTGAGTTGGTGATGCC |
| | | AAAAACATATTTTAAAATATACTCAATAGTTTACTT | | ATCCAACCATCTCATCCTCTGTCATCCCCTTCTCCT |
| | | ACAAAACATCTAAGTTACTCTTGTCATTGTAAATAT | | CATGCCTTTAATCTTTCCCAGCATCAGGGTCTTTTC |
| | | AACTTCATTTCTATACCTACAGATGTGTATTGACAT | | CAATGAGTCAGTTCTTCAGATCAGGTGGCCAAAATA |
| | | GAGGCAGTTCAGTTCAGTTCAGTTTATTCGTTCA | | TTGGAGTTGGACATTATTACTGTAATCATCAGTT |
| 352752 | SCAFFOLD | ATCACTGTGGATGTTAGAAATCAATATAGAATGATC | A/C | TGGAATTTCTGATTAGAAGGACATTACTGACATTGG |
| | 240147_59288 | TCTTATATACAAAAACTAAACAGTCAAGATATATGC | | GGACAACGTCAATGGAATAAGAGAGTAAAAAGCAGA |
| | | ATGGAATGAATGAATGACCATAATTGTGGGGATACA | | TTGCAGGAAGTTAGGAAATTAGAATAAAAACAATAG |
| | | TATAATGTCATAAAGATGTTGTGGAGTTAAAAAAGA | | AGAAAACACAAAGAGACACTTCTGGGAAGTTTAGCA |
| | | AAATATATGTAAAGTGCTTAAAATAGAACAATGCTC | | GAGAAGAGATTAAGAAAAGGTATGGTGGGTCAAAGG |
| | | GACACAATAAATGTTTTCCATTGTGATATTTATTTC | | AAGGCTGGCTGATTGTTTATTTTAGGGAAGTGGCAG |
| | | TATTATTAGAATAAAACTGTTAAGTAATCAATGA | | GTAAGGAGAGACATGGAACACTGCTTGCCCCAGA |
| 353446 | SCAFFOLD | TGTATTATAAACACACTATTGATTTGATTGATAACT | A/G | ATGAAGACCTCAGAGAAGTACAAGTATAGGTGATTT |
| | 10927_1021 | ACCTCATAAACATGAAATAACAATAATGAATGAAAC | | TTCTTCTTTTTTCTTTGGAAATCTTTTCTAAAATGT |
| | | ATGGCAGAAGAACCTTTATTAACAAACCATATAAGA | | CAACAATAGTATGACACTTTTAATATGATTAGAAAA |
| | | ATAATCATGACATTGAACCTTCACAGAGTGATATCA | | ATGTTGGTTTTATAAGTTATACTGATGGTATTGAAG |
| | | ATAAGCCAAGTTAAACAAACCTTAAAAATGTAATTT | | TGTCTAGATACCAGAGGATAAAACTAGAGGACCATA |
| | | GAATGGAATTACCAAAGTTTTCCTGTTTGTAAAAAT | | ATGTATGGCTTTATTATATCAAGGAAGTGAATTATA |
| | | TCTGGAAAAATAAACAATAAGATCTAAATATTAG | | TCCATCTTAAATTCACCATGAGAAAGAAATATGA |
| 344286 | SCAFFOLD | TGTGAGTCTGTTTCTGACCAGTTCATTTGTACATTT | T/G | TTGCTTACATGTCTTGGCTCCATCTTAACCATCTTC |
| | 315271_6985 | GTATCATGTATTTTAGATTGCAGATATAAGTATATC | | ACCTTGTTGGGCAACCATCAGCACCATCCATTTCCA |
| | | ATATGGTATTTGTCTTTGTCTGACTTAATGTGCTAA | | GAAGTCTTTTGATCTTGTTAAAATGCAAACTGTGTC |
| | | TCGCTAGGTTCATCCATTTTGCTACAGTTGGCAGAG | | CTTTTGAAACAGTAATTTCCCATTCTCCATCTCCCA |
| | | TTTCATCCTTTTTATGGATGAATAATATTCCATTGT | | TCCCTAGCAACCACTGATCTACCTTTTTGTCTTTCT |
| | | GCCTGCACACACGTGTATACACACACACCCTCCCAC | | GAACTTCGCTACTCGAGATATTTCATATAAGTAGAA |
| | | TTCTTTATTCATGTATCGGCCCATGGACATTTAG | | TCATACAGTGTTATCTTTTTGTGACTGGCTGATT |
| 346919 | SCAFFOLD | TCTGCTGCTGCTGCTAAGTCGCTTCAGTCGTGTCCG | A/G | GGATAGCATTCAGAGCCAATGGAAGGGATGATGTGG |
| | 51554_10006 | ACTCTGTGCAACCCCATAGATGGCAGCCCACTAGGC | | GGATTATTCGCCAGACTCTGGAGTGAGAGAGGTTGA |
| | | TCTTCTGTCCCCTCTGGATTACTTCCATTTTCTTAG | | ATAAGAACCCAGCCTCCACCAGCTTCCACCTATGTT |
| | | AGACCTGGAAAACAAAGTCATGAGCTGAGAATGCAT | | ATCTGGGCAAGTTAGCTAACCTCTCTCTCTAACCCT |
| | | GCATGCAGGAGAGAAGATAGTGGAGGCTCAAGGAAA | | CCCTTTCCTGCTCCATAAATTAGTGTGCTAAGTTAC |
| | | AAGGAGATGTATAAAATAATCCTATAGGAGAGAAGA | | TTCAGTCATGTCCGACTCTCTATGACCCCATGGACT |
| | | GGAAATGAAACGCACCAGGGACTAGTATATGATA | | GTAGCCCACCAGGCTCCTCTGTCCATGGAATTCT |
| 347396 | SCAFFOLD | TTCAGTATTCTTGGCTGGGAAATCTCAGGAACAGAG | C/T | TGTCAGGAATCACAGATGTGAAGCAATTAGTTCTAT |
| | 210732_8549 | AAGCCTGGTGGGCTACAATCCATGGGGGTCGCGAAG | | CTTGGGAAGTAGGAAAGGCTTTGCAGATTGTTGTTG |
| | | AGTAGGACATGATTCAGTGACTGAGCACATGCACAT | | CTGTTTAGGCTTTGCAGAGCTGATATTTACATCAGT |
| | | ACAATATTATATATTTATCTTTATACCAGTAAGTCA | | TATTTGAGGATAAATATGTTTTCTACAGGTGAAGAT |
| | | TAAATGTAACCTGTAACATTTGTTGTAGCCCTGGGT | | AGTGGGAAGGGCATTTCTGGGAGAAAAAGCAGCATA |
| | | AAGCTAAGATGAATTCTTTGAGGAGTCCAGTTATCT | | AACCAAGGAATAGGGTTATGGAAAGAGGGTGTTGGA |
| | | GAAGTTCAAGGCACACGAAGACATTGTTCTAACA | | TCTTGGCAGTGGTCAATAACCAATAATGCCTTTC |
| 349561 | SCAFFOLD | CATGTTCTTTCTCTCACCTTCTGGCTGAATTATTCA | T/C | TACTGATGTCTTTTACACAGTTCTTTCTCCAGCAAA |
| | 321295_876 | GGCTCTTTTCTTCACTGAATTTTCCTACTGAGCTAT | | ACATCTGTGCCTCATAGTTTTTAAAAATCAAATCAG |
| | | CCTTATTCTATTACTCTATATCCTTAATTAACGTTT | | TGTACAAAAAATAGCAGTTTTTTTATACACACCCTC |
| | | AATTAAGCAGTTGTTTCCTGGTCCTCGCCGACGCCG | | AGGGATTGCCTCCTTTAGCTGGTGCTGTGGTATGTT |
| | | TCCTCGCTTCAAATTCCCTGGATCCACCGGGGCTGG | | TCCAGGGTACATGACATGAAGGTGTCAAGGGCAATA |
| | | ACACCGGCAGAAGAGGGTTCCCAAGTTCTTCTTTTT | | ATTCACCCAGCTGCTCTTTGTAAGGTTCATGCCCTT |
| | | CTTATTCATGCGTGACCCCTTTCCAGTAACATCT | | GGTTTTGGGAAGACATTATAGGCCTGCCCAGATA |
| 344385 | SCAFFOLD | TCAAGAGAAGCTACCAGGCTCCCCAGAGGAAGGGGA | G/C | AGCTGGGGGTTAGGTTTCCCATATATTACAAATCAA |
| | 70164_24298 | TGACCCAGCCAGGTCTAAGCCAGGCACAGTTTGGGC | | AATTAAAGATAGATGGGATGGGGGGTGGTGCTTGGC |
| | | AGCACTTTGAAAATCCCAAGAGCTGCTAATCAATGG | | GAAGGGGGGTAGGGGGAAAAGCGATCTGAGGTTTTT |
| | | GGACCCAGAGAGCGTCAGCACCCTCTCCTCCACCCC | | CTCTTTAACTTGGCAAATGTGAAGTTAAAATGTTTC |
| | | CCACCCCCTACCCTCCTACGTCCCGCCCCCGCAAAG | | TTTTAAGTTAGAACGACCATATCATTCCACCCCCTG |
| | | CCAACCTCTCTCTAGCAGGGACGAAACTGCACAGTG | | CCCTGTGAACCTGGTTCTGATTATTAGCTGAAGTTC |
| | | GTTTGAGTCTGAAGCCAAGCAACACTCAGCATGA | | AGCACACGTCAAAGGCGTTTCTGGTTCGGCCTCC |
| 352343 | SCAFFOLD | CTGTAAGAATAAAAAGTCCCCAGTGTCTTAGGGTAG | G/A | TTCTAATTTTCTCAGCAAAGATAATATTTTAAAAAC |
| | 305461_2890 | CTGGAAAGAGAACTTGAGGAAAGGATAGATGGGGTA | | TACAAACTGAAAATCACTTTTTGTGACAGCACGTTT |
| | | ACCCAAGCCAGGACCACAGCTGAACAAACAAAAGCC | | AATTTGTGTAGCTATTCTCATTGCAGGCTGATTTGC |
| | | AATGGCTGCTCAATTAAGAGTGAGTCAGTAGCACCA | | TAAAATATGAGCGAACAAGTCTAATGTAGGGAGTTG |
| | | TACTCCTGTATCAAGGGTAGAACATATAATTTCTAA | | AACAGAGCACTATGGTATCGAGTGTGTAGTTTCTTG |
| | | ATGTCTAAAGCCTGGAATTAATGTCTGTGACCTCGG | | CCTTACGATGCCTACTATGATTTTAAAGTTACTTAA |
| | | TAATACCAAGAGATGAATTGTGAGAATGAAGATT | | AATTGTTTCTAAAGACTTCCCCTGAACTTGACAT |
| 348965 | SCAFFOLD | CACTCTGGCACTTTTATGACCCTGGGAGTGAGCACC | A/T | ATGACATCCCCTATATGTGGACTCTAAAAAGAAATG |
| | 120193_988 | TCCTCAAATTTTGAGCACTAGGCATCTTCCCTTACT | | ACACAAATGAACATACTTACAAAACAGAAAGAGACT |
| | | TCCCCCTACTCCTGGTACTGGCCAAGAACACCTTCA | | TAAAAAATGAACTTATGGTTGCCAGGAGAAAGGACT |
| | | CCTGTGACTTTGCCTATAGGATATACCTAGGAATGG | | GGGGAAGGGATAGTTAGGAAGTTTGGGACGGACATG |
| | | AACCGATAGATCACAGGATCCATGATGAGCAATTTG | | TACACACTGTTGTATTTAAAATGGATAACCAACAAG |
| | | CAGCAACATGGATGGACCTAGAAAGTGTCACACTGA | | GACCTACTGTATAGCACATGAAACTCAAATGTTTTG |
| | | GTGAAGTCAGTCAGACAGTGAAGGAGAAATATTG | | TGGCAGCCTGGAAGCGAGGGGAGTTTGGAGGGGC |
| 349048 | SCAFFOLD | ACTATAGCCCACCAGGCTTCTTTGTCCATGGGTTTC | G/A | TCAATTCAGTTACTCACTCAGTTCCAACTCTGCGGC |
| | 151694_4517 | CCAAGGAAAAATACTGGAGTGGGTTGATATTTCCTT | | CTCATGGACTGCAGCACACCAGGTTTCCTTGTCCAT |
| | | CTCCAGGGGATCTTCCCAGCCCGGGTCTGGGAAAAC | | CACCAACTCCCAGAACTTGCTCAAACTCGTCAATCG |
| | | CGGCTCCTGCTTGGCAGGCAGATTCTCTTATCACTG | | AGTTGGTAATGCCATCCAACCATCTCATCCTCTGTC |
| | | AGCCACCTGGGAAGGTCCTCTGTGTAGAGTACAACT | | CCCTTCTCCTCCTGCCTTCAATCTTTCCTAGCATCA |
| | | ATTTATCATAGCATACTAGGCTCTGTACTTTCTAGA | | GGGTCTTTTCAAATGACTCAATTCTTCACATCAGGT |
| | | TCCTGTGTGTCTCCGTAGCTCATTTTTCTGTTCA | | AGTCGAGTTTCAGCTTCAGCTCAGCCCTTCCAAT |
| 347878 | SCAFFOLD | AAAGTACATTTATATAAGTCATTAAATATACAATTT | T/C | CAACAACCTGTTCATGGAGAAACACACCTTTCAAAG |
| | 6869_6083 | TCTAAATATTTGAGATATTCTTTTGTTGTCTTAGAC | | CTGGGAAATACATGGAGATGGAGTGATACTGTTACC |
| | | TTATTTTTGTGTTTTCTTATTAATCAATTCCTGGTT | | CTCAGAACAGTTCATTCTGTTGCCAAGACAGCCAAT |
| | | ACTATCATTAAGGTACCTACCAAACTCATTTCTTAT | | AAGCAATGTATTTTTGTTCCCTGTTAATGTTTCCAT |
| | | GATGACTATGAAGATGAGCTATGCCTAAGGCTGGCT | | TCTTGTTTATCCATGCATATAAATTATATAACATAT |
| | | GCAAACAGAATCCCTGAGAAGCACCCCCAGTAGAGC | | ATTTTACTGTAGTCGAGAAAATAGTAATGCTTGGAT |
| | | CCTGCGAAGTGATGCCATGGACCTTACGTTTATA | | TTGAAAGCTACCAGAAACATTCTGCCCCTTCCAA |
| 347662 | SCAFFOLD | CAATCACCTTTGATCTACAAAATCCACTGTTTAATG | G/A | GCTAAGCATAACACAACTAAAAGAGAATAAGAAGAG |
| | 295083_24352 | TACTTCAACTAAAATAAAATAGGCTTCTCTCGTGGC | | ATGTCATAAGATTAATCTCATGCATTTCAGTCTGTA |
| | | TCAGATGGTAAAGAATCTGCCTGCAATGCAAGAGAC | | GTCTTATTTAGAATAAGAAAATACCTTAATTAACTG |
| | | CTGGGCTAGATCCCTGGGTGGGGAAGATCCCCTCGT | | GTAATCCAAGGATTTTCAGTTGTAGTTCATAGTTAA |
| | | GTGGCAGCCCACTCTAGTATTCTTGCCTGGAGAATC | | AATTATCTTTCAAAGTAACCTTACATCCAAGACCTA |
| | | CTCATGGACAGAGAAGCCTGGCAGGCTGCAGTCCAT | | TATATATAGCAGGTTTAATGAAGCTTCCTGTGATTC |
| | | AGAGTTGCAAACAAAAAGTCAGACATGACTGAGC | | ACACCAGGAACAATCCTATATTCTTGACTCCCATG |
| 350012 | SCAFFOLD | TAAGAGGTGAAATAGGAAACAAGAAATAATAAAAGA | C/T | GGTTCTTTCCTCCTGTAGTAAATATGCTTATTGATT |
| | 185915_514 | GGGTCAGAGACCTATAGGACTCAGATTGGAAAGGAT | | ACTCAGAAGTTATGTCTACTTGTGCCCTCTGGCTTT |
| | | ATGTGGGGAGACGACATGAGGAGGAACACCTGTTGT | | GCTCTACCTAAAAGGCACTAATATTTGTGCTAATTA |
| | | CAACAGTCAGAGACTATCCATGTCCCATTGTGACAC | | CACAAGCTCTTTTGTTCTACATATCCCAGTGACAAG |
| | | AGCCCAGAGCTGTGGAGAATGTAAATCAGCACTTGT | | AAGGGCAGTAAACCAAAGAACAAAGAAAATAAAGAA |
| | | CTGTCCATCTCAATTTGAAAATACAGGTGGCAGAGT | | AGATATTGATAAGTTCTATAGCAGCTTATATCATTT |
| | | CTATGCTTGCCATCTTTATTCTGGTCATCTAGAA | | AATATAATTGGGTTTTCTTTAAAGATGAACTT |
| 347705 | SCAFFOLD | TCATTTATGGAATAGAATGTGTGACTCTTTTTGTGT | A/T | ACAACAGCCGGGACATGGAAGTAACCTAAATGTTCA |
| | 306025_6030 | TTATGTGTTAAAGTAACTTGTCAGGCAAACTAATTG | | TGGATGGAGGAATGGGTAAAGAAGATGTGGTACATT |
| | | CAACTATACCTCAAAGGTATTTTTCAAACCCACTCC | | TTTACAATAGAATATCACTCAGCCATAAAAAAAGAA |
| | | AGAAGAGGCTTATGAACACACTAACTTAAGTATTTC | | CAAAATAATGTCATTTGCAGCAACATGGACAGACCT |
| | | TTAATTGATTCATACTTTTAGGGAAAAATAGTTTAG | | AGAGATTGTCACACTGAGTGAAGTAAGTCAGACAGA |
| | | ATAATCACTACTAGGCTGCATTTTAAAAAAATGTTT | | AAAAGACAAATATCATATGATATCACTTATATGTGG |
| | | GTGCATATACCCTAATGTTGATTATAGCACTTTT | | CGTCTTAATGAACTTACCTCCAAAACAGAAGTAC |
| 345331 | IBISS4snp1079 | CTCTTGTTCCTGAGAACTCCAGTGCCTCCTCTGAGC | G/A | GAACAGCAGAAGTTGTTCAGACTGTACAGCCTTTGT |
| | | CTAGCTTGTGAGAT | | ACACAAGCATGGAC |
| 348114 | SCAFFOLD | GATGGGATGAATCACCCAGTTTCTGTGCTTTCCTCC | T/G | TCCTGATGAATTATATGCATTCCAGTTATGCTATGA |
| | 125205_4038 | AGATATGCACAGCAGTATCAGTATAGGTTCAGGAGA | | TACTAAAAATTTGGAGTTAAGACTCCTAAGATGGAA |
| | | GTGAGTGGGTTTCCTAGGTTTTTAGGTATGGATAAA | | AAGTCCAATGCCTGGAAATGTCCAAGGAGTCACTGA |
| | | CGGAAGGAGAAAAGTAGGAGGAAATGGAAGGTGCTG | | AATGAAGTGGAGATAGTTATTGGAATTTAGACACAC |
| | | ACAAGACAGGGGTTTATAAGTAGGAGCCTCACTTAA | | CAAGGGGCCAAGAGCCTGGTATAGGATGTAGAAAGT |
| | | GAGAGAAGAAAACAAAAAAAGTTTCTTGAAATATTT | | GAAAGTGTTAGTCACTTAGCTATGTGACCCCATGGA |
| | | AGCAGCAATGAAATACAGAAATGAGCTGCATAGT | | CTGTAACCCACCAGGCTCCTCTGTCCATGGAATTC |
| 348008 | IBISS4snp941 | TATAATCCTCGAATCCAGGAGATCCAAGTTGTCAAA | T/C | GACGATAGTTTGCTGTACTTGCGAGATGCCCTTCCT |
| | | TTAGAGAAACGGCT | | GAATACAGCACTTT |
| 349469 | SCAFFOLD | ATATGTTTAAGATATGACTAAATGACTGACTCCCTC | C/T | TAACCATGGCTAGTTCAGACACCTAAGAACAGAAGA |
| | 290227_13905 | AGGGACCTGAGATACAATATCAGTTAACACCCAAAA | | CAAATTGGCACAATTAGCTTATCAGGTACTGTGCAT |
| | | CATCACAAAAAAGGAACTGGCACTCCACTGGAATAG | | CTATGCCAAGCCAATGGATGCTTGAAGAAAAGTACA |
| | | ACAAGCCATAAAAAATAGCAAAAAGTACTTGTTTCC | | TAATACTTCCCCTACATGAATTACAGCCTACAAAAT |
| | | AAAATGTCTCAGTGATTGCCTCAATGCTTTTTTGCC | | GAGCATCATTAGCTGAAGGGGTTTATTGAACCTGTA |
| | | ACAGAGAAAAAGCATGGTCCAGTAACAATGCTGCTT | | TAATTTAGCTCATAACTCAAAGGGCAAGGAATATTA |
| | | CTTTACCTGTCTTCTTACACCTTATTCAACAACC | | TTAAGAATTACAATCTTATGCTATTATTTAATT |
| 351890 | SCAFFOLD | ATCTTGATTAAAAAGAGATCAATAGCACGTGGGAGG | C/T | TCTCAATAATAAGCCCTAGCCTGCATCAGCACTAAC |
| | 70749_425 | GAGGAAGGGAAACATATTTCTCCTTAAGACTCTCCT | | TAGCACTGCACAGTGACCCATACATATAAGACCCAA |
| | | TCCCTCTCTCGAGTTATTGAATTTTCATCTTGTGTG | | TTGGGTGGGCCATGACGTCTGAGAAGAGCATTACAG |
| | | CATTTCCCCATTGCCAAACTGTACAAAGCACGAAGC | | CCACATCTCTTTCATCAAGAGTCAGATGACAACTTC |
| | | AGGTGAGTAAAAGCAACCAACTCTCGGAGTTTTTTC | | ATTCATGTCTCAAAGAAATTCCAAAAACATTAAAAA |
| | | CATGGAGGCTGCCTCTGAAACATGCCATCCATAGTT | | ATCCAACACCCAAGAGCTAATTCCCCAAGAAGTAAA |
| | | GAGTAAAAGGTTATTAAAATAATTCCATCAGCAC | | GCTAGGTAAACTTTAGAGAGTATCATAAAAATCA |
| 353496 | SCAFFOLD | CAAAGACCCAGTTCAGAAAGAGGTTATCAGGTACCA | T/A | AAGTATTTTTGAAATTGCAGACAAGGGTCTTTTATT |
| | 155611_5257 | ACACCATAGTATGTTCTCCTGTGTTCTTTTTTAATT | | TTTAAAGCCTGTGTTTAAACTGGTCCACACAGTGAC |
| | | AGGGTGTGGAGGTGAAAGCCTTTAAGACATTTTTTA | | TTCATCTTACACAGTCATGTTTAGATATTTATATTT |
| | | AGAGGACGATGGAAAAAGATGATTCTAAGAAGTTAT | | GTTTGGCCTTTTCCTTAGAGTAATTTATTCAATGTA |
| | | GCTGTACATTGTAAAGGTAAAACTCTTTTTAGTTTT | | TTTAGTATTTGTTAAGTGCAAAACACTCAACCTGGC |
| | | CTGCTTGCTCTTTGTCTTGTTCTTTGTATTTTTGAG | | CTGTAGAAAATTTAAAGATATCAGACAAGGGCCCTG |
| | | AGATTGCTTGTTTAAAGATTAAAATTGCTTTTTT | | TTTCCAAGGAGTTTAAGCTGGTATAATTACCATT |
| 348966 | SCAFFOLD | GGGCTGAACTTTTTATTAGAATTTTCAGTAAGAAGA | T/C | CAAGCCCATTAGTAGTCTGAATCTAGTAAGTCCCTT |
| | 120244_2770 | TTACTGGTTACTCACTGAGAGAGCTACTGCAGTACA | | CCTTATGAACCTTCAAGTTAGAAACTTCCAAAAATA |
| | | GCATGATGAGAGGAGCCAGACTGCAGAGGACCGAGG | | CGAATGTGTGTTCCATCAACATCAGGTGTAACTGAA |
| | | AAGGAATGGATGGTAAAGGAGGAAAGGGCGTATTGG | | ATTTCAGCTTGTGCTCCATCTCCTGGGCTTCCCTCA |
| | | AAATACATGCTGATTGAAGATGCAGAGACAAGATCA | | CAGCTCAGTTGGTAAAGAATTTGCCTGCAGTGCAGG |
| | | GAGTAGACAGAACAAGTTGGTGGCTAGATGAAAGCC | | AGACCCCAGTTCAATTCCTTGGTCGGGAAGATCCCC |
| | | CAATCCAGGAAGAAGTGCTTAAATTGGGAAAATC | | TGGAGAAGAGATAGGCTACCCACTCCAGTATCCT |
| 350113 | SCAFFOLD | AATTATAATTGGTTAAATCTCGAATGCATTTTTGGG | C/T | GCAAAACTGTGGACAAAAAAAAAATCGGATATTTCA |
| | 221823_1577 | CTTCCCTGGTGGCTCAGAGGTTAAAGTGTCTGCCTG | | TTGCTTCTTAATATTTTTCAGTAAAAAAAAAAAAGC |
| | | CAATGCGGGAGACCCAGGTTCTATCCCTGAGTTGGG | | ATAAACAATAAGACAAATGATTCACCACTGGTACTT |
| | | AAGATCCCCTGGAGAAGGAAACGGCAACCCACTCCA | | ATTGCAAAAACTAGACCGTGAGTGAGCCAGAAGACT |
| | | GTATTCGGGATGCATTTTAGTTCTCCATCATTTGGC | | AGCTGTATGATGTTTGGTGACACTTTCTTCACAGTT |
| | | AAAAACTAATACTGAATCATTCTAGAAATGGTGAAA | | TTCCATGTTCTCAGTGTTCCTACTACTAGAGCTAAT |
| | | TAATGTACAGTCAAATAAACAATGACACGGAGAA | | GAACAGTTGGTCCTACTATTACACATGTTCCAAA |
| 346777 | SCAFFOLD | AGAAGCTGTCTGAAGAGTGAACTTGGGAAGGAGACA | C/T | GACAAATGATGCACCCATATGTCATCCGCCTCAGAC |
| | 296111_2645 | GTGGAGGACAGCCGACCTCTAAGGTCGCGTGATCCC | | AGTGCTTGGATTCACACCCAAGCATTGGTGATTTCA |
| | | CCCAACACCTCGATGTGCTGAACCTGGACAGATGTC | | CTGACAGTTGAGTAGAGTAACAGACAAAGCTCCCAC |
| | | CCTGTAGGACAGACGTACCTCCTGTCTTCCCACAGT | | TAGTTGAGCCCTGCCTGTGTGCTGGATGCTGTGCTC |
| | | GCAGGAGGCTGCCACATCCCTCTTCAGTGGCGGTGG | | TTTCATGTGCAGTAGCTCAATCAAACATCATGATGA |
| | | TCCTCAGTCCAGTAGCCTTCCGGCATCTTGTGTTGC | | TGAGGCTCAGAAAAGTTATAGCAACCATATGACCCA |
| | | TCTGTGCCTTGTTGGACTCTAGAGAGACGATCCA | | CAGTTGTGCTTTTTGGTATTTACCCAGATGAATT |
| 354015 | SCAFFOLD | TCCATGGTAGGTGAGCAGAGCCTCTACCAGTGGTTC | C/T | TGAGGTTATAACTACCAATATTCATCACATTTGCAA |
| | 20780_6337 | AAAATCTGGCAAAGTAAGAAGTGGGACACAGATTCG | | TTAAAACAAAGACACCTAAAATATTTATTAACTAGC |
| | | TGACAGGGACCTTACTCGTTCAGGAGACATCCTATG | | TAAAAGTAACAAAAAAAAATCCATTTGATGTTAACA |
| | | GAATAAATAAAATGAGCAGTAAGTACCAAAATCTTA | | TGAATAACATTTTTTGTTAAAAAATAACTATATTTT |
| | | GAGCCACACATAGTTAAAGGCATATAAAATCTTCAT | | ACAAAACAAAAAAATATTTAGTGAGAAGAGTGGCAC |
| | | GCTGAAAGAATTTAATTATTTCTTTGAGTTATTTTT | | TATTTTATATGTTTACAAATCTCTTTCCTATATGGT |
| | | TGATTTTAATTTCTTTGAATTATTTCTTTCATTT | | TTGAAAGAAGACAGTGAATTCTTATATTTAGTTG |
| 346294 | SCAFFOLD | CTGGAGCCAAGCTCTTGCTTCAGAGAAGAACAAGCC | T/G | CATTTTGCTGACAGTGGAATTAAAATCCAGACCAGT |
| | 130087_22009 | AGAAAAGCCCCTTAAAAACATTGGAAATTAAATAAA | | TAAAAGAGCCTCTTCATCTAGATAGTCTAGGGAGCT |
| | | ATGTAAAACCTTTCTAGAGAACTGCTCTTGAGGTTC | | CAAGTACAGAACCAGAATTTCTGCCTATTTACTTTT |
| | | TGTAAACTTTCACAGCTCAAAACTACCTGAAAGCCA | | GAGATGTTTACTTTAACCACTTGGGCCATTAGACTT |
| | | GAATCTACTATGAAGACTCTGAAGACAGTTAACTTT | | CACCTGTGGTCAGCTGAGATTCTACTTGATTTTTTG |
| | | TAAGGGAGTATTTTACAAGTCTACGGCGTCTGCCTT | | CTGAACAAACTTGACTATCCAAGCAGGTAGTAAATA |
| | | GAGACCTTAATGTACCAATGCTGTACGAACATAC | | ACAGACTTGGCCTCTCTTTTCTGGAATAAGCAAG |
| 344344 | SCAFFOLD | ACATCACATATACTAATTATCTTAGCTGTCGTTACT | C/G | AGACAACCAGTGTTACCAGACAACAGACAAGAAGCC |
| | 46716_2213 | ATGACAAATGCTCAGTCAGGGACAGTGCCAGTGACT | | CATTCACCCGAGAGTCCACGCGGACATCTGAATCAC |
| | | TCGTGTACACCCTTTCATTTACTCGTAACAATCCGG | | CTACAACGCCTGACTTAGGAGTTCCGAATTCTGAGC |
| | | GGAAGGGGAGGGGCACTGGCCCTTCTCAGAGACACT | | CAAACACCAGATTTTGCACACCGTATTGCATGAGCC |
| | | GACCTGCCTGGCAACACACAGCTGCCAACTGCAGAG | | GCAGAAATGTGTGATTTTAAGGAGCATGCTGGGACT |
| | | CAGAAACACCAACCCAGATCATCCTGACCCCAAAGA | | CTCGTCGAAGGCTGTCTATAGTCGGATTGATCAGAT |
| | | ACACAGGCGTCTCCGTGCAAACACGGGAGCCACA | | TCAGATGTGAGGTTTGAGGTTGGCCTGCCCTTGG |
| 345199 | SCAFFOLD | GAGCACACACTTGGTTCTCATGCTGCCCTGCAGTGT | T/C | AAGCTCCTTCTCTGAGAGTTAAGTGTCCTTCTCGCA |
| | 74865_2495 | GTGTCTTAGCCTTGTGCTGCAGACGAGGAAACACAA | | TACTCTGATCTCATTCTGCTTTGTGCTTTTTAACAT |
| | | GCTCCGATAAATTAAGCAACATGTCCAGGGGTATGA | | TTGCAAATTCTATTTTGCATGAGCATGGGGATGGGG |
| | | GAGCTATCAAGGTGGGTTTCAGCTGCTATGCCCTAT | | GAGGTTCACATTCCTCCTAGCCCTAGAATCTACCCC |
| | | GCCTTTGTTCTTTAAGACGCTTTGGGGAAATAAGGT | | ATGAGAGATGATCAAGAAAAGTCATTTTCTGTTATT |
| | | AAATTTACATGTGGCTGATGTTTATGTATCTCATTG | | CAGTCTGTCACCTATGCCTGTCATAGGGTAATTCCT |
| | | AAGGTCACTCTGGGGCATCTTCCTCATCAATCCC | | GAAATTCAATAGTTCACTTGCTTTCCTGTAAAGG |
| 344104 | SCAFFOLD | TAGTCATTACTTTTCCATTATTACAATAACTCTGAA | C/T | CTGGTGCCAAGGAACTGAACTCCTCACAAACATACT |
| | 230631_15643 | ACAAGTTATAATTGTTTACCCAATCTGGTTGAAGTA | | CAGTGACTCAAATGAGATAAAGGGAGATTATGACAA |
| | | AAAGTAACCATAAAGAAAACAAGGCAAAAACACTAC | | AGATTAAAGATGAACTCAGAGTACTCATATAGTTCT |
| | | AGATCAACACAGGTAACTGTAAAATATTTCTGTTAA | | GAACCCTCAAGTGGCTCACAGTTGGGAGAATAGTGG |
| | | TGGGCATAACCTAATACTGCTCTGGTAAAGCCATTT | | ATAAAATGGATCCTACAAAGGTATATGTTCAGGGTA |
| | | TCTCTTAATAGGATGGATTTTCTCTTTTGGACTTTT | | TATGAACTCTGTACATACAAGTTAATGAAACTTTTA |
| | | GTTGCCATTTTCCTTCCTGCTGTAGGAAAGACT | | TGATATCAACTACTCAATCCTTCATGGCATCTTC |
| 348749 | SCAFFOLD | TAGTTTATAATCCACAAAATCTCCTATTGCTCTTGA | C/A | TTCTAGCATTTCTTATACAGCTTTATGATTTTTTAA |
| | 55701_1292 | GTATTTTAATGAATGAAATAACTACTCTGTGTATCA | | ACTTTCTTTAGACATAAACAATGGAATTCTCAAATA |
| | | AGATTAAGTTATTATGGTTTATAATAATACCTCTTT | | CAGGGACAGTGTCTTACTCCTATTTATTTCTATAAA |
| | | TCTACATCACTTAATAATTTTGTTTCTTTCATTGTA | | GTATAGACCAAGGTCCCATCAGTAGTTGGTACTTGA |
| | | ACTCCACTGAATCCTCTGTGAAATGTACTGTGGGTG | | TACATGTTTGGTGGGTGAATGGAGAGATGAAATTTT |
| | | TTCGTAAAATTTAGTACATGGCAAAGTCTGGGCTTG | | ATCTGATGGTAATATAAATTTTCATCTCTGGAGTCA |
| | | AAATTGGCTTTCCTCGTATCCATTAGCAACTCT | | CTAAAGTTTACATACCATTTTGATTTCCAGGGTA |
| 353947 | IBISS4snp181 | CAGGTTGCATGTGGACACTCAGCCACATTAACAACT | A/T | AAAAAATGGCAATGTTATAATGAATTCTCAGGTGAA |
| | | TGGAGAAAAAAAAT | | CTTTTTTTTCAGTT |
| 349492 | SCAFFOLD | CTCCAATAATGTTTATTCTTCAAGTTCCAGAAGGGC | T/C | GGTAACATCTCAGGTTTTTGAAGTGTGGGTTGTCCC |
| | 295327_16712 | CTTTTTTTTAAAAAAGCCATTTCACTTTATTAGAAA | | ACACTATCCTAAAGTACAAAAGTCATGAGGACAAAA |
| | | TCAACGGTGCCCTGTGGGCCACTAGGCTGGTCTCTA | | ATTCCCTTCTGCTGTTGCAGCTGTAGGGTCACCTAG |
| | | AATGGAACTTCAGGGCGTCTTTACGTTTAGCAATTT | | TGCCTTATTTAATCAGCTGATAAACCAGCATATTCC |
| | | ATCAAGGACTATTGAAGACTAGTGCTAGAGATGTCG | | TCTCCAGCCATAAAGCAAGATAAGAGCTCAGAAGTC |
| | | CACTGAGCAAACAAAGCCTCATAGAAACAAAGCTAC | | ATCATTCCCCTCCCTTGCTTCTCTCTCTACACATGT |
| | | ATAGTTATCTTCTGGGAGTTTTTGTGTGTTCTCA | | ACCTGTATCTATTATATACTTAACTCAGAGTCTG |
| 349894 | SCAFFOLD | TTTTTTCTTTCATTCAGTGATATGCTGGAGTTATTG | G/A | CCAATTATGGTTGCCTACATCACCTAAGAAACAGAA |
| | 140160_3611 | TAATTCTTCTCATTTTTTTTATCTAATCTCTTAGAA | | AGTAGCTGTACAGTATCCATTTGACACTAACAACAG |
| | | AATATGGTCACCACTGTTTACAGTACTAAAAATTAA | | TGTTACTTTTAGCCCCCTTTTGGGGGCTACCTTAGT |
| | | ATGTTATTTCAGTACAGAACACAGCAGTTAATCCAG | | GTTTTGTACTTCTCAGAGCCTTCTGTGTTCATCACC |
| | | AAAGTCAAAGGCAGGCTATTGATCACAATTACAACC | | AAAAATTAGGACCTCTTGTCTCCTGTGGATACTAAC |
| | | CACTTCAGTTGTGCACTTAAGTCATGTTACTTGGAG | | TTTGAGTACTAAAATTGGAGCAGCCCTAGGCAAGTT |
| | | TGATGCAACAATGGATGATCCATTATTGTGAACT | | GGGACTTGTGGACCCTGTGCCTCCTCCCAAGCCA |
| 345270 | SCAFFOLD | AAGAGGCTGTAAGCCAATGTCCTAAAAAAACTAAAT | C/G | CTGGGACAGTGGCATAGACATTTTGAGTTCATTGCT |
| | 97036_665 | GGTGGATGCATTTCTGGAGTAGCCAAGAAAAAGCCC | | TGGATTACTGTTTGTCTACCACTGAATCAGGTTTCA |
| | | ATTTAGCCATAACAGCTTTTATTAACAAGAAAAACA | | TAGAATAGTTAAAATAAAAAGATTCTGGCTATTAAG |
| | | GAGTAAGAAGAAATAATATTAAAGATACAAAGCATG | | AAATTCCCTACCCATGAAAAGCTAACTGTATAGCTT |
| | | ATATGGACTGTCTTATTCGAATGATTAAAAAATATT | | CAAGAACACTGGGATCTTAGAGCCTTCATTAGACTG |
| | | ATACTTACCTAATCCATTACTCTCTCTCTATTACTA | | TGAGATATAGGTTTATCTATCAAATTTTTATTTAAT |
| | | ACACTAACAGGCCTCCCCACTCCTCCTTTTCTTT | | AATATTTAGTATATATTTAATAATATTTAACAGT |
| 347032 | SCAFFOLD | AATATGCCTCATGAGGAAGGGCAGCCAAGGGAGGGC | A/G | TGGGAGGTGGTTCTCCTTGATGGAATTGTTGGCTTA |
| | 92148_4975 | ATTTTTCAGGCCCCTTCCCGGTTCAGAGCATCCCAA | | TGAATTTGAATCAATTATTTCAACTTTTGTCTTCAG |
| | | AGAGGTGCCAATCGTCATGATGGCTTCTGTACCCAT | | GGTCCTGGTAAAGAGGGACTTTTTAAAGACCCCAGT |
| | | GGCCATCTGCTTACTGGGCTTTGGGCAGATGCCAGC | | GTACATGGATGTTTGATGCCAGTTTATATGAGAATC |
| | | GTATTTTTCTCTTCATGACGATGCAGTCACCATTAG | | TTAACTTTGAACAGCAGGCCAATCAGGAGAGAGATT |
| | | GTAGCAGTGCTGTCTTCTCTCCATTACTGACCTGGC | | TTAGTCTACATGTAGCCAGAACCTCCACGAGGCCAT |
| | | AAGATTGGACCTTAGATCTCTTGCTGTAAACAAT | | ATTCTTCCCTTGCTGCTGGACTCTATGTGTGTAA |
| 350408 | SCAFFOLD | AGAATAGCCCTCGATCACCAAAACCAGAGAAAAGCC | G/A | GTAACAAGTATATAGGCAGCTCCTTGGTAGAGCAGA |
| | 36638_10144 | TGTCAGCAACGAAGACCCAGCATAGCCAAAAATAAA | | ACTGGAGTCAGTCATGCCTTTAGGACCATCTAAGTG |
| | | TAAGGGAAAAAAAGCAGCAGCTGCCCCAGGACTCTA | | TGGGAAACAGCCATGGCCAATGCAAAACCAGGTAAA |
| | | GAATCAGAACCTGAAACCAAACCGTGCCCTTAACAT | | AAGTGCCAGCAGATTGGCAGGAAGTTAATCTGAGGA |
| | | CAGTCCACATACAAGTGCTGCCTTTGTTTTCCTTAT | | TTCTGGATTTTGAACTTGAAGGGCTAAAGAATTCAT |
| | | CTCTTCTCCTTTATATAGCTCCATTAGAAATTAGAC | | CTATGACATCCTCTGCTCAAAACAGTCATTTCAGAA |
| | | TTATATAGATGACAGTAATTAGGGAATAGTGTCT | | GTTCTCCTGCCCATTGATTTCTGATGTTGGAGAC |
| 344006 | SCAFFOLD | AGTCCATGGGGTCGCAAAGAGATGGACATGACTGAG | T/A | TTTTGGACGCTTTAAAGCCACAGTTTCAATTATAGT |
| | 181577_4518 | CGACTGAACTGAACTGAATCATTCCCAAAGAAAGCA | | ACTTATGATTTGGCTGTTTATATTTTCCATTTCTAT |
| | | TCATAAACTTATCTATATCAAGTTGTCACTTAGGTG | | CTTTGTGTAGTCTTGGATGGTTGTACCTTTCTAAGA |
| | | TGGTATCAGAAACACAGCAGATCATGAGGCTATTCA | | ATTTGTCCATTTCTTCTAGGTTGTCCATTTTATTGG |
| | | GGGAGCAAGAGTAATGCAACTGGTGATGTGCTTGGT | | CTTATAGTTGCTTTCAGGAGTCTCTTAATTCTGTCC |
| | | TAAGTGGTGATATGCGGGGTTAAGTCATGGTAGCAT | | ATTTCTGTGGTGTCAGTTGTAACTTCTACTTTATCA |
| | | TTGCCTATAAAGCCATCTGGTCCTGGACTTCTGT | | TTTCTAATTTCATAGATTTGAGTCCTATTGGCTT |
| 349539 | SCAFFOLD | AGGCCAAAGAGGTGTCCACTGAACCCTGCACAATGT | C/T | CTGATGTTAGTGCAACAAAGGTTCTGTTAATTTTTA |
| | 311123_515 | CTGCCCCATGGCAACTGTCCACTAGGTATTCGTTGA | | AGTGAAATGAAAAAGTGATTCCCACTATGTTTTCTC |
| | | GTGATGAATGAACAAAGAAAACAATGCCAGAAGGAA | | TTTTTCATACTTTTGTGGGGTAGGATTGTCATTTAG |
| | | GAAGAATGGGAATAAACAATATCAAGGTCCTTAGAA | | AGTGAAGAAAATAAAAGAGGTTAAGGTTTTCATAGG |
| | | GATATAGGAATACATTAAATGACCCAATGGGGAGAA | | CACTATTAAATATATGAAAAAAAGGTAGAATAAGAA |
| | | TCAGGAAAATCCTGTATTGACCCTAAGGAGATTCCC | | TAGTTTTTTTTCTTTTTCTTCCTTTAAGTTGTTCTG |
| | | TAGAAAGGGGTCACACTAACTTACATGTTGGAGT | | ATATATAAGTGGAGGATAAGTAGGACAAGTCTTC |
| 344585 | SCAFFOLD | TCACTGCTGCATGGTGAATTTTACAAATAAGAGTTT | C/T | CCCTACTCCTGGTACTGGCCAAGAACACCTTCACCT |
| | 120193_812 | ACTCAACCTATTCTACAGTTGATGGATATTTGGGTG | | GTGACTTTGCCTATAGGATATACCTAGGAATGGAAC |
| | | TTTTGACTCTTATGAACACTGATGCCAAGACCAGAG | | CGATAGATCACAGGATCCATGATGAGCAATTTGCAG |
| | | ATGACAGTAGGTGAGGCAAGAAAGGTGTTTGGGGTG | | CAACATGGATGGACCTAGAAAGTGTCACACTGAGTG |
| | | CAAAATTTAAGGAAGCACTCACTGATCAAGATCACT | | AAGTCAGTCAGACAGTGAAGGAGAAATATTGAATGA |
| | | CTGGCACTTTTATGACCCTGGGAGTGAGCACCTCCT | | CATCCCCTATATGTGGACTCTAAAAAGAAATGACAC |
| | | CAAATTTTGAGCACTAGGCATCTTCCCTTACTTC | | AAATGAACATACTTACAAAACAGAAAGAGACTTA |
| 348608 | SCAFFOLD | CCCAAGCAGGCTAAATTTCCCGTGCCAGAGATAGTT | A/G | AGAAACACACAGGGAAGTTACTACTAAGCCCTCAGG |
| | 296838_517 | TTGGGGTTGGTGTCCTGTTCCCCAGTTTCCTCCTGG | | TGAGAAGCTCACCACCAGACAGGCAGCAAAGGGGCT |
| | | CTCCCAACCTCCCTGCTGCTTTCTAACCTACTCTCT | | GAACTGACCCCTATTTGCATTAAGTCACTCAGTGTT |
| | | CTGCTTTGGTGTCTGTGCTTCTCTTTGTAGTTCCTT | | CCTCACTCTTCTTTCCCAGCTTGGCTGGTATACTCC |
| | | GCCTGGATCTCTGTCTTTAGATCTCTCTCTTCACCT | | TAGGCATGTGTATGTCTGTGAATGTGTGTGTATGTG |
| | | ACACTCCTCTGCTGGTCTCTATTTCTCTACATTTGT | | TAAGTGTGTGCATGAACACAGATGTGCTCTTCTGTC |
| | | GCCTTTTTCTGTGGGCCTTGTTTTAACACTCAAT | | TGAGGCAAGACTAAGACTAGAGGTCTAAATAAAG |
| 352602 | SCAFFOLD | GTGAAACAGTTGTTAAATCTTTGCCCATTTTAAAGT | C/T | CCTAAAAGATTTGTAGCTTAGGTTTTACTTTTATAT |
| | 150884_15150 | TGGGTTATTTTCTTACTGTCGTGTTTTAAATGTCTT | | TTAAAAGGGCTTCCCTGGTGGCTCAGAGGGTAAAGT |
| | | TTCTATGATCTGGGTACAAGTCCTTGTCCGGTGCTG | | GTCTGCCTGCAATGCAGGAGACTAGGGTTCGATCCC |
| | | TGCTTCGCAGATGTTCTCTCACAGTCTGAGTTTTTA | | TAAGTTGGGAAAATCCCCTGTAGCAGGAAATGGCAA |
| | | TTAAGCTCAGTTTATTGTTTTTAACTTTTATGGATT | | CCCACTCCAGTACTCTTGCCTGGAGAATCCCATAGA |
| | | GTGCTTTTGGTGTTATATCTAGAATGATTTCCGTAA | | CGGAGGAGCCTGGTAGGGAACAGTCCACGGGGTCGC |
| | | TCAAGTTCATGAAGATTTTCTCATGTTTTTT | | ACAGAGTCGGTTTATATTTAAGATCCATTTTGA |
| 349734 | SCAFFOLD | CTTCCCAGTATTTTAATATCAAGCCTGCTGAGGTCA | C/G | TCCTTCATATTGTACTGACCCTTCATGGCTCCTTCA |
| | 94906_761 | CACACAGTCTAATCCAGGCTTGGGTGCAGTTCTCTA | | TGGGTCCCTGTGGCCTGATTCAAGGCAATTAAAAAG |
| | | TGTGGGCAGAATGCATGTGTGACAGGTTTCAGTGGG | | AAAAATATATCTGCTTCTCATACCTCCCACGTATTT |
| | | AGAGATCTAATTCTTGGCACTTTTAGTTATGTTGTT | | TTGCTTGAATGGGACTTCCATGAATGATGGTCACCA |
| | | CCTGGGACTTTAATGTGTCTTGAGTTTTGATAGATC | | GCTTGGACCATCTATTTTCTTTCTCTTTTTGATTGT |
| | | ATTTGCAAAATTCGTGGTAGCTATTTCTCTGGCAAA | | GCTGAGTCTTCGTTGCAGTACATGGGCTCTAGAGTA |
| | | TATAAGCCCAATGGATGCCTAAATAAACTTTGCT | | AGTGGGCTCAGTAGCTGCAGTATGTCTGTGGGCT |
| 351979 | SCAFFOLD | AGATGATTTGTCAAAAAAATTTTCATGTGATTGAGA | C/A | AACTTCTGGGGGGGTAGTAGTATATAGTATAGTTAA |
| | 110152_17110 | GTTTTTTATTGGGAATTGAGGGTGGAAGGAGATAAT | | GATAAAAAAAGACAACTGGTATTTTTTTGAGAATTG |
| | | CGATTATACCTCCCGATGGAGGAAAAATCTGTGTGT | | TTTGGTATATTACTAGAGTATGTTTTATAAGTGTAA |
| | | GCTGTTCACGCAGCTAAGGCTCCTCTGGCTGCTCAG | | TAGTATTAGGTTTATATATGCAGTTGAATCATGCAT |
| | | GGAAAATGTGACAAAATCAAAATGTCTGGTTGTGAA | | ATACTTGCAGCCCCTATGAAATTTGATCTGTCTTTA |
| | | ATTATCTTCCAGTAAGTTTGTTGCCTTTATTGTGCC | | TTATAAACATTATATAAATAAACAAAGTTTAAAAGA |
| | | TATAAAAAATACCATTCATATTTATGTCATTAAT | | AATTAAATAATACAATAACATCTAGCAAGTGGAG |
| 348875 | IBISS4snp509 | TTTATAGCTTTCGTTTTGATAACATTTGGTCAGTTC | G/A | AAAGCGGAAGTGTATGTGAGCTTTTTGTAAAGGATT |
| | | ATTTTAATAGTTGG | | TAAAAGATTACCTA |
| 344551 | SCAFFOLD | TTTCCCAGCATCAGGGTCTTTTCAAATGAGTCAGCT | A/G | CAGCAAAGTCAAGGTCCTAATGACACCACAGCTTCC |
| | 103644_2311 | CTTCACATCAGGTCTAGTCAAAGATGATCAGAATGA | | AGACCAAGTGTCACCTGAAGGCCACTCTACCTTTGG |
| | | ATCTTGGGCTGATCCGGGAATGAAGCTGATCCAGGA | | CCTTGGAGAGTTACATGACTGGTACATCCCCTCTGT |
| | | ATGAAGAAACAAGGGCACAGGTGCCCTCTCTGAACA | | TGTCTGAGCCAGTCCGAGCTGATTTCACTGTTATTT |
| | | GGGCCAGGAGCGGACAGGGTACCTGGAGCCACTGCA | | TCCAAGATTCCATTCTGTTACATATTCTCTCCCTGA |
| | | ACCATTTGCTCAGGCCAGGCCAAAGGGCATGGAAAC | | TTTGTATTTACAGTATGCCTAAGGCAAGGAGATGAA |
| | | ACCAGGGTAGATGTGCGGCCCAGGATAGTCATAT | | ATGAATGTTTAAAATGGTCCTAACACTTTTATCT |
| 349124 | SCAFFOLD | AACTTGTTCAATCAGCATGTCATATGTCAATGTAAG | A/G | ATTGAGGCAGCTAGTTTCATCTCATCATTTTGTATC |
| | 175661_7851 | ATACACGCTTACACGTAGTTGGCTTTTTGTACCAGT | | CTTATTTGGGTTGCCAAGGCACAGCTTACACACTTA |
| | | TTCCTCACTGTGAATATTGAGAGTCTAGAAAAGTCT | | GTGTGCTTCCACAATTCTTTGCCATTCTAGAGCATG |
| | | ATTCTACAATATCTTTGATAGTATTTATAAATTCTT | | TACTTCTGACATTTTTATTAGGCTTTTGTGTTTGGT |
| | | TATGACTATTTTACACATTTGTGTGTGTGATTTTAT | | CACTGTATTAAATCCAGCAATTTATTCCTTTCCACT |
| | | TTAATTTGCAAATAAGGCAATAGTGCTACAGAATTA | | TTAATTGTCTTCCCTGTACAAGGGGTGGAGAGGAAT |
| | | CTACAAAATTGAAATTGCTAAAATAATAGGAAGC | | TCACAGTGCCTGCACATAAAAGGCCATTATCTTA |
| 350131 | SCAFFOLD | AAACACTACAGATCAACACAGGTAACTGTAAAATAT | A/T | TATAGTTCTGAACCCTCAAGTGGCTCACAGTTGGGA |
| | 230631_15544 | TTCTGTTAATGGGCATAACCTAATACTGCTCTGGTA | | GAATAGTGGATAAAATGGATCCTACAAAGGTATATG |
| | | AAGCCATTTTCTCTTAATAGGATGGATTTTCTCTTT | | TTCAGGGTATATGAACTCTGTACATACAAGTTAATG |
| | | TGGACTTTTGTTGCCATTTTCCTTCCTGCTGTAGGA | | AAACTTTTATGATATCAACTACTCAATCCTTCATGG |
| | | AAGACTTCCTGGTGCCAAGGAACTGAACTCCTCACA | | CATCTTCCATATAATATTCTGCCAAAGAATGTCTCT |
| | | AACATACTCAGTGACTCAAATGAGATAAAGGGAGAT | | TTTTATATTCCATAACTATTTTGCCAGATCTCAGGA |
| | | TATGACAAAGATTAAAGATGAACTCAGAGTACTC | | AGAAGAAGGAGGGAGATAGGGAAAAAATATTCTG |
| 350504 | SCAFFOLD | GGGCGATCAGGAAGGACACTGTGAGGGCGGCCCTGG | A/G | GGAATGGTGTATTTTTGGATTTCAGAAAGAATGGGG |
| | 71111_2800 | ATGGATGTGTCTTTGTCCAGGTTTCTGAGGGGTGGG | | TCTCTGGCAAGGAGTCACTGAGATCATGCCTTGCGA |
| | | TAAAGTGAAAACAAGCCCCCTTTGGGTCTCTGCGAC | | ACAGGAAGGATGTATCGGGTCACTTACATTGAGCCA |
| | | CTGAGGGCCCCTCTGTTACTCTCACTTTCCCTCTTT | | CTGGTGAACGTTTAACGTTAGCTGGGTCTTGGGGGG |
| | | CAGCGTTTTCAAATCTCAGATGTTTTGTTCAGACGA | | AAAAGCCCCAATTTGTATGTAGCGTTTCCCTATTCC |
| | | CGTGCCTCTGGCTCTCCTCATCAGGAAGTTGGAAAT | | TGTGGTGTAAATACTCCCACCAGGGCACACTGCAGG |
| | | TTCAGGAAGGAAATCTGGATCACTGCATATCACA | | GTACCAAATAGCTGTCCTTATACACAGAGCTGGG |
| 351458 | SCAFFOLD | GATAAATTTGTCAACCCATATATACCTTGTGCATGT | T/C | AAACTAAAAATTGCTCTTTAACAGAGTGTCTAGTGA |
| | 160642_20511 | TGCTTCAGGAAATTCCCAAACAGTAAAGGAAATGGG | | TGATTCACAGAGTTTTGTAAAAAGTGTGACAATCTT |
| | | ACAAAGGCTAACATATTTCGGCTTAGGCCTTTTGGT | | TTTGAAGACATGTTGAGTGATAATTAAACACAATAT |
| | | TCTCCACCTTAAAAGAGCCACCTTAGACCACTTGAG | | GTGTTGTTAATGTAGCTAAGTTGATTTAATGGTAAT |
| | | CTCAGAGTTCTCCAAATCCTTATAAAGATCCCACTT | | GTGGATAGCAATGATTATCTTTGCACAAGTGTAGGT |
| | | CTGATTTCATTCTACTAAGTTAATTCTAAGACTATC | | CACAGCTACTNNNNNNNNNNNNNNNNNNNNNNNNNN |
| | | AAGGGGGTGTTTTCTCTTATTGCAATGAGGTCAAA | | NNNNNNNNNNNNNNNNNNNNNNNNNNNNNNNNNN |
| 351719 | SCAFFOLD | TGACAAAAAGTGCACTAGAGCCATAGCCATGGGGCT | C/G | TATATATGATGGGTCAGAAAAATATTTCTGATCCTT |
| | 295058_29918 | GTTCTTTATCTGCTACCAAGACTGGCTGATTCAGAA | | TACCTTGCCCCTCTTTTAGACTGGCCTATCACCATT |
| | | CAGTCCTCGCTATTTTCTGACCTTGGCCTCTTCTAT | | TCAGGCTTGGTTTATTTTTGCAGCCAACTAAGCAGT |
| | | GATCCAGTTCAAGGCTGGGCTTAATGACTTTGTTGT | | CTTCTCTGCTTCTGGTTCTTCTTCACCTTGAAATAC |
| | | TTATCTTTTTAATGTATGCGGAAGAAAATTGTCTGG | | GTCCATTTCACTGTTATCAAAATTATAATAGGAGTT |
| | | TATCAGTTTTCAAATGAAATAAATTCACCTTAGTCA | | GTAATAACAGACGATTATTTCTGTGCTCAGAAGAGT |
| | | ATACCTTCAACTTATGCTGTGAAAGTAACTCCAC | | ATTCTGTGCCCCACCATGCGTAAAACAAAGAATA |
| 351968 | SCAFFOLD | TACCTGCACATGTTACCAGATGTTTTATTTAACAGA | A/C | AGTTCTTAAGGGTATATGCATGCATGCTCAGTCATG |
| | 105346_12308 | TTTGGAACTAAAAATCAATTTGGTTGTCATTCATTG | | TTGGACACTTTGCGACCCCATGGACTGTAGCCCTCC |
| | | CCCTTTTGAGGATTTAGCCATAAAATGAATCAATGA | | AGGCTTTTCTGTCCATGGAATTTTCCAGGCAAGAAT |
| | | AAATAATAGAGAGATATACAAGAGTCTCTATTTAGC | | ACTGGAGTGGCTTGTCATTTCTTAAATCAGGGGATC |
| | | ATTCAATATAAAAGGGATTTTTTGTAACATAAATCT | | TTACTGACCCAGGGATCAAACCCACATCTCTTGCAT |
| | | ATAAATGTGACAGGATTTTTTATATAGATTAATAGT | | CTCCTGCATTGGCAGGCAGATTATTTACCACTGAGT |
| | | TAATAGGTAAGTACACATATTCATAAACTCCTAA | | CATCTGGGAAGCCCTACTTGGTATTAAAAATATC |
| 349807 | SCAFFOLD | TATAGGATTAAGAAAAAAATAGCTATTAAGTGTTAT | G/C | ACCAGGCAATAGTGGCAGTAAGGTTTATTTTTGAAT |
| | 110246_20081 | CTATCCAATCACACTTTTTCTGCAGCTGAACTACAT | | GGTCACAATCAACTAAGAAGAATGTGCACTTACACA |
| | | TCTTATGACTGTCAGTGGTGATTGTGATTTTGCTTC | | AAATAGTAAATACCAAAATAAGTGTCAGTGCTGAGT |
| | | CATCTTTGGCTGACGAAAATTTGGAAGTTACAAAGC | | ATCGACTATGTGCCATAGACTAAAGTTCTCTGGAGG |
| | | TACTGTTGTGTACTTCCTGAGTATGGCCCCAGGTAG | | TTCAGGAGAGGAAAGAATAGTTAAACAGTCTCTATA |
| | | GCACTGAAAGGCTTTTCAATTACCAACACATCTAGG | | ATATACTCAGATTTTTTAGTTTTTTTTTTTTTTTAA |
| | | TGTTTCTTAAACATACAATCTATACAAAAACTTC | | TATATAGTATGGGAGTAGATGATGTGAACCAAGA |
| 352770 | SCAFFOLD | AAACAGAAATAAAAGCAATTTTGCTTATAGAGTTGT | A/C | ATTTATTAGGCATCCATCATTTCTAAGTTTTGTAAA |
| | 251470_3902 | GGTTTCCATTGCTACTCATAAATTCGTATAGTATTA | | GAAACTTTAAAGACGCACCCTAGGCTCAAGACTCGT |
| | | GTAATAATAGCACCTCCTGAAAATTTAATAATACCT | | GCCATCTAAGAGTTTAGATGTGCAAACACATCCAGC |
| | | TTGCATTTGAACAGGGCTTTTTGAGATTTTTCAAAG | | TACAAATTAAGAGTATGAAAGCACTGTGCAAACAAT |
| | | GCTTTTAATACTCATTGTTAAGAGGTCACCAGACTA | | GCCTGCCTAAAACATCCCGGCAACCAGGCAACAGGC |
| | | TTGAAAAACATTGCTACAATAACATGAAAGACCAAT | | CAGGAGGTCATTCTAGGATTTTTCTGCCTTAATAAT |
| | | GCAGAGAAAGCCTGAATTCTATCTGTATATCTAG | | AATCTCTTCAAAGTGAGCAGTGCCTTAGAGGTTC |
| 345571 | SCAFFOLD | CAATGGACATGAGTTTGAATAGGTTCCAGGAGTTGG | G/A | TGATATGCGGGGTTAAGTCATGGTAGCATTTGCCTA |
| | 181577_4454 | TGATGGATAGGGAAGCCCAGCGTGCTGCAGTCCATG | | TAAAGCCATCTGGTCCTGGACTTCTGTTTTTTGGAC |
| | | GGGTCGCAAAGAGATGGACATGACTGAGCGACTGAA | | GCTTTAAAGCCACAGTTTCAATTATAGTACTTATGA |
| | | CTGAACTGAATCATTCCCAAAGAAAGCATCATAAAC | | TTTGGCTGTTTATATTTTCCATTTCTATCTTTGTGT |
| | | TTATCTATATCAAGTTGTCACTTAGGTGTGGTATCA | | AGTCTTGGATGGTTGTACCTTTCTAAGAATTTGTCC |
| | | GAAACACAGCAGATCATGAGGCTATTCAGGGAGCAA | | ATTTCTTCTAGGTTGTCCATTTTATTGGCTTATAGT |
| | | GAGTAATGCAACTGGTGATGTGCTTGGTTAAGTG | | TGCTTTCAGGAGTCTCTTAATTCTGTCCATTTCT |
| 347013 | SCAFFOLD | AATGTTTGCACATCCTCTTGATTTGATCCCCACAAT | G/A | GATAGGGCCTCTAAAGAGGTCATGAAGAGGTTATGT |
| | 85585_6225 | CAACCCCATGGTGACACCGAAGCGTTATCTCTCCCA | | GATGGGGCCCCAATGCAAAAGGGTTAGTGTCCTTCT |
| | | TTTTAGAAAGAAGGAAAGAATTCACATTTCACAGAG | | AAGAGGAGACACCAGAGGGAATACACAGGGAGATGG |
| | | CCTTTATTCCTAGACACATTCTGACATTTTTTTCCC | | CAGCCACCTACAAGACAGGAAGAAAGGCCTCACTGG |
| | | CCCTGAGCTTAAAATAGGTAATTAAATATTCCATAG | | ACACTGACCCCATGGGCACCTTGATCGTGGACTTCC |
| | | TTGTGCTATGGACTGAACTGTCCCCACATTTATAAG | | AGCCTCCAGAATTTGTGAGAAAACAAAAGTTTGGTT |
| | | TTGAAGCCCTGACCCTCAGTGTGACTGTATTTGG | | TTTTTTTGGCGCCATCCAGGCTATGGTACAGTAT |
| 352481 | SCAFFOLD | CAACCAACACTGAAGGGGGTAAAAAACATGGAATCC | G/A | AAGAATGATCAGTATAAAACAAAAAGGGATTTACTT |
| | 90027_18395 | ATGTATTCAATGAAATACTACACAGCAGTGAATAAA | | TGGGAAAGAGCAAGGGAGATTTAACAGGCTGCTCAG |
| | | CTTGATAACAGCCTTAAGCATCACATGGATTAAACT | | GTGAGGTAAAGTTCTATTTCTTAGCCCAGGTGTTGG |
| | | AAAAAACTAGTGGTCAACGAACTGACTTATAAGAAG | | GAACATAGATGCTCAGCATATTATTTTTCCTTAACT |
| | | ACTTACACTGACTTCTATATAATGTTTAAAATATAC | | GTAAGGTATACTTTCATATACTCCTGTAATCTTATA |
| | | AGATTCAAATAATACTTGGTTAGGACTGAATAGAGA | | TCTCACATACTGGAAAACCAATAAACTGCTGAAGTA |
| | | TTCATAAAGATGGCAAAGACTAACATGGAAAGCA | | ATTGTAATTGAAGATATTTTAAAGATCAGACAAC |
| 346806 | SCAFFOLD | CAATGGTGTCATAACAAGTTGAAAAGAATTTTTACG | T/C | TGAGACCTTCACCAAGAAAGCACGTGAGTCTCAATA |
| | 307284_3460 | ACTGAAGTGTCTGAATCATGAAAAAACCTTACTTAA | | TGTGTGTTATATACTTGCCAAAATTCCCGATGGAAT |
| | | CTTGCATGTTTGCAGATTCGGTACTGGGCTTCCCAT | | GATACAGGTACAGGTGAGAGAAATTTACTACTAGCA |
| | | GACAAAGAAGCAGCTGCACACAGAGTTCAAGTTGCT | | ATCAGCACATTTGGGTTTCAACTTTAGCCTTTAAAA |
| | | AGCCAAGAATACTCGGCCAGGCTGGAGAACCTTCTG | | TCATATAAGTTTTAAAATACATCAAAAACAACAAAA |
| | | CCTGACACCCAGTATTTTGTGGAAGTCAGGGCGTGC | | CCATTGTTAGTTATTGTAGATGAAAAAAGTGGCAAT |
| | | AATAGTGCAGGGTGTGGACCTCCCAGTGATATGA | | TACTTTCACATAATTAATATCCATTTCAGAAATA |
| 352035 | SCAFFOLD | ACCACAGTGTTCGAATTCATGATATGAGTGGACTAT | C/A | ATTTTTTTCTTGCATTTATCTTTTTGAAAAACAATT |
| | 130001_59457 | TTCTGAGGGGGAGAAAAAAAAATCATCAATGATGAT | | GTTGATTGGTTTAGAACTTTAAGCAGTAATTTACTA |
| | | CCTTTTGACTCCTACTAATATTTAGCCAAATGACAT | | AGGGATGAAAGGATTCCTAAAGTTTCTAAAATGTCT |
| | | AGTTGCCAGAGGCTAGACCTAGCGAAATCTAATGAA | | ATTTTGCAACCAGATGTGTTTTAAAAGGTAAAGTTA |
| | | AGGGGTCACATTTCAGAATAATTTTGATAACTATTC | | ATCTGTGCTTAAATAGAATAATATTAGGGCAGATCA |
| | | AAGCCTTAATGAGCTCTTCTAAAACAAGTAAAGCCA | | GATACTTGAAGAGCCGTGTTATACACTCCCTAGTAC |
| | | CTTTCACTCAAAGGCATAGGAGACAATTAGGAAG | | CTAAGCATTCACCATGTGACTTGGTGGTCATCAT |
| 354080 | SCAFFOLD | GATTTTTTCTTTGTCTTTCAACATTTTTACTATTAT | T/C | TTTATTGATATATTTTCATATTTGCTGTTTCTTTCT |
| | 47202_2678 | TAGTCTAGACTAGAATCTCTTTCATTTATCCTGTTT | | CCTGTTAACACAAATACACTCTTTAAATTTCAGAAT |
| | | AGAGTTAGTTGAGATTCCTTCATGTGTGGATTAATG | | TTCCATTTGGTTCTCTTTCATATTTCTTTCTCTTTA |
| | | ATTTTCATATAATTTTGGAAACTTTCTGTTATTATT | | CTGACATTTTCTATTTGATAAAACATGGTCATTATA |
| | | TATTCTTCTTTTTCTGCTCCTGTTTCTCTCCTCTGC | | CCTTTCTATACACTTGTACACAAAGGTTCTTTTAGT |
| | | ATCTGGTAAGATTTCTCTGAGGTTTTCTGAGTAATT | | TTTTTTGAATATATTTATAATAGTGATGTCAGTATT |
| | | GTTATATTGCTTTCTTTTATTCACTTTGCAAAAT | | TTATCTGCTAGAACAAAGCATGTTTTGTATGCTT |
| 345391 | SCAFFOLD | GTTCAGACTATTGGAAAATGAGCTCTCGGTTGGCAG | T/C | GAAGGGATAGGTTGTCCTAGGAGTGGCCACTTAAAG |
| | 120838_12637 | GGATATTTTCATGCTTTTGCCCTCCTTTCCATTTAC | | GTCACACCCTTCGCCAACTGCTTAAGTATTATTCTG |
| | | TTGCAATTAGGGTGATTTCCTATTTTCGGCCATTGC | | GTGATCACATCACTTGGCAATTTCTGGGCAGCTACT |
| | | GTTTCTGTGTGTTGAGGTCTCACGTGAAGCTTGGGA | | CAACTTCCTCTCTCAGGAAAAACTCCAAAACTGAAA |
| | | AGAGGGGAGGGGCACACATATGGCTTATAACCGCTT | | TGGAAAAGGGCCTGATCTTTTTAATAAAGGAAAAGG |
| | | TATCCAGCAATGACCCTGGTCCCAGAGAACATGACT | | AGCTGAACGTCTGTTACTTCTGCAAAACAAAATAGA |
| | | GATCTTGGCTTCTTCAGTTCTTTTCTGTGGCCAG | | TTCTAGTAAAAGAGAGTGTTTATAGGAAATGGAG |
| 350770 | SCAFFOLD | TACAAAAGAATAAATTTATGTATATGGCATATGGGG | C/T | GTGGATAACTGAATCACTTGGCTGTACACTTGAAAC |
| | 164_17176 | GCTTCCCTGGTGGCTCAGTGGTAAAGCATCTGCCTG | | TAATACAACATTGAAAATCAACTATACTTTAATAAA |
| | | CCAATGCAGGAGATGCAGATTCAATCCCTGGGTCAG | | ATTAAAATATTAAAAATAAAATCAAAATATTAACAA |
| | | GAAGATCCCCTGGAGAAGTAAATGCAACTCACCCCA | | AAAAATAAAAATAAAAACCAAGAAAACACACACACA |
| | | GTATTCTTGCCTGGGAAATCAAAGGACAGAGAAGCC | | TACACATACACAAGAACTGATTGGGAGAAAAGTGTT |
| | | TTACAGCAGACAGTCCACGGGGTCACAGTGAGTTGG | | GGAAGTTCAGATTACCAGTCTCCAGAACAGAGATGC |
| | | ACCCAACTTAGCAACTAAACAACAACAATACGTA | | TCTGAATTTCTCTGGGCTTAGTTTTGTTTTATTT |
| 350931 | SCAFFOLD | ATGGATTGCAGCCTACCAGGCTCCTCTGTCCATGGG | T/C | TTCAGTTCTATGTCCCAAACAAAAGTACAATAAATT |
| | 243543_741 | ATTTGCCAGGCAAGAGTATTGAAGTGGGGTGCCATT | | AAAATTGAATGAAAATTGTGATAAAGCCTCATGTCT |
| | | GCCTTCTCCGATGCTTCATGCTAGGTTACTGCTTTG | | AATGAATTTGAGATGAGAAGGAATTATGCTAATTTG |
| | | GGTACTGGGCATCTTATAGCACCATGCAACACCTGA | | GGTACTTGGTTGTACCCATCATGTTAGGCTTTCCTG |
| | | ATAATAGCCATCACCTTACCAGAGATCCAAGTACTC | | AATCTGTGTAGAAACACAGGTCCTATATGCAATATA |
| | | AAGGCCCTGTATATTTTGGCCATTGAAATGATAAAT | | AACAGGGATATTACTGCTACTCAGTCCTTTGCCACA |
| | | TATTGAAAAAACAGAAACTAATTTCTTTTCCATG | | AAGAGCAAATAATTTGACTGACTATGGATATGTT |
| 348309 | SCAFFOLD | CTTTTTAATCATCATGCCCCTGATTGCTGATTATTT | T/C | GGCTATAGTTTCAATACACGGGTCTACAGCAGAAGG |
| | 196149_5586 | ACTCTTCTGCTGTTTCCTCTCATGATTACACAGCCT | | AAGACTACTGTTATTTTATAGATTGTTCTGGAGGAT |
| | | GCTATGGAGCCAGGCATCATGCCTCTGCGAGTCCCT | | GCTGCTACACTGGTACATGATCTCAAGGTGTTGAAG |
| | | GTAGCATCTCTCTCCACTTTAGTTTGTCATAAAGCA | | ATATTGGCCTACTACTTGATTTATACACAGCTTCCA |
| | | GCTGGTTTCTTCTCTCATCCATCCACTAAGTATGCC | | TAAATTTTAGATAATCCAATGACAACTTTCAGTGCT |
| | | CTGCAGAATTGTCTAACCAAGATTCTACATGATATG | | ACAAGGCTAATTAATTTTTTTAAAGGTATTCGGAAT |
| | | GTTGCATTCTACTCACAGCAACCTAAAACCTACA | | AGCAAATTTGCTTTATATAAAGGCCTTAGCAATG |
| 346200 | IBISS4snp688 | ACAGACAACTGCCACAAGTTTGTTGACATTCTTGGC | C/T | CCCCTTTTTATGAAGTCTCCCAGGAAGATCAAGAAA |
| | | TTACGGACCATCTT | | GTGGGAACCACTGA |
| 350054 | SCAFFOLD | TTTCCTTCTCCAATGCATGAAAGTGAAAAGTGAAAG | C/T | TGAATTTGGTCTAAAGTCTTTTCCTTGTTCTTAGTG |
| | 200776_731 | TGAAGTCGCTCAGTCGTGTCTGACTCCTAGCGACCC | | GATCTGCATCTGGGCAACCTAAATATTCATTTTGAT |
| | | CATGGACTGCAGCCCACCAGGCTCCTCCATCCATGG | | TTCTTGCTTTCTTTCTTTTTTTTTTTTTTTTACTGG |
| | | GATTTTCCAGGCAAGAATACTGGAGTGGGGTGCCAT | | GAGGAATGGTTATAAAGCCAGTGTCATTTTATTTTT |
| | | TGCCTTCTCCACTGAGGCATGACCAAATCGTATTCA | | AACAGAAAATTTTCTAAGGGAGATTCCTAGAGGCAC |
| | | GCAGTCAGAGTGATGTCTAGGAGAATGTTGGAGGTG | | AATATAACACTTAACGTTAGATTTGTGTTAGAGAAT |
| | | TCTACCCATGAAAACAGTGTCTACCCACGAAAAC | | TAAATGCAGATCTCAGATTTATAATTGCAAAATA |
| 342661 | IBISS4snp18 | TTCCGGGACCAGATCCTGGCTGGAAGCCCTGAAGCC | T/C | GCTGACGTCTGCTCCGACTTCCCCTTGAGCGCCATG |
| | | TTCTTCGTGCTCAA | | TTGGATGTCCACAG |
| 347669 | SCAFFOLD | CCCATTCTGCAGCATATGTGGGCCCCTTTCCTGTAG | A/G | TTTCTCTCCTACCATAGGCTCTGTACACATATCACC |
| | 295811_2321 | CCACCTGAGGGCAGAGCACTCATGAGGAGATAGCTC | | TCTTTTAGTCCAAGGGAAGCATTAATATCCACATTT |
| | | AGAGAGGGTAGGTGTCTTGTCTATGCAACACAGCTA | | TACAAATTAGGAAATCAGAACCCTGGATTGTGGATT |
| | | ACGTGTGTTGGAACTAGAATCAGGACCCAGGTCTCC | | AGGGCTCCAATAGGGTTGTTGAGAAGGCAATGGCAA |
| | | TGACAAAATCACTGCATAGTTTTCTTTACACCTTTG | | CCCATTGCAGTACGCTTGCCTGGAAAATCTCATGGA |
| | | TTCTCAGTCAGCCAGCCTTCAGTGTGATGATACCTA | | CAGAGGAGCCCGGTAGGCTGCAGTCCATGGGGACAC |
| | | AGCCGATGAACAGAAGGTACTGATCATTCATCGA | | GACTGATTGACTTCACTTTCACTTTTCACTTTCA |
| 345058 | SCAFFOLD | CCTGGAGAATCCCACGGACAGAGGAGCCTGGTGGGC | T/C | GGAAGTGTTTGGATGGACGTGAGGGTTTTCACACTG |
| | 30862_2553 | TACAGTCCATGGGGTCGAAAAGAGTCGGACATGACT | | GCTCTGATGAGGAATGGACAGTCCTGGAGGAATGCG |
| | | GAGAGCCTGAGCACCCACTGGCTCTTCTACGGGAGG | | GCTGATCAAGCAGTAGCAGGTAAGTGTCGCCGACAG |
| | | AAAAAGTTTTCTTCTACCCATTTAGGGTTCCTGAGT | | CGCCTGTTGGTTGGGATTTCTTTTACCTTTTCATCT |
| | | GGGTCTGAAAGTTACGCTGACAAAGACAGACCAACA | | TATATTGTAGTACAGCCAGTTAGCAGTGTTGTGACA |
| | | GGAGGAAAGCATACATGTTGACTTAATGTTTTACAT | | ATTTCAGGTGAACACCAAAGAGATCAGCCACACATA |
| | | GAAATGGAAACCTTCACAAGGAAACGAAGGTCTG | | CGCATGTATCCATTCTCCCCCAAACTCCCCTCCC |
| 350721 | SCAFFOLD | ACTTTATTAATAGACACACAGACACTTGTGAGTATT | T/C | GCCTAGGAATCAGTATTTTTAAATTTTTTATAGCTG |
| | 146466_8003 | GCAGATCCTGCTTTTCTAGATGTAGGATGGAGTTGA | | ATAAGGGCTTCCTAGGTGTCGCTAGTGATCAGGAAC |
| | | GGTTCTGCATTACCTGCAAGTTCCTAGATGAACCAT | | CCACCTGCCAAGCAGGAGACATAAGAGACACTGTTG |
| | | GCTTCTGGTCCATGGGCCACTCTTTAAGAGTATAAA | | CCATCTCCAGGGGTCAGGAAGATCCCCTGGAGGAGG |
| | | GAGATGGCTCCCAAACTTGGTTGCGTATTAGAGCTG | | TTATGGCCACCCATTTCAGTATTCTTGCCTGGAGAA |
| | | CTTGGAGAGCTTTTAAAAATCAGGATGCCAGGCCAC | | TCCCATGAACAGAGGAGTCTGGTGGGCTACAGTCCG |
| | | ACCCCTGACCAGTTAGATCAGAATCTCAAAGTGA | | TGGGGGTCATACAGAGTCGGACACGACTGAAGCG |
| 344481 | IBISS4snp320 | GTCATTGCAATCCAGGCCCATTGCCAGGGCTGGACA | A/C | GCTGATCAGCTCTAACAAAGCCATGATTCCGGAGAT |
| | | GGAAGGGGCTCTGA | | GTGATTGGTATATT |
| 352723 | SCAFFOLD | TAAGGTCTCTGTCATAACCACTCAACTCTGCTGCAG | G/C | AGCACTTATAATGACCATTTTAATCACAAGGTGTTA |
| | 217082_459 | AAGGAAGCAGCCATTAACATGTAAACAAATGAGCCT | | ACGTTAAGAGATTAACATAAACTTCAGATGTCATAG |
| | | GGTTGTGTTCAATAAAACCGGACTGGCAGTGAGCCA | | AAACAGTTCAACAAAATACAGAAATAACTTGTATCA |
| | | GATGTGGCCCACAGGCTATTAGACTGCCAACTCTGA | | ATTAAACCGGAGCAAACATTTTGGAGACTTCAACGC |
| | | ACTAGAACTAAACTCCATTTTAAAATCTGCCAGATG | | ACTGGATGGAATTCTTCAAAAACTTCCCCGTAAGTT |
| | | TCTTCTCAAAACTAGCTAAATGGAAACTGACCATTC | | TTCTTATCTATAAAAAGAAAAATCACAAAAGATTTT |
| | | TCTGATAAACTGTTTTTTTGACAAAAAGGAGTCA | | AAAATCATGTTTAAAAAATCACACTTCCTAAAGA |
| 346573 | SCAFFOLD | CAGTCTCAAGACTCCCCCAGATCTACTGAGTTGGAT | A/G | CTTGAAGTCTTAGTTCAAACATCTGTAGTTCCACAG |
| | 226597_2085 | CTTGCGTTTTAATAAGATCCTCAGATCATCAATAAG | | CTTTCCAGGATGGCCACTGGCTCCCCTGCCCCACTC |
| | | ATTCCCAGATCCCCTTGGCTAAAAGGTGACTGCACA | | AGCATCCACTACTGCCCTGTGCCCTCGGCAGGGACC |
| | | AAGCCCTGTCTCAAAAGGGGTGCCCCATGCTTGGGG | | TTGAACATTGGTATGGTGACGTCAAGGTCAGATACG |
| | | TATAATGCTCTGTGGTGACTGACTTGACATTCTTGC | | AATGCCCCATGTGCTCAATCATGAGTGATGCTTTGG |
| | | TAATTTTATCTTTGAAATTATATTTTGTGAGTGAAA | | GCACCTTTGAAGGCCTGTTTCTCTGTCGGCATCCCT |
| | | TCTGGTGGGACACTGAAACTTGTGAGGGAAGCGG | | GTATGAAAGCAGCACAGTGTTAAATGGCAAATAA |
| 346994 | SCAFFOLD | CACGCTGAGCATCACCTCCAGCCCATCCTTGATCTC | C/T | TGCTCAAGTCAGGATGTTTCTTAGGCTTCAGAGGAA |
| | 80682_16217 | CCCCTTCCCCTCCTCGCAGCACGTGAGCAGCTCCTG | | ACCAAATGCAGTCATAGTTGAAGGTGAGTTTAGTGT |
| | | TGGGAGACCAAGACCGTGAGATGTTCCACTAAGGAG | | AATGTTAACTACTGAGCCTCAAAAATGACAAACAAT |
| | | GTCTTCACAGAAACCACAGAGTTTGTGAAAATCCGT | | TTAATCAGGGTAGCAAAGCACAGTCACCTAGAGGTT |
| | | GACGTTTGTGGTAAGAACAGATGTGGTCAACTGGGC | | ACACTCGAAGGTGAAGTCAGAGTCCAGTGAGTCCTG |
| | | AGAGATTCTCATAAGGGAATGCAGGATAAATTTGAA | | CTCTGCTGGCCACAGGATCCAGCACGATCCTGAGCT |
| | | TTGCCTCTTTAGCCAGTGGATGGAAGGGTTTGTC | | CTATCTGCCTGCAATGCAGGAGACCCAGGTTCAA |
| 352253 | SCAFFOLD | AGTGACTAAACAACAACAAGTGGATAATACCTATGTG | C/T | GCCTATTAGTATGAATATGCTTTCAGGCGGGTTATC |
| | 256648_4880 | GATAAATGAGAGCTGATCCCATCTCTTTAAAGAGAT | | CAGGAACGGACATAGTACTGGCTAAAAAGCAGATGA |
| | | CTGTACCAAGCCATATACATATTTACATGTGTACAT | | TGAAAAAAATTGAGAAAAAAGACGACAGGGGGTACT |
| | | AAAACCAATTTACATTTATAATACAGTCACTGGAGT | | TCCCTGGTGGTCCAGCGGTTAAGACTCTGCACTTCC |
| | | CTTATGAGAATGTTTTAAAACGTACTGGACGATTCA | | AACACAGGGGGCAGGAGCTCCATCCCTGGTTGGGGA |
| | | CTTGGTCTGATAATTTAGCTTCTTAACATTGTATTT | | ACTAAGATCCCATGTGCCATATGGAGCAGCCAAACC |
| | | CTTACATATCATTATAATTCTGAGTGAACGTAA | | AACAAACAAAAAGACAATGGGGACCCCTGGACAA |
| 345744 | SCAFFOLD | CTTCCATGGCAGGGGGCACAGGTTCCATCCTTGGTC | A/G | CTGTGTATTATTTTTTGCTCCCTCCCCTGCATCTGG |
| | 245054_35388 | AGGGAACTAAGATACTGAATGTCTTGTGGAATAGCC | | CACACAATACACACTCATTATACATGTAGGTTGAAG |
| | | AAAAAACAAACAAAGACCACCTCTCTCCTTCTTATA | | TAGAAAACTATTCCCTAAGAAAGCTGTTTCTTGAGA |
| | | GACTATTAGTCTGACAGACAATCCCTAAAATTTCTG | | ATTGATTTGGAATTAGAAATCTTGGATGATCTGTTG |
| | | GGTGTGGGACCTATGGTGTATGCCAGCAAACTATAA | | AACTGTTTTCTGTAATCTTGCCTCTTCTTACATTAT |
| | | ATGACTAACATGAGTTGAAATAATCTGATCTCTCTC | | TTTACAGGGTATATCCCACTTGGGGAAAGCTGAGCA |
| | | AGGGACTCCTCAAGGAAAGCACTCATGGGCCAAG | | GGGCTTCTGGGGTGCCTGGTCTTAACCTGGGCAG |
| 345999 | SCAFFOLD | CCCTCTCTGTTAATCCCTCACCTGCCTAGAAACAGA | G/A | AAATCCCATGGACAGAGGAGCCTGGTGGGTAACAGT |
| | 40204_33814 | GCCACAGATGTAGAGGACTAATATATGGATACCAGC | | TCATGGGGTCACAGAGAGTCAGACATGACTGAGCAT |
| | | GAGGAAGAGGGGTGGGATGAACCGGGAGATGAGGAC | | GAGCGTGCACACACACACACACACACACACGTACAA |
| | | CGACACGTACACACCACTGCGGATACCACGGGGCTT | | ACAGATGGCTAATGAGCACCTACTATACAGCACAGG |
| | | CCCAGGTGGCACAGTGGTAAAGAATCAGGAGATGCG | | CGGGGGAG |
| | | GGTTCCAACCCTGGATCGGGAGGATCCCCTGGAGTA | | |
| | | GGAAACAGCAACTTGCTCCAATATTCATGCGTGG | | |
| 342582 | SCAFFOLD | CATCTGTGTTCAACATTACGAAGGTGAGATCCCCGG | A/G | TGCTTCAGCCCTGGAGAAGGAAGGAGGCCGGGCTGG |
| | 281406_2591 | CTCCTTCGTTGACTCGGCTGGCATGTGTCGATGGGC | | AAGCGCCTGCAGGTAAGGCTGGGTAAATGTGAGGGA |
| | | GTGTTATCACACACCAGACACTAACACGTGAGGAGC | | CCAGATTGGGCCGGGTTCCTACCATTTAATTCTCAC |
| | | GGTGGCTCTTATAAAAGCACGTCACGGGGGACCCTG | | TTACTTCCACAACGACTCTATAGATGGCACTCCCGT |
| | | ACCGAACGCGGATCTAAGGCTTCCGTGAGCTGTGAT | | TAGACAGATGAGGCAACGTTATGTGCACAGGAGAGT |
| | | GCTCGGATCGAGTTCCGAAGGATGGCTACAAAGTAA | | CTGCTCAGAAGCCATCATCCTACCCTTTTTCCTTGC |
| | | CCAGGCATGGAGGGGAGGCACATTAGAGGGCAGC | | CATCAGATCTTGCCTTTCACCCTAGAAACACAAA |
| 353285 | SCAFFOLD | CATCTAGAAAGGGGAAATCATAGCTGTATTTTAAAA | G/A | ATATAATGATCTTACTGGAGTTTCTTTCTTTACAGT |
| | 291058_5948 | ACCAAGTGAAATAATTTTCTAAAAACAGATTTTAAA | | TTAAACAGAATAGATTCTTTTTATTAAACTGTTAAT |
| | | AAAATTTAAAAATTGCTTAAAGCCTCAAAAACCAAT | | CTTCACAATTTTTTAATTTCTGTAACAATTGAACAG |
| | | GCCAATAGTTAAAAATATCTCATCTCCAAGTTCTTG | | ACTATTCTCTATTTATGAATCCCATCACTCAACATC |
| | | CTTACCTCTGATTGAAATTCTTTTTATAAATCCAGT | | TGTGAAACTGATAAGATAACCCTCTCTAGGGTTTTA |
| | | TTTTCTTTTTTTATCTTTAGCCGACACAGAAAGCAA | | TTTTGTAACCTTTATCTCCTGAAACATACTGGTCAT |
| | | CTAATCGCTGTTCTCAGAGATTTAACATTCATAC | | TTTGCCCAACTACAGAATGCAGGAAATGTGGATT |
| 344225 | SCAFFOLD | AAGCTGTGGAGGAGAGAGCGAGCTAGGGCAGGGTAA | C/G | AGAAAGCGAGCACGTGAGTGAGAGAGACAGCGAGAG |
| | 286624_6678 | TTGAAGGCTTTCAGTGTTGCTTGCTTTCAGATGTGA | | TGACAAAGTGAAAGAAGATGGATGGAAAAGGGCAGG |
| | | GTGATTTAAGCTTTTTACTCGCTGAGAAGAAAGAGA | | TTATTTTGCAGCTAGAAACTCTGGAAGTGGCTCAGT |
| | | AGAGAAAGTTTTTGAAGACATAAAAAGAAAACAGAA | | TCAGAATGCCTCATTTTCTCTGAAATCACAACCTCC |
| | | AACTATGTGGTTAGATTGAGGTTCAGAGCTCAGATA | | GGTGTGGATGGTGAGTGGACGAGATACAGAGACTGC |
| | | AAAGAATTATGCTTGGTTGGAGGAGAGAGAAACTCT | | AAAATTGTTGTGAAGAATAAGAAAAAGAGAAAGAAA |
| | | GTGTGTGTGTGTGTGTGTGTGTGTGAAAGAGAGA | | AGCAATAGACGAGGACATTCAAAGACTGACTAGG |
| 346776 | SCAFFOLD | AGAAGCTGTCTGAAGAGTGAACTTGGGAAGGAGACA | C/T | GACAAATGATGCACCCATATGTCATCCGCCTCAGAC |
| | 296111_2645 | GTGGAGGACAGCCGACCTCTAAGGTCGCGTGATCCC | | AGTGCTTGGATTCACACCCAAGCATTGGTGATTTCA |
| | | CCCAACACCTCGATGTGCTGAACCTGGACAGATGTC | | CTGACAGTTGAGTAGAGTAACAGACAAAGCTCCCAC |
| | | CCTGTAGGACAGACGTACCTCCTGTCTTCCCACAGT | | TAGTTGAGCCCTGCCTGTGTGCTGGATGCTGTGCTC |
| | | GCAGGAGGCTGCCACATCCCTCTTCAGTGGCGGTGG | | TTTCATGTGCAGTAGCTCAATCAAACATCATGATGA |
| | | TCCTCAGTCCAGTAGCCTTCCGGCATCTTGTGTTGC | | TGAGGCTCAGATAAAGTTATAGCAACCATATGACCC |
| | | TCTGTGCCTTGTTGGACTCTAGAGAGACGATGCA | | ACAGTTGTGCTTTTTGGTATTTACCCAGATGAATT |
| 343060 | SCAFFOLD | CCTCTAAACCACCAGTGAAGCCCTTCTAATACTTTA | G/C | CCCGTGCTCTGCAATGAGAAGCCATGACAGTGAGAA |
| | 36638_9838 | AATGGCTCTATTTCATCTCCTTGACTGTGAGGAGCT | | GCCCGTGCACCGCAACTGGAGAATAGCCCTCGATCA |
| | | TGACAGCAGATTCTGTTCTTGAAATAAGAAGCTGCC | | CCAAAACCAGAGAAAAGCCTGTCAGCAACGAAGACC |
| | | CCAGGGGCTTCCCTGGTGGCTCAGTGGTAAAGAATC | | CAGCATAGCCAAAAATAAATAAGGGAAAAAAAGCAG |
| | | CTGCCAATGCAAGAGACAGGGGTTTTATCTCTGATC | | CAGCTGCCCCAGGACTCTAGAATCAGAACCTGAAAC |
| | | CAGGAAGATCCCACATGCCACGGAGCAACTAAACCC | | CAAACCGTGCCCTTAACATCAGTCCACATACAAGTG |
| | | AGGAGCCATAACTACTGAGTCCATGTGCCCTAGA | | CTGCCTTTGTTTTCCTTATCTCTTCTCCTTTATA |
| 354110 | SCAFFOLD | TAGCAATAATGTAAAGTAAAAACCAAGTTGTAGAAC | C/T | AAAACTTTTTGGATCAACAACCAGTATTGCTTCTCA |
| | 104_13107 | AACATACATAGAGATACCATATGTATAAAACAAAGA | | AGTAAAAAGTAATTTTAAAATTCCTACCTGGTCAAA |
| | | GACCTGTATGTGTACACATACACACACACATATTTT | | GGTAAGAAAAATCTACCAGCATTTTAACCTTGGATT |
| | | TTCTTCAATGTTTACAGAAGTCTGGAAGGATATATA | | CTGAAGTACGAAGCTTTTTTCTATCAGAAGTGTTGA |
| | | CCAAACTTAAAAGCAAAATTTCCATATAAGGAGAAA | | GAATAACAACTCTGGTATTCTTTCTAGCCCCACTTT |
| | | AATGGATTGCAGTTAAAGGTAAATATAGAGAAAGGT | | TACTCCTACCCCCAGGAAAAGAGTAGTAACTCTTAT |
| | | TTTATTAACTTGATATATACTACTATATTATTAA | | AATTGAAATGGAAGACATGCTGCTCTTTTCCTGC |
| 347357 | SCAFFOLD | CTGATTGCTGATTATTTACTCTTCTGCTGTTTCCTC | C/T | GGGTCTACAGCAGAAGGAAGACTACTGTTATTTTAT |
| | 196149_5605 | TCATGATTACACAGCCTGCTATGGAGCCAGGCATCA | | AGATTGTTCTGGAGGATGCTGCTACACTGGTACATG |
| | | TGCCTCTGCGAGTCCCTGTAGCATCTCTCTCCACTT | | ATCTCAAGGTGTTGAAGATATTGGCCTACTACTTGA |
| | | TAGTTTGTCATAAAGCAGCTGGTTTCTTCTCTCATC | | TTTATACACAGCTTCCATAAATTTTAGATAATCCAA |
| | | CATCCACTAAGTATGCCCTGCAGAATTGTCTAACCA | | TGACAACTTTCAGTGCTACAAGGCTAATTAATTTTT |
| | | AGATTCTACATGATATGGTTGCATTCTACTCACAGC | | TTAAAGGTATTCGGAATAGCAAATTTGCTTTATATA |
| | | AACCTAAAACCTACATGGCTATAGTTTCAATACA | | AAGGCCTTAGCAATGACCACTTATACCCGGACAT |
| 343987 | SCAFFOLD | GACGGAGGAGCCTGGTAGGCTGCAGTCCGTGGGGTC | G/T | CTGCTGCTATTCTAATGGCTTTGTCCTTGGGCCCTT |
| | 170330_7466 | GCTAAGAGTCCAACACAGCTGAGCGACTTCACTTTC | | GATGAGGAACCACATTTGAGACAACACAATGTTTTA |
| | | ACTTTTCACTTTCATGCACTGGTGGAGGAAATAGCA | | CCTGCAAGTCCATTCATTATTATCCTATGAAAAGTC |
| | | ACCCACTCCAGTGTTCTCACCTAGAGAATCCCATGG | | ATGTTTTGGTAAGGGAAGTGTTGCATTGACAAGTGT |
| | | ACGGCCAGGCCTGGTGGGCTGCCGTCTATGGGGTTG | | TTTTAAGCAAACCAGATGCTAGGGGTTTCCCCACCT |
| | | CACAGAGTCGGGCATGACTGAAGCGACTTAGCAGCA | | CTTCTGGCTCTCCCAGGNNNNNNNNNNNNNNNNNNN |
| | | GCAGGGAACCCAAGCCTGAATTTTGCCCGTCCCC | | NNNNNNNNNNNNNNNNNNNNNNNNNNNNNNNNCA |
| 343337 | SCAFFOLD | GGTCACAAAGAGTCGGACACAACTGAGCGACTGAAC | C/T | CACCAACCAGTCAGAGGAAGGTCAGACACTCGGCAG |
| | 164_20850 | TAAACTGAGTGCAGTGATAAGGCAGAGTCCTCGTTC | | CCCTCCCCTCCAAATTTGCCCTTCCTTGATAACCGT |
| | | CTCCTCAAGGGATGTACACTGAACAGTCTCACACAG | | TGGGGAGTTTGGGTCTTTTCAGCGTGAGCCGCCAGC |
| | | ATCTCTGAGTTCTTCTGGAGGAATAAGGCCCCCACC | | ATTCTTTGCTCTGCCCAACAATAAACCTTTCTCTGC |
| | | CAGATGAAGGATGGTGACTTGGGACTGAACACAGTA | | TCCACACTCAGACGTGTCCGTTTGTTTGGCCTCACC |
| | | TGTGAACTCAGAACTGGCTGGACCCAGAAGGCTGAT | | GTGCATCAGGCACATGAATACAGCCCGTAAATGACA |
| | | GATTGAGATCCTGGAACCTCACCCTGTTACCTCC | | ACTCAGTAATGCTATTTTTCAGAACAGTGAACTG |
| 346674 | SCAFFOLD | TCACTAGCTACCTGGAAGGCATTGGAGGCACTAAGC | G/A | TAGCCCTTGTGCCTGACTACAGTGGGAATTCCGAGG |
| | 265658_20008 | ACCATTGGCAACCCCAGTTCATTTTGAGTTTCTAAG | | AACTATCTTGAGAATTCCCAAAGCTATTTTAGGCTA |
| | | GGAGTGGATGAAAAATAAAGCTGATCTCGATTCCAT | | ATAATAGAAAACCCACACCAAGCACAGTGTTTTAGT |
| | | TTATAGGACAGATTTGCAGAAAAGTAGGAAAATGGT | | TTGGACAAAGGTTATTCTACCTTAAGCAGTGGCTGA |
| | | TGACACCCCATTTCATTCTGGTGACATATTGGAGCT | | AAGGCAGTGATTGATGTTTCCCCATCATGAAGCACA |
| | | GCTTGCTCATAACAGTGTTGTAAGTGTCCTACAAAC | | GCATGTTAGTTCCTTACTTCGTCTCAAATTTATTGT |
| | | AAGGCGATTTGCACTAGCAAATGATATGAATGTC | | CACGGGAGGACATTTGTTTGTTACAGTATGCCAA |
| 347830 | SCAFFOLD | CTTGACCCCTAGGTAATTTTTTCCTAAATTAAAATA | C/T | TGACATCTGATAGACTCTCAGAATAAACTGGGCTTC |
| | 525_10998 | GTCACCTTATACAATTTGTAGTGTAGATTAGTTTAA | | CCTGGTGGCTCAGATGGTAAAAGTGTCTGCCTGCAA |
| | | TGAATTAGGCCAGATGATATATATAAAGTTCCTGGT | | TGTGGGAGACCCGGGTTTGATCCCTGGGTCAGGAAG |
| | | TCTGGGAAAGATCCTAGTAAATACATATTTTTGTTA | | ATGCCCTGGAGAAGGAAATGGCAACCCACTCCAGTA |
| | | TTGTCTATAATCAAAATAGTTAAGAGTAGAAATTAA | | CTCTTGCCTGGGAAATTCCATGGATGGAGGAGCCTG |
| | | TTTAAAAGTTCTATCCGTGAAAATTTCAGGTGCATC | | GTGGGCTACAATCCATGGCGTCACAAAGAATCAGAC |
| | | TGGAACAAGAGGGACGCCATTTAAAGTTGGAACA | | ACAACTGAGAGGCTTCATTTTCACTTTCAGAATA |
| 350100 | SCAFFOLD | TCACCATATGCCATAAAGCATTGGCTGGCAAACTTT | T/G | GTTTGGTGATAGAGTTTCTATCCTGGCCGGCCATTT |
| | 215548_7400 | TTTTCTATAAATGGGAACATAGTTAATGTTCACACT | | GTCAGCTGAATGGCGTTGGATGAGTCATTGACCCTC |
| | | TGTGCACCATATCATCTCTATCACAGCTACTTAACT | | TCTGAGCCACCATTTCTCAGCTGCTAAGTGGAGAAA |
| | | TTGCAGCCAAAGAAGTCACAGGCCATGCACAAATGA | | TGGTTACTTACCTTATTAGNNNNNNNNNNNNNNNNN |
| | | GTGTGGCTATGTTACAGTAATTCTGTATTTTCAACA | | NNNNNNNNNNNNNNNNNNNNNNNNNNNNNNNNNCCT |
| | | ACAGACGTCAAGACAGATTTTGCCCTCAGGTCATGC | | GCAATGTGGGAGACCTGGGTTTGATCCCTGCGTTGG |
| | | TTTAATGACCCCTCTGATGTAATGGAAAGGTTGG | | GAAGATCCCTTGGAGGAAACTGTGGCAACCCACT |
| 346790 | SCAFFOLD | ATGTGCAGAACCATCTGGAAACAACGACAGTATGTT | C/T | GGTTCACTTTGCGGGGAGGAAGTTGACTAAAACCTA |
| | 30077_8965 | TTAATGGTCAAGAACTTGACATCCAATGAGAAGTAA | | TGTGTCAAGTTTGAGGCTTTAGGGAGAACTGAGACC |
| | | ATAACCCTTGTAGTTTTTAAGTCTCAAAGCACATAT | | TCATGCTCTTTGTTTCTTCCTAGTAGAAAATTAGTG |
| | | AAAGCATTCACTGCATTATATTGATTATCAATAGAA | | GTTAATATGGAAGGCAATCATTGATTTTTTAAAAAA |
| | | GCTGATGATACTGATTTTTAGTTCAAGGTAAACTGG | | TTCTGAACATTGTTATTTCATTTATTCAACAAGGAT |
| | | GAAACCTAAAGGATTCTGGATGTACTTGAGGACACA | | TTATAGAATGTCCTTGTTGAGAACATTCTATAAGAA |
| | | GAGTGGATGCCATCCAACTGAACTTTTTCATCTA | | CTTATACTATGTTCAATAAACTGTTCATATAGAC |
| 348375 | SCAFFOLD | ATCTCAAAAAGCAGAGACTGTAGACAACATATGTCT | G/A | GTGAAGAGAGTTCCATTATGAGGCTAAACTTGAGGA |
| | 215141_19889 | TCATTCTTCCACCTTCTGGAGCACTTAGGTCAGAAG | | TTTAGTAACTGAAATGCCCTCAGGAAAGTTTGGGTA |
| | | AACTAGTCTGCTTCCTAATTTGGTAAGTCACAAGCT | | CCTTGAGCCCTGGTTGGATTACTGTGTTCCACCTCC |
| | | TATATCATGTAATGTTTAGAAGATCTCAGTAAAACT | | CCTGGAAGCCAGTGAGGACCCAGCCTCAGACTCTGT |
| | | AAAATGTAGTTACCTATAGATTGTTTTTAATTCTTC | | CCTGGGGGCTACGGCCTGTTTCTTGCACTTCTGTGA |
| | | ATTAAGAAATCAATTCCAGTTCAACTCTTGTTAGTA | | TTTCTTTTCCCATTGTGAGCAGTGGTGTCCAGCATA |
| | | TGATCTTTGATATTCCATGTCTAGTCCATAGAGG | | TGGCCAAAAGTTTTCAGTTAAACACACTATATTA |
| 350976 | SCAFFOLD | AAGATTTGTGTCATGGATGTCCAACATACTCATTAT | A/G | TAAAGACCCCTTAATGCAGGGGAATGCAAAGCACAT |
| | 256426_385 | TTTCCAGATTTCCTTTTAGCCTTCTTCCTCTTCTCT | | GGACTTTAAACAAAATTTCAACTCTGATACTTTAAT |
| | | GACATCCCTGGTTGCCAAAAGATCTTCAGTTTGGTA | | TTATGGCTGAGTAAATTACTTAATAAATGATCAGTC |
| | | ACCATCCAGGCACATGCCCTAAATTCTGAAAATATT | | TCAGTATTAGGCTCACCATGTTCAATCATGGTTTTA |
| | | CTGATCACTAATATATATTACCCCATTCCCCATTTA | | GGTGCTTGGTGCACTTCAATTTATGTAGTTCTCATA |
| | | AGTCTCTTAATGTTATTGCTTAAAATGCAGTGTTTT | | ACAACCCTCTGAGAAGGTCCTAATTATGACAAGAGA |
| | | TTAAAGAAGCCCTTAATGTAAGACTGTATGTAAT | | ATTGAGACACAGAGAATGTAAGTTACTTTCCCAG |
| 347335 | SCAFFOLD | TGCTAGAATTAATGTTCATGACCTTGGGGGAATGCA | C/T | GTTGTTGTCCAGTCTCTGGTAGGTGTATTTCCTGCT |
| | 186240_11564 | AAAAAAAAAAGCCTTAGGAGAGTCTCTCAAAGAGTA | | CACCTCTTCCGTACTAAAGTGCATACCTTAATTGGG |
| | | GGCCTTTTGGTAGAATTCCAAAAGGCCGTCTACAAA | | GTAGGGTGGGCGATGTCACACTGTTAATGAGAATTT |
| | | ATGCTTGACTGGGAATGCTGTCTAGCGGGGTCTTAG | | TTAGAGGAGTCATGATATAATCCTTGTCTTTATTGG |
| | | GTTCTGTCTCTTACAGGTATGTGTTGAGACGGATTC | | CCTAAAATCTGTGTTTCCCATTTGCCAGGGAGATGC |
| | | TCCGCCGGGCTGTTCGATACTCTCACGAGAAACTCA | | GTTTCCTGAGCTGAAGAAGGACCCAGATATGGTGAA |
| | | ACGCCAGCAGGGGTTTCTTTGCTACGTTAGTAGA | | GGACATCATTAATGAAGAAGAAGTGCAGTTTCTC |
| 345053 | SCAFFOLD | CTTCATGCTGATAAGACTATTTCCATATACAAATTC | C/T | GATTTTTGGTGGCCCCAAAAATCTGCTGGCTTCTTT |
| | 305765_6934 | TGTCCAGAGGGACAATGGAGGAAGAGAAGCAAGGTT | | ATGAAATATGAACATTTACTGCTGTCTGAAAAGAGT |
| | | GATGGAGAAGAAAGAGGGACTATTTGGTTCTGCAGT | | CTGATGCTGAAGCTGAAGCTCCAGTACTTTGGCCAC |
| | | AGGACAACCTAGAGTAGCCTGAAGACTAGAAGAGTT | | CTGATGTGAAGAGCCGACTCATTCAAAAAGACTCTG |
| | | AAGTAGAATAACTTAACTATTTGGCCCCTTTATTGT | | ATGCTGGGAAAGACTGAAGGCAGAAGAAGAGGGCAG |
| | | TTGTGACTATCTGTCAAGGACTAAGAATTCCAAGCA | | CAGAGGATGAGACGATTGGATGGCATCACTGACTCA |
| | | AGTAAAAGCCAACTTAGGAAAGACAGCCCAGTGA | | ATGGGCAAGAGTTTGAGCAAGCTCAAGGAGATAG |
| 347260 | SCAFFOLD | TGCCCCGGACTCGTCGGCTTAAAAAAAAAAAAAAAC | A/T | CTTTCTCTTGGGATTTGCCTCCTCTGGACATTTCAT |
| | 161870_661 | AGCTTTATTGAGATATAATCCACATACCATAAATAA | | TAAATAGAATTAGGCAACATGCAAATTTTCTGTCTT |
| | | ACCACTTTAAAGTGTACAAATCAGTGATTTTTAGTA | | AAAGTTTCATTTTTTTCTGTTATCAATTATTGTTAT |
| | | TATTTCTAAGGTTGTGCAACCTTCATCACTGTCTAA | | TATTATCATGATTATTATTATTACTTTTGGCCACAG |
| | | TTTCAGGACATTTTAAATCACCCCAACAAGACACCC | | CTTGAGGGATCTAAGTTCCCCAACCAGAGATCTAAC |
| | | CCCCAAACCCATTAGTAGTCAATCCTCACTCCCCAC | | CCTATTCCTCAGCAGTGAAAGTGAGGAGGAGTCTTA |
| | | TTCTCCCTAGTCCCTTGCAACCACTAATCTACTT | | ACCACTGGACTGCCAGGAAATTCTCTCCTGATTT |
| 348171 | SCAFFOLD | TAAGCCTTATAGGAGAACTTGACCTAAATAATTATG | T/C | GCAGAGAAACCCAGAGAAGGTGACGCTTCATTGTGT |
| | 140539_4990 | GAGTGGTAGTCAGTGTTCTAAAGTTTGTGTTAATAA | | TAGCAGTGGGATGAGGTCAGAAGGGAGGGAGTTGGG |
| | | AACCTTGTACATATTCACAGATTAATAATGGACTCA | | GAGAGTTGCCCTGAGGAAATGAAATTTGAACAAGAT |
| | | GCGAATATTTACTGCACAGGTATTAGCGAATGGGCC | | CTTCAGGAAGTTTGGAGGTGGCTGGGGAGAGAGGGC |
| | | CCTATCACTGGAACAAAGACAGACTGGTCCTTAGAA | | ATTTCAGGCAAAGAGGCCAGCACAGGCCCAGCCTTG |
| | | TTCCCATTGTTCTGTTAGTATCACATAGAGTAAGTG | | GAGGTGTGACCCTTCCGTTTGGCCTTGTGAGTGTTG |
| | | ATGAGTCTGGGATAACAAAACTGACAGAAACAGA | | ATTTATGGGGCGAGGGAGAGGAGCAGACCAGGAG |
| 352009 | SCAFFOLD | ACTGTAATCCATGGGGTTGCAAAGAGCTGGACATGA | G/A | AAAGGAAACACTGGTCTATATCATATAATATCTTTT |
| | 120670_4420 | GTTGGTGACTGAAAAACAACAGCAACAATCTATAAT | | ACATTGGTAGATTAGATTACTGTTCAAAAATATTCT |
| | | ATAGTTACCTTGAGACTAGAGGTTAGGTGGGAGAAG | | CTATATTCATGGAAAGCGTATATTTCCCCTATCTTC |
| | | TGAAGAGGAGGGATTATAAAGGTACATGATGAAACT | | AACTTGAACTTAGCTCTGTCACTTTCTTTTGCCATT |
| | | TTTTGAGGTGTTGCATGTACTTATTATCTTGATTAT | | GAATATTGGCAGGTATAACACAAGGAGGTTGAAATG |
| | | GATGATGGTTACATGAATATATACATATATTAAAAT | | TTGTTGTATATCTGGACTCCTTCTCTCTTTACTTTG |
| | | TTATTAAATTTTATACTTCAGAAAAACAGATCAC | | TATCATTAGGAGAACGGCTTCTTCTGGGTCACTG |
| 348433 | SCAFFOLD | GGATAAAATTAAACCAGAGGACATAGTAGGTATTTC | T/C | GGTTCATCAGATTTTGTGTAGACCATTTCCTAAATG |
| | 235739_16691 | ATTTTATCTGAGGACATTTTGCTTATACCCCATATA | | AAACATCATTTTGGGAGTGGACACTTTCCACCGCAA |
| | | ATGCCTGGGAATCCTATTATTTCATGCATTTCAATT | | GATTTAAAGAACTCTTGTAGGATTATTCATTATTTA |
| | | CCATCTCCCATGTTCATTAAAGAAATTTCATCTGAA | | TGATATCATCCACATAAAAGGAAAGGAGTTGCCTTT |
| | | GCTAGTATATTACGGTAAATCCACTATGTTTTAATT | | CATCTCACTCGGGAATTCATTTCTCTACCTGTGTTT |
| | | TTTTTTTTAAAGAATATTTTAAACTTTCCCCATTAC | | TCCCCTATTCTCTCCTGGCATCAGAATGTATGTTAT |
| | | CACTCAATCTCCAAGGGCTCATCTTTTTCCCTTA | | AATTTAAAACGCATGCACTGTATTTTATTTTCAT |
| 350229 | SCAFFOLD | GATTTGCACTAGCAAATGATATGAATGTCGTAGCCC | G/A | GAGGACATTTGTTTGTTACAGTATGCCAAAAAGGGA |
| | 265658_20229 | TTGTGCCTGACTACAGTGGGAATTCCGAGGAACTAT | | AAACAAGTTGTTAAAGGTCAGATAGTTATTAATGAT |
| | | CTTGAGAATTCCCAAAGCTATTTTAGGCTAATAATA | | TTTAACTGAAGAAAGGATTTAAAAATCAACTCCATG |
| | | GAAAACCCACACCAAGCACAGTGTTTTAGTTTGGAC | | GTATGGACTCTTGAATGCCTTACCAATGATCCATTC |
| | | AAAGGTTATTCTACCTTAAGCAGTGGCTGAAAGGCA | | TAAAAAAAAATCTTCATTTATAAGTGACAGTTTTCT |
| | | GTGATTGATGTTTCCCCATCATGAAGCACAGCATGT | | CCCCTAGTTGTCAAGAGGCAAGATTTTTTTCTCCTC |
| | | TAGTTCCTTACTTCGTCTCAAATTTATTGTCACG | | CTTATTATCCTGTGGTCCAGATGCACTAGTTATG |
| 349668 | SCAFFOLD | AAAAAATACCTTGGTGAAAGAACAGGGTCAGTTCTT | C/T | TATGAAACTGGCCCAAAGTGCAATTTCTTGATAGTT |
| | 66599_4817 | TGCTCAGCGTCTTCACTAGAGGATAAATCCACAAGG | | GAGCTCTTCTCCTTCATATGTAATATCTTTAACCGT |
| | | TCATCTGTGTCCTGTACACAGGGAAGCTCTTGTTCC | | AAGCCACAAAAGAGCATAATATTATGATGCCTATAA |
| | | TTACTGGATTCATCTTCATTAATCTCAGGACTTAAA | | ATCACACTATATTACCTATCAGTATTCACTTAATAT |
| | | GACAGGCCTTTATCTATTAATACAAAACACATATTA | | CTTGGTGAAGCTGAACCACATAAAATTTAAATTCTA |
| | | TTATAAAAATGATAGCAAAAACATTTCAAAAAGCAC | | CAGAAAGATTGTTCTAGTCTGCTACTATCTTTTCCT |
| | | AAAGGAATTCACATACAACCCTATAAAGTAAGGA | | CCTCTCTTACTTGGTTATCTGCTTTAAGTAATCT |
| 352468 | SCAFFOLD | CGGGCGTGGCCCATTCTGCAGGCATGGAGAGTCTAG | A/C | AAGTTGTGAACTTTCAAAAGATGCAAATGTGCATTC |
| | 81013_3562 | ACCACAGGATTCAGCGCACTGTTCGTGGTCTGAACT | | CAGCAATGTGAGGCATGAGTGACATTGTAGCTTGCC |
| | | CAAGCACGCTCAGGGCAGCCCACAGCAACTGCAGGA | | CTCCATCTCCTGTTTGCTGACCGTCCTTCAGCTCTA |
| | | GAGTGGGGGAGACGAAAACCACAGCAGGGGATTAGC | | CCATCTCCCACTTCCTCTCGCTCCTCCAGTTAGTAA |
| | | TCACGCATCTTGCTTCATTTATTTTATTGACTAAAA | | CTCTTCTTGCCTGTTTACTCAATGCCAGCCCCTGTA |
| | | AAGAGCTGAGAGCAAATACGGTTAAAATTGCATTTA | | TGCCAGCTGTTGTACTGAGCTACTGTACTGTAAGAT |
| | | CTTATTCCCATTAAGTCCTGTACATATGAAACTT | | TTTAAAATGTTTTCTTTATTTTGTGTTTGTTTTG |
| 346765 | SCAFFOLD | TTGTACTCCACCAAAATTCATGTGTTGAAACCCTAA | A/T | GATAAGCACCTTGATCTTGCACTTCCCACCTTCTAG |
| | 295127_4459 | CTTCCAGTGTGGCTATATTTGGAAAAGGGACCTCTT | | AATTCTGAGAAAGTTTGTGTTGTTTAAGCCACTGTG |
| | | AAAAAACAGTGGTTAAGGTTAAATGAGGTCATTAGT | | GTATTTTGCTACAGCAGACCAAGCAGATGAATACAC |
| | | GTGGAGCCCTGATCCAAGAGTATTAGTGTCCTTATG | | CCTCTTAGAGGTAAAGTAACTTCAAGAATAAAGCAT |
| | | AGAAGAGACACTGAAGTTTGCTCTTTCTCTCTTCCA | | TATTTGAACCTCAGTTCTGCCACTTGCTACTGGGAG |
| | | TGTGAAGACACAGCAGCAAGAAGGCAGGTATCTGCA | | GACCTCGGGCGCATTGTTTTTTTCTCTCTGACTCTC |
| | | AGCCAGGAAGATAGCCCTCACTAGCAACTGAATC | | AGTGATCTCAACACCTACCATGGAAATCTCTTGT |
| 345883 | SCAFFOLD | GCCCCTGACTGATTGTTTAATATTCACTTAGTAACA | C/A | TTCTCTAGTCAAGAATACTGGAGTGGGATGCTATTT |
| | 290369_7877 | GTATGAGCTTGGCTATATACTTTGCTACTGAACTTA | | CCTTCTCCAGGGAATTCTTAGAATACTTGTTGTTAA |
| | | GAGATCTTTGTGTTACATTTGAGCAGAGCATATCCT | | TTTAAACTAGCAAATGCAAGCCCTTGTATAGTGCTA |
| | | ACTTATACTGTTGCTACTCTCCTGCAAGAGTGCATA | | GAAACAAAGATAAAATAAACATATGCATAATTATCA |
| | | ATATCTGAATCAAATATATTCTTAGGTGTGGATGTG | | TTGCACCTTAAGCATATCTGTGTAGAAAAACTCTAG |
| | | AAGTCATTTCAGATATGTCCAACTGTTTGCAACACT | | GCTAAAAAGCTGGCAAGAGTTAGTGGTTTTTTTATG |
| | | ATGGACTATAGCCCACCAGGCTCCAGAGTCCATG | | CCTAGAGCATTCATTTCAGTTCAGTTGCTCAGTT |
| 347412 | SCAFFOLD | AGGCCTTCCCTGGCTGTGCTGTTTACACACACCCCT | G/A | TGTGAATTTGGCAGTGTTTACCAAAATTTTAAATGT |
| | 215141_21240 | CTTCCTGTGATTTCTTTCTGTTTAAAATACTTATTG | | TTACCTGTTGATCCAGTAATTTCTTGGAATTTACCC |
| | | CCAACTGCCATATTTTACTAAAATTGTCTCTTCTCA | | TATAAATTAGCTTACACAGGTGTGCAAATATGCATA |
| | | ATTGGAGTGATGGGAATGTAAAATCCATAGGGGCAA | | CATGTTTCTCTCCAAACTTGTTTTTAATTGTAAAAC |
| | | GATTTTTTTCTGTTTTGTTCACTGCTGTGCTTCCCT | | ACCTGAGCAGCCACACAAAGTATATCCATTTGTACT |
| | | GTAACAACAAACTGTTCATTAACTCATGGTGTATGC | | AATACAGAAGAGTCCTTCAAAAGAGTCTGATCCCAT |
| | | AGAGGCTAAGGAAACAAGTACACGCTTACATGTT | | TCTTGTAGAAAATAGACACAGAGCATATACACAT |
| 353889 | SCAFFOLD | TTAGACTCCAAAAGACTAATTTTTAAAATTTTCTCT | G/A | TTTTGGTTAAAGATAATCTGTAATCTGTGTTTTATT |
| | 317313_8969 | ATGATCTCAAACAATCCACTCTATTTTTTGATTAGG | | TCATTTATATAAGATGAAAAGCAATGCCTGCTTAAA |
| | | CTGAAGGATTATTCAGAAGCACACAGTGAATGCTTT | | TAACTTGGTCTTTTTTTCTCTTTCTCTTTTTTTAAA |
| | | CTAGAAGTTATATCTTATAGAATATAGTACTGACAA | | AAAATTTAAACAACTCGTGGCCAAAACTGTATCCAG |
| | | AGGAAAACATATTAACAGGTTTATCAGAATAATAAG | | TATTACCCAGATGACAAATAACATATCATTTTATCT |
| | | AAAACATCCGAAAGATATTGAATATTTTAGGCAAAC | | GTAAATATTACATTCTGTACCTTGAAAATTGGCTTA |
| | | CAAAATGTATAGATAATAACGTAATGGATAACAC | | GTCATTCAGTCATATCTGACTCTTTGCTACCCCA |
| 343366 | SCAFFOLD | ATTGTAAGTCTTCCTTTGAGACTTGGAGTTTCTTTC | G/A | CCCGATCCTTCCTGCTTCCCTAGATTTTATATAAAA |
| | 180197_3478 | TGGCTTCTGTGCCACAATTGGGGCACTAGGACATGT | | TACCTTTCTCTGTGTCTGGGGTTTCAGAGTTCGGCA |
| | | AGCAAGGCCACATTCTCTTGGAGTAGATGTGATATT | | AGCAGAATCCAGGATCATTTGCACACTAATTATATT |
| | | CTCTTGTCACGGGTCCTGTTGAACCCCAGGAAGCAT | | CTGTTCCAGGATGGGCATTTCTAAGGCCAATAGAAT |
| | | CATAGACAGCAGCATTTGCGTTTGGACCGCACAGGT | | GGAGCTGATGACTGAGACGAATGAAGAAAAGGAAAC |
| | | TTCTGTGGTCTCTCTTCTGTCCTCTCTCCGGCCAGG | | AGGGGAACTATAGGAGGGGAGTGAAGAGGATTTAGA |
| | | AGCATCTCTTTTGAGTCTGATGGAATGTCGGAGA | | TCAAAGACCACTTCCATGACGCCTGCCCAAGGGA |
| 354069 | SCAFFOLD | ATTATATATACTAAGATATAGTATAGCATAGTGAAG | T/G | ATATATATATGGAACCTTTAAAGATCTGACATATAC |
| | 322611_3027 | TCGCTCAGTCGTGTCCAACTCTTTTCGACCCCATGG | | CAATGCATTTGCTGTCTCTATTTACTTGAATAATCA |
| | | ACTGTAGCCTACCAGGCTTCTCCGTCCATGGGACTT | | TTAACTACATTCTGAGTGCCGAAGAATTGATGCTTT |
| | | TCCAGGCAAGAGTACTGGAGTGGGTTGCCATTTCCT | | TGAACTTGTGGTGTTGGAGAAGACTCTTGAGAGTCC |
| | | TCTCCAGGGGATCTTTCTGACCCAGGGATCGAACCT | | CTTGGGCTGGAAGGAGATCCAACCAGTCCATTCCAA |
| | | GGGTCTCCTGCATTGTAGGCAGACGCTTTACCATCT | | AGGAGATTAGTTCTGGGTATTCTTTGGAAGGAATGA |
| | | GAGCCACTAGGGAAGATGAATACTAAGATAGATA | | TGCTAAAGCTGAAACTCCAGTACTTTGGCCACCT |
| 348745 | SCAFFOLD | GCGGATTCTTTGCCATCTGAGCCACCAGGGAAGCAC | A/C | GAATACTACTGTGATTTGGTTAAAATAGTGCCCAAT |
| | 5512_10383 | AAGAGTACTGGAGTGGTAGCTATCCCTTCTCCAGGG | | GTTTAGAATAGCATTCTTTGAATTTATCTATTTGTA |
| | | GGGCTTCCTGACCCAGGAAATGAACCATGGTCCCCT | | AAAAAAAAAAAATTAAGCTCAGTTTCTATGAACTGT |
| | | GCATTGCAGGCAGATTCTTTACCAGCTGAGCTACCA | | ACATACAGTATGTTGTTGAAGGACATCTTCCGTCTG |
| | | GGGAAGCGCCATGTAAACCATACAATGACTTATTTA | | GACCTCTAGATATTTCTACATGCTCTTGAAATAACC |
| | | CACACACTTGTGTGCATATACACAAACAACTATTCT | | TAAAATATAGAATACAGGAAAAAATACCAGGATGCA |
| | | TTCACTAATATTTCCTGATGCGAAGGGAAGAGGG | | CAAGTGTTTATACAATATTCAATATTTCTTAAGC |
| 345345 | SCAFFOLD | GTACAGGCAGGCACTCAGGCACAGTGTGCACTCTTC | T/G | CCAAGGCAGCCAATTTCTGTACCTGGGGAGTTCCTT |
| | 101819_1771 | TCCTTGCCACTGTTAAACTTCTACCTAATTAATGTC | | GAAGCACTGACTCTTGCTTTGGAATTGGGAAAACTG |
| | | TGTGGGGAAATGTTTTCTCTTGCACTCAAGCAAACT | | CTCTGGTACCCGGTATCAAGGTCAGTGTAGAATCTT |
| | | AACATAAAAGGAGATGGTTTCCAAGGCGACTGCCTC | | CTGCAGATGCTATCCTGACTCTTGGAAAACATCAAA |
| | | TGCTGAGGTTACAGGAGAAAATTAGGGGAGAGCTTT | | AGTAGTCATACACAACTAAAGATTCATTTTTAAAGT |
| | | CTGAAGTCATGTCCCCAAGAGGGTGGGGGTAAAGAT | | CTAAAGTATAAAAAATGGCAGTTTCATAACACAGGG |
| | | AGAGGAGGGCTAAGATGTTAGTTAGTGAGCTGTC | | ACCAATTCTGACTCCTGACTCTTGTGAAACTTTG |
| 351367 | SCAFFOLD | GTCTTTGATTTTGGTGAAGTTTCAAAATTTTAAATC | G/A | TAAGTATGAAGAACTGTGAAGAGCTGGGGTCAGTTA |
| | 108770_1519 | TCTGCTCTGTTCTGCGTGTGCTTAGACCCTTAATTA | | TGGGGAATGCCATGAATATTTAGGACACTAAATAAT |
| | | CCAGTGCATTGCCAGGTTAAATTCTCCTCCTCAAGA | | ATCTTTATCACAGAATCATTGACCTGAATTTAATAT |
| | | CCAAGTTTAGATGGGCAAGCCATTCTGCTGATCATA | | GTAAGTTTGTGATCTTGGAATTTTCTTCACCCATAC |
| | | TCCCCGTCCTTTGTTCTAAACCCACTCCTTGTTCTT | | AATGACGGGATTTACCTAAATTACCCCTGAGATCTC |
| | | CCAGCTCCAGAATTCCAAACCTGCTTAACCCACATC | | TTTCAGTATGAAAATTCAGTGGTAAATTTCCTGTGT |
| | | CTTCACTTGGAAACAAAAGAATTTTATCCTAGCC | | CAGACAGGACAGTTTGCAGTTTGCAGATTTCTGC |
| 350048 | SCAFFOLD | GGTTCTCTCCCTGCTCTAACCATGAAGATGCTAACA | G/A | CAGCTTCTAGAAAACTGCTCAGAGACCCCAATCCCC |
| | 200025_43136 | AAGGGCAAGAGTCAGTACTCGACCTCAGGTCCCTTC | | AGGGTCAGTGTCACCCCCAGGATCCAGGTCACAAGG |
| | | CGGGAAGCCTGAGCCACAGCTGGTGTGGTCATCTCT | | TCACAGGTCCTTCCAGTTAATGGGCTCTTTGCCGGA |
| | | AGCTGAATAAAGCCGCCACTCCCAGGCAGCCTGGCT | | CTTCTGCAGCAGCTCGTTGGTTTTAGAGAAATGCCT |
| | | CATGGCTGCTCCACTTGAAACATGATCCCTCGTGCA | | GCATCCGAAGAAGCCCCTGTACACCGACAGCGGCGA |
| | | GGGGGCTTGAGAATCACAAGGGAGATCTTCTTACTT | | GGGGTGCGCCGTCTGCAGCACGTGGTGCCGCTTCTG |
| | | TGACTTCATAAACAACCAATACTTCTATTTC | | AAAAAGGGGGAGACGGGTGGTGAGACCGGGGGGG |
| 350311 | SCAFFOLD | AGTTGCTCCAATCTCCCTCACGTTCTCCTCATCACC | G/A | CATGTGAAAGACTTACCTATATTTCCACATTGTTGG |
| | 295336_4515 | ACTCCCCACCTTTGCTCAACCCTGAAAATTGTATTG | | CTATCACTAACGTTCTCCACTTCACCCTCATCACCA |
| | | ATTTGATGATCATGGGGCCCATGTTTATTTTATTTT | | CAGAATTTTGTTCCTTCTTTCCCGGGCATTCCTAAC |
| | | ATTTATTTATTTTTTTTGAGATGTAGGGTGTGGCTC | | ATCTTTCTTACAACCTTTCTTTGAAACTCAAGCCCC |
| | | CTGTCAGCATCTATGTGGTCCTGGACAAGTAGTTAA | | CTGATCTCATTTCCTTAAACTTTGACAAAGATGATA |
| | | CCAAACTCTCAGTGTCTAGTGGGGATGGCAGCTCTA | | GCTCTTGAGTTCATACCTGATTTTATTCTTCAGAGG |
| | | GTTTCATAAGGCGGCTGCAGAGAAATGAGTCAGT | | TTTGCTATGTCCTTGAGGCTGGATCTGAGTTGTC |
| 342784 | IBISS4snp897 | ACCTGGATGGTGTGTCGAGAGCGTTGTCCCTGCCTT | A/G | TCCCTCTGGTGTGGGGGTGGGGAGGATGAACTAGTG |
| | | GGGGCAGTCCTGCA | | CCTTTGGCTCCTGT |
| 346622 | SCAFFOLD | GAATCCGCCTACCAATGCAGGAGACACAAGGGCCCT | T/G | CAAGCATATGCCAAAATACAAGTCTAACAAAAAGAA |
| | 245872_6977 | GGGTTCTATCCCTTGGTTGGGATTCCCCCTGGAGGA | | AATAAATCCCTATTGTATTTTTTTTCAGGCTTCTGG |
| | | GGAAATGGCAAGCCACTCCAGTATTCTTGCCTGGAA | | CACCTTGGCCTGTGTCACAGCCCTGGAGCCTGAGCA |
| | | AATTCCATGGACAGAGGAGCGTGGTGGGCTACAGCC | | GGCTTGGTCTCATGGCCAGAGGGTGGTAAAGCTAAG |
| | | CACGGGGTCACAGAGTCAGACACAGCTGAGCGCACA | | GGGGCTAGGGAGGCTGTGTCATCAGAGAACCTCCCT |
| | | CACAAAGGATACCAGTGTATGTATAACTGAATCATG | | TGGCCCTGTAGTGCCTGCTCCCCTAAGATCACAGTC |
| | | TCGCTGGACGCCTTGACCAACGCTACGTGATCAA | | TCCAGATCCAGTCAGACCCTCCTATAGCTAAGGC |
| 344417 | SCAFFOLD | GATCTGGAGGGAGCAGAAGGAAGGGACTACGTGAGT | A/G | AGAGGATGAGGGGTAATGGGTGAGGACCTTGGGAAG |
| | 81013_3923 | TGCCTCTGGTCCCTCAGGTGGAAACGCTCACCAGAC | | GAGCAGCGGGTGAAGTGGGAGCCTGGGGCCTGGGGG |
| | | ATCCAATGGGCAGCTGGGTGTATGGGGGTAGAATCT | | AGGAGGTGTCCTGGGAGAGGGGGAAGGATCCCAGGA |
| | | GGTCAGAGTATTGGAGGGAGATATTTAATTTGGATG | | GGCGGGCGTGGCCCATTCTGCAGGCATGGAGAGTCT |
| | | TCAATTGGCAAATAAATGGACTGAAGCCTGGAAACT | | AGACCACAGGATTCAGCGCACTGTTCGTGGTCTGAA |
| | | GGATAAGATCACCCAGAGGGTGCAAACAGATCTGAG | | CTCAAGCACGCTCAGGGCAGCCCACAGCAACTGCAG |
| | | AAGGACAGAGCCCAGGCCTCCAGCAGAATCAGTC | | GAGAGTGGGGGAGACGAAAACCACAGCAGGGGAT |
| 351991 | SCAFFOLD | TTAAATAAATGATGATGTTAGGCTAAAATTGTTTTA | A/G | AGGAAACAAGAAATAAACATCTTTGTAAGTCAGGGA |
| | 112707_2297 | ACTTTCAAAGAAAAGGGTTTGTAATTAAAATAATGA | | CAAACTGAAAAATGGCATTCAGCAGAGTGATAGTTT |
| | | TAGACTCTCCTTGGAAAACAGGTTGTTTCTTTTTAA | | GTATTACAAATTAAATACCCAACTAAAATGCCTGCA |
| | | GGGGAGTAAGAAATTGAATTTTGCCCTCCAATTAAA | | GAAGAAAGGTTAATTTAGCTTCTTGCATTCAATTCT |
| | | TTTTCAAAATAATAATGAAAAATGTGGTAAAAAATG | | GATAAGTAAAGCTTGAGTCATTTTATAAATTGAAGT |
| | | ATAAGTATGATCATGGAAAAATGCCTGATTTCATTC | | ACATCTGACATATAAAACTGTTTTAGGGCAGAATTT |
| | | TGGATGCAGAATTTGTTCAAACTGGAAAACAAGA | | TCTCTCTTTAAAATTTTTCAGTATTATCAGTAAAC |
| 352036 | SCAFFOLD | GGCAGATTCTTAACCATTGGACCACCAGAGAAGTCC | G/A | ATACATAAATCAGATAAAAGTAGAGTTAGTGCCCTC |
| | 130012_24057 | CTCCTCCTCCTTTTTGCTTCTTAATTCCTGAATGTT | | TCTCTGGAGAGGAGGTGCCACCCTAGGACCCCAGGG |
| | | GGGCCTGGCCGCTATTCCAAGTTCACATTGTCAAAA | | CCTTCCAGGTCAGTTTGTGCAGTTTGATTCCTTTCC |
| | | AAATCTCAGCATGTCAGAGCAAGAGGGGTCAGTGGG | | TCCCCCTCCATGGAGGAGGAAACAGGCAGAGTCTTA |
| | | TTTCCAGCCTGCTCAGTCCGTCCTGCACTTCAAGAG | | AAGGGGGCAGAGCTTGGATTCCTGCTCTGACTCTCT |
| | | TTCCAAGAATCAAGGTCCAAATAATCAAAATTGCCC | | CTGCCCTGATGTGTGGCTGGGTTGGATCTTGGGTCC |
| | | TTTAGCAGTGGAGTTACTCTTCAGATGAAACCCC | | AACCAAAGTGATCATCAGGTTCACCTGATGAAGC |
| 354022 | SCAFFOLD | AAGAAAAATCATCAAGCAAAGTACAACATCCCTACT | C/T | ACTTTTAAAAAATCATTTTAATGAGTCAGATACCCA |
| | 225349_12937 | TATCCACAAAGACAGTTCATTTTAATAGATCCTGTG | | GAGCTAAGCATCAGCGCATCACTGTTTCCAGTCCAG |
| | | ACCCACTCAGGTTTCCTGAGAAACAACAAAGTTATT | | CTTTTGCAGTTTTCCCTGCCCTCTCCACAGTATTAT |
| | | TCTCTGAGCTACTATTCATTTTCCATTTGATATTTA | | TATGACTGTGCTCTGTGTCTTATGCTCTTTTCAAAC |
| | | TTCCCAAACCCATGTCTGAACTGCAATTCCCACTGA | | AGGGAACCAAGTTTGGAGTGATCCTCTGGATCAGGA |
| | | AATGCAAACATAACTATTTGATGTTCAGAACAAATA | | TTTGTTTTACTGAGTCTGAAGTTGAAATGAGCAAAT |
| | | CATTGTTCTCAGAATAAAACATCAATTTACACA | | TAGCCATGAGAAAATACAATCTTATTTTCTTAAC |
| 346155 | SCAFFOLD | GCCCGCAGGAGCTAGAGGACGCCCATGCGCAGCAAC | G/A | ATGTCGCCCCAGATTGGAAGGGTGGCTCCTGACAGT |
| | 95040_3552 | AAAGACCGCAGCCAACAAAATGTATGTGCATAGGCG | | CGAGGGAGGGTGTGTGTGTGTCGGGTGGCGTGCGTT |
| | | CCTCCTAGGAACCGGTAGCACCATGTACTCGCCTTA | | TCCCGCATGGCCTGGCTCAGCCAGGTATACTTCTCC |
| | | GATGGCTGAGAGTGATGTGAACATTGTAACACTTGA | | ACACACACCCCTTGTTACTCCTGGGCAAAATCCTGT |
| | | CCTTGGAGAAGACCTGAGGTTCACCTGTGGACATGG | | CTTATTAGCGAATGCAGAATCCACACCACGCTCAGA |
| | | GTGGGGGAAGGTTGCAGCCTGCGGGTCACTGTGAGG | | TCATTCTTTAGCATTTGTCCCAAACAGATGTCTGTT |
| | | TACCAGGAGGGAGTTTCCTAAAATGTGATGGGAG | | TAAAACAGGCCTTGGAGCATGGCTGACTCTTTCT |
| 349426 | SCAFFOLD | TCATCAGTGAGAGGTCCAACATTTCAGGTCCTCAGT | G/C | TGGAGTAGATTTTTGAAAGCAATGTGTCTGTGTGTA |
| | 271708_1981 | CTGGCTTGACTGGCGTCAGTGTGGGCCTTAGCGCTG | | TATGAAGGAAGCGAGGGCTGAAGTGGAGAAAGAGAA |
| | | CATAGGAGATCCTCAGTTGGCCTCTGGTTGTCCCAG | | AGGTTTGAGGACTTGAGGCTTCTCTTGGCAGTCACC |
| | | AGACTTGCTTAGCWTAGTAGACACATAAACACATGC | | ATCCTGTCAGCCACCTCCGCCTCCAGGCAGGTCACC |
| | | CTTCATGACCAAAGCGGTGAATGATGATGTTCTAAA | | CTGCAGGCCAAAAGCCTAGCCAGTCTNNNNNNNNNN |
| | | ATCATCATACTTTTTTTTTAATGCTAATCTCCCAGT | | NNNNNNNNNNNNNNNNNNNNNNNNNNNNNNNNNNNN |
| | | GAATTTTGGAAGTGGCTTTTAAAAGGTAAAGA | | NNNNNNNNNNNNNNNNNNNNNNNNNNNNNNNNNN |
| 347424 | SCAFFOLD | ATGAACAAAAACACCTTCAAAAGAACAAAGAGTCAC | T/C | AGATTCACCCAGACTGGATGAAGCAGAAGGAGTTCC |
| | 216837_2933 | AATTTTATAGATCAAAGAATATATTTAGGGCTTGGT | | TTTTATTAGTTATTGTTGCAGAACTCTTAATCAGCT |
| | | TTTTAGGGATATTTTCCAAAACTGGGTCACAATATG | | TAAGATTTTGTGGATCTTTTAAAATCATTATCACAG |
| | | CCCCAGCCTGAGAGACATGAGGTGACAGAGTCCATG | | AAACACACAAACAGGTCCTGTGGTTTAACTATCTGT |
| | | CTTTATCCCAGGTGGATGAGAAAGAGGGGAGTTAGA | | GTGTTGAGGAGTGGGACTGGGTGAGCGAGGCTGGGA |
| | | AAACTTCCTATGTCATTTCTGAAGTTATCCTGGGAA | | GGGTGGTCCGTAGGACTGAGCTCTTGGGACTGATAT |
| | | GGCAGGTGTAAAAAGAAATTGACATGGCCCAATT | | TTACTTTTGACCCTTGGCCCTTCAAACTGCCCTG |
| 346793 | SCAFFOLD | TGAGATATGAACATTTGGAGTTATGTGTTTTTGAAA | G/C | TTGCTCTTGGTGGAGGGAAGACTGTCAGAAGTGGTC |
| | 301627_1872 | GAAAATGAAATGTAGATGGGTATTTTGTACCTTCAA | | AGCCCGACCTTACTGACTGTGGGCTGCACGTGGCTG |
| | | AATAAGAAACAAATAGCACTTTCTAATATGTTTGAA | | TTGGCCAGCTAGGTGACCGTGTTGATTTTATATGAA |
| | | AATTGTTTCAGATTCAAATTTGTTTTGCAGAGTCAA | | ATACAGTGAAAGAAACGAAATGGGAGCCGCTGCAAC |
| | | ACAAGTGTTGCTTGTCATCTTTATTGTATTAATTGT | | TGCTGCTTCCCTGTGAGCTCTTTTCCTTTGTTGCTC |
| | | TTGGAAAATGAGCTAGGCTCCGCAGTGTGCTCTGAC | | AGAATCCAGATGGAGACAGCATCTTTGAAACTTGAG |
| | | TCACAGCATTTGCATCCTGTTTTTTTCTTATACA | | AAAATATGACATTTTCCTCTGCCTCTACCTTCTC |
| 347268 | SCAFFOLD | TCAAAAACTCAAACCTCTGAATCCACAATTACAAGT | C/G | GTCAAGAGAAAAGGAATCTGCCTTCAGAATGACAAA |
| | 165440_10591 | TGGTAAGAGCATATTGATTTTGTCTATGAAGTTAGC | | TATTCTTTTACCTGGAGAAACTGTCATCCCAGTTAT |
| | | CACTTTAAAGCTTCCAAAGTTGCCCTCTGACAAAGG | | GCAGTGACATCCTTAGAGCTTTATTTTCAAAGCGAA |
| | | CAACATCTGGTCTGCCGAGGACTGTCCCCCAGTTGA | | CTTTGAGAACGCTTGGAAGCCAACTCTGTCTGGGAG |
| | | CCTCCAATAACCTAATATTGCTGGATGCTCAGTCAC | | CGTGCTCTGCCATCTCCTGATCCTGCAGTTACATTC |
| | | TCAGGGCTTTGCTGAGTTTGTTCCACATAAATTCTA | | TGAATTTTCCACATCTTTTGCATTAAAGAAACATTA |
| | | TGCAGCTCCTAAGAGAAGGCTGAGTTTCCAGATT | | TTCCCTCTAACTCTGTCTGTTGGCCTCTTTCCAG |
| 353660 | SCAFFOLD | TGATTGGTATTTTGTGAATCTATAGACATCTGAGGA | T/A | ATTTAGCTTTAAACTCTTTGATTATAATGTGACCAG |
| | 47506_946 | AAACTGGCATCTTTATTGCGTTGATTCTGCTAATTC | | AATTGGATTTTTTGGGTTTGTTCTGTTTGGGGTCCA |
| | | ATATAACTCAGTATGTTCATTTATTTACATCTTCTT | | CTCAGCTTCTTTTTATTTGTATGTTTATGTCAGATA |
| | | GGGTTATTTCATCAGCATTTTGTATTTTTCAGAATT | | ATTGGCCACATTTGGAACATTTTCAGTCATTATTTC |
| | | CAGCTTCTGTACATGTCTTATTATATATATACCTTG | | TTTGAATACTCTTTCAGCACCATCATTTTTTTCTTG |
| | | TTAGATGTATACCTAAGTCCTTTGGAGGAACTGTTG | | TCCTTTTGAGACTTTGATTATATAAAGTTTAGACAC |
| | | TGTCTTCTAAAATTTGATTTCCAATTGTTCTTTG | | TTTGTGCTGTCCTACAGGTCCCTAAGGCTTCATT |
| 348539 | SCAFFOLD | AGTTGATCAACTAGAAATCTAATGCGTATGTAAAAA | G/A | TATACTGGACATTTAATCCCACTACATGGGATGTGA |
| | 270490_18616 | TGATGTCCGTCATGATACAATTGCACAAATGAAATA | | GTTTATTTCAGTCATGTTTTTATAAATGAGAAATGT |
| | | AGTACCTCATTTTGGTCCCCAGTAGTAAAAGATTTC | | TATATTATAGTTATGAAAAGCCCATGAATTCCTTGT |
| | | TTTGTAAATATAAATGAGATAAAATACCAGTGAAAA | | CACACACTCCTGAGTCAGACTGTTGGTCAATATTTA |
| | | ATTCTTCTCTAATTTACCATAAGCAAACTTCCATAG | | GTGAATATACACTCTGGTACCAGGCATGATATCAAT |
| | | AACAATCTGGGATTTTTGAGTTGCATGAGGCAAACA | | ATTAAGACAAATGTGGAGTATTCATTTATTCGTTTT |
| | | ACTGAGGTAGTAGGGCTTGCCCTGAAGTTCTTGG | | TTCTAAATCTAGTAAGAAAGTTAAGTTTTTGTTG |
| 347616 | SCAFFOLD | CAGATGAAGCAGCTTTAGTGAAAGGAGCCAAGAAAC | C/T | AATATTCTTACCAGAGGAGGGCACTGTCATCTCATC |
| | 280066_782 | TTGGCTTTGTTTTCACTACAAGGATGCCGAACTCGG | | AGACTTGCAGGAAAGCTTTATTTTGAAAAAGATATG |
| | | TCACCATAGAAGCTGTGAGTAATACTTGATATACTT | | TATTTGGAGGGTGAAACTTGTTCATGATTGGGAGAG |
| | | TCTAGCGGTTTACATGCACCTGCTCATCCAGAGACA | | TGAGTGACTTTGTGAGACAAATACTTTTTTCCTAAG |
| | | CACGTGTACAATTTTTCTAATCATGCTGATTTTAGA | | GTAAGAATATGTTGTCTTATTTTCCTGTAGATGGGA |
| | | TTGGAGAGGTGTTTCAATCTTAATCTGTTGCTATAG | | GAAGAACTCACATTTGAAATTCTTAATGTACTGGAA |
| | | GAAGCACTTTTGTTAATACTGTCCAGGTTGCAAA | | TTTTCTAGGTAAGCTATCTTTTTGATCTGTATGT |

### REFERENCES

Barendse, W. and R. Fries, 1999 Genetic linkage mapping, the gene maps of cattle, and the lists of loci, pp. 329-364 in The genetics of cattle, edited by R. Fries and A. Ruvinsky. CABI Publishing, Wallingford.
Boerwinkle et al. 1986 Ann. Hum. Genet. 50, 181-194.
Chen, X. N., B. Zehnbauer, A. Gnirke and P. Y. Kwok, 1997 Fluorescence energy transfer detection as a homogeneous DNA diagnostic method. Proceedings of the National Academy of Sciences of the United States of America 94: 10756-10761.
Dieffenbach and Dveksler (Eds) (In: PCR Primer: A Laboratory Manual, Cold Spring Harbour Laboratories, NY, 1995).
Hardenbol, P., J. Baner, M. Jain, M. Nilsson, E. A. Nahsaraev et al., 2003 Multiplexed genotyping with sequence-tagged molecular inversion probes. Nature Biotechnology 21: 673-678.
Hardenbol, P., F. L. Yu, J. Belmont, J. MacKensie, C. Bruckner et al., 2005 Highly multiplexed molecular inversion probe genotyping: Over 10,000 targeted SNPs genotyped in a single tube assay. Genome Research 15: 269-275.
Hardenbol P., Baner, J., Jain, M., Nilsson, M., Namsaraev, E. A., Karlin-Neumann, G. A., Fakhrai-Rad, H., Ronaghi, M., Willis, T. D., Landegren, U. and Davis, R. W. “Muliplex genotyping with sequence-tagged molecular inversion probes” Nature Biotechnology 5 May 2003.
P.-Y. Kwok (Ed) (In: Methods in Molecular Biology Vol 212 Human Press, Totowa N.J., 2003
Livak, K. J., 2003 SNP genotyping by the 5′-nuclease reaction, pp. 129-147 in Methods in Molecular Biology, edited by P.-Y. Kwok. Humana Press, Totowa N.J.
Lynch and Walsh, 1997 (Sinauer Associates).
Maxam-Gilbert method” (Maxam, A. M., et al. Proc. Natl, Acad. Sci (U.S.A.) 74:560 (1977)
Sanger, F., et al. J. Molec. Biol. 94:441 (1975); Prober et al. Science 238:336-340 (1987).
Storm, N., B. Darnhofer-Dehar, D. van den Booh and C. P. Rodi, 2002 MALDI-TOF mass spectrometry-based SNP genotyping. Methods in Molecular Biology 212: 214-262.
Willadsen “Cloning of sheep and cow embryos,” Genome 31:956 (1989).

1. A method for assessing tick resistance in a bovine animal, comprising the steps of:
(1) providing a nucleic acid from the bovine animal;
(2) assaying for the occurrence of a single nucleotide polymorphism (SNP) identified in any one of SEQ ID Nos: 1 to 210,
wherein the identification of said nucleotide occurrence is associated with increased tick resistance in the animal.

2. A method as claimed in claim 1 further comprising selecting a bovine animal exhibiting increased tick resistance.

3. A method as claimed in claim 1 wherein the nucleic acid is DNA.

4. A method as claimed in claim 3 wherein a DNA hybridization assay is used to detect the nucleotide occurrence.

5. A method as claimed in claim 3 wherein an amplification assay is used to detect the nucleotide occurrence.

6. A method as claimed in claim 5 wherein the amplification assay is PCR.

7. A method as claimed in claim 3 wherein the DNA comprises the sequence set forth in any one of SEQ ID Nos: 1 to 210, or a fragment thereof of at least 10 contiguous nucleotides which contains the polymorphism.

8. A method as claimed in claim 1 wherein the bovine animal is a cow.

9. A method as claimed in claim 8 wherein the cow is a pure breed selected from the group consisting of Ayrshire, Brown Swiss, Australian commercial dairy cow, Dairy shorthorn, Holstein, Guernsey, Sahiwal, Illawarra, Jersey, Meuse-Rhine-Issel, Red Poll, Simmental, Australian Red breed, Australian Friesian Sahiwal and Australian milking zebu, or crosses thereof.

10. (canceled)

11. A kit for use in a method as claimed in claim 1 comprising an oligonucleotide probe, primer or primer pair, or combinations thereof, for determining the nucleotide occurrence of the SNP.

12. (canceled)

13. A method for assessing tick resistance in a bovine animal, comprising the steps of:
(1) providing a nucleic acid from the bovine animal;
(2) assaying for the occurrence of a polymorphism in a gene, including in the coding sequences, the introns, promoters and other regulatory sequences of said gene, or a polymorphism in linkage disequilibrium with a polymorphism in said gene, wherein said gene is selected from the group consisting of COL1A2, SSFA2, ITGA4, TSTA, TSTA3, RPS13, RDH10, ELTD1, RNASE1, SEMA3C, FLJ12572, NELL1, RIPK2, GPR39, LAMP2 and ARL6, wherein the identification of said nucleotide occurrence is associated with increased tick resistance.

14. A method as claimed in claim 13 further comprising selecting a bovine animal exhibiting increased tick resistance.

15. A kit for use in a method as claimed in claim 13 comprising an oligonucleotide probe, primer or primer pair, or combinations thereof, for determining said nucleotide occurrence.
